(19) United States
(12) Patent Application Publication
(10) Pub. No.: US 2004/0096950 A1
(43) Pub. Date: May 20, 2004

(54) Crystal structure of beta site APP cleaving enzyme (BACE) and methods of use thereof

(76) Inventors: Laurent Michel Marie Vuillard, Cambridge (GB); Sahil Joe Patel, Cambridge (GB); Jeffrey Roland Yon, Cambridge (GB); Anne Cleasby, Cambridge (GB); Bruce John Hamilton, Cambridge (GB); Aleem Shah, Cambridge (GB)

Correspondence Address: FROMMER LAWRENCE & HAUG, 745 FIFTH AVENUE- 10TH FL., NEW YORK, NY, 10151, US

(21) Appl. No.: 10/627,473

(22) Filed: Jul. 25, 2003

Related U.S. Application Data
(60) Provisional application No. 60/398,681, filed on Jul. 26, 2002.

(51) Int. Cl. C12N 9/99; C07H 21/04

(52) U.S. Cl. 435/184; 435/69.2; 435/320.1; 435/325; 536/23.2

(57) ABSTRACT

The present application discloses and claims mutant BACE proteins, recombinant BACE proteins, processes for crystallizing BACE and in particular to its crystal structure and to the uses of this structure in drug discovery.

### RELATED APPLICATIONS

This application claims priority to U.S. Provisional Patent Application Serial No. 60/398,681 filed Jul. 26, 2002, and corresponds to International Patent Application number ______ (Attorney docket number AHB/CP6162168) filed Jul. 25, 2003.

All documents cited in this text, and all documents cited or referenced in documents cited in this text, and any manufacturer's instructions or catalogues for any products cited or mentioned in this text or in any document hereby incorporated into this text, are hereby incorporated herein by reference. Documents incorporated by reference into this text or any teachings therein may be used in the practice of this invention. Documents incorporated by reference into this text are not admitted to be prior art. Furthermore, authors or inventors on documents incorporated by reference into this text are not to be considered to be “another” or “others” as to the present inventive entity and vice versa, especially where one or more authors or inventors on documents incorporated by reference into this text are an inventor or inventors named in the present inventive entity.

### FIELD OF THE INVENTION

The present invention relates to the mutant BACE proteins, recombinant BACE proteins, processes for crystallizing BACE and in particular to its crystal structure and to the uses of this structure in drug discovery.

### BACKGROUND TO THE INVENTION

Alzheimer's Disease

Alzheimer's disease (AD) is estimated to afflict more than 20 million people worldwide and is believed to be the most common form of dementia. Alzheimer's disease is a progressive dementia in which massive deposits of aggregated protein breakdown products—amyloid plaques and neurofibrillary tangles accumulate in the brain. The amyloid plaques are thought to be responsible for the mental decline seen in Alzheimer's patients.

Aβ or amyloid-β-protein is the major constituent of the plaques which are characteristic of Alzheimer's disease (De Strooper et al, 1999). Aβ is a 39-42 residue peptide formed by the specific cleavage of a class I transmembrane protein called APP, or amyloid precursor protein. A β-secretase activity cleaves this protein between residues Met671 and Asp672 (numbering of 770aa isoform of APP) to form the N-terminus of Aβ. A second cleavage of the peptide is associated with β-secretase to form the C-terminus of the Aβ peptide.

Beta Site APP Cleaving Enzyme (BACE) and Alzheimer's Disease

Several groups have identified and isolated aspartate proteases that have β-secretase activity (Hussain et al., 1999; Lin et. al, 2000; Yan et. al, 1999; Sinha et. al., 1999 and Vassar et. al., 1999). β-secretase is also known in the literature as Asp2 (Yan et. al, 1999), Beta site APP Cleaving Enzyme (BACE or BACE1 ) (Vassar et. al., 1999) or memapsin-2 (Lin et al., 2000). BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches led to the identification of the same enzyme, making a strong case that BACE is a β-secretase. Mention is also made of the patent literature: WO96/40885, EP871720, U.S. Pat. Nos. 5,942,400 and 5,744,346, EP855444, U.S. Pat. No. 6,319,689, WO99/64587, WO99/31236, EP1037977, WO00/17369, WO01/23533, WO0047618, WO00/58479, WO00/69262, WO01/00663, WO01/00665, U.S. Pat. No. 6,313,268.

BACE is a membrane bound type 1 protein that is synthesized as a partially active proenzyme, and is abundantly expressed in brain tissue. It is thought to represent the major β-secretase activity, and is considered to be the rate-limiting step in the production of Aβ. It is thus of special interest in the pathology of Alzheimer's disease, and in the development of drugs as a treatment for Alzheimer's disease.

BACE was found to be a pepsin-like aspartyl proteinase, the mature enzyme consisting of the N-terminal catalytic domain, a transmembrane domain, and a small cytoplasmic domain. BACE has an optimum activity at pH 4.0-5.0 (Vassar et al, 1999) and is inhibited weakly by standard pepsin inhibitors such as pepstatin. It has been shown that the catalytic domain minus the transmembrane and cytoplasmic domain has activity against substrate peptides (Lin et al, 2000). Consequently, this soluble catalytic domain is suitable for crystallization studies and a crystal structure of this will give a representative structure of the BACE active site for the design of inhibitor molecules.

The likelihood of developing Alzheimer's disease increases with age, and as the aging, population of the developed world increases, this disease becomes a greater and greater problem. In addition to this, there is a familial link to Alzheimer's disease and consequently any individuals possessing the double mutation of APP known as the Swedish mutation (in which the mutated APP forms a considerably improved substrate for BACE) have a much greater chance of developing AD, and also of developing it at an early age (see also U.S. Pat. Nos. 6,245,964 and 5,877,399 pertaining to transgenic rodents comprising APP-Swedish). Consequently there is a strong case for developing a compound that can be used in a prophylactic fashion for these individuals.

Hence, drugs that reduce or block BACE activity would reduce Aβ levels and levels of fragments of Aβ in the brain or elsewhere where Aβ or fragments thereof deposit and thus slow the formation of amyloid plaques and the progression of AD or other maladies involving deposition of Aβ or fragments thereof (Yankner, 1996; De Strooper and Konig, 1999). BACE is therefore an important candidate for the development of drugs as a treatment against Alzheimer's disease and/or against such other maladies.

The therapeutic potential of inhibiting the deposition of Aβ has motivated many groups to isolate and characterize secretase enzymes and to identify their potential inhibitors (see, e.g., WO01/23533 A2, EP0855444, WO00/17369, WO00/58479, WO00/47618, WO00/77030, WO01/00665, WO01/00663, WO01/29563, WO002/25276, U.S. Pat No. 5,942,400, U.S. Pat. No. 6,245,884, U.S. Pat. No. 6,221,667, U.S. Pat. No. 6,211,235, WO02/02505, WO02/02506, WO02/02512, WO02/02518, WO02/02520, WO02/14264).

The gene encoding APP is found on chromosome 21, which is also the chromosome found as an extra copy in Downs syndrome. Downs syndrome patients tend to acquire Alzheimers disease at an early age, with almost all those over 40 years of age showing Alzheimers-type pathology (Oyama et al., 1994). This is thought to be due to the extra copy of the APP gene found in these patients, which leads to overexpression of APP and therefore to increased levels of APPβ causing the high prevalence of Alzheimers disease seen in this population. Thus inhibitors of BACE could be useful in reducing Alzheimers-type pathology in Down's syndrome patients.

It would therefore be useful to inhibit the deposition of Aβ and portions thereof by inhibiting BACE through inhibitors designed from the BACE structure as provided herein. The determination of the three-dimensional structure of BACE provides a basis for the design of new and specific ligands for BACE. For example, knowing the three-dimensional structure of BACE, computer modelling programs may be used to design different molecules expected to interact with possible or confirmed binding cavities or other to structural or functional features of BACE or structure-based design approaches may used such as those described in Blundell et al (Nature Reviews, Drug Discovery, Vol 1, pg 45-54, 2002).

Ideally it would be desirable to have an abundant supply of this enzyme in homogenous form. It would also be preferable to solve the structure of a form of BACE with an unoccupied active site. This could be used to soak in small molecule inhibitors of the enzyme and to investigate their binding modes. We describe here the high yielding production of BACE from bacterial cells in homogenous form, and the generation of protein suitable for crystallisation and structure determination of BACE in Apo form

Protein Crystallisation

It is well known in the art of protein chemistry that crystallising a protein is an uncertain and difficult process without any clear expectation of success. It is now evident that protein crystallization is the main hurdle in protein structure determination. For this reason, protein crystallization has become a research subject in and of itself, and is not simply an extension of the protein crystallographer's laboratory. There are many references, which describe the difficulties associated with growing protein crystals (Kierzek A M. and Zielenkiewicz P. (2001) Biophysical Chemistry 91 1-20 Models of protein crystal growth, Wiencek J M (1999) Annu Rev Biomed Eng 1 505-534 New Strategies for crystal growth).

The reasons why it is commonly held that crystallization of protein molecules from solution is the major obstacle in the process of determining protein structures are many; proteins are complex molecules, and the delicate balance involving specific and non-specific interactions with other protein molecules and small molecules in solution, is difficult to predict.

Each protein crystallizes under a unique set of conditions, which cannot be predicted in advance. Simply supersaturating the protein to bring it out of solution will not work, the result would, in most cases, be an amorphous precipitate. Many precipitating agents are used, common ones are different salts, and polyethylene glycols, but others are known. In addition, additives such as metals and detergents can be added to modulate the behaviour of the protein in solution. Many kits are available (e.g., from Hampton Research), which attempt to cover as many parameters in crystallization space as possible, but in many cases these are just a starting point to optimize crystalline precipitates and crystals which are unsuitable for diffraction analysis. Successful crystallization is aided by knowledge of the proteins behaviour in terms of solubility, dependence on metal ions for correct folding or activity, interactions with other molecules and any other information that is available. Even so, crystallization of proteins is often regarded as a time-consuming process, whereby subsequent experiments build on observations of past trials.

In cases where protein crystals are obtained, these are not necessarily always suitable for diffraction analysis; they may be limited in resolution, and it may subsequently be difficult to improve them to the point at which they will diffract to the resolution required for analysis. Limited resolution in a crystal can be due to several things. It may be due to intrinsic mobility of the protein within the crystal; this can be difficult to overcome, even with other crystal forms. It may be due to high solvent content within the crystal, which consequently results in weak scattering. Alternatively, it could be due to defects within the crystal lattice, which means that the diffracted x-rays will not be completely in phase from unit to unit within the lattice. Any one of these or a combination of these could mean that the crystals are not suitable for structure determination.

Some proteins never crystallize, and after a reasonable attempt it is necessary to examine the protein itself and consider whether it is possible to make individual domains, different N or C-terminal truncations, or point mutations. It is often hard to predict how a protein could be re-engineered in such a manner as to improve crystallisability. Sometimes the inclusion of a ligand in the crystallisation mixture is essential for the production suitable crystals. Our understanding of crystallisation mechanisms is still incomplete and the factors of protein structure, which are involved in crystallisation, are not well known.

BACE Production for Crystallisation

Beta secretase (BACE) is an integral membrane protein containing a signal sequence, a pro-peptide, a catalytic aspartyl protease domain, a transmembrane region and a C-terminal cytoplasmic region. During transit through the endoplasmic reticulum, Golgi apparatus and trans Golgi network the pro-peptide is cleaved by a furin-like protease (Bennett et al 2000, Creemers et al 2001) and N-glycosylation is added and matured (Haniu et al 2000). The protein contains 4 potential N-linked glycosylation sites, all of which are used (Bennett et al, 2000).

Certain active recombinant BACEs—different from those of the herein invention—have been produced using heterologous expression systems for mammalian cells (Vassar et al, 1999, Hussain et al, 1999), insect cells (Mallender et al, 2000) and bacterial cells (Lin et al 2000). Preferred constructs for crystallisation would be soluble and lack glycosylation: the former can be achieved by C-terminal truncation of the protein to remove the transmembrane and cytoplasmic regions; while glycosylation could be removed either by use of a deglycosylating agent such as PNGase F, by expression of the protein in bacteria or by mutation of the glycosylation sites.

The protein used for BACE crystallisation by Hong et al (2000) was produced in bacteria and was truncated at the C-terminus. Their protein was produced as insoluble inclusion bodies and required refolding to give soluble, active protein. Refolding of BACE is made more complex by the presence of 3 disulphide bonds in the native protease domain, which require careful control of redox conditions to form during in-vitro refolding. The protein produced by Hong et al was a mixture of products and was crystallised with inhibitor bound (see WO 01/00663, WO 01/00665, and U.S. Pat. No. 6,545,127).

Mention is also made of WO 02/25276, which describes the crystallisation of BACE produced in mammalian cells. The protein produced also was a mixture of protein species and was also crystallized with an inhibitor bound.

Mention is also made of WO03/012089, which describes the crystallisation of BACE produced from insect cells. The co-ordinates of BACE with an inhibitor bound are provided.

### SUMMARY OF THE INVENTION

In general aspects, the present invention is concerned with the provision of a new, high resolution, apo, crystal form of BACE and the use of this structure in identifying or obtaining agent compounds (especially inhibitors of BACE) for modulating BACE activity, and in preferred embodiments identifying or obtaining actual agent compounds/inhibitors. Crystal structure information presented herein is useful in designing potential inhibitors and modelling them or their potential interaction with the BACE binding cavity. Potential inhibitors may be brought into contact with BACE to test for ability to interact with the BACE binding cavity. Actual inhibitors may be identified from among potential inhibitors synthesized following design and model work performed in silico. An inhibitor identified using the present invention may be formulated into a composition, for instance a composition comprising a pharmaceutically acceptable excipient, and may be used in the manufacture of a medicament for use in a method of treatment.

Thus, according to a first aspect of the present invention there is provided a mutant BACE protein, which protein lacks one or more proteolytic cleavage sites recognized by clostripain (or another protease which recognizes the same cleavage site as clostripain). In particular, the protein is a BACE protein, which comprises the sequence set out in residues 45 to 455 of SEQ ID NO:2 (43 to 453 SwissProt P56817), or a fragment thereof comprising residues corresponding to 58 to 398 of SEQ ID NO:2, modified by the following changes: (a) substitution or deletion of at least one residue which is a proteolytic cleavage site recognised by clostripain; and (b) optionally the replacement of from 1 to 30 other amino 25 acids by an equivalent or fewer number of amino acids. It will be understood that when the BACE protein comprises a fragment as defined above, the fragment will comprise at least feature (a) and optionally feature (b).

The modification is such that the BACE protein preferably retains at least one proteolytic cleavage site recognised by clostripain so that it may be cleaved to provide homogeneous location at which cleavage occurs.

According to a second aspect of the present invention there is provided a mutant BACE protein which is truncated at the N-terminal up to and including R42, R45, G55, R56 or R57. In a preferred aspect, when the protein is truncated up and including R56 the residue at position 57 is not arginine. It may for example be lysine.

In a third aspect the invention provides a mutant BACE protein selected from: (a) SEQ ID 6; (b) SEQ ID 8; (c) SEQ ID 10; (d) SEQ ID 12; (e) SEQ ID 14; (f) SEQ ID 16; (g) SEQ ID 18; (h) SEQ ID 19; (i) SEQ ID 20; (j) SEQ ID 21.

In another aspect, the invention contemplates a nucleic acid (e.g. DNA or RNA) sequence encoding the BACE protein of the invention, as well as the complementary nucleic acid sequence counterpart.

The nucleic acids of the invention may be isolated, or may be present in the context of a vector or host cell. Thus, in another aspect, the invention contemplates a vector comprising the nucleic acid of the invention.

The nature of the vector of the invention is not critical to the invention. Any suitable vector may be used, including expression vectors, plasmid, virus, bacteriophage, transposon, minichromosome, liposome or mechanical carrier.

The expression vectors of the invention are DNA constructs suitable for expressing DNA which encodes the desired peptide and which may include: (a) a regulatory element (e.g. a promoter, operator, activator, repressor and/or enhancer), (b) a structural or coding sequence which is transcribed into mRNA and (c) appropriate transcription, translation, initiation and termination sequences. They may also contain sequence encoding any of various tags (e.g. to facilitate subsequent purification of the expressed protein, such as affinity (e.g. His tags).

Particularly preferred are vectors which comprise an expression element or elements operably linked to the DNA of the invention to provide for expression thereof at suitable levels. Any of a wide variety of expression elements may be used, and the expression element or elements may for example be selected from promoters, enhancers, ribosome binding sites, operators and activating sequences. Such expression elements may comprise an enhancer, and for example may be regulatable, for example being inducible (via the addition of an inducer).

The vector may further comprise a positive selectable marker and/or a negative selectable marker. The use of a positive selectable marker facilitates the selection and/or identification of cells containing the vector.

In another aspect, the invention contemplates a host cell comprising the vector of the invention. The nucleic acid of the invention may be intrdoduced into the host cell by any of a large number of convenient methods, including calcium phosphate transfection, DEAE-Dextran mediated transfection, electroporation or any other method known in the art.

Any suitable host cell may be used, including prokaryotic host cells (such as Escherichia, coli, Streptomyces spp. and Bacillus subtilis) and eukaryotic host cells. Suitable eukaryotic host cells include insect cells (e.g. using the baculovirus expression system), mammalian cells, fungal (e.g. yeast) cells and plant cells. Preferred mammalian cells are animal cells such as CHO, COS, C 127, 3T3, HeLa, HEK 293, NIH 3T3, BHK and Bowes melanoma (particularly preferred being CHO-K1, COS7, Y1 adrenal and carcinoma cells).

Cell-free translation systems can also be used to produce the peptides of the invention. Appropriate cloning and expression vectors for use with prokaryotic and eukaryotic hosts are described in Sambrook et al., Molecular Cloning: A Laboratory Manual, Second Edition, Cold Spring Harbor, N.Y., (1989).

Prokaryotic host cells are preferred in circumstances where the BACE protein is required in an unglycosylated state.

According to another aspect of the invention there is provided a process for producing the BACE protein of the invention comprising the steps of: (a) culturing the host cell of the invention under conditions suitable for expression of the BACE protein; and optionally (b) isolating the expressed recombinant BACE protein.

In a further aspect the invention provides a method of making BACE protein which comprises protedlytically cleaving a BACE protein which lacks one of more proteolytic cleavage sites as described above, the cleavage desirably occurring at (and including) one of position 42, 45, 55, 56 or 57, preferably 42, 56 or 57. Clostripain, or another protease which recognises the same cleavage site as clostripain, may be used.

Thus the resulting BACE protein of this aspect of invention will be a protein whose N-terminal corresponds to 45, 48, 58, 59 or 60 of SEQ ID NO:2, and whose C-terminal region extends to and includes at least 398 of SEQ ID NO:2. Preferably the C-terminal region terminates at a residue between a point corresponding to and including 398 up to and including 455. This BACE protein may additionally comprise a C-terminal tag, such as a tag comprising from 5 to 15 residues, such as a his tag or the like.

In another aspect of the invention there is provided a process for producing refolded recombinant BACE protein comprising the steps of: (a) solubilising the recombinant BACE; (b) diluting the solubilised BACE into an aqueous buffer containing sulfobetaine (for example at a concentration of 10 to 50 mM, for example 10 mM); and (c) maintaining the diluted solution at low temperature (for example, 3 to 6° C.) and at high pH (e.g. 9 to 10.5) for at least 2 weeks (typically 3 weeks, more typically 4 weeks).

In another aspect the invention provides a process for producing a crystal of BACE comprising the step of growing the crystal by vapour diffusion using a reservoir buffer that contains 18-26% PEG 5000 MME (for example, 20-24% PEG 5000 MME, e.g. 20-22.5% PEG 5000 MME), 180-220 mM (e.g. 200 mM) ammonium iodide and 180-22-mM (e.g. 200 mM) tri-sodium citrate (pH 6.4-6.6). In a further aspect the reservoir buffer may additionally comprise from 0 to 5% (v/v) glycerol, for example 2.5% v/v.

In another aspect the invention provides various BACE crystals, including a crystal of BACE having a hexagonal space group P6₂₂ (and optionally having unit cell dimensions of a=b=103.2 Å, c=169.1 Å,α=β=60°, γ=120 °, and a unit cell variability of 5% in all dimensions); a crystal of BACE having a resolution better than 3 Å (for example, better than 2.5 Å, e.g. better than 1.8 Å), and a crystal of BACE comprising a structure defined by all or a portion of the co-ordinates of Table 1.

In another aspect the invention provides a three-dimensional representation of BACE or of a portion of BACE, which representation comprises all or a portion of the coordinates of Table 1. The representation is preferably a BACE model.

The invention also contemplates a three-dimensional representation of a compound which fits the BACE model of the invention.

The invention also contemplates a computer-based method for the analysis of the interaction of a molecular structure with a BACE structure of the invention, which comprises: (a) providing a BACE model; (b) providing a molecular structure to be fitted to said BACE model; and (c) fitting the molecular structure to the BACE model to produce a compound model.

In another aspect the invention provides a computer-based method for the analysis of the interaction of a molecular structure with a BACE structure of the invention, which comprises: (a) providing the structure of a BACE as defined by the coordinates of Table 1; (b) providing a molecular structure to be fitted to said BACE structure; and (c) fitting the molecular structure to the BACE structure of Table 1.

In another aspect the invention provides a computer-based method for the analysis of molecular structures which comprises: (a) providing the coordinates of at least two atoms of a BACE structure as defined in Table 1 (“selected coordinates”); (b) providing the structure of a molecular structure to be fitted to the selected coordinates; and (c) fitting the structure to the selected coordinates of the BACE structure.

In another aspect the invention provides a computer-based method of rational drug design comprising comprising: (a) providing the coordinates of at least two atoms of a BACE structure as defined in Table 1 (“selected coordinates”); (b) providing the structures of a plurality of molecular fragments; (c) fitting the structure of each of the molecular fragments to the selected coordinates; and (d) assembling the molecular fragments into a single molecule to form a candidate modulator molecule.

In another aspect the invention provides a method for identifying a candidate modulator (e.g. candidate inhibitor) of BACE comprising the steps of: (a) employing a three-dimensional structure of BACE, at least one sub-domain thereof, or a plurality of atoms thereof, to characterise at least one BACE binding cavity, the three-dimensional structure being defined by atomic coordinate data according to Table 1; and (b) identifying the candidate modulator by designing or selecting a compound for interaction with the binding cavity.

In another aspect the invention provides a method for identifying an agent compound (e.g. an inhibitor) which modulates BACE activity, comprising the steps of: (a) employing three-dimensional atomic coordinate data according to Table 1 to characterise at least one (e.g. a plurality of) BACE binding site(s); (b) providing the structure of a candidate agent compound; (c) fitting the candidate agent compound to the binding sites; and (d) selecting the candidate agent compound.

In another aspect the invention provides a method of assessing the ability of a candidate modulator to interact with BACE which comprises the steps of: (a) obtaining or synthesising said candidate modulator; (b) forming a crystallized complex of BACE and said candidate modulator; and (c) analysing said complex by X-ray crystallography or NMR spectroscopy to determine the ability of said candidate modulator to interact with BACE.

In another aspect the invention provides a method for determining the structure of a compound bound to BACE, said method comprising: (a) mixing BACE with the compound to form a BACE-compound complex; (b) crystallizing the BACE-compound complex; and (c) determining the structure of said BACE-compound(s) complex by reference to the data of Table 1.

In another aspect the invention provides a method for determining the structure of a compound bound to BACE, said method comprising: (a) providing a crystal of BACE; (b) soaking the crystal with one or more compound(s) to form a complex; and (c) determining the structure of the complex by employing the data of Table 1.

In another aspect the invention provides a method of determining the three dimensional structure of a BACE homologue or analogue of unknown structure, the method comprising the steps of: (a) aligning a representation of an amino acid sequence of the BACE homologue or analogue with the amino acid sequence of the BACE of Table 1 to match homologous regions of the amino acid sequences; (b) modelling the structure of the matched homologous regions of said target BACE of unknown structure on the corresponding regions of the BACE structure as defined by Table 1; and (c) determining a conformation for the BACE homologue or analogue which substantially preserves the structure of said matched homologous regions.

In another aspect the invention provides a method of providing data for generating structures and/or performing rational drug design for BACE, BACE homologues or analogues, complexes of BACE with a potential modulator, or complexes of BACE homologues or analogues with potential modulators, the method comprising: (i) establishing communication with a remote device containing computer-readable data comprising at least one of: (a) atomic coordinate data according to Table 1, said data defining the three-dimensional structure of BACE, at least one sub-domain of the three-dimensional structure of BACE, or the coordinates of a plurality of atoms of BACE; (b) structure factor data for BACE, said structure factor data being derivable from the atomic coordinate data of Table 1; (c) atomic coordinate data of a target BACE homologue or analogue generated by homology modelling of the target based on the data of Table 1; (d) atomic coordinate data of a protein generated by interpreting X-ray crystallographic data or NMR data by reference to the data of Table 1; and (e) structure factor data derivable from the atomic coordinate data of (c) or (d); and (ii) receiving said computer-readable data from said remote device.

In another aspect the invention provides a computer system containing one or more of: (a) atomic coordinate data according to Table 1, said data defining the three-dimensional structure of BACE or at least selected coordinates thereof; (b) structure factor data (where a structure factor comprises the amplitude and phase of the diffracted wave) for BACE, said structure factor data being derivable from the atomic coordinate data of Table 1; (c) atomic coordinate data of a target BACE protein generated by homology modelling of the target based on the data of Table 1; (d) atomic coordinate data of a target BACE protein generated by interpreting X-ray crystallographic data or NMR data by reference to the data of Table 1; or (e) structure factor data derivable from the atomic coordinate data of (c) or (d).

In another aspect the invention provides a computer-readable storage medium, comprising a data storage material encoded with computer readable data, wherein the data are defined by all or a portion of the structure coordinates of BACE of Table 1, or a homologue of BACE, wherein said homologue comprises backbone atoms that have a root mean square deviation from the Cα or backbone atoms (nitrogen-carbon_{α}-carbon) of Table 1 of less than 2.0 Å, preferably less than 1.5 Å, more preferably less than 1.0 Å, even more preferably less than 0.74 Å, even more preferably less than 0.72 Å and most preferably less than 0.5 Å when superimposed on the coordinates provided in Table 1 for the residue backbone atoms.

In another aspect the invention provides a computer-readable data storage medium comprising a data storage material encoded with a first set of computer-readable data comprising a Fourier transform of at least a portion (e.g. selected coordinates as defined herein) of the structural coordinates for BACE according to Table 1; which, when combined with a second set of machine readable data comprising an X-ray diffraction pattern of a molecule or molecular complex of unknown structure, using a machine programmed with the instructions for using said first set of data and said second set of data, can determine at least a portion of the structure coordinates corresponding to the second set of machine readable data.

In another aspect the invention provides a computer readable medium with at least one of: (a) atomic coordinate data according to Table 1 recorded thereon, said data defining the three-dimensional structure of BACE, or at least selected coordinates thereof; (b) structure factor data for BACE recorded thereon, the structure factor data being derivable from the atomic coordinate data of Table 1; (c) atomic coordinate data of a target BACE protein generated by homology modelling of the target based on the data of Table 1; (d) atomic coordinate data of a BACE-ligand complex or a BACE homologue or analogue generated by interpreting X-ray crystallographic data or NMR data by reference to the data of Table 1; and (e) structure factor data derivable from the atomic coordinate data of (c) or (d).

In another aspect the invention provides a method for determining the structure of a protein, which method comprises; providing the co-ordinates of Table 1, and either (a) positioning the co-ordinates in the crystal unit cell of said protein so as to provide a structure for said protein or (b) assigning NMR spectra Peaks of said protein by manipulating the coordinates of Table 1.

In another aspect the invention contemplates BACE modulator molecules, medicaments, pharmaceutical compositions and drugs obtainable by, or obtained by, the processes and methods of the invention, and to methods of therapy (e.g. the treatment of Alzheimer's disease) using such products.

It is to be understood that, except where explicitly stated otherwise, references herein to “BACE protein” or “BACE peptide”, “mutant BACE protein” or “mutant BACE peptide” and to “BACE protein” or “BACE peptide”, as well as references to any of the foregoing which are further defined inter alia by reference to one or more specific amino acid sequences, are intended to cover BACE homologues, allelic forms, species variants, derivatives and muteins thereof (as defined below).

Thus, references to mutant BACE proteins having particular amino acid sequences may optionally be interpreted to cover the corresponding homologues, allelic forms, species variants, derivatives and muteins (as defined below) of that particular BACE amino acid sequence.

### DEFINITIONS

Where used herein and unless specifically indicated otherwise, the following terms are intended to have the following meanings in addition to any broader (or narrower) meanings the terms might enjoy in the art:

The term “isolated” is used herein to indicate that the isolated moiety (e.g. peptide or nucleic acid) exists in a physical milieu distinct from that in which it occurs in nature. For example, the isolated peptide may be substantially isolated with respect to the complex cellular milieu in which it naturally occurs. The absolute level of purity is not critical, and those skilled in the art can readily determine appropriate levels of purity according to the use to which the peptide is to be put. The term “isolating” when used a step in a process is to be interpreted accordingly.

In many circumstances, the isolated moiety will form part of a composition (for example a more or less crude extract containing many other molecules and substances), buffer system, matrix or excipient, which may for example contain other components (including proteins, such as albumin).

In other circumstances, the isolated moiety may be purified to essential homogeneity, for example as determined by PAGE or column chromatography (for example HPLC or mass spectrometry). In preferred embodiments, the isolated peptide or nucleic acid of the invention is essentially the sole peptide or nucleic acid in a given composition.

The proteins and nucleic acids of the invention need not be isolated in the sense defined above, however. For example, more or less crude culture supernatants (e.g. “spent” medium) may contain sufficient concentrations of the proteins or nucleic acids of the invention for use in several applications. Preferably, such supernatants are fractionated and/or extracted, but in many circumstances they may be used without pretreatment. They are preferably derived from spent media used to culture the host cells of the invention (for example, the bacterial sources described infra). The supernatants are preferably sterile. They may be treated in various ways, for example by concentration, filtration, centrifugation, spray drying, dialysis and/or lyophilisation. Conveniently, the culture supernatants are simply centrifuged to remove cells/cell debris and filtered.

The term “pharmaceutical composition” is used herein to define a solid or liquid composition in a form, concentration and level of purity suitable for administration to a patient (e.g. a human or animal patient) upon which administration it can elicit the desired physiological changes.

The term “recombinant” as applied to the proteins of the invention is used herein to define a protein that has been produced by that body of techniques collectively known as “recombinant DNA technology” (for example, using the nucleic acid, vectors and or host cells described herein).

The term “synthetic” as applied to the peptides of the invention is used herein to define a peptide that has been chemically synthesised in vitro (for example by any of the commercially available solid-phase peptide-synthesis systems).

As used herein in relation to the vectors of the invention, the term “operably linked” refers to a condition in which portions of a linear nucleic acid sequence are capable of influencing the activity of other portions of the same linear nucleic acid sequence. For example, DNA for a signal peptide (secretory leader) is operably linked to DNA for a polypeptide if it is expressed as a precursor which participates in the secretion of the polypeptide; a promoter is operably linked to a coding sequence if it controls the transcription of the sequence; a ribosome binding site is operably linked to a coding sequence if it is positioned in the correct reading-frame so as to permit translation.

By “apo-structure” we mean the three-dimensional structure of the protein that contains no ligand, e.g. substrate or product or cofactor or inhibitor i.e. the active site of the protein is empty.

In the following by “binding site” or “binding cavity” we mean a site (such as an atom, a functional group of an amino acid residue or a plurality of such atoms and/or groups) in a BACE binding cavity, which may bind to an agent compound such as a candidate inhibitor. Depending on the particular molecule in the cavity, sites may exhibit attractive or repulsive binding interactions, brought about by charge, steric considerations and the like.

Binding sites are sites within a macromolecule, or on its surface, at which ligands can bind. Examples are the catalytic or active site of an enzyme (the site on an enzyme at which the amino acid residues involved in catalysing the enzymatic reaction are located), allosteric binding sites (ligand binding sites distinct from the catalytic site, but which can modulate enzymatic activity upon ligand binding), cofactor binding sites (sites involved in binding/co-ordinating cofactors e.g. metal ions), or substrate binding sites (the ligand binding sites on a protein at which the substrates for the enzymatic reaction bind). There are also sites of protein-protein interaction.

In the following by “active site” we mean a site (such as an atom, a functional group of an amino acid residue or a plurality of such atoms and/or groups) in a BACE binding cavity, which is involved in catalysis.

By “fitting”, is meant determining by automatic, or semi-automatic means, interactions between one or more atoms of a candidate molecule and at least one atom of a BACE structure of the invention, and calculating the extent to which such interactions are stable. Interactions include attraction and repulsion, brought about by charge, steric considerations and the like. Various computer-based methods for fitting are described further herein.

By “root mean square deviation” we mean the square root of the arithmetic mean of the squares of the deviations from the mean.

By a “computer system” we mean the hardware means, software means and data storage means used to analyse atomic coordinate data. The minimum hardware means of the computer-based systems of the present invention typically comprises a central processing unit (CPU), input means, output means and data storage means. Desirably a monitor is provided to visualise structure data. The data storage means may be RAM or means for accessing computer readable media of the invention. Examples of such systems are microcomputer workstations available from Silicon Graphics Incorporated and Sun Microsystems running Unix based, Windows NT or IBM OS/2 operating systems.

By “computer readable media” we mean any medium or media, which can be read and accessed directly by a computer e.g. so that the media is suitable for use in the above-mentioned computer system. Such media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

The term “homologue” is used herein in two distinct senses. It is used sensu stricto to define proteins that share a common ancestor. In this sense it covers orthologues (species variants which have diverged in different organisms following a speciation event) and paralogues (variants which have diverged within the same organism after a gene duplication event). Thus, there is a direct evolutionary relationship between such homologues and this may be reflected in structural and/or functional similarities. For example, orthologues may perform the same role in each organism in which they are found, while paralogues may perform functionally related (but distinct) roles within the same organism.

The term is also used herein sensu lato to define proteins which are to some extent structurally similar (i.e. not necessarily evolutionary related and/or structurally and functionally equivalent). In this sense, homology is recognised on the basis of purely structural criteria by the presence of amino acid sequence identities and/or conservative amino acid changes and/or similar secondary, tertiary or quaternary structures.

The term “analogue” is used herein to define proteins with similar functions and/or structures and which are not necessarily evolutionary related. Protein analogues which share function but which have no or little structural similarities are likely to have arisen by convergent evolution. Conversely, protein analogues which share structural similarities but which exhibit few or no functional similarities are likely to have arisen by divergent evolution. Protein analogues may be identified, for example, by screening a library of proteins to detect those with similar function(s) but different physical properties, or by screening for proteins which share structural features but not necessarily any functions (e.g. by immunological screening).

The term “equivalent” is used herein to define those protein analogues which exhibit substantially the same function(s) and which share at least some structural features (e.g. functional domains), but which have not evolved from a common ancestor. Such equivalents are typically synthetic proteins (see below) and may be generated, for example, by identifying sequences of functional importance (e.g. by identifying conserved or canonical sequences, functional domains or by mutagenesis followed by functional assay), selecting an amino acid sequence on that basis and then synthesising a peptide based on the selected amino acid sequence. Such synthesis can be achieved by any of many different methods known in the art, including solid phase peptide synthesis (to generate synthetic peptides) and the assembly (and subsequent cloning) of oligonucleotides. Some synthetic protein analogues may be chimaeras (see below), and such equivalents can be designed and assembled for example by concatenation of two or more different structural and/or functional peptide domains from different proteins using recombinant DNA techniques (see below).

The BACE protein homologues of the invention therefore include proteins and peptides having at least 75%, 80%, 85%, 90%, 95%, 97%, 98% or 99% sequence identity with the reference protein, and include truncated forms of the BACE proteins of the invention. Such truncates are preferably at least 25%, 35%, 50% or 75% of the length of the corresponding specifically exemplified proteins and may have at least 60% sequence identity (more preferably, at least 75%, 80%, 85%, 90%, 95%, 97%, 98% or 99% sequence identity) with that specifically exemplified protein.

Particularly preferred homologues are truncates that contain a segment preferably comprising at least 8, 15, 20 or 30 contiguous amino acids that share at least 75%, 80%, 85%, 90%, 95%, 97%, 98% or 99% sequence identity with that specifically exemplified protein.

A “conservative amino acid change” is one in which the amino acid residue is replaced with an amino acid residue having a similar side chain. Families of amino acid residues having similar side chains have been defined in the art. These families include amino acids with basic side chains (e.g. lysine, arginine and histidine), acidic side chains (e.g. aspartic acid and glutamic acid), non-charged polar side chains (e.g. glycine, asparagine, glutamine, serine, threonine, tyrosine and cysteine), non-polar side chains (e.g. alanine, valine, leucine, isoleucine, proline, phenylalanine, methionine and tryptophan), beta-branched side chains (e.g. threonine, valine and isoleucine), and aromatic side chains (e.g. tyrosine, phenylalanine, tryptophan and histidine).

Thus, references herein to proteins and peptides that are to some defined extent “identical” (or which share a defined extent of “identity”) with a reference protein or peptide may also optionally be interpreted to include proteins and peptides in which conservative amino acid changes are disregarded so that the original amino acid and its changed counterpart are regarded as identical for the purposes of sequence comparisons.

The term “allelic form” is used herein to define a naturally-occurring alternative forms of the sequence present in the BACE protein which reflect naturally-occurring differences in the BACE gene pool. Preferably, allelic variants of the proteins of the invention have at least 60% sequence identity (more preferably, at least 75%, 80%, 85%, 90% or 95% sequence identity) with the corresponding specifically exemplified BACE protein, where sequence identity is determined by comparing the nucleotide sequences of the polynucleotides when aligned so as to maximize overlap and identity while minimizing sequence gaps.

The term “species variant” (or orthologue) is used herein to define the corresponding protein from a different organism. Thus, species variants share a direct evolutionary relationship.

The term “derivative” as applied herein to the BACE proteins of the invention is used to define proteins which are modified versions of the specifically exemplified proteins of the invention. Such derivatives may include fusion proteins, in which the proteins of the invention have been fused to one or more different proteins, peptides or amino acid tags (for example an antibody or a protein domain conferring a biochemical activity, to act as a label, or to facilitate purification). Particularly preferred are derivatives in which the peptides are modified by a polyHis (6×His) tag to facilitate purification of the peptide derivative on Ni²⁺ agarose beads.

The derivatives may also be products of synthetic processes that use a peptide of the invention as a starting material or reactant.

The term “mutein” is used herein to define proteins that are mutant forms of the BACE proteins of the invention, i.e. proteins in which one or more amino acids have been added, altered, deleted, replaced, inserted or substituted. Thus, the terms “BACE mutein” and “mutant BACE protein” are used interchangeably herein. The muteins/mutant BACE proteins of the invention therefore include fragments, truncates and fusion proteins and peptides (e.g. comprising fused immunoglobulin, receptor, tag, label or enzyme moieties).

The muteins of the invention therefore include truncated forms of the BACE proteins of the invention. Such truncates are preferably least 25%, 35%, 50% or 75% of the length of the corresponding specifically exemplified BACE protein and may have at least 60% sequence identity (more preferably, at least 75%, 80%, 85%, 90% or 95% sequence identity) with that specifically exemplified protein.

Particularly preferred are truncates that contain a segment preferably comprising at least 8, 15, 20 or 30 contiguous amino acids that share at least 75%, 80%, 85%, 90% or 95% sequence identity with that specifically exemplified protein.

For the purposes of the present invention, sequence identity is determined by comparing the amino acid sequences of the proteins when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin and Altschul (1990) Proc. Natl. Acad. Sci. USA 87: 2264-2268, modified as in Karlin and Altschul (1993) Proc. Natl. Acad. Sci. USA 90: 5873-5877.

Another example of a mathematical algorithm used for comparison of sequences is the algorithm of Myers and Miller (1988) CABIOS 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85: 2444-2448.

Preferred for use according to the present invention is the WU-BLAST (Washington University BLAST) version 2.0 software. WU-BLAST version 2.0 executable programs for several UNIX platforms can be downloaded from ftp://blast.wustl.edu/blast/executables. This program is based on WU-BLAST version 1.4, which in turn is based on the public domain NCBI-BLAST version 1.4 (Altschul and Gish, 1996, Local alignment statistics, Doolittle ed., Methods in Enzymology 266: 460-480; Altschul et al., 1990, Basic local alignment search tool, Journal of Molecular Biology 215: 403-410; Gish and States, 1993, Identification of protein coding regions by database similarity search, Nature Genetics 3: 266-272; Karlin and Altschul, 1993, Applications and statistics for multiple high-scoring segments in molecular sequences, Proc. Natl. Acad. Sci. USA 90: 5873-5877; all of which are incorporated by reference herein).

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

The muteins of the invention also include peptides in which mutations have been introduced which effectively promote or impair one or more activities of the protein, for example mutations which promote or impair the function of a receptor, a recognition sequence or an effector binding site.

Muteins may be produced by any convenient method. Conveniently, site-directed mutagenesis with mutagenic oligonucleotides may be employed using a double stranded template (pBluescript KS II construct containing nucleic acid encoding the BACE protein), (e.g. Chameleon™ or QuikChange™-Stratagene™) or cassette mutagenesis methods my be employed. After verifying each mutant derivative by sequencing, the mutated gene is excised and inserted into a suitable vector so that the modified protein can be over-expressed and purified.

### BRIEF DESCRIPTION OF THE DRAWINGS

Table 1, provides the coordinates of the BACE structure. The numbering of the residues used in this Table (see Section (D) below) correspond to the numbering of used by Hong et al, ibid. Elsewhere—unless indicated to the contrary—in the specification the numbering of the SwissProt database entry P56817 is used. Residue 1 of Table 1 corresponds to 62 of SwissProt P56817, and residue 385 corresponds to 446 of SwissProt P56817. In the sequence listing below, the SwissProt P56817 residues 14-453 are shown as 16-455 of SEQ ID NO:2.

FIG. 1 represents the packing arrangements of the BACE monomers within the P6₁,22 crystal lattice.

FIG. 2 shows the superposition of BACE in complex with OM99-2 (1FKN), in black, with BACE, of the invention, in the absence of ligand (grey). The position of OM99-2 is defined by a stick representation of the inhibitor.

### DETAILED DESCRIPTION OF THE INVENTION

A. Construct Design

BACE protease is expressed, at high levels, as insoluble inclusion bodies in bacterial cells. To prepare functional protein appropriate for enzyme assay and structural studies these inclusion bodies are solubilised using denaturants and the slow removal of these denaturants results in the formation of the correct tertiary structure. In addition BACE is expressed as a pro-sequence and requires activation by a protease before it is fully functional.

One of the problems of the techniques described in the art (Tang et al) for isolation of BACE from inclusion bodies is the generation of a mixture of products from the uncontrolled cleavage process. Choppa et al describe the isolation of BACE from mammalian cells and the subsequent cleavage with protease, which also gives a mixture of protein species. Thus there is a need in the art for a method of generating active BACE as a homogenous species.

A further problem with the prior art techniques is the low yield of crystallisable material obtained. The inventors surprisingly found that the present invention results in a high yield from bacterial cells, in particular E. coli.

The inventors utilized clostripain as an activating protease to perform this cleavage in a controlled manner but this produced multiple species of BACE, as determined by mass spectrometry. In order to obtain a uniform homogenous protein after activation, a number of different constructs were produced. These constructs focused on the mutation of two of the clostripain cleavage sites (R56 and R57).

The sequences of the invention were designed to achieve a single cleavage point upon activation by clostripain, as activation of wild type sequence in this way resulted in a non-crystallisable protein with heterogeneous N termini.

The BACE constructs of the invention contain successful modifications of the BACE sequence to allow generation of homogeneous protein product from the use of clostripain. The sequence of the invention contains substitution for another amino acid residue or deletion of the arginine 56 and/or arginine 57 (numbering based on wild type full length sequence, SWISS_PROT P56817). In a preferred aspect of the invention this is a conserved substitution. Conservative amino acid substitutions are well known in the art, and include substitutions made on the basis of similarity in polarity, charge, solubility, hydrophobicity, hydrophilicity and/or the amphipathic nature of the amino acid residues involved. For example, positively charged amino acids include lysine and arginine and histidine. In a preferred aspect the mutation introduced is substitution of arginine to lysine at position 56 and/or 57, more preferably 56 and 57. This results in, as oppose to the wild type, the production of a single species of activated protein upon limited digest with clostripain. Clostripain cleavage occurs at a single site and is thus specific and generates a single species in minutes.

The advantage of these mutations is that they allow the controlled cleavage at arginine residue 42 and hence provides a single N-terminus.

This controlled cleavage thus provides a means to produce a substantially homogeneous composition of a BACE protein of the invention. By substantially homogeneous, it is meant that at least 95%, preferably at least 98% and more preferably at least 99% of the BACE protein in the composition has the same N-terminus. The N-terminus may be selected from residues 43 (i.e. by cleavage at 42), 46, 56, 57 or 58, preferably from 43, 56, 57 or 58, more preferably 43, 56 or 57.

These mutations can be introduced onto any sequence of BACE by site-directed mutagenesis techniques, to facilitate the generation of homogeneous material for structural or activity studies. Thus proteins of the invention are BACE proteins with residues 56 and/or 57 either mutated or deleted. Proteins of the invention also include BACE mutants described below in section (C).

The invention is exemplified by several constructs (SEQ ID 5-18). These were built based 15 on the wild type sequence (BACE WT, SEQ ID 2) where R56 and/or R57 were mutated to K or deleted. These were BACE WT R56KR57K (SEQ ID 6), BACE WT R57K (SEQ ID 8), BACE WT R57del (SEQ ID 10). This was also performed on the BACE construct BACE N->Q to give BACE N->Q R56KR57K (SEQ ID 12), BACE N->Q R57K (SEQ ID 16), BACE N->Q R57del (SEQ ID 18). The BACE N->Q construct contains 4 additional mutations of asparagines to glutamine and a C-terminal His tag as well as the arginine mutations. BACE N->Q without the His tag was mutated at 56 and 57 to give BACE N->Q R56K R57K no His (SEQ ID 14).

SEQ ID 19 is the activated from of SEQ ID 6, SEQ ID 21 the activated form of SEQ ID 12 and SEQ ID 20 the activated form of SEQ ID 14, i.e. the form in which the protein is crystallized:

The three BACE constructs BACE WT R56KR57K, BACE N->Q R56KR57K, and BACE N->Q R56KR57K no His gave higher expression levels.

Thus the invention concerns any BACE proteins with one or more of: a mutation at 56, and mutation at 57, or a deletion at 56 or a deletion at 57, but preferably 56 and 57 mutated, and crystals thereof i.e. any BACE protein comprising residues 56-396 of BACE (based on numbering of SwissProt P56817) and containing these mutations.

B. Refolding Protocol

The protein was expressed in E. coli as inclusion bodies, as outlined above. In an improvement of existing techniques BACE isolated from inclusion bodies was refolded by the use of high pH, a sulfobetaine refolding agent, and a longer duration at high pH. This refolding protocol increased the yield of refolded protein obtained and also gave high and reproducible yields of refolded BACE suitable for crystallisation.

The use of high pH in refolding (Burton et el, 1989) and of sulfobetaines as solubilising molecules in folding experiments (Goldberg et al, 1996) has previously been described. Here we describe the use of a combination of these technologies to give an unprecedented high yield of BACE. In addition to this combination of high pH and sulfobetaine, in another deviation from existing protocols for refolding BACE, the pH is maintained at high pH for at least 2 weeks. This is in comparison to the method of Tang et al, where BACE is solubilised at high pH and then the pH lowered before protein recovery at least 2-3 weeks later, preferably 3-4 weeks later.

Another aspect of the invention therefore concerns a novel method of producing soluble BACE proteins of the invention, utilizing a refolding protocol comprising the combined techniques of high pH buffer and the use of sulfobetaine, and also maintaining this high pH over at least two weeks.

More specifically, a method for producing refolded recombinant BACE comprising refolding the BACE under conditions which denature and then slowly renature the enzyme into a soluble form wherein: (a) the BACE is solubilised using a chaotrope such as urea or guanidine at 8-10M (typically 8 M urea solution) including one or more reducing agents at a pH of greater than 8.0 e.g. pH 9.0-10.5; (b) the BACE is then diluted into an aqueous buffer, like 20 mM-Tris, pH 9.0, containing sulfobetaine, preferably 10 mM sulfobetaine, where the sulfobetaine is preferably NDSB256 (3-(benzyldimethylammonio) propanesulfonate); (c) the solution is maintained at low temperature, e.g. 3-6° C. typically 4° C., and at high pH, typically approximately pH 9.0, for at least 2 weeks (typically 3 weeks, more typically 4 weeks) before proceeding with purification.

C. Protein Crystals.

Described herein is a crystal of BACE having a hexagonal space group P6₁22, and unit cell dimensions a=b=103.2 Å, c=169.1 Å, α=β=60°, γ=120°. Unit cell variability of 5% may be observed in all dimensions. Such crystals contain one copy of BACE in the asymmetric unit.

Such a crystal may be obtained using the methods described in the accompanying examples.

The crystal may be of the BACE protein of SEQ ID 19 although as explained earlier any homologue, allelic form, species variant, derivative or mutein (as hereinbefore defined) may be used. Thus, it will be understood by those of skill in the art that some variation to the primary amino acid sequence may be made without significant alteration to the resulting crystal structure. Such minor variations include the replacement of one or more amino acids, for example from 1 to 30, such as 1, 2, 3, 4, 5, 6, 7, 8, 9 or 10 amino acids by an equivalent or fewer number of amino acids.

The methodology used to provide a BACE crystal illustrated herein may be used generally to provide a human BACE apo crystal resolvable at a resolution of at least 3 Å.

The invention thus further provides an apo BACE crystal having a resolution better than, i.e. numerically lower than, 2.5 Å.

The invention also provides a BACE crystal having a resolution better than, i.e. numerically lower than, 1.8 Å.

The invention also provides apo crystals of BACE resolvable to at least 2.5 Å capable of being soaked with compound(s) to form co-complex structures.

The proteins may be wild-type proteins or variants thereof, which are modified to promote crystal formation, for example by N-terminal truncations and/or deletion of loop regions, which prevent crystal formation.

The methods described herein may be used to make a BACE protein crystal, particularly of a BACE protein of SEQ ID 19-21, which method comprises growing a crystal by vapour diffusion using a reservoir buffer that contains 18-26% PEG 5000 MME, preferably 20-24% PEG 5000 MME, more preferably 20-22.5% PEG 5000 MME, with 180-220 mM (e.g. 200 mM) ammonium iodide and 180-220 mM (e.g. 200 mM) tri-sodium citrate (pH 6.4-6.6). In a preferred embodiment, this reservoir buffer may also contain from 0 to 5% glycerol, e.g. about 2.5% glycerol. The growing of the crystal is by vapour diffusion and is performed by placing an aliquot of the protein solution on a cover slip as a hanging drop above a well containing the reservoir buffer. The concentration of the protein solution used was approximately 7 mg/ml.

Other crystals of the invention include crystals which have selected coordinates of the binding pocket, wherein the amino acid residues associated with those selected coordinates are located in a protein framework which holds these amino acids in a relative spatial configuration corresponding to the spatial configuration of those amino acids in Table 1. By “corresponding to”, it is meant within an rm.s.d. of less than 2.0 Å, preferably less than 1.5 Å, more preferably less than 1.0 Å, even more preferably less than 0.74 Å, even more preferably less than 0.72 Å and most preferably less than 0.5 Å from the Cα or backbone atoms of Table 1, preferably the Cα atoms.

Crystals of the invention also include crystals of BACE mutants (muteins). In addition, BACE mutants may be crystallized in co-complex with known BACE substrates or inhibitors or novel compounds.

As explained herein, a mutant BACE (or BACE mutein) is a BACE protein characterized by the replacement or deletion of at least one amino acid from the wild type BACE. Such a mutant may be prepared for example by site-specific mutagenesis, or incorporation of natural or unnatural amino acids.

As explained herein, the present invention therefore contemplates BACE mutants (or muteins) as hereinbefore defined.

For example, the BACE mutants may define a polypeptide which is obtained by replacing at least one amino acid residue in a native or synthetic BACE with a different amino acid residue and/or by adding and/or deleting amino acid residues within the native polypeptide or at the N- and/or C-terminus of a polypeptide corresponding to BACE, and which has substantially the same three-dimensional structure as BACE from which it is derived. By having substantially the same three-dimensional structure is meant having a set of atomic structure co-ordinates that have a root mean square deviation (r.m.s.d.) of less than or equal to about 2.0 Å (preferably less than 1.5 Å, more preferably less than 1.0 Å, even more preferably less than 0.74 Å, even more preferably less than 0.72 Å and most preferably less than 0.5 Å) when superimposed with the atomic structure co-ordinates of the BACE from which the mutant is derived when at least about 50% to 100% of the C_{α} atoms of the BACE are included in the superposition. A mutant may have, but need not have, enzymatic or catalytic activity.

To produce homologues or mutants, amino acids present in the said protein can be replaced by other amino acids having similar properties, for example hydrophobicity, hydrophobic moment, antigenicity, propensity to form or break α-helical or β-sheet structures, and so. Substitutional variants of a protein are those in which at least one amino acid in the protein sequence has been removed and a different residue inserted in its place. Amino acid substitutions are typically of single residues but may be clustered depending on functional constraints e.g. at a crystal contact. Preferably amino acid substitutions will comprise conservative amino acid substitutions. Insertional amino acid variants are those in which one or more amino acids are introduced. This can be amino-terminal and/or carboxy-terminal fusion as well as intrasequence. Examples of amino-terminal and/or carboxy-terminal fusions are affinity tags, MBP tag, and epitope tags.

Deletional variants are those in which one or more amino acids are removed. This can be amino-terminal and/or carboxy-terminal, or in an internal region (for example a loop region), for example to remove or shorten that region.

Amino acid substitutions, deletions and additions that do not significantly interfere with the three-dimensional structure of the BACE will depend, in part, on the region of the BACE where the substitution, addition or deletion occurs. In highly variable regions of the molecule, non-conservative substitutions as well as conservative substitutions may be tolerated without significantly disrupting the three-dimensional structure of the molecule. In highly conserved regions, or regions containing significant secondary structure, conservative amino acid substitutions are preferred.

As explained earlier, conservative amino acid substitutions are well known in the art, and include substitutions made on the basis of similarity in polarity, charge, solubility, hydrophobicity, hydrophilicity and/or the amphipathic nature of the amino acid residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine. Other conservative amino acid substitutions are well known in the art.

In some instances, it may be particularly advantageous or convenient to substitute, delete and/or add amino acid residues to a BACE binding pocket or catalytic residue in order to provide convenient cloning sites in the cDNA encoding the polypeptide, to aid in purification of the polypeptide, to modify compound binding etc. Such substitutions, deletions and/or additions which do not substantially alter the three dimensional structure of BACE will be apparent to those having skills in the art.

It should be noted that the mutants (BACE muteins) contemplated herein need not exhibit enzymatic activity. Indeed, amino acid substitutions, additions or deletions that interfere with the catalytic activity of the BACE but which do not significantly alter the three-dimensional structure of the catalytic region are specifically contemplated by the invention. Such crystalline polypeptides, or the atomic structure co-ordinates obtained there from, can be used to identify compounds that bind to the protein.

The crystallization of such mutants and the determination of the three-dimensional structures by X-ray crystallography relies on the ability of the mutant proteins to yield crystals that diffract at high resolution. The mutant protein could then be used to obtain information on compound binding through the determination of mutant protein/ligand complex structures, which may be characterized using the BACE crystal structure of Table 1.

The mutations can be introduced by site-directed mutagenesis e.g. using a Stratagene QuikChange™ Site-Directed Mutagenesis Kit or cassette mutagenesis methods (see e.g. Ausubel et al., eds., Current Protocols in Molecular Biology, John Wiley & Sons, Inc., New York, and Sambrook et al., Molecular Cloning: a Laboratory Manual, 2nd ed., Cold Spring Harbor Laboratory Press, Cold Spring Harbor, N.Y., (1989)).

To the extent that the present invention relates to BACE-ligand complexes and mutant, homologue, allelic form, species variant, derivative, mutein and analogue proteins of BACE, crystals of such proteins may be formed. The skilled person would recognize that the conditions provided herein for crystallising BACE may be used to form such crystals. Alternatively, the skilled person would use the conditions as a basis for identifying modified conditions for forming the crystals.

Thus the aspects of the invention relating to crystals of BACE, may be extended to crystals of mutant/mutein, homologue, allelic form, species variant or derivative (as defined herein).

D. Crystal Coordinates

In a further aspect, the invention also provides an apo crystal structure of BACE having the three dimensional atomic coordinates of Table 1. An advantageous feature of the structure defined by the atomic coordinates is that it has a high resolution of about 1.75 Å. A further advantageous aspect is the provision of an apo structure of BACE, which contains no ligand bound, unlike those previously described in the art. This is particularly advantageous as ligands can then be easily soaked into the crystal to provide co-complex data without the need for removal of any ligand already present, and without the need for time-consuming co-crystallisation experiments.

The BACE structure set out in Table 1 is a monomer structure. This is the first time that a monomer has been observed crystallographically for this protein.

Table 1 gives atomic coordinate data for BACE. In Table 1 the third column denotes the atom type, the fourth the residue type, the fifth the chain identification, the sixth the residue number (the atom numbering as described in Hong et al, 2000) the seventh, eighth and ninth columns are the X, Y, Z coordinates respectively of the atom in question, the tenth column the occupancy of the atom, the eleventh the temperature factor of the atom, the twelfth the chain identification, and the last, thirteenth column, the atom type.

Each of the tables is presented in an internally consistent format. For example, in Table 1 the coordinates of the atoms of each amino acid residue are listed such that the backbone nitrogen atom is first, followed by the C-alpha backbone carbon atom, designated CA, followed by the carbon and oxygen of the protein backbone and finally side chain residues (designated according to one standard convention). Alternative file formats (e.g. such as a format consistent with that of the EBI Macromolecular Structure Database (Hinxton, UK)) which may include a different ordering of these atoms, or a different designation of the side-chain residues, may be used or preferred by others of skill in the art. However it will be apparent that the use of a different file format to present or manipulate the coordinates of the Tables is within the scope of the present invention.

The coordinates of Table 1 provide a measure of atomic location in Ångstroms, to 3 decimal places. The coordinates are a relative set of positions that define a shape in three dimensions, but the skilled person would understand that an entirely different set of coordinates having a different origin and/or axes could define a similar or identical shape. Furthermore, the skilled person would understand that varying the relative atomic positions of the atoms of the structure so that the root mean square deviation of the residue backbone atoms (i.e. the nitrogen-carbon-carbon backbone atoms of the protein amino acid residues) is less than 2.0 Å, preferably less than 1.5 Å, more preferably less than 1.0 Å, even more preferably less than 0.74 Å, even more preferably less than 0.72 Å and most preferably less than 0.5 Å when superimposed on the coordinates provided in Table 1 for the Cα atoms or residue backbone atoms, will generally result in a structure which is substantially the same as the structure of Table 1 in terms of both its structural characteristics and usefulness for structure-based analysis of BACE-interactivity molecular structures.

Likewise the skilled person would understand that changing the number and/or positions of the water molecules and/or substrate molecules of Table 1 will not generally affect the usefulness of the structure for structure-based analysis of BACE-interacting structure. Thus for the purposes described herein as being aspects of the present invention, it is within the scope of the invention if: the Table 1 coordinates are transposed to a different origin and/or axes; the relative atomic positions of the atoms of the structure are varied so that the root mean square deviation of residue backbone atoms is less than 2.0 Å, preferably less than 1.5 Å, more preferably less than 1.0 Å, even more preferably less than 0.74 Å, even more preferably less than 0.72 Å, and most preferably less than 0.5 Å when superimposed on the coordinates provided in Table 1 for the Cα or residue backbone atoms; and/or the number and/or positions of water molecules and/or substrate molecules is varied.

Reference herein to the coordinate data of Table 1 and the like thus includes the coordinate data in which one or more individual values of the Table are varied in this way unless specified explicitly to the contrary. In a preferred aspect, reference herein to the coordinates of Table 1 or parts thereof (e.g. selected coordinates) should be taken to include coordinates having a root mean square deviation of less than 0.72 Å, and preferably less than 0.5 Å, from the Cα atoms of Table 1 or corresponding parts thereof.

By “root mean square deviation” we mean the square root of the arithmetic mean of the squares of the deviations from the mean.

Protein structure similarity is routinely expressed and measured by the root mean square deviation (r.m.s.d.), which measures the difference in positioning in space between two sets of atoms. The r.m.s.d. measures distance between equivalent atoms after their optimal superposition. The r.m.s.d. can be calculated over all atoms, over residue backbone atoms (i.e. the nitrogen-carbon-carbon backbone atoms of the protein amino acid residues), main chain atoms only (i.e. the nitrogen-carbon-oxygen-carbon backbone atoms of the protein amino acid residues), side chain atoms only or more usually over C-alpha atoms only. For the purposes of this invention, the r.m.s.d. can be calculated over any of these, using any of the methods outlined below.

Methods of comparing protein structures are discussed in Methods of Enzymology, vol 115, pg 397-420. The necessary least-squares algebra to calculate r.m.s.d. has been given by Rossman and Argos (J. Biol. Chem., vol 250, pp7525(1975)) although faster methods have been described by Kabsch (Acta Crystallogr., Section A, A92, 922 (1976); Acta Cryst. A34, 827-828 (1978)), Hendrickson (Acta Crystallogr., Section A, A35, 158 (1979) and McLachan (J. Mol. Biol., vol 128, pp49 (1979). Some algorithms use an iterative procedure in which the one molecule is moved relative to the other, such as that described by Ferro and Hermans (Ferro and Hermans, Acta Crystallographic, A33, 345-347 (1977)). Other methods e.g. Kabsch's algorithm locate the best fit directly.

It is usual to consider C-alpha atoms and the rmsd can then be calculated using programs such as LSQKAB (Collaborative Computational Project 4. The CCP4 Suite: Programs for Protein Crystallography, Acta Crystallographica, D50, (1994), 760-763), MNYFIT (part of a collection of programs called COMPOSER, Sutcliffe, M. J., Haneef, I., Carney, D. and Blundell, T. L. (1987) Protein Engineering, 1, 377-384), MAPS (Lu, G. An Approach for Multiple Alignment of Protein Structures (1998, in manuscript)), QUANTA (Jones et al., Acta Crystallography A47 (1991), 110-119 and commercially available from Accelerys, San Diego, Calif.), Insight (commercially available from Accelerys, San Diego, Calif.), Sybyl® (commercially available from Tripos, Inc., St Louis), O (Jones et al., Acta Crystallographica, A47, (1991), 110-119), and other coordinate fitting programs.

In, for example the programs LSQKAB and O, the user can define the residues in the two proteins that are to be paired for the purpose of the calculation. Alternatively, the pairing of residues can be determined by generating a sequence alignment of the two proteins, programs for sequence alignment are discussed in more detail in Section G. The atomic coordinates can then be superimposed according to this alignment and an r.m.s.d. value calculated. The program Sequoia (C. M. Bruns, I. Hubatsch, M. Ridderström, B. Mannervik, and J. A. Tainer (1999) Human Glutathione Transferase A4-4 Crystal Structures and Mutagenesis Reveal the Basis of High Catalytic Efficiency with Toxic Lipid Peroxidation Products, Journal of Molecular Biology 288(3): 427-439) performs the alignment of homologous protein sequences, and the superposition of homologous protein atomic coordinates. Once aligned, the r.m.s.d. can be calculated using programs detailed above. For sequence identical, or highly identical, the structural alignment of proteins can be done manually or automatically as outlined above. Another approach would be to generate a superposition of protein atomic coordinates without considering the sequence.

It is more normal when comparing significantly different sets of coordinates to calculate the r.m.s.d. value over C-alpha atoms only. It is particularly useful when analysing side chain movement to calculate the r.m.s.d. over all atoms and this can be done using LSQKAB and other programs.

Varying the atomic positions of the atoms of the structure by up to about 0.5 Å in a concerted way, preferably up to about 0.3 Å in any direction will result in a structure which is substantially the same as the structure of Table 1 in terms of both its structural characteristics and utility e.g. for molecular structure-based analysis.

Also, modifications in the BACE crystal structure due to e.g. mutations, additions, substitutions, and/or deletions of amino acid residues (including the deletion of one or more BACE protomers) could account for variations in the BACE atomic coordinates. However, atomic coordinate data of BACE modified so that a ligand that bound to one or more binding sites of BACE would be expected to bind to the corresponding binding sites of the modified BACE are, for the purposes described herein as being aspects of the present invention, also within the scope of the invention. Reference herein to the coordinates of Table 1 thus includes the coordinates modified in this way. Preferably, the modified coordinate data define at least one BACE binding cavity.

Those of skill in the art will appreciate that in many applications of the invention, it is not necessary to utilise all the coordinates of Table 1, but merely a portion of them. The term portion is intended to define a sub-set of the coordinates, which may or may not represent contiguous amino acid residues in the BACE structure. For example, as described below, in methods of modelling candidate compounds with BACE, selected coordinates of BACE may be used, for example at least 5, preferably at least. 10, more preferably at least 50 and even more preferably at least 100 atoms of the BACE structure. Likewise, the other applications of the invention described herein, including homology modelling and structure solution, and data storage and computer assisted manipulation of the coordinates, may also utilise all or a portion of the coordinates of Table 1.

E. Homology Modelling

The invention also provides a means for homology modelling of other proteins (referred to below as target BACE proteins). By “homology modelling”, it is meant the prediction of related BACE structures based either on X-ray crystallographic data or computer-assisted de novo prediction of structure, based upon manipulation of the coordinate data of Table 1.

“Homology modelling” extends to target BACE proteins, which are analogues or homologues of the BACE protein whose structure has been determined in the accompanying examples. It also extends to BACE protein mutants of BACE protein itself.

The term “homologous regions” describes amino acid residues in two sequences that are identical or have similar (e.g. aliphatic, aromatic, polar, negatively charged, or positively charged) side-chain chemical groups. Identical and similar residues in homologous regions are sometimes described as being respectively “invariant” and “conserved” by those skilled in the art.

In general, the method involves comparing the amino acid sequences of the BACE protein of Table 1 with a target BACE protein by aligning the amino acid sequences (Dunbrack et al., Folding and Design, 2, (1997), 27-42). Amino acids in the sequences are then compared and groups of amino acids that are homologous (conveniently referred to as “corresponding regions”) are grouped together. This method detects conserved regions of the polypeptides and accounts for amino acid insertions or deletions.

Homology between amino acid sequences can be determined using commercially available algorithms. The programs BLAST, gapped BLAST, BLASTN, PSI-BLAST and BLAST 2 sequences (provided by the National Center for Biotechnology Information) are widely used in the art for this purpose, and can align homologous regions of two amino acid sequences. These may be used with default parameters to determine the degree of homology between the amino acid sequence of the Table 1 protein and other target BACE proteins, which are to be modeled.

Analogues are defined as proteins with similar three-dimensional structures and/or functions with little evidence of a common ancestor at a sequence level.

Homologues are defined as proteins with evidence of a common ancestor, i.e. likely to be the result of evolutionary divergence and are divided into remote, medium and close sub-divisions based on the degree (usually expressed as a percentage) of sequence identity.

A homologue is defined here as a protein with at least 15% sequence identity or which has at least one functional domain, which is characteristic of BACE.

There are two types of homologue: orthologues and paralogues. Orthologues are defined as homologous genes in different organisms, i.e. the genes share a common ancestor coincident with the speciation event that generated them. Paralogues are defined as homologous genes in the same organism derived from a gene/chromosome/genome duplication, i.e. the common ancestor of the genes occurred since the last speciation event.

The homologues could also be mutants as described in section (C).

Once the amino acid sequences of the polypeptides with known and unknown structures are aligned, the structures of the conserved amino acids in a computer representation of the polypeptide with known structure are transferred to the corresponding amino acids of the. polypeptide whose structure is unknown. For example, a tyrosine in the amino acid sequence of known structure may be replaced by, a phenylalanine, the corresponding homologous amino acid in the amino acid sequence of unknown structure.

The structures of amino acids located in non-conserved regions may be assigned manually by using standard peptide geometries or by molecular simulation techniques, such as molecular dynamics. The final step in the process is accomplished by refining the entire structure using molecular dynamics and/or energy minimization.

Homology modelling as such is a technique that is well known to those skilled in the art (see e.g. Greer, Science, Vol. 228, (1985), 1055, and Blundell et al, Eur. J. Biochem, Vol. 172, (1988), 513). The techniques described in these references, as well as other homology modelling techniques, generally available in the art, may be used in performing the present invention.

Thus the invention provides a method of homology modelling comprising the steps of: (a) aligning a representation of an amino acid sequence of a target BACE protein of unknown three-dimensional structure with the amino acid sequence of the BACE of Table 1 to match homologous regions of the amino acid sequences; (b) modelling the structure of the matched homologous regions of said target BACE of unknown structure on the corresponding regions of the BACE structure as defined by Table 1; and (c) determining a conformation (e.g. so that favorable interactions are formed within the target BACE of unknown structure and/or so that a low energy conformation is formed) for said target BACE of unknown structure which substantially preserves the structure of said matched homologous regions.

Preferably one or all of steps (a) to (c) are performed by computer modelling.

The aspects of the invention described herein which utilise the BACE structure in silico may be equally applied to homologue models of BACE obtained by the above aspect of the invention, and this application forms a further aspect of the present invention. Thus having determined a conformation of a BACE by the method described above, such a conformation may be used in a computer-based method of rational drug design as described herein.

The absence of a ligand from our structure is particularly advantageous for modelling of other proteins as this structure reveals the native structure of the protein unaffected by conformational changes upon ligand binding.

F. Structure Solution

The structure of the human BACE can also be used to solve the crystal structure of other target BACE proteins including other crystal forms of BACE, mutants, and co-complexes of BACE, where X-ray diffraction data or NMR spectroscopic data of these target BACE proteins has been generated and requires interpretation in order to provide a structure.

In the case of BACE, this protein may crystallize in more than one crystal form. The structure coordinates of BACE, or portions thereof, as provided by this invention are particularly useful to solve the structure of those other crystal forms of BACE. They may also be used to solve the structure of BACE mutants, BACE co-complexes, or of the crystalline form of any other protein with significant amino acid sequence homology to any functional domain of BACE.

In the case of other target BACE proteins, particularly the BACE proteins referred to in Section C above, the present invention allows the structures of such targets to be obtained more readily where raw X-ray diffraction data is generated.

Thus, where X-ray crystallographic or NMR spectroscopic data is provided for target BACE-ligand complex, or a BACE homologue or analogue of unknown three-dimensional structure, the structure of BACE, as defined by Table 1, may be used to interpret that data to provide a likely structure for the other BACE by techniques which are well known in the art, e.g. phasing in the case of X-ray crystallography and assisting peak assignments in NMR spectra.

One method that may be employed for these purposes is molecular replacement. In this method, the unknown crystal structure, whether it is another crystal form of BACE, a BACE mutant, or a BACE co-complex, or the crystal of a target BACE protein with amino acid sequence homology to any functional domain of BACE, may be determined using the BACE structure coordinates of this invention as provided herein. This method will provide an accurate structural form for the unknown crystal more quickly and efficiently than attempting to determine such information ab initio.

Examples of computer programs known in the art for performing molecular replacement are CNX (Brunger A. T.; Adams P. D.; Rice L. M., Current Opinion in Structural Biology, Volume 8, Issue 5, October 1998, Pages 606-611 (also commercially available from Accelerys San Diego, Calif.) or AMORE (Navaza, J. (1994). AMoRe: an automated package for molecular replacement. Acta Cryst. A50, 157-163).

Thus, in a further aspect of the invention provides a method for determining the structure of a protein, which method comprises; providing the co-ordinates of Table 1, and either (a) positioning the co-ordinates in the crystal unit cell of said protein so as to provide a structure for said protein or (b) assigning NMR spectra Peaks of said protein by manipulating the coordinates of Table 1.

In a preferred aspect of this invention the co-ordinates are used to solve the structure of target BACE particularly homologues of BACE for example aspartic proteases such as BACE2 or cathepsin E (69% and 37% similarity, respectively).

G. Computer Systems

In another aspect, the present invention provides systems, particularly a computer system, the systems containing either (a) atomic coordinate data according to Table 1, said data defining the three-dimensional structure of BACE or at least selected coordinates thereof; (b) structure factor data (where a structure factor comprises the amplitude and phase of the diffracted wave) for BACE, said structure factor data being derivable from the atomic coordinate data of Table 1; (c) atomic coordinate data of a target BACE protein generated by homology modelling of the target based on the data of Table 1; (d) atomic coordinate data of a target BACE protein generated by interpreting X-ray crystallographic data or

NMR data by reference to the data of Table 1; or (e) structure factor data derivable from the atomic coordinate data of (c) or (d).

For example the computer system may comprise: (i) a computer-readable data storage medium comprising data storage material encoded with the computer-readable data; (ii) a working memory for storing instructions for processing said computer-readable data; and (iii) a central-processing unit coupled to said working memory and to said computer-readable data storage medium for processing said computer-readable data and thereby generating structures and/or performing rational drug design. The computer system may further comprise a display coupled to said central-processing unit for displaying said structures.

The invention also provides such systems containing atomic coordinate data of target BACE proteins wherein such data has been generated according to the methods of the invention described herein based on the starting data provided by Table 1.

Such data is useful for a number of purposes, including the generation of structures to analyze the mechanisms of action of BACE proteins and/or to perform rational drug design of compounds which interact with BACE, such as compounds which are inhibitors of BACE.

In another aspect, the invention provides a computer-readable storage medium, comprising a data storage material encoded with computer readable data, wherein the data are defined by all or a portion (e.g. selected coordinates as defined herein) of the structure coordinates of BACE of Table 1, or a homologue of BACE, wherein said homologue comprises backbone atoms that have a root mean square deviation from the Cα or backbone atoms (nitrogen-carbon_{α}-carbon) of Table 1 of less than 2 Å, such as not more than 1.5Å, preferably less than 1.5 Å, more preferably less than 1.0 Å, even more preferably less than 0.74 Å, even more preferably less than 0.72 Å and most preferably less than 0.5 Å.

The invention also provides a computer-readable data storage medium comprising a data storage material encoded with a first set of computer-readable data comprising a Fourier transform of at least a portion (e.g. selected coordinates as defined herein) of the structural coordinates for BACE according to Table 1; which, when combined with a second set of machine readable data comprising an X-ray diffraction pattern of a molecule or molecular complex of unknown structure, using a machine programmed with the instructions for using said first set of data and said second set of data, can determine at least a portion of the structure coordinates corresponding to the second set of machine readable data.

In a further aspect, the present invention provides computer readable media with with at least one of: (a) atomic coordinate data according to Table 1 recorded thereon, said data defining the three-dimensional structure of BACE, or at least selected coordinates thereof; (b) structure factor data for BACE recorded thereon, the structure factor data being derivable from the atomic coordinate data of Table 1; (c) atomic coordinate data of a target BACE protein generated by homology modelling of the target based on the data of Table 1; (d) atomic coordinate data of a BACE-ligand complex or a BACE homologue or analogue generated by interpreting X-ray crystallographic data or NMR data by reference to the data of Table 1; and (e) structure factor data derivable from the atomic coordinate data of (c) or (d).

By providing such computer readable media, the atomic coordinate data can be routinely accessed to model BACE or selected coordinates thereof. For example, RASMOL (Sayle et al., TIBS, Vol. 20, (1995), 374) is a publicly available computer software package which allows access and analysis of atomic coordinate data for structure determination and/or rational drug design.

On the other hand, structure factor data, which are derivable from atomic coordinate data (see e.g. Blundell et al., in Protein Crystallography, Academic Press, New York, London and San Francisco, (1976)), are particularly useful for calculating e.g. difference Fourier electron density maps.

A further aspect of the invention provides a method of providing data for generating structures and/or performing rational drug design for BACE, BACE homologues or analogues, complexes of BACE with a potential modulator, or complexes of BACE homologues or analogues with potential modulators, the method comprising:

(i) establishing communication with a remote device containing computer-readable data comprising at least one of: (a) atomic coordinate data according to Table 1, said data defining the three-dimensional structure of BACE, at least one sub-domain of the three-dimensional structure of BACE, or the coordinates of a plurality of atoms of BACE; (b) structure factor data for BACE, said structure factor data being derivable from the atomic coordinate data of Table 1; (c) atomic coordinate data of a target BACE homologue or analogue generated by homology modelling of the target based on the data of Table 1; (d) atomic coordinate data of a protein generated by interpreting X-ray crystallographic data or NMR data by reference to the data of Table 1; and (e) structure factor data derivable from the atomic coordinate data of (c) or (d); and (ii) receiving said computer-readable data from said remote device.

Thus the remote device may comprise e.g. a computer system or computer readable media of one of the previous aspects of the invention. The device may be in a different country or jurisdiction from where the computer-readable data is received. The communication may be via the internet, intranet, e-mail etc. Typically the communication will be electronic in nature, but some or all of the communication pathway may be optical, for example, over optical fibres. Additionally, the communication may be through radio signals or satellite transmissions.

H. Uses of the Crystals of the Invention

The crystal structures obtained according to the present invention (including the structure of Table 1 as well the structures of target BACE proteins obtained in accordance with the methods described herein), may be used in several ways for drug design.

By identifying conditions under which high quality crystals of apo-BACE can be produced (i.e. crystals which can diffract X-rays for the determination of atomic coordinates to a resolution of better than 2.5 Å), the present invention facilitates the identification of modulators of BACE activity.

The invention is particularly suitable for the design, screening, development and optimization of BACE inhibitor components. It is thus a preferred aspect of the invention that modulators are inhibitors.

In a further aspect, the invention provides a method for determining the structure of a compound bound to BACE, said method comprising: (a) providing a crystal of BACE according to the invention; (b) soaking the crystal with said compounds; and (c) determining the structure of said BACE compound complex by employing the data of Table 1.

Alternatively, the BACE and compound may be co-crystallized. Thus the invention provides a method for determining the structure of a compound bound to BACE, said method comprising; mixing the protein with the compound(s), crystallizing the protein-compound(s) complex; and determining the structure of said BACE-compound(s) complex by reference to the data of Table 1.

A mixture of compounds may be soaked or co-crystallized with the crystal, wherein only one or some of the compounds may be expected to bind to the BACE. As well as the structure of the complex, the identity of the complexing compound(s) is/are then determined.

In either case, substrate or a substrate analogue thereof may optionally be present.

The method may comprise the further steps of: (a) obtaining or synthesising said candidate modulator; (b) forming a complex of BACE and said candidate modulator; and (c) analysing said complex by X-ray crystallography or NMR spectroscopy to determine the ability of said candidate modulator to interact with BACE.

The analysis of such structures may employ (i) X-ray crystallographic diffraction data from the complex and (ii) a three-dimensional structure of BACE, or at least selected coordinates thereof, to generate a difference Fourier electron density map of the complex, the three-dimensional structure being defined by atomic coordinate data according to Table 1. The difference Fourier electron density map may then be analyzed, to identify the binding mode of the modulator.

Therefore, such complexes can be crystallized and analyzed using X-ray diffraction methods, e.g. according to the approach described by Greer et al., J. of Medicinal Chemistry, Vol. 37, (1994), 1035-1054, and difference Fourier electron density maps can be calculated based on X-ray diffraction patterns of soaked crystals of BACE or co-crystallized BACE and the solved structure of uncomplexed BACE. These maps can then be analyzed e.g. to determine whether and where a particular compound binds to BACE and/or changes the conformation of BACE.

Electron density maps can be calculated using programs such as those from the CCP4 computing package (Collaborative Computational Project 4. The CCP4 Suite: Programs for Protein Crystallography, Acta Crystallographica, D50, (1994), 760-763.). For map visualization and model building programs such as “O” (Jones et al., Acta Crystallographica, A47, (1991), 110-119) or “QUANTA” (1994, San Diego, Calif.: Molecular Simulations can be used.

The crystal structures of a series of complexes may then be solved by molecular replacement and compared with that of the BACE of Table 1. Potential sites for modification within the various binding sites of the enzyme may thus be identified. This information provides an additional tool for determining the most efficient binding interactions, for example, increased hydrophobic interactions, between BACE and a chemical entity or compound.

All of the complexes referred to above may be studied using well-known X-ray diffraction techniques and may be refined against 1.5 to 3.5 Å resolution X-ray data to an R value of about 0.30 or less using computer software, such as CNX (Brunger et al., Current Opinion in Structural Biology, Vol. 8, Issue 5, October 1998, 606-611, and commercially available from Accelerys, San Diego, Calif.), X-PLOR (Yale University, ©1992, distributed by Accelerys), as described by Blundell et al, (1976) and Methods in Enzymology, vol. 114 & 115, H. W. Wyckoff et al., eds., Academic Press (1985).

This information may thus be used to optimize known classes of BACE substrates or inhibitors, and more importantly, to design and synthesize novel classes of BACE inhibitors.

Analysing the complex by X-ray crystallography will determine the ability of the candidate compound to interact with BACE. Analysis of the co-complexes of BACE may involve e.g. phasing, molecular replacement or calculating a Fourier difference map of the complex as discussed above. However, with the high resolutions obtainable with the crystal, it can also be possible to determine the ability of the candidate modulator to interact with BACE merely by comparing the intensities and/or positions of X-ray diffraction spots from the. complex with e.g. diffraction spots of uncomplexed BACE or a previously identified BACE-ligand complex. Thus the step of analysing the complex may involve analysing the intensities and/or positions of X-ray diffraction spots from the complex to determine the ability of the candidate modulator to interact with BACE.

Having obtained and characterized a modulator compound according to the invention, the invention further provides a method for modulating the activity of BACE which method comprises: (a) providing BACE under conditions where, in the absence of modulator, the BACE is able to synthesize amyloid β-peptide from amyloid precursor protein (APP); (b) providing a modulator compound; and (c) determining the extent to which the activity of BACE is altered by the presence of said compound.

T. Structure-based Drug Design

Determination of the three-dimensional structure of BACE provides important information about the binding sites of BACE, particularly when comparisons are made with similar enzymes. This information may then be used for rational design of BACE inhibitors, e.g. by computational techniques which identify possible binding ligands for the binding sites, by enabling linked-fragment approaches to drug design, and by enabling the identification and location of bound ligands using X-ray crystallographic analysis. These techniques are discussed in more detail below.

Greer et al. (1994) describes an iterative approach to ligand design based on repeated sequences of computer modelling, protein-ligand complex formation and X-ray crystallographic or NMR spectroscopic analysis. Thus novel thymidylate synthase inhibitor series were designed de novo by Greer et al., and BACE inhibitors may also be designed in the this way. More specifically, using e.g. GRID on the solved 3D structure of BACE, a ligand (e.g. a potential inhibitor) for BACE may be designed that complements the functionalities of the BACE binding sites. The ligand can then be synthesised, formed into a complex with BACE, and the complex then analysed by X-ray crystallography to identify the actual position of the bound ligand. The structure and/or functional groups of the ligand can then be adjusted, if necessary, in view of the results of the X-ray analysis, and the synthesis and analysis sequence repeated until an optimised ligand is obtained. Related approaches to structure-based drug design are also discussed in Bohacek et al., Medicinal Research Reviews, Vol. 16, (1996), 3-50.

Linked-fragment approaches to drug design also require accurate information on the atomic coordinates of target receptors. The basic idea behind these approaches is to determine (computationally or experimentally) the binding locations of plural ligands to a target molecule, and then construct a molecular scaffold to connect the ligands together in such a way that their relative binding positions are preserved. The ligands may be provided computationally and modelled in a computer system, or provided in an experimental setting, wherein crystals according to the invention are provided and a plurality of ligands soaked separately or in mixed pools into the crystal prior to X-ray analysis and determination of their location.

The binding site of two or more ligands are determined and may be connected to form a potential lead compound that can be further refined using e.g. the iterative technique of Greer et al. For a virtual linked-fragment approach see Verlinde et al., J. of Computer-Aided Molecular Design, 6, (1992), 131-147, and for NMR and X-ray approaches see Shuker et al., Science, 274, (1996), 1531-1534 and Stout et al., Structure, 6, (1998), 839-848. The use of these approaches to design BACE inhibitors is made possible by the determination of the BACE structure.

Many of the techniques and approaches to structure-based drug design described above rely at some stage on X-ray analysis to identify the binding position of a ligand in a ligand-protein complex. A common way of doing this is to perform X-ray crystallography on the complex, produce a difference Fourier electron density map, and associate a particular pattern of electron density with the ligand. However, in order to produce the map (as explained e.g. by Blundell et al. (1976)) it is necessary to know beforehand the protein 3D structure (or at least the protein structure factors). Therefore, determination of the BACE structure also allows difference Fourier electron density maps of BACE-ligand complexes to be produced, which can greatly assist the process of rational drug design.

The provision of the crystal structures of the invention will also allow the development of compounds which interact with the binding pocket regions of BACE (for example to act as inhibitors of a BACE) based on a fragment linking or fragment growing approach.

For example, the binding of one or more molecular fragments can be determined in the protein binding pocket by X-ray crystallography. Molecular fragments are typically compounds with a molecular weight between 100 and 200 Da (Carr et al, 2002). This can then provide a starting point for medicinal chemistry to optimize the interactions using a structure-based approach. The fragments can be combined onto a template or used as the starting point for ‘growing out’ an inhibitor into other pockets of the protein (Blundell et al, 2002). The fragments can be positioned in the binding pocket of BACE and then ‘grown’ to fill the space available, exploring the electrostatic, van der Waals or hydrogen-bonding interactions that are involved in molecular recognition. The potency of the original weakly binding fragment thus can be rapidly improved using iterative structure-based chemical synthesis.

At one or more stages in the fragment growing approach, the compound may be synthesized and tested in a biological system for its activity. This can be used to guide the further growing out of the fragment.

Where two fragment-binding regions are identified, a linked fragment approach may be based upon attempting to link the two fragments directly, or growing one or both fragments in the manner described above in order to obtain a larger, linked structure, which may have the desired properties.

The previous aspects of the invention relate also to fragment linking or fragment growing approaches to rational drug design. Thus the step of providing the structure of a candidate modulator molecule in the previous aspects may be performed by providing the structures of a plurality of molecular fragments and linking the molecular fragments to form a candidate modulator molecule. Furthermore the step of fitting the structure of the candidate modulator molecule in the previous aspects may be performed by fitting the structure of each of the molecular fragments (before or after the molecular fragments are linked together).

For example, the-computer-based method of rational drug design may comprise:

(a) providing the coordinates of at least two atoms of the BACE of Table 1; (b) providing the structures of a plurality of molecular fragments; (c) fitting the structure of each of the molecular fragments to the selected coordinates of the BACE; and (d) assembling the molecular fragments into a single molecule to form a candidate modulator molecule.

In practice, it will be desirable to model a sufficient number of atoms of the BACE as defined by the coordinates of Table 1, which represent a binding pocket. Thus, in this embodiment of the invention, there will preferably be provided the coordinates of at least 5, preferably at least 10, more preferably at least 50 and even more preferably at least 100 preferably at least 500 selected atoms of the BACE structure.

A further aspect of the invention provides a compound having a chemical structure selected using the method of any one of the previous aspects, said compound being an inhibitor of BACE.

J. Uses of the Coordinates of the Invention in In Silico Analysis and Design

Although the invention will facilitate the determination of actual crystal structures comprising BACE and a compound, which modulates BACE, current computational techniques provide a powerful alternative to the need to generate such crystals and generate and analyze diffraction data. Accordingly, a particularly preferred aspect of the invention relates to in silico methods directed to the analysis and development of compounds, which interact, with BACE structures of the present invention.

The approaches to structure-based drug design described below all require initial identification of possible compounds for interaction with target bio-molecule (in this case BACE). Sometimes these compounds are known e.g. from the research literature. However, when they are not, or when novel compounds are wanted, a first stage of the drug design program may involve computer-based in silico screening of compound databases (such as the Cambridge Structural Database) with the aim of identifying compounds which interact with the binding site or sites of the target bio-molecule. Screening selection criteria may be based on pharmacokinetic properties such as metabolic stability and toxicity. However, determination of the BACE structure allows the architecture and chemical nature of each BACE binding site to be identified, which in turn allows the geometric and functional constraints of a descriptor for the potential inhibitor to be derived. The descriptor is, therefore, a type of virtual 3-D pharmacophore, which can also be used as selection criteria or filter for database screening.

Thus as a result of the determination of the BACE three-dimensional structure, more purely computational techniques for rational drug design may also be used to design BACE inhibitors (for an overview of these techniques see e.g. Walters et al (Drug Discovery Today, Vol.3, No.4, (1998), 160-178; Abagyan, R.; Totrov, M. Curr. Opin. Chem. Biol. 2001, 5, 375-382). For example, automated ligand-receptor docking programs (discussed e.g. by Jones et al. in Current Opinion in Biotechnology, Vol.6, (1995), 652-656 and Halperin, I.; Ma, B.; Wolfson, H.; Nussinov, R. Proteins 2002, 47, 409-443), which require accurate information on the atomic coordinates of target receptors may be used to design potential BACE inhibitors.

The aspects of the invention described herein which utilize the BACE structure in silico may be equally applied to both the BACE structure of Table 1 and the models of target BACE proteins obtained by other aspects of the invention. Thus having determined a conformation of a BACE by the method described above, such a conformation may be used in a computer-based method of rational drug design as described herein. In addition the availability of the structure of the BACE will allow the generation of highly predictive pharmacophore models for virtual library screening or compound design.

Accordingly, the invention provides a computer-based method for the analysis of the interaction of a molecular structure with a BACE structure of the invention, which comprises: (a) providing the structure of a BACE of the invention of Table 1; (b) providing a molecular structure to be fitted to said BACE structure; and (c) fitting the molecular structure to the BACE structure of Table 1.

In an alternative aspect, the method of the invention may utilize the coordinates of atoms of interest of BACE, which are in the vicinity of a putative molecular structure binding region, for example within 10-25 Å of the catalytic regions or within 5-10 Å of a compound bound, in order to model the pocket in which the structure binds. These coordinates may be used to define a space, which is then analyzed “in silico”. Thus the invention provides a computer-based method for the analysis of molecular structures which comprises: (a) providing the coordinates of at least two atoms of a BACE structure of the invention (“selected coordinates”); (b) providing the structure of a molecular structure to be fitted to said coordinates; and (c) fitting the structure to the selected coordinates of the BACE.

In practice, it will be desirable to model a sufficient number of atoms of the BACE as defined by the coordinates of Table 1, which represent a binding pocket. Thus, in this embodiment of the invention, there will preferably be provided the coordinates of at least 5, preferably at least 10, more preferably at least 50 and even more preferably at least 100 and preferably 500 selected atoms of the BACE structure.

In order to provide a three-dimensional structure of compounds to be fitted to a BACE structure of the invention, the compound structure may be modelled in three dimensions using commercially available software for this purpose or, if its crystal structure is available, the coordinates of the structure may be used to provide a representation of the compound for fitting to a BACE structure of the invention.

The step of providing the structure of a candidate modulator molecule may involve selecting the compound by computationally screening a database of compounds for interaction with the binding cavity or cavities. For example, a 3-D descriptor for the potential modulator may be derived, the descriptor including geometric and functional constraints derived from the architecture and chemical nature of the binding cavity or cavities. The descriptor may then be used to interrogate the compound database, a potential modulator being a compound that has a good match to the features of the descriptor. In effect, the descriptor is a type of virtual pharmacophore.

In any event, the determination of the three-dimensional structure of BACE provides a basis for the design of new and specific ligands for BACE. For example, knowing the three-dimensional structure of BACE, computer modelling programs may be used to design different molecules expected to interact with possible or confirmed binding cavities or other structural or functional features of BACE. Examples of this are discussed in Schneider, G.; Bohm, H. J. Drug Discov.Today 2002, 7, 64-70.

More specifically, the interaction of a compound with BACE can be examined through the use of computer modelling using a docking program such as GOLD (Jones et al., J. Mol. Biol., 245, 43-53 (1995), Jones et al., J. Mol. Biol., 267, 727-748 (1997)), GRAMM (Vakser, I. A., Proteins, Suppl., 1:226-230 (1997)), DOCK (Kuntz et al, J.Mol.Biol. 1982, 161, 269-288, Makino et al, J.Comput.Chem. 1997, 18, 1812-1825), AUTODOCK (Goodsell et al, Proteins 1990, 8, 195-202, Morris et al, J.Comput.Chem. 1998, 19, 1639-1662.), FlexX, (Rarey et al, J.Mol.Biol. 1996, 261, 470-489) or ICM (Abagyan et al, J.Comput.Chem. 1994, 15, 488-506). This procedure can include computer fitting of compounds to BACE to ascertain how well the shape and the chemical structure of the compound will bind to the BACE.

Also computer-assisted, manual examination of the binding site structure of BACE may be performed. The use of programs such as GRID (Goodford, J. Med. Chem., 28, (1985), 849-857)—a program that determines probable interaction sites between molecules with various functional groups and an enzyme surface—may also be used to analyse the binding cavity or cavities to predict partial structures of inhibiting compounds.

Computer programs can be employed to estimate the attraction, repulsion, and steric hindrance of the two binding partners (i.e. the BACE and a candidiate modulator). Generally the tighter the fit, the fewer the steric hindrances, and the greater the attractive forces, the more potent the potential modulator since these properties are consistent with a tighter binding constant. Furthermore, the more specificity in the design of a potential drug, the more likely it is that the drug will not interact with other proteins as well. This will tend to minimise potential side-effects due to unwanted interactions with other proteins.

In another aspect, the present invention provides a method for identifying an agent compound (e.g. an inhibitor) which modulates BACE activity, comprising the steps of: (a) employing three-dimensional atomic coordinate data according to Table 1 to characterise at least one BACE binding site and preferably a plurality of BACE binding sites; (b) providing the structure of a candidate agent compound; (c) fitting the candidate agent compound to the binding sites; and (d) selecting the candidate agent compound.

Preferably sufficient binding sites are characterised to define a BACE binding cavity or cavities.

A plurality (for example two, three or four) of (typically spaced) BACE binding sites may be characterised and a plurality of respective compounds designed or selected. The agent compound may then be formed by linking the respective compounds into a larger compound which preferably maintains the relative positions and orientations of the respective compounds at the binding sites. The larger compound may be formed as a real molecule or by computer modelling.

In one embodiment a plurality of candidate agent compounds are screened or interrogated for interaction with the binding sites. In one example, step (b) involves providing the structures of the candidate agent compounds, each of which is then fitted in step (c) to computationally screen a database of compounds (such as the Cambridge Structural Database) for interaction with the binding sites, i.e. the candidate agent compound may be selected by computationally screening a database of compounds for interaction with the binding sites (see Martin, J. Med Chem., vol 35, 2145-2154 (1992)). In another example, a 3-D descriptor for the agent compound is derived, the descriptor including e.g. geometric and functional constraints derived from the architecture and chemical nature of the binding cavity or cavities. The descriptor may then be used to interrogate the compound database, the identified agent compound being the compound which matches with the features of the descriptor. In effect, the descriptor is a type of virtual pharmacophore.

In a related aspect, the present invention provides a method for identifying a candidate modulator (e.g. potential inhibitor) of BACE comprising the steps of: (a) employing a three-dimensional structure of BACE, at least one sub-domain thereof, or a plurality of atoms thereof, to characterise at least one BACE binding cavity, the three-dimensional structure being defined by atomic coordinate data according to Table 1; and (b) identifying the candidate modulator by designing or selecting a compound for interaction with the binding cavity.

Detailed structural information can then be obtained about the binding of the compound to BACE, and in the light of this information adjustments can be made to the structure or functionality of the compound, e.g. to improve its interaction with BACE. The above steps may be repeated and re-repeated as necessary.

K. Compound Selection

In another aspect, in place of in silico methods, high throughput screening of compounds to select compounds with binding activity may be undertaken, and those compounds which show binding activity may be selected as possible candidate modulators, and further crystallized with BACE (e.g. by co-crystallization or by soaking) for X-ray analysis. The resulting X-ray structure may be compared with that of Table 1 for a variety of purposes.

L. Compounds of the Invention

Having designed or selected possible binding candidate modulators (e.g. by in silico analysis, “wet” chemical methods, X-ray analysis etc.) by determining those which have favourable fitting properties (e.g. strong attraction between candidate and BACE), these can then be screened for activity.

Consequently all the methods of compound design and identification outlined above can optionally include the step of: (a) obtaining or synthesising the candidate modulator; and (b) contacting the candidate modulator with BACE to determine the ability of the candidate modulator to interact with BACE.

More preferably, in the latter step the candidate modulator is contacted with BACE under conditions to determine its function.

For example, in the contacting step above the candidate modulator is contacted with BACE in the presence of a substrate, and typically a buffer, to deter mine the ability of said candidate modulator to inhibit BACE. The substrate may be e.g. APP. So, for example, an assay mixture for BACE may be produced which comprises the candidate modulator, substrate and buffer.

Detailed structural information can be obtained about the binding of the candidate modulator to BACE, and in the light of this information adjustments can be made to the structure or functionality of the candidate modulator, e.g. to improve binding to the binding cavity or cavities. The above steps may be repeated and re-repeated as necessary.

Following identification of such compounds, it may be manufactured and/or used in the preparation, i.e. manufacture or formulation, of a composition such as a medicament, pharmaceutical composition or drug. These may be administered to individuals.

Thus, the present invention extends in various aspects not only to a compound as provided by the invention, but also a pharmaceutical composition, medicament, drug or other composition comprising such a compound e.g. for treatment (which may include preventative treatment) of disease; a method comprising administration of such a composition to a patient, e.g. for treatment of disease; use of such an inhibitor in the manufacture of a composition for administration, e.g. for treatment of disease; and a method of making a pharmaceutical composition comprising admixing such an inhibitor with a pharmaceutically acceptable excipient, vehicle or carrier, and optionally other ingredients.

Thus a further aspect of the present invention provides a method for preparing a medicament, pharmaceutical composition or drug, the method comprising:

(a) identifying a BACE modulator molecule (which may thus be termed a lead compound) by a method of any one of the other aspects of the invention disclosed herein; (h) optimising the structure of the modulator molecule; and (c) preparing a medicament, pharmaceutical composition or drug containing the optimised modulator molecule.

The above-described processes of the invention may be iterated in that the modified compound may itself be the basis for further compound design.

By “optimising the structure” we mean e.g. adding molecular scaffolding, adding or varying functional groups, or connecting the molecule with other molecules (e.g. using a fragment linking approach) such that the chemical structure of the modulator molecule is changed while its original modulating functionality is maintained or enhanced. Such optimisation is regularly undertaken during drug development programmes to e.g. enhance potency, promote pharmacological acceptability, increase chemical stability etc. of lead compounds.

Modification will be those conventional in the art known to the skilled medicinal chemist, and will include, for example, substitutions or removal of groups containing residues which interact with the amino acid side chain groups of a BACE structure of the invention. For example, the replacements may include the addition or removal of groups in order to decrease or increase the charge of a group in a test compound, the replacement of a charge group with a group of the opposite charge, or the replacement of a hydrophobic group with a hydrophilic group or vice versa. It will be understood that these are only examples of the type of substitutions considered by medicinal chemists in the development of new pharmaceutical compounds and other modifications may be made, depending upon the nature of the starting compound and its activity.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa. 15th Edition, 1975.

Compositions may be used, e.g. for treatment (which may include preventative treatment) of a disease such as Alzheimer's disease or Alzheimer's-type pathology in Downs syndrome. Thus the invention provides a method comprising administration of such a composition to a patient, e.g. for treatment of a disease such as Alzheimer's disease; use of such an agent compound in the manufacture of a composition for administration, e.g. for treatment of a disease such as Alzheimer's disease; and a method of making a pharmaceutical composition comprising admixing such an agent compound with a pharmaceutically acceptable excipient, vehicle or carrier, and optionally other ingredients.

### EXEMPLIFICATION

The invention will now be described with reference to specific Examples. These are merely exemplary and for illustrative purposes only: they are not intended to be limiting in any way to the scope of the invention described. These examples constitute the best mode currently contemplated for practicing the invention;

BACE protease was expressed at high levels in bacterial cells as insoluble inclusion bodies. To prepare functional protein for enzyme assay and structural studies these inclusion bodies were solublised using denaturants; the slow removal of these denaturants allowed the formation of the correct tertiary structure. In the method described here, BACE was expressed as a pro-sequence and required activation by a protease before becoming fully functional. Clostripain was used as an activating protease but produced multiple species of BACE as determined by mass spectrometry. In order to obtain a uniform homogenous protein after activation by clostripain, a number of different constructs were produced. These constructs focused on the mutation of two undesireable clostripain cleavage sites (following residues R56 and R57).

Cloning of BACE WT and BACE N->Q

The full-length DNA coding sequence of BACE was cloned from human cerebellum and human dorsal root ganglion (DRG) cDNA by PCR using oligonucleotide primers based on the published BACE sequence (EMBL accession no. AF190725). The full-length template sequence was obtained by PCR amplification using the following primers: hBACE-sp1 and -ap1 I were used for primary amplification, hBACE-sp2 and -ap2 for nested PCR.

The primers were as follows:

| | |
|---|---|
| hBACE-sp1 | |
| 5′-AGCTCCCTCTCCTGAGAAGCCACC-3′ | (SEQ ID NO: 22) |
| hBACE-ap1 | |
| 5′-CCACAGGTGCCATCTGTGTCTCC-3′ | (SEQ ID NO: 23) |
| hBACE-sp2 | |
| 5′-CACCAGCACCACCCAGACTTGG-3′ | (SEQ ID NO: 24) |
| hBACE-ap2 | |
| 5′-AACCACGGAGGTGTGGTCCAGG-3′ | (SEQ ID NO: 25) |

A cDNA construct encoding a modified BACE form was made as follows. A partial BACE cDNA fragment was amplified using the full-length BACE clone as a template with primers hBACE_EC(Bam-M-14)_FOR (5′-CGG GAT CCA TGG CGG GAG TGC TGC TGC CTG CC-3′) and hBACE_EC(Bam-453)_REV (5′-CGG GAT CCT TAT GAC TCA TCT GTC TGT GGA ATG TTG TAG C-3′). The resulting 1342 bp PCR fragment was subcloned in vector pCR2.1-TOPO using the TOPO TA cloning® kit (Invitrogen) according to the manufacturer's instructions. The inserts of several resulting clones were fully sequenced and a clone containing no PCR mistakes was selected. The insert of this clone was excised from the pCR2.1-TOPO construct using the BamHI restriction endonuclease and subcloned to vector pET11a (Novagen) linearized with BamHI. The BACE coding sequence (BACE WT, SEQ ID 1) in the resulting clones was confirmed by sequence analysis and the resulting correct construct was named M-T7-RGSM(BACE14-453)/pET11a.

Plasmid M-T7-RGSM(BACE14-453)/pET11a encodes a 455 amino acid residue protein named BACE WT containing a T7 epitope tag encoded by the pET11a vector sequence (AA 1 to 11), a linker sequence (AA 12-15; RGSM) and the partial BACE amino acid sequence from residue 14 to 453 (AA 16 to 455)(numbering based on SEQ ID 2). The calculated molecular mass of the resulting protein is 50.2 kDa.

The insert from construct Plasmid M-T7-RGSM(BACE14-453)/pET11 a was amplified by PCR to incorporate a His₆ tag (CAT CAC CAT CAT CAC CAC) just upstream of the stop codon and BamH1 site. Following cloning of this amplified fragment back into the original expression vector, the asparagine residues at positions -153, -172, -223 and -354 (numbers refer to the database BACE sequence BACE_HUMAN, P56817 in Swissprot) were mutated to glutamine (AAC to CAA) using the Quikchange™ mutagenesis system (Stratagene, used according to the manufacturers instructions), to generate BACE N->Q (SEQ ID 3).

Introduction of Activation Site Mutations

BACE WT and BACE N->Q, described above, were mutated using the Quickchange™ site directed mutagenesis protocol (Stratagene). Two complimentary oligonucleotides were designed which spanned the site of the mutation and which incorporated the amino acids changes to be made. These oligonucleotides were then used as primers in a PCR reaction producing each of the strands of the plasmid with the mutation present; the parental plasmid is digested with the methylation sensitive restriction endonuclease DpnI and then transformed into competent E. coli cells.

Primers were applicable for the mutation of both BACE WT and BACE N->Q due to their high sequence homology. Seven constructs were produced; these are detailed below with the oligonucleotide sequence used to make the constructs.

1) BACE WT mutating arginine 56 to lysine and arginine 57 to lysine (SEQ ID 5)

(SEQ ID NO: 26)

5′-CCCGAGGAGCCCGGCAAGAAGGGCAGCTTTGTGGAGATG-3′

(SEQ ID NO: 27)

5′-CATCTCCACAAAGCTGCCCTTCTTGCCGGGCTCCTCGGG-3′

2) BACE WT mutating arginine 57 to lysine (SEQ ID 7)

(SEQ ID NO: 28)

5′-CCCGAGGAGCCCGGCCGGAAGGGCAGCTTTGTGGAGATGG-3′

(SEQ ID NO: 29)

5′-CCATCTCCACAAAGCTGCCCTTCCGGCCGGGCTCCTCGGG-3′

3) BACE WT deleting arginine 57 (SEQ ID 9)

(SEQ ID NO: 30)

5′-CCCGAGGAGCCCGGCAGGGGCAGCTTTGTGGAGATGGTGGAC-3′

(SEQ ID NO: 31)

5′-GTCCACCATCTCCACAAAGCTGCCCCTGCCGGGCTCCTCGGG-3′

4) BACE N->Q mutating arginine 56 to lysine and arginine 57 to lysine (SEQ ID 11)

(SEQ ID NO: 32)

5′-CCCGAGGAGCCCGGCAAGAAGGGCAGCTTTGTGGAGATG-3′

(SEQ ID NO: 33)

5′-CATCTCCACAAAGCTGCCCTTCTTGCCGGGCTCCTCGGG-3′

5) BACE N->Q mutating arginine 57 to lysine (SEQ ID 15)

(SEQ ID NO: 34)

5′-CCCGAGGAGCCCGGCCGGAAGGGCAGCTTTGTGGAGATGG-3′

(SEQ ID NO:35)

5′-CCATCTCCACAAAGCTGCCCTTCCGGCCGGGCTCCTCGGG-3′

6) BACE N->Q deleting arginine 57 (SEQ ID 17)

(SEQ ID NO: 36)

5′-CCCGAGGAGCCCGGCAGGGGCAGCTTTGTGGAGATGGTGGAC-3′

(SEQ ID NO: 37)

5′-GTCCACCATCTCCACAAAGCTGCCCCTGCCGGGCTCCTCGGG-3′

7) BACE N->Q mutating arginine 56 to lysine and arginine 57 to lysine and removing the C terminal poly histidine tag (SEQ ID 13)

(SEQ ID NO: 38)

5′-CCCGAGGAGCCCGGCAAGAAGGGCAGCTTTGTGGAGATG-3′

(SEQ ID NO: 39)

5′-CATCTCCACAAAGCTGCCCTTCTTGCCGGGCTCCTCGGG-3′

(SEQ ID NO: 40)

5′-CCACAGACAGATGAGTCATGACACCATCATCACCACTAAG-3′

(SEQ ID NO: 41)

5′-CTTAGTGGTGATGATGGTGTCATGACTCATCTGTCTGTGG-3′

After transformation of the plasmid the protein coding region was checked by DNA sequencing.

Protein Production (1)

Plasmid constructs were transformed into BLR(DE3) as follows: 1-2 μl DNA was added into 25 ul BLR(DE3) competent cells. Cells were then heat shocked at 42° C. for 45 secs, followed by incubation for 30 mins at 4° C. The sample was placed on ice for 2-3 mins before addition of 125-250 ul HOC medium and left for 60 mins at 37° C. Cells were plated out onto agar containing carbenicillin & incubated at 37° C. for 16 h. Transformations were stored at 4° C. Transformed cells could be used up to after 8 weeks storage.

Colonies were inoculated in 100 ml LB broth with 1 mM carbenicillin, and shaken for 16 h at 25° C. 12 ml of this culture was added to 1 L of the same medium in baffle flasks. The typical total culture volume was 12, 20 or 24 L. Cells were induced by addition of 1 mM IPTG at approximately OD₆₀₀ 1.0. Cells were harvested 3 to 4 hours after induction by centrifugation for 7 min at 16000 g. Cell pellets were resuspended in 1 liter TN buffer (150 mM NaCl, 50 mM Tris, pH 7.5) before addition of 10 mg lysozyme per liter of bacterial culture. The suspension was left for 20 mins under vigorous stirring then frozen at −70° C.

The lysates were thawed & adjusted to 1 mM MgCl12 and 20 μl 10 mg/ml DNAse, incubated 30-60 mins at 20° C., then 0.1% Triton X-100 was added. Inclusion body washes were performed in 11 wash steps, spun down at 13,000-16,000 g for 20 mins at room temperature then resuspended by sonication in TNT buffer (TN buffer +0.1% Triton 100). The washing step with TNT was repeated at least three times (up to seven times) until an almost homogenous dark cream precipitate was obtained. At this stage the pellet was washed twice with TN buffer. The typical yield for a 12 L culture of BACE WT constructs was 4.5 g washed inclusion body material.

Protein Refolding (1)

Each g of inclusion bodies was solubilised with 22.5 ml of 8 M urea, 50 mM Tris, 0.1 M beta-mercaptoethanol, 10 mM DTT, 1 mM EDTA. After 2 to 3 hours under gentle stirring, this was spun at 48400 g for 25 mins. This was then diluted 1 in 10 in 8 M Urea, 0.2 mM oxidized glutathione, 1.0 mM reduced glutathione. This is the starting solution for refolding

Refolding was accomplished by dilution into 20 volumes 20 mM Tris, 10 mM NDSB256 (3-(benzyldimethylammonio)propanesulfonate). The addition was achieved by slowly dripping from a burette into a strongly stirred solution. Addition was carried out at room temperature.

The pH was adjusted to approximately 9 using 13.5 ml 1 N HCl per 5 liter of refolding mix either immediately after dilution or 16 h after dilution. This was left at 4° C. for 2-3 weeks. The refolding mix was then adjusted to pH 8.2 16h before concentrating. In instances where a longer incubation was applied it appeared that yields were slightly better. No precipitation was seen when attempting to refold BACE, even in totally unsuccessful conditions. Constructs BACE WT R57K, BACE WT R57DEL, BACE N->Q R57K, and BACE R57DEL refolded with lower yields.

Protein Purification of BACE from Refolding Step (1)

The refolded protein sample was concentrated by ultrafiltration using two parallel Vivaflow 200 cells (MWCO 30 Kda), fed by a single pump. The concentration factor was not more than 200 times: if exceeded, precipitation occurred.

Concentrated refolded BACE was loaded and eluted on a 1.75 L Sephacryl 300 column run at a flow of 0.2 cm-1/min in 0.4 M Urea, 20 mM Tris, 10 mM HCl. Typical loading volume was 2% bed volume. From reconcentrated material three peaks are observed, the first one near the void volume (large aggregates), which merges into a second peak of aggregated inactive material. The third peak (elutes at approx 40% of column volume) constitutes active BACE. For BACE WT constructs, the active fraction elutes at approximately 800 ml.

Activation by Clostripain (1)

Clostripain (Cp; EC 3.4.22.8, from Worthington or Sigma C7403) was activated before use by solubilising the freeze dried material to 1.25 mg/ml in: 20 mM Calcium Acetate, 8 mM DTT, 100 mM Tris, pH 8 at 1.25 mg/ml 4 ° C for at least I h. The preparation was then stable at 4 ° C for up to four weeks.

The third peak (typically 100 ml at an average of 0.3 mg ml) from Sephacryl 300 elution was treated with activated Cp, (1/100 dilution) for between 30-90mins at room temperature.

Activation of BACE WT R56KR57K, BACE N->Q R56KR57K & BACE N->Q R56KR57K no His by clostripain was performed as described above except that prior to activation the solution was concentrated ten fold using Vivaspin 20 ml 30 KDa MWCO.

The reaction was stopped by loading onto a Mono Q HR5-5 column equilibrated in 0.4 M Urea, 20 mM Tris, 10 mM HCI, 1 mM EDTA followed by washing using the same buffer. The protein was eluted with a 0 to 1 M NaCl gradient over 10 column volumes. A typical final yield of active soluble BACE WT R56KR5!K is 1-2 mg of protein per liter of culture grown. The eluted protein was characterised and used in crystallisation assays.

### Protein Production (2)

BLR (DE3) competent cells were transformed as described earlier and plated onto agar containing ampicillin (Amp). A colony was picked into 250ml LB +100 ug/ml Amp and grown overnight ( 37° C, 1 85rpm. Following overnight growth (OD₆₀₀ varied between 2.0- 2.5) 10 ml of this culture was used to inoculate IL of fresh LB+100 ₁₁g/ml Amp in a 2L baffled flask. Routinely 24L of fresh LB+Amp would be inoculated from the overnight growth. Following inoculation, the 24L prep would be grown at 37° C., 185rpm until an OD₆₀₀ =1.0 was obtained. Protein expression was induced by the addition of IPTG to a final concentration of 1 mM. Cultures were incubated for a further 3 hours (at 37° C., 185rpm) before harvesting by centrifugation at 8000 rpm for 10 mins (JLA 8.1000). Cell pellets could be stored at -800C or processed immediately.

All following protein production procedures were performed at room temperature unless stated otherwise. Cell pellet was re-suspended in 500ml of TN buffer (TN buffer - 1 5OmM NaCl, 50 mM Tris, pH7.5). 240mg of egg lysozyme (10 mg/L of bacterial culture) was added to the re-suspended pellet. The suspension was left stirring for 20mins. Following this, 100 ul of DNase 1 (10 mg/ml stock) was added to the suspension and this was left stirring for 20mins. This lysate was clarified by centrifugation at 8000rpm for 20mins (JLA8. l000).

The supernatant was discarded and the pellet was re-suspended in 100 ml TNT buffer (TNT buffer—150 mM NaCl, 5 mM Tris, pH7.5, 0.1% Triton X-100). Effort was made to break up any lumps present in the pellet so that a homogenous re-suspension was obtained. Following this, the re-suspension was sonicated for 2 mins (20 sec pulses). 400 ml of TNT buffer was added to bring the volume of the suspension up to ˜500 mls. This was centrifuged for 20 mins at 8000 rpm and the supernatant discarded. The re-suspension in TNT buffer and sonication steps, as described above, were repeated twice. Following these three TNT washes, the pellet was re-suspended in 100 ml of TN buffer and sonicated for 2 mins (20 second pulses). The suspension was centrifuged for 20 mins at 8000 rpm. This wash in TN buffer was repeated once. Approximately 12-15 g of inclusion bodies was obtained from the 24L of culture.

Protein Refolding (2)

The inclusion body preparation was solubilised by addition of 100 mls of solubilisation buffer (Sol. Buffer—8M urea, 50 mM Tris, 0.1M beta-mercaptoethanol, 10 mM DTT, 1 mM EDTA). Effort was made to break up the inclusion body pellet using a pipette/spatula. The solution was left stirring gently overnight. The suspension was centrifuged for 30 mins at 25,000 rpm (JA25). The supernatant (˜100 mls) was diluted by the addition of 900 mls of 8M urea, 0.2 mM oxidised glutathione, 1.0M reduced glutathione.

The 1L of solubilised inclusion bodies as prepared above were refolded by a further 20× dilution. A 250 ml aliquot of solubilised inclusion body prep was added drop-wise to 4.75L of refolding buffer (Refolding buffer—20 mM Tris, 10 mM NDSB256 (3-(benzyldimethylammonio)propanesulfonate). The 4.75L of refolding buffer was stirred vigorously (not foaming) and the 250 mls of inclusion body prep was added using a peristaltic pump. Care was taken to add the 250 mls at a fast drop rather than a continuous pour. The remaining 750 mls of inclusion body prep was diluted in the same way (250 mls into 4.75L of refolding buffer). The four 5L vessels were placed at 4° C. overnight.

Following overnight incubation at 4° C., the pH of each 5L vessel was adjusted to pH9.0 by addition of conc HCl. The vessels were then placed back at 4° C. and left for 3 weeks.

Protein Purification of BACE from Refolding Step (2)

Two parallel Vivaflow 200 cells (MWCO 30 Kda) fed by a single peristaltic pump were used. Each 5L of refolding mix was concentrated to ˜50 mls. Over concentrating leads to precipitation and should be avoided. The concentration of 5L of refolding mix took ˜2 hours. The 50 mls of concentrated refolding mix was centrifuged for 25 mins, at 25,000 rpm. The supernatant was then ready for gel filtration using a Sephacryl S-300 column (100×3.5). This method is limited by the volume of concentrated refolding mix than can be loaded onto the gel filtration column (50 mls) per run. Sephacryl S-300 column was equilibrated with 0.4M urea, 20 mM Tris, 10 mM HCl (at a flow rate of 4 ml/min). 50 ml of sample can be loaded per run. The column was run at a flow rate of 4 ml/min. SDS PAGE analysis of peaks 1,2 and 3 showed the presence of BACE (50 Kda band) however activity assay of all three peaks showed only active BACE in peak 3. Fractions from Peak 3 were pooled and kept on ice.

Activation by Clostripain (2)

Clostripain (Sigma C7403) was prepared by dissolving protein to a final concentration of 1.25 mg/ml in 20 mM Calcium acetate, 8 mM DTT, 100 mM Tris pH 8.0. The clostripain was activated by incubating on ice for 1 hour prior to use.

Pooled fractions from peak 3 (˜100 ml at 0.2 mg/ml) were activated by the addition of 1/100 dilution of 1.25 mg/ml clostripain. The reaction was incubated at 37° C. in a water bath for 90 minutes. The reaction was stopped by addition of 1 mM EDTA and placed on ice. Note: With each fresh batch of Sigma Clostripain, a time trial was performed on a small amount of BACE to verify the length of incubation needed at 37° C. The length of incubation varied from 30-90 mins. Analysis by SDS PAGE clearly showed the appearance of the lower molecular weight activated species (˜47 Kda) from the larger inactivated species (˜50 Kda).

A Mono Q 5/5 ion exchange column was pre-equilibrated in 0.4M urea, 20 mM Tris, 10 mM HCl. The activated BACE (˜50 mls at ˜0.2 mg/ml) was loaded onto the Mono Q column at a flow rate of 1.0 ml/min. Activated BACE was purified by applying a linear salt gradient (0.4M urea, 20 mM Tris, 10 mM HCl, 1.0M NaCl) over 20 column volumes. Following analysis by SDS PAGE and subsequent activity assay, fractions corresponding to activated BACE were pooled and buffer exchanged into crystallisation buffer (20 mM Tris, pH8.2, 150 mM NaCl, 1 mM DTT).

Protein Purification of BACE from Refolding Step (3)

By using method 3 in conjunction with the S-200 INDEX gel filtration column, all 20L of refolding mix could be processed in one go.

A Sartocon filtration cassette (MWCO 30 Kda) was used in conjunction with a Watson Marlow 623S high speed pump. This assembly was set up as described in the manufactures operation manual. The 20L of refolding mix was concentrated down to ˜500 mls in less than 1 hour. Due to the dead volume in the assembly tubing, the volume could not be reduced further. At this stage the 500 mls of concentrated refolding mix was filtered using a 0.2 um filter. The filtered sample was then ready for gel filtration using an S-200 INDEX gel filtration column (100×10.0). A S-200 INDEX column pre-equilibrated in 0.4M urea, 20 mm Tris, 10 mM HCl was used. The column run was at a flow rate of 10 mls/min.

SDS analysis of peaks 1,2 and 3 showed that BACE was present in all fractions. Activity assay showed that only peak 3 contain some BACE activity. Fractions from peak 3 were pooled (˜250 mls at 0.1 mg/ml).

Prior to clostripain activation, the BACE sample was concentrated using a Resource Q ion exchange column. A 6/1 Resource Q column was pre-equilibrated in 0.4M urea, 20 mM Tris, 10 mM HCl. The Bace sample was loaded onto the column at 7 ml/min. BACE was eluted off the column using a linear salt gradient (0.4M urea, 20 mM Tris, 10 mM HCl, 1M NaCl) over 5 column volumes. This step has the effect of dramatically reducing the sample volume size. Prior to clostripain activation, the protein sample is diluted with 0.4M urea, 20 mM Tris, 10 mM HCl to reduce the salt concentration to enable further purification using Mono Q. A dilution factor of 5:1 has been used successfully.

This is then followed by Clostripain Activation and Mono Q purification as outlined above.

Protein Characterization

The quality of the final preparation was evaluated by:

(a) SDS polyacrylamide gel electrophoresis, performed using commercial gels (Novagen) followed by Coomassie Brilliant Blue staining according to the manufacturer's instructions. The purity as estimated by scanning a digital image of a gel was estimated to be at least 95%.

(b) Mass Spectroscopy: The eluted peak(s) were analysed using ESI-TOF-MS. Mass spectroscopy was performed using a Bruker “BioTOF” electrospray time of flight instrument. Samples were either diluted by a factor of 1000 straight from storage buffer into methanol/water/formic acid (50:48:2 v/v/v), or subjected to reverse phase HPLC separation using a C4 column. Calibration was achieved using Bombesin and angiotensin I using the 2+ and 1+ charged states. Data were acquired between 200 and 2000 m/z range and were subsequently processed using Bruker's X-mass program. Mass accuracy was typically below 1 in 10000.

MS Analysis of BACE WT R56KR57K (SEQ ID NO: 6)

Full-length protein: MASMTGGQQMGRGSMAGVLPAHGT . . .

Predicted mass of full-length protein: 50147

Cleavage position:

MASMTGGQQMGR⇓GSMAGVLPAHGT . . .

Predicted mass of BACE protein: 48911. This is the first intermediate fragment and is obtained very quickly and can be obtained as a stable fragment at lower enzyme concentration.

Cleavage position:

MASMTGGQQMGRGSMAGVLPAHGTQHGIRLPLRSGLGGAPLGLR↓
LPRETDEEP . . .

Predicted mass of BACE protein: 45781. This is the final fragment obtained in the conditions described above. Observed ES-MS spectra of this fragment deconvolutes to a parent mass of 45783. The fragment typically elutes as a single peak from the Mono Q 5.5.

Mass Spec Analysis of BACE N->Q R56KR57K (SEQ ID NO: 12)

Predicted mass of full-length protein: 50895

Cleavage position:

MASMTGGQQMGRGSMAGVLPAHGTQHGIRLPLRSGLGGAPLGLR↓
LPRETDEEP . . .

Predicted mass of BACE protein: 46660.65. This is the final fragment obtained in the conditions described above. Observed ES-MS spectra of this fragment deconvolutes to a parent mass of 46655. The fragment typically elutes as two peaks from the Mono Q 5.5, the first corresponding to the desired fragment.

Mass Spec Analysis of BACE N->Q R56KR57K no His (SEQ ID NO: 14)

Predicted mass of full-length protein: 50072.73

Cleavage position:

MASMTGGQQMGRGSMAGVLPAHGTQHGIRLPLRSGLGGAPLGLR ↓
LPRETDEEP . . .

Predicted mass of BACE protein: 45837.80. This is the first intermediate fragment, obtained rapidly between 30-60 minutes post activation and is suitable for crystallisation. Observed ES-MS spectra of this fragment deconvolutes to a parent mass of 45838.30. Typically elutes as 2 peaks from the Mono Q 5.5, the first peak corresponding to the desired fragment.

MASMTGGQQMGRGSMAGVLPAHGTQHGIRLPLRSGLGGAPLGLRLPRETD
EEPEEPGK ↓ KGSFVEMV . . .

Cleavage position:

Predicted fragment mass: 44230.11. Further digestion beyond 60 minutes promotes the formation of the above fragment, not suitable for crystallisation. Observed ES-MS spectra of this fragment deconvolutes to a parent mass of 44228.03. This typically elutes as peak 2 from the Mono Q 5.5.

Method for Determining Activity of BACE

A fluorimetric assay was used to measure the activity of the refolded proteins. Activity of the BACE enzyme was measured using the fluorescent peptide R-E(EDANS)-E-V-N-L-*D-A-E-F-K(DABCYL)-R-OH (Bachem) as substrate. Assays were carried out in 96-well black, flat-bottomed Cliniplates in a final assay volume of 100 ul. The reaction rate was monitored at room temperature on a Fluoroskan Ascent plate reader with excitation and emission wavelengths of 355 nm and 530 nm respectively.

To determine the pH profile for the enzyme 8 nM BACE was incubated with 10 μM substrate in 50 mM sodium acetate (pH 3.5-5.5) or MES (pH 5.5-6.5) buffers at varying pHs and 5% DMSO.

For kinetic characterization of the enzyme 8 nM BACE enzyme was incubated with varying concentrations of the substrate (2.5-80 μM) in 50 mM sodium acetate, pH 5, 5% DMSO and the reaction monitored as described above. Kinetic parameters were determined by the standard Michaelis-Menten equation, using Prizm (GraphPad) software. 1 mM OM 99 completely inhibits activity.

Protein Crystallisation

The sample of BACE was buffer exchanged into 20 mM Tris.HCl pH8.2, 150 mM NaCl, 1 mM DTT and concentrated down to approximately 7 mg/ml as determined by its theoretical extinction coefficient. Prior to crystallisation, the sample was spun at 55,000 rpm for 30 min using a Beckman benchtop ultracentrifuge. DMSO was added to a final concentration of 3% (v/v).

Crystals of BACE from BACE WT R56KR57K, BACE N->Q R56KR57K & BACE N->Q R56KR57K no His were obtained by the hanging-vapour diffusion method at 20° C. using 1.5 μl of protein and an equivalent volume of reservoir solution. The reservoir solution contained 20-24% PEG 5000 MME, 180-220 mM (e.g. 200 mM) ammonium iodide, 180-220 mM (e.g. 200 mM) tri-sodium citrate (pH 6.4-6.6). In an alternative, the reservoir solution may additionally contain 2.5% v/v glycerol.

Diffraction quality single crystals of BACE WT R56KR57K were obtained by the hanging-vapour diffusion method at 20° C. using 1.5 μl of protein and an equivalent volume of reservoir solution. The reservoir solution contained 20-22.5% PEG 5000 MME, 180-220 mM (e.g. 200 mM) ammonium iodide, 180-220 mM (e.g. 200 mM) tri-sodium citrate (pH 6.4-6.6).

Crystals appear within the first week and grow to maximum dimensions within 14 days. The crystals were hexagonal rods with approximate dimensions of 0.2×0.05×0.05 mm. They belonged to the hexagonal space group P6₁22 with cell parameters a=b=103.2 Å, c=169.1 Å and accommodate one enzyme molecule per asymmetric unit, and a solvent content of 66%.

Inhibitor Soaking

BACE inhibitors were dissolved in DMSO to a concentration of 500 mM and then diluted 1 in 10 in a harvesting solution composed of 220 mM ammonium iodide, 220 mM sodium cacodylate pH 6.4 and 22% PEG 5K MME or 100-200 mM sodium citrate pH 5.0, 200 mM ammonium iodide and 30% PEG 5K MME. Apo-BACE protein crystals were transferred into the harvesting solution for a period of up to 24 hours prior to being dipped in cryoprotectant (20% PEG 5000 MME, 200 mM ammonium iodide, 200 mM sodium cacodylate pH 6.4 and 20% _{(v/v)} glycerol or 200 mM sodium citrate pH 5.0, 200 mM ammonium iodide, 30% PEG 5K MME and 20% _{(v/v)} glycerol) containing the inhibitor and frozen in liquid nitrogen.

Data Collection & Processing

The structure of apo-BACE was solved from BACE WT R56KR57K to 1.75 Å resolution using the method of molecular replacement. Prior to data collection, crystals were exposed, briefly, to cryoprotectant, described previously, before flash freezing. Data was collected at 100°K. on beamline ID14-1 at the European Synchrotron Radiation Facility using an ADSC Quantum4 CCD detector, with a wavelength of 0.934 Å and processed using MOSFLM (Leslie, A. G. W. (1992). In Joint CCP4 and EESF-EACMB Newsletter on Protein Crystallography, vol. 26, Warrington, Daresbury Laboratory). The dataset was scaled using SCALA (CCP4—Collaborative Computational Project 4. (1994) The CCP4 Suite: Programs for Protein Crystallography. Acta Crystallographica D50, 760-763) and the intensities converted to structure factor amplitudes with TRUNCATE (Evans, P. R. (1997). Scaling of MAD data. In Recent Advances in Phasing (ed. K. S. Wilson, G. Davies, A. W. Ashton and S. Bailey), pp. 97-102. Council for the Central Laboratory of the Research Councils Daresbury Laboratory, Daresbury, UK), from the CCP4 suite of programs (CCP4—Collaborative Computational Project 4. (1994) The CCP4 Suite: Programs for Protein Crystallography. Acta Crystallographica D50, 760-763). Statistics for the processing are shown in Table 2.

TABLE 2
Data collection statistics for apo-BACE.

| Resolution | 1.75 Å |
|---|---|
| Mosaicity | 0.34° |
| Completeness | 95.9% |
| Multiplicity | 6.3 |
| Rmerge | 0.097 |

Structure Determination and Refinement

The structure of apo-BACE was solved by molecular replacement using the program EPMR (Kissinger C R, Gehlhaar D K, Fogel D B, Acta Crystallogr D Biol Crystallogr, 1999,vol 55 (Pt 2), 484-91). Initially, it was impossible to know whether the correct space group was P6₁22 or P6₅22, therefore molecular replacement attempts were performed against both. Default parameters and a resolution range of 15-4Å were used in conjunction with the A chain of 1FKN (Hong et al, 2000) as the search model. A solution was found for P6₁22 with an Rfactor of 0.458 and a correlation coefficient of 0.543. In an attempt to reduce model bias, the molecular replacement solution was used as the starting point for ARP/wARP (Morris R J, Perrakis A, Lamzin V S, Acta Crystallogr D Biol Crystallogr, 2002,vol 58,(Pt 6 No 2), 968-75) to perform automated backbone tracing using warpNtrace and side chain building via the Side_dock procedure. This produced a discontinuous model composed of 244 out of 385 residues spanning 12 amino acid chains. Cycles of structural refinement with REFMAC5 (Murshudov, G. N., Vagin, A. A. and Dodson, E. J. (1997). Refinement of macromolecular structures by the maximum-likelihood method. Acta Crystallographica, 1997 D53, 240-255) were alternated with manual rebuilding of the model using QUANTA (Jones et al., Acta Crystallography A47 (1991), 110-119 and commercially available from Accelerys, San Diego, Calif.). The model was extended to 329 residues with chain breaks between 156-170, 255-280 and 311-325. CNX (Brunger et al., Current Opinion in Structural Biology, Vol. 8, Issue 5, October 1998, 606-611, and commercially available from Accelerys, San Diego, Calif.) composite omit maps were generated to allow further building of the structure and finally water molecules added using DenInt (Astex internal software library). Refinement statistics are shown in Table 3.

TABLE 3
Final refinement statistics for apo-BACE

| | |
|---|---|
| Rwork | 0.251 |
| Rfree | 0.284 |
| RMS bond deviation from ideality | 0.011 |
| RMS bond angle deviation from ideality | 1.30 |
| Average Bfactor for structure | 32.99 |

This data indicates that the final structure is of good quality; the Rfactors indicating that the refined model has a good agreement with the experimental data. The RMS deviations from ideality indicate that the geometry of the model is good.

Description of the Apo Structure of BACE

The structure of BACE we present here has been solved in the absence of substrate or inhibitor. This is the first time that such a structure has been described. The solution of this structure has been possible as we have, for the first time, crystallized BACE without compound in a form suitable for diffracting X-rays, and hence allowed the determination of the apo structure of BACE. Under our conditions it crystallizes in space group P6₁22 with a monomer in the asymmetric unit. This is a novel crystal form of BACE.

The protein chain has been traced in the electron density from residue Phe47p to Ala157, and then from Ala168 to Asn385. There is no indication as to the position of residues 158 to 167 in the electron density map. In addition to the protein atoms, the model contains 3 iodine atoms and 285 water molecules in its present state of refinement.

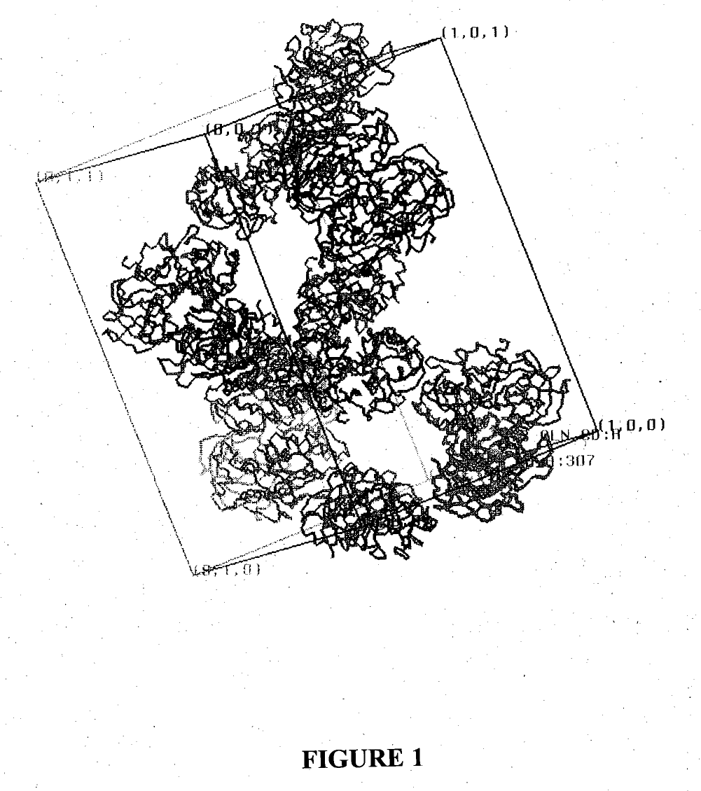

The majority of the residues in this form of BACE are well defined, the exceptions being some exposed residues. Parts of the protein surface are exposed to solvent, as a consequence of the molecular packing within the crystal lattice (FIG. 1). Residues 255-259, 271-277 and 310 to 317 are exposed and have high B-factors relative to the body of the protein. In addition, residues 304 to 309 pack against an exposed loop and are poorly ordered with high b-factors. There are three disulphide bonds in BACE, two of these are well defined in the electron density, the third, between Cys269 and Cys319 has high temperature factors. This is probably a consequence of its proximity to exposed parts of the protein.

BACE as it has been solved in this form, is a compact globular protein, which is formed by two domains; domain 1 being comprised of residues 47p-146 and domain 2 of residues (146-385)(numbering from Hong et al, 2000). The active site lies between these two domains, and contains the two conserved aspartic acid residues, Asp32 and Asp228, which define the active sites of aspartic proteinases. In our structure, a single water molecule is coordinated between these two residues.

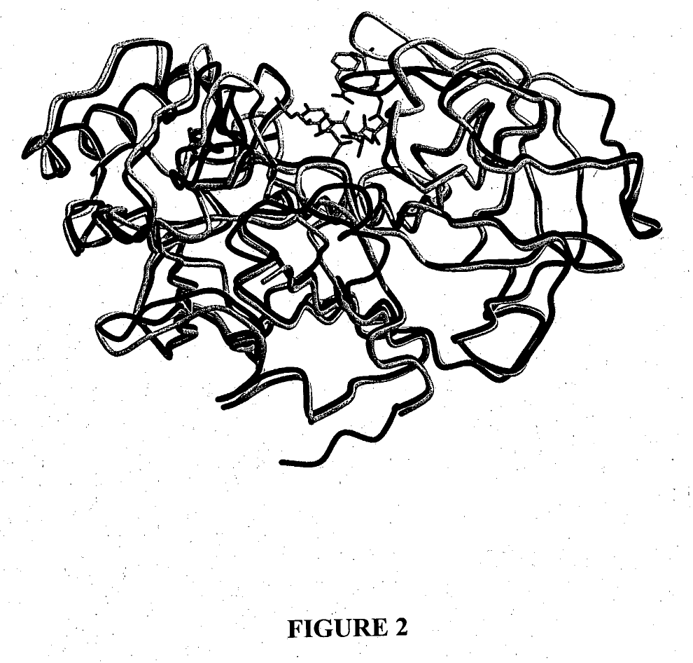

The overall fold of the protein is similar to that of 1FKN (Hong et al, 2000), with a few minor, but potentially significant changes. Residues 158-166 are ordered in the structure of BACE in the presence of OM99-2 (in the P2₁ form), and consist of a loop plus a short helix. In the P6₁22 unliganded form, these residues cannot be seen, and are assumed to be mobile. This may be a consequence of the crystal packing arrangement in this form. Residues 69-75 have a different arrangement in the crystal form described here, to their arrangement in the crystal structure of the OM99-2 complex. The residues are displaced upward relative to the active site in the structure without OM99-2. The two molecules can be superposed over all residues using the program MAPS (MAPS-Multiple Alignment of Proteins Structures Version 0.2, Sep.-7-1999, Guoguang, Lund University, Sweden and Lu, G. An Approach for Multiple Alignment of Protein Structures (1998, in manuscript) to give an r.m.s.d. of 0.74 Å. This results in close alignment of the N-terminal residue prior to residue 69 and subsequent to 75. In contrast the CA atoms of residue 71 are displaced by 3.3 Å, those of residue 72 by 4.3 Å, and those of residue 73 by 6.0 Å. (FIG. 2) The reason for this difference is postulated to be the interaction of OM99-2 backbone residues with the protein residues, in an arrangement analogous to a beta sheet. This interaction pulls the loop down over the substrate in the active site, and locks it in position. In the absence of substrate, or peptidic inhibitor, the loop moves back up again.

In addition to these local changes in structure, on binding of inhibitor, there appears to be a slight shift in the domain positions relative to each other, resulting in an average difference in position in the C-terminal domain CA atoms of about 2.0 Å, when the molecules are superposed using the N-terminal CA atoms.

The symmetry of the P6₁22 crystal system has resulted in a packing arrangement which brings part of a symmetry related molecule very close to the active site entrance of BACE. Gln73 from a symmetry related molecule lies very close to the entrance to the active site of BACE in this crystal form, and overlaps with the position occupied by P4 Glu in OM99-2. However, this does not interfere with the usefulness of this crystal system to soak in inhibitors, as we have shown that these crystals can be used to soak BACE inhibitors into the active site.

### INCORPORATION BY REFERENCE

The entire contents of all patents, published patent applications and other references cited herein are hereby expressly incorporated herein in their entireties by reference. Particular reference is made to the references listed below:

Bennett B D, Denis P, Haniu M, Teplow D B, Kahn S, Louis J C, Citron M, Vassar R. J Biol. Chem. 2000; 275(48):37712-7.

Blundell T L, Jhoti H, Abell C, Nature Reviews Drug Discovery, 1, 45-54, 2002.

Blundell, T L and Johnson, L N, in Protein Crystallography, Academic Press, New York, London and San Francisco, (1976)

Burton S J, Quirk A V, Wood P C, European Journal of Biochemistry, 179, 379-87, 1989.

Carr, R; Jhoti, H; Drug Discov. Today, 2002, 7(9), 522-527

Choppa R, Svenson K, Annis B, Akopian T, Bard J, Stahl M, Somers W; PCT International Publication Number WO 02/25276, (28 Mar. 2002).

Collaborative Computing Project N., 1994 Acta Crystallogr. D. 50 760-763.

Creemers J W, Ines Dominguez D, Plets E, Serneels L, Taylor N A, Multhaup G, Craessaerts K, Annaert W, De Strooper B., J Biol. Chem. (2001) 276(6), 4211-7

De Strooper, B. and Konig. G., 1999, Nature 402, 471-472.

Ghosh A K., Shin D., Downs D., Koelsch G., Lin X., Ernolieff J. and Tang J. (2000) J. Am. Chem. Soc. 122 3522-3523.

Goldberg M E, Expert-Bezancon N, Vuillard L, Rabilloud T, Folding & Design, 1, 21-27, 1996.

Greer J, Erickson J W, Baldwin J J, Varney M D, J. of Medicinal Chemistry, Vol. 37, (1994), 1035-1054,

Haniu M, Denis P, Young Y, Mendiaz E A, Fuller J, Hui J O, Bennett B D, Kahn S, Ross S. Burgess T, Katta V, Rogers G, Vassar R, Citron M., J Biol. Chem. (2000), 275(28), 21099-106.

Hong L., Koelsch G., Lin X., Wu S., Terzyan S., Ghosh A K., Zhang X C. and Tang J. (2000) SCIENCE 290 150-153.

Hussain I, Powell D, Howlett D R, Tew D G, Meek T D, Chapman C, Gloger I S, Murphy K E, Southan C D, Ryan D M, Smith T S, Simmons D L, Walsh F S, Dingwall C, Christie G., Mol. Cell Neurosci. (1999)14(6):419-27

Lin X., Koelsch G., Wu S., Downs D., Dashti A. and Tang J. (2000) PNAS 97 1456-1460.

Mallender W D, Yager D, Onstead L, Nichols M R, Eckman C, Sambamurti K, Kopcho L M, Marcinkeviciene J, Copeland R A, Rosenberry T L., Mol Pharmacol, (2001) Mar;59(3), 619-26

Navaza J., 1994 Acta Crystallogr. A. 50 157

Pflugrath, J W. (1999) Acta Crystallogr. D. 55 1718-1725 Sinha S., Anderson J P., Barbour R., Basi G S., Caccavello R., Davis D., Doan M., Dovey H F., Frigon N., Hong J., Jacobson-Croak K., Jewett N., Keim P., Knops J., Lieberburg I., Power M., Tan H., Tatsuno G., Tung J., Schenk D., Seubert P., Suomensaari S M., Wand S., Walker D., Zhoa J., McConlogue L. and Varghese J. (1999) NATURE 402 537-540

Tang J, Lin X, Koelsch G; PCT International Publication Number WO 01/00663, (04 Jan. 2001).

Vassar R., Bennett, B D., Babu-Khan S., Kahn S., Mendiaz, E A., Denis P., Teplow D B., Ross S., Amarante P., Loeloff R., Luo Y., Fisher S., Fuller J., Edenson S., Lile J., Jorosinski M A., Biere A L., Curran E., Burgess T., Louis J-C., Collins F., Treaner J., Rogers G. and Citron M. (1999) SCIENCE 286 735-741

Yan R., Bienkowski M J., Shuck M E., Miao H., Tory M C., Pauley A M., Brashier J R., Stratman N C., Mathews W R., Buhl A E., Carter D B., Tomasselli A G., Parodi L A., Heinrikson R L. and Gurney, M E. (1999) Nature, 402 533-537

Yankner B A., Nature Medicine, (1996) 2(8), 850-2

### EQUIVALENTS

The foregoing description details presently preferred embodiments of the present invention which are therefore to be considered in all respects as illustrative and not restrictive. Those skilled in the art will recognize, or be able to ascertain, using no more than routine experimentation, many equivalents, modifications and variations to the specific embodiments of the invention described specifically herein. Such equivalents, modifications and variations are intended to be (or are) encompassed in the scope of the following paragraphs:

1. A mutant BACE protein, which protein lacks one or more proteolytic cleavage sites recognized by clostripain (or another protease which recognizes the same cleavage site as clostripain).

2. The protein of paragraph 1 wherein BACE residues R56 and/or R57 (based on numbering of SwissProt P56817) are mutated or deleted.

3. The protein of paragraph 2 wherein R56 or R57 are mutated by the substitution of arginine for lysine.

4. The protein of paragraph 2 wherein R56 and R57 are mutated by the substitution of arginine for lysine.

5. The protein of any one of the preceding paragraphs which comprises BACE residues 56 to 396 (based on numbering of SwissProt P56817).

6. A mutant BACE protein (for example, a mutant BACE protein as defined in any one of the preceding paragraphs) which is truncated at the N-terminal up to and including R42, R45, G55, R56 or R57.

7. The protein of any one of paragraphs 1 to 6 truncated at the C-terminal such that at least residues 454 et seq. are absent.

8. The protein of paragraph 7 truncated at the C-terminal such that at least residues 447 et seq. are absent.

9. The protein of any one of the preceding paragraphs wherein the asparagine residues at positions 153, 172, 223 and 354 are mutated to glutamine residues.

10. The protein of any one of the preceding paragraphs which is un- or deglycolsylated.

11. A mutant BACE protein selected from: (a) SEQ ID 6; (b) SEQ ID 8; (c) SEQ ID 10; (d) SEQ ID 12; (e) SEQ ID 14; (f) SEQ ID 16; (g) SEQ ID 18; (h) SEQ ID 19; (i) SEQ ID 20; Q) SEQ ID21.

12. Nucleic acid encoding the protein of any one of the preceding paragraphs.

13. A vector comprising the nucleic acid of paragraph 12.

14. A host cell comprising the vector of paragraph 13.

15. A process for producing the protein of any one of paragraphs 1 to 11 comprising the steps of: (a) culturing the host cell of paragraph 14 under conditions suitable for expression of the protein; and optionally (b) isolating the expressed recombinant BACE protein.

16. A process for producing refolded recombinant BACE comprising the steps of: (a) solubilising the recombinant BACE; (b) diluting the solubilised BACE into an aqueous buffer containing sulfobetaine (for example at a concentration of 10 to 50 mM); and (c) maintaining the diluted solution at low temperature (for example, 3 to 6° C.) and at high pH (e.g. 9 to 10.5) for at least 2 weeks.

17. The process of paragraph 16 wherein the recombinant BACE is produced according to the process of paragraph 15.

18. Refolded recombinant BACE produced by, or obtainable by, the process of paragraph 16 or paragraph 17.

19. A process for producing a crystal of BACE comprising the step of refolding recombinant BACE protein according to the process of paragraph 16 or paragraph 17.

20. A process for producing a crystal of BACE comprising the step of growing the crystal by vapour diffusion using a reservoir buffer that contains 18-26% PEG 5000 MME (for example, 20-24% PEG 5000 MME, e.g. 20-22.5% PEG 5000 MME), 180-220 mM (e.g. 200 mM) ammonium iodide and 180-22-mM (e.g. 200 mM) tri-sodium citrate (pH 6.4-6.6).

21. The process of paragraph 20 wherein the BACE is recombinant and the process further comprises the preliminary step of refolding the recombinant BACE according to the process of paragraph 16 or paragraph 17.

22. The process of any one of paragraphs 18 to 20 further comprising the step of activating the BACE by clostripain digestion.

23. The process of paragraph 21 wherein the BACE is as defined in any one of paragraphs 1 to 10.

24. A crystal of BACE produced by, or obtainable by, the process of any one of paragraphs 18 to 22.

25. A crystal of BACE having a hexagonal space group P6₁22.

26. The crystal of paragraph 25 having unit cell dimensions of a=b=103.2 Å, c=169.1 Å, α=β60°, γ=120°, and a unit cell variability of 5% in all dimensions.

27. The crystal of paragraph 25 or paragraph 26 which contains one copy of BACE in the asymmetric unit.

28. A crystal of BACE (e.g. a crystal according to any one of paragraphs 24 to 27) having a resolution better than 3 Å.

29. The crystal of paragraph 28 having a resolution better than 2.5Å.

30. The crystal of paragraph 29 having a resolution better than 1.8Å.

31. A crystal of BACE (e.g. a crystal according to any one of paragraphs 24 to 30) comprising a structure defined by all or a portion of the co-ordinates of Table 1.

32. The crystal of paragraph 31 comprising a structure defined by a portion of the coordinates of Table 1 which coordinates relate to: (a) the BACE catalytic domain; and/or (b) a BACE active site; and/or (c) a BACE binding cavity; and/or (d) selected, amino acid residues of a BACE binding cavity located in a protein framework which holds the selected amino acids in a relative spatial configuration which corresponds to the spatial configuration of those amino acids in Table 1; and/or (d) a BACE binding site.

33. The crystal of paragraph 32 wherein the portion of the coordinates of Table 1 comprise (or consist essentially of) those relating to residues SER71, GLY72, LEU91, ASP93, GLY95, SER96, VAL130, PRO131, TYR132, THR133, GLN134, ILE171, ILE179, ILE187, ALA188, ARG189, PRO190, TRP258, TYR259, ASP284, LYS285, ASP289, GLY291, THR292, THR293, ASN294, ARG296 and ARG368 (based on the numbering of SwissProt P56817).

34. The crystal of paragraph 33 wherein the portion of the coordinates of Table 1 comprise (or consist essentially of) those relating to residues LYS70, SER71, GLY72, GLN73, GLY74, TYR75, LEU91, VAL92, ASP93, THR94, GLY95, SER96, SER97, ASN98, TYR129, VAL130, PRO131, TYR132, THR133, GLN134, GLY135, LYS136, TRP137, LYS168, PHE169, PHE170, ILE171, ASN172, SER174, TRP176, GLY178, ILE179, LEU180, GLY181, ALA183, TYR184, ALA185, GLU186, ILE187, ALA188, ARG189, PRO190, ASP191, ASP192, ARG256, TRP258, TYR259, TYR283, ASP284, LYS285, SER286, ILE287, VAL288, ASP289, SER290, GLY291, THR292, THR293, ASN294, LEU295, ARG296, GLY325, GLU326, ARG368, VAL370, LYS382, PHE383, ALA384, ILE385, SER386, GLN387, SER388, SER389, THR390, GLY391, THR392, VAL393, GLY395, ALA396 and ILE447 (based on the numbering of SwissProt P56817).

35. The crystal of any one of paragraphs 24 to 34 which is capable of being soaked with compound(s) to form co-complex structures.

36. The crystal of any one of paragraphs 24 to 35 which is soaked with one or more compound(s) to form co-complex structures.

37. The crystal of any one of paragraphs 24 to 36 wherein the BACE is co-crystallized with one or more compound(s) to form co-crystallized structures.

38. The crystal of any one of paragraphs 24 to 35 which is an apo crystal.

39. The crystal of any one of paragraphs 24 to 38 wherein the BACE is a wild-type BACE.

40. The crystal of paragraph 39 wherein the BACE is a human BACE.

41. The crystal of paragraph 40 wherein the BACE is a homologue of a human BACE.

42. The crystal of paragraph 41 wherein the homologue is an orthologue or a paralogue of a human BACE.

43. The crystal of any one of paragraphs 24 to 38 wherein the BACE is a mutant and/or recombinant BACE.

44. The crystal of paragraph 43 wherein the BACE: (a) lacks the BACE transmembrane and/or cytoplasmic domain(s); and/or (b) lacks one or more glycolsylation sites; and/or (c) comprises one or more peptide tags (for example a his tag); and/or (d) lacks one or more protease cleavage site(s); and/or (e) is truncated at the N-terminus; and/or (f) is truncated at the C-terminus; and/or (f) lacks the BACE pro-sequence.

45. The crystal of paragraph 44 wherein the BACE mutant lacks one or more clostripain cleavage sites.

46. The crystal of paragraph 45 wherein BACE residues R56 and/or R57 (based on numbering of SwissProt P56817) are mutated or deleted.

47. The crystal of paragraph 46 wherein R56 or R57 are mutated by the substitution of arginine for lysine.

48. The crystal of paragraph 46 wherein R56 and R57 are mutated by the substitution of arginine for lysine.

49. The crystal of any one of paragraphs 43 to 48 wherein the BACE mutant is truncated at the N-terminal up to and including R42.

50. The crystal of any one of paragraphs 43 to 49 wherein the BACE mutant is truncated at the C-terminal such that at least residues 396 et seq. are absent.

51. The crystal of paragraph 50 wherein the BACE mutant is truncated at the C-terminal such that at least residues 387 et seq. are absent.

52. The crystal of any one of paragraphs 43 to 51 wherein the asparagine residues at positions 153, 172, 223 and 354 of the BACE mutant are mutated to glutamine residues.

53. The crystal of any one of paragraphs 24 to 52 wherein the BACE is un- or deglycolsylated.

54. The crystal of paragraph 43 wherein the BACE mutant is selected from: (a) SEQ ID 19; (b) SEQ ID 20; (c) SEQ ID 21.

55. The process of any one of paragraphs 19 to 23 wherein the process produces a crystal of BACE as defined in any one of paragraphs 24 to 54.

56. A three-dimensional representation of BACE or of a portion of BACE, which representation comprises all or a portion of the coordinates of Table 1.

57. The three-dimensional representation of paragraph 56 which is a model constructed from all or a portion of the coordinates of Table 1.

58. The model of paragraph 57 wherein the portion of BACE is a BACE binding cavity and the portion of the coordinates of Table 1 comprise those of atoms defining a binding site within the binding cavity (for example, wherein the coordinates are as defined in paragraph 33 or paragraph 34).

59. A three-dimensional representation of a compound which fits the model of paragraph 57 or paragraph 58.

60. The three-dimensional representation of paragraph 59 which is a model of the compound.

61. The model of paragraph 60 wherein the compound is a pharmacophore.

62. The model of any one of paragraphs 57, 58, 60 or 61 which is: (a) a wire-frame model; (b) a chicken-wire model; (c) a ball-and-stick model; (d). a space-filling model; (e) a stick-model; (f) a ribbon model; (g) a snake model; (h) an arrow and cylinder model; (i) an electron density map; (j) a molecular surface model.

63. The model of any one of paragraphs 57, 58, 60, 61 or 62 which is in physical form.

64. The model of any one of paragraphs 57, 58, 60, 61 or 62 which is in electronic form.

65. The model of paragraph 64 which comprises a graphical image display on a computer screen.

66. A computer-based method for the analysis of the interaction of a molecular structure with a BACE structure of the invention, which comprises: (a) providing a BACE model as defined in paragraph 57, 58 or 62 to 65; (b) providing a molecular structure to be fitted to said BACE model; and (c) fitting the molecular structure to the BACE model to produce a compound model as defined in paragraph 60, 61 or 62 to 65.

67. A computer-based method for the analysis of the interaction of a molecular structure with a BACE structure of the invention, which comprises: (a) providing the structure of a BACE as defined by the coordinates of Table 1; (b) providing a molecular structure to be fitted to said BACE structure; and (c) fitting the molecular structure to the BACE structure of Table 1.

68. A computer-based method for the analysis of molecular structures which comprises: (a) providing the coordinates of at least two atoms of a BACE structure as defined in Table 1 (“selected coordinates”); (b) providing the structure of a molecular structure to be fitted to the selected coordinates; and (c) fitting the structure to the selected coordinates of the BACE structure.

69. The method of paragraph 68 wherein the selected coordinates represent a binding pocket.

70. The method of paragraph 68 or paragraph 69 wherein the selected coordinates are of at least 5, 10, 50 or 100 atoms.

71. The method of paragraph 69 or paragraph 70 wherein the selected coordinates are as defined in paragraph 33 or paragraph 34.

72. A computer-based method of rational drug design comprising the method of any one of paragraphs 66 to 71.

73. A computer-based method of rational drug design comprising comprising: (a) providing the coordinates of at least two atoms of a BACE structure as defined in Table 1 (“selected coordinates”); (b) providing the structures of a plurality of molecular fragments; (c) fitting the structure of each of the molecular fragments to the selected coordinates; and (d) assembling the molecular fragments into a single molecule to form a candidate modulator molecule.

74. A method for identifying a candidate modulator (e.g. candidate inhibitor) of BACE comprising the steps of: (a) employing a three-dimensional structure of BACE, at least one sub-domain thereof, or a plurality of atoms thereof, to characterise at least one BACE binding cavity, the three-dimensional structure being defined by atomic coordinate data according to Table 1; and (b) identifying the candidate modulator by designing or selecting a compound for interaction with the binding cavity.

75. The method of paragraph 74 wherein the three-dimensional structure of BACE is a model as defined in paragraph 57 or paragraph 58.

76. A method for identifying an agent compound (e.g. an inhibitor) which modulates BACE activity, comprising the steps of: (a) employing three-dimensional atomic coordinate data according to Table 1 to characterise at least one (e.g. a plurality of) BACE binding site(s); (b) providing the structure of a candidate agent compound; (c) fitting the candidate agent compound to the binding sites; and (d) selecting the candidate agent compound.

77. The method of paragraph 76 wherein in step (a) the three-dimensional atomic coordinate data are employed to create a model as defined in paragraph 57, 58 or 62 to 65.

78. The method of any one of paragraphs 73 to 77further comprising the step of: (a) obtaining or synthesising the candidate agent or modulator; and (b) contacting the candidate modulator with BACE to determine the ability of the candidate modulator to interact with BACE.

79. A method of assessing the ability of a candidate modulator to interact with BACE which comprises the steps of: (a) obtaining or synthesising said candidate modulator; (b) forming a crystallized complex of BACE and said candidate modulator; and (c) analysing said complex by X-ray crystallography or NMR spectroscopy to determine the ability of said candidate modulator to interact with BACE.

80. A method for determining the structure of a compound bound to BACE, said method comprising: (a) mixing BACE with the compound to form a BACE-compound complex; (b) crystallizing the BACE-compound complex; and (c) determining the structure of said BACE-compound(s) complex by reference to the data of Table 1.

81. A method for determining the structure of a compound bound to BACE, said method comprising: (a) providing a crystal of BACE; (b) soaking the crystal with one or more compound(s) to form a complex; and (c) determining the structure of the complex by employing the data of Table 1.

82. A method of determining the three dimensional structure of a BACE homologue or analogue of unknown structure, the method comprising the steps of: (a) aligning a representation of an amino acid sequence of the BACE homologue or analogue with the amino acid sequence of the BACE of Table 1 to match homologous regions of the amino acid sequences; (b) modelling the structure of the matched homologous regions of said target BACE of unknown structure on the corresponding regions of the BACE structure as defined by Table 1; and (c) determining a conformation for the BACE homologue or analogue which substantially preserves the structure of said matched homologous regions.

83. The method of paragraph 82 wherein steps (a) and/or (b) and/or (c) are performed by computer modelling.

84. A method of providing data for generating structures and/or performing rational drug design for BACE, BACE homologues or analogues, complexes of BACE with a potential modulator, or complexes of BACE homologues or analogues with potential modulators, the method comprising: (i) establishing communication with a remote device containing computer-readable data comprising at least one of: (a) atomic coordinate data according to Table 1, said data defining the three-dimensional structure of BACE, at least one sub-domain of the three-dimensional structure of BACE, or the coordinates of a plurality of atoms of BACE; (b) structure factor data for BACE, said structure factor data being derivable from the atomic coordinate data of Table 1; (c) atomic coordinate data of a target BACE homologue or analogue generated by homology modelling of the target based on the data of Table 1; (d) atomic coordinate data of a protein generated by interpreting X-ray crystallographic data or NMR data by reference to the data of Table 1; and (e) structure factor data derivable from the atomic coordinate data of (c) or (d); and (ii) receiving said computer-readable data from said remote device.

85. A computer system containing one or more of: (a) atomic coordinate data according to Table 1, said data defining the three-dimensional structure of BACE or at least selected coordinates thereof, (b) structure factor data (where a structure factor comprises the amplitude and phase of the diffracted wave) for BACE, said structure factor data being derivable from the atomic coordinate data of Table 1; (c) atomic coordinate data of a target BACE protein generated by homology modelling of the target based on the data of Table 1; (d) atomic coordinate data of a target BACE protein generated by interpreting X-ray crystallographic data or NMR data by reference to the data of Table 1; or (e) structure factor data derivable from the atomic coordinate data of (c) or (d).

86. The computer system of paragraph 85 comprising: (i) a computer-readable data storage medium comprising data storage material encoded with the computer-readable data; (ii) a working memory for storing instructions for processing said computer-readable data; and (iii) a central-processing unit coupled to said working memory and to said computer-readable data storage medium for processing said computer-readable data and thereby generating structures and/or performing rational drug design.

87. The computer system of paragraph 86 further comprising a display coupled to said central-processing unit for displaying said structures.

88. A computer-readable storage medium, comprising a data storage material encoded with computer readable data, wherein the data are defined by all or a portion of the structure coordinates of BACE of Table 1, or a homologue of BACE, wherein said homologue comprises backbone atoms that have a root mean square deviation from the backbone atoms (nitrogen-carbon_{α}carbon) of Table 1 of not more than 1.5 Å.

89. A computer-readable data storage medium comprising a data storage material encoded with a first set of computer-readable data comprising a Fourier transform of at least a portion (e.g. selected coordinates as defined herein) of the structural coordinates for BACE according to Table 1; which, when combined with a second set of machine readable data comprising an X-ray diffraction pattern of a molecule or molecular complex of unknown structure, using a machine programmed with the instructions for using said first set of data and said second set of data, can determine at least a portion of the structure coordinates corresponding to the second set of machine readable data.

90. A computer readable medium with at least one of: (a) atomic coordinate data according to Table 1 recorded thereon, said data defining the three-dimensional structure of BACE, or at least selected coordinates thereof; (b) structure factor data for BACE recorded thereon, the structure factor data being derivable from the atomic coordinate data of Table 1; (c) atomic coordinate data of a target BACE protein generated by homology modelling of the target based on the data of Table 1; (d) atomic coordinate data of a BACE-ligand complex or a BACE homologue or analogue generated by interpreting X-ray crystallographic data or NMR data by reference to the data of Table 1; and (e) structure factor data derivable from the atomic coordinate data of (c) or (d).

91. A method for determining the structure of a protein, which method comprises;

providing the co-ordinates of Table 1, and either (a) positioning the co-ordinates in the crystal unit cell of said protein so as to provide a structure for said protein or (b) assigning NMR spectra Peaks of said protein by manipulating the coordinates of Table 1.

92. A process for producing a medicament, pharmaceutical composition or drug, the process comprising: (a) identifying a BACE modulator molecule according to the method as defined in any one of paragraphs 73 to 79; (b) optimising the structure of the modulator molecule; and (c) preparing a medicament, pharmaceutical composition or drug containing the optimised modulator molecule.

93. A medicament, pharmaceutical composition or drug produced by, or obtainable by, the process of paragraph 92.

94. A compound identified, produced or obtainable by the process or method of any one of paragraphs 73 to 79.

95. A pharmaceutical composition, medicament, drug or other composition comprising the compound of paragraph 94.

96. The medicament, pharmaceutical composition or drug of paragraph 93, compound of paragraph 94 or composition of paragraph 95 for use in medicine, for example for use in therapy or prophylaxis.

97. The medicament, pharmaceutical composition, drug or composition of paragraph 96 wherein the therapy or prophylaxis comprises inhibiting BACE or the production of Aβ or fragments thereof or the treatment of Alzheimer's disease.

98. A method of inhibiting BACE or the production of Aβ or fragments thereof or treating Alzheimer's disease comprising administering the miedicament, pharmaceutical composition, drug or composition of paragraph 96 to the patient.

99. The method of paragraph 84, wherein the computer readable data is transmitted form the remove device.

100. The method of paragraph 99, wherein the data is transmitted electronically or optically.

TABLE 1

| | | | | | | | | | | | | |
|---|---|---|---|---|---|---|---|---|---|---|---|---|
| ATOM | 1 | N | PHE | A | 47p | 65.730 | 61.598 | −17.857 | 1.00 | 56.68 | A | N |
| ATOM | 2 | CA | PHE | A | 47p | 66.426 | 61.383 | −16.552 | 1.00 | 54.16 | A | C |
| ATOM | 3 | C | PHE | A | 47p | 67.801 | 60.738 | −16.734 | 1.00 | 54.30 | A | C |
| ATOM | 4 | O | PHE | A | 47p | 68.258 | 59.983 | −15.869 | 1.00 | 52.46 | A | O |
| ATOM | 5 | CB | PHE | A | 47p | 65.566 | 60.500 | −15.635 | 1.00 | 54.61 | A | C |
| ATOM | 6 | CG | PHE | A | 47p | 64.161 | 61.008 | −15.429 | 1.00 | 54.65 | A | C |
| ATOM | 7 | CD1 | PHE | A | 47p | 63.110 | 60.508 | −16.186 | 1.00 | 56.27 | A | C |
| ATOM | 8 | CD2 | PHE | A | 47p | 63.887 | 61.970 | −14.463 | 1.00 | 55.01 | A | C |
| ATOM | 9 | CE1 | PHE | A | 47p | 61.812 | 60.972 | −15.995 | 1.00 | 57.39 | A | C |
| ATOM | 10 | CE2 | PHE | A | 47p | 62.596 | 62.435 | −14.266 | 1.00 | 56.06 | A | C |
| ATOM | 11 | CZ | PHE | A | 47p | 61.556 | 61.938 | −15.035 | 1.00 | 56.47 | A | C |
| ATOM | 12 | N | VAL | A | 48p | 68.468 | 61.048 | −17.845 | 1.00 | 54.26 | A | N |
| ATOM | 13 | CA | VAL | A | 48p | 69.737 | 60.395 | −18.200 | 1.00 | 54.45 | A | C |
| ATOM | 14 | C | VAL | A | 48p | 70.910 | 60.742 | −17.276 | 1.00 | 53.21 | A | C |
| ATOM | 15 | O | VAL | A | 48p | 71.847 | 59.947 | −17.128 | 1.00 | 56.35 | A | O |
| ATOM | 16 | CB | VAL | A | 48p | 70.156 | 60.691 | −19.662 | 1.00 | 57.43 | A | C |
| ATOM | 17 | CG1 | VAL | A | 48p | 69.222 | 59.972 | −20.636 | 1.00 | 58.42 | A | C |
| ATOM | 18 | CG2 | VAL | A | 48p | 70.204 | 62.208 | −19.944 | 1.00 | 57.43 | A | C |
| ATOM | 19 | N | GLU | A | 1 | 70.860 | 61.925 | −16.668 | 1.00 | 49.17 | A | N |
| ATOM | 20 | CA | GLU | A | 1 | 71.845 | 62.329 | −15.674 | 1.00 | 46.84 | A | C |
| ATOM | 21 | C | GLU | A | 1 | 71.857 | 61.373 | −14.479 | 1.00 | 42.66 | A | C |
| ATOM | 22 | O | GLU | A | 1 | 72.901 | 61.125 | −13.891 | 1.00 | 45.10 | A | O |
| ATOM | 23 | CB | GLU | A | 1 | 71.532 | 63.740 | −15.171 | 1.00 | 48.32 | A | C |
| ATOM | 24 | CG | GLU | A | 1 | 70.180 | 64.053 | −14.545 | 0.00 | 50.15 | A | C |
| ATOM | 25 | CD | GLU | A | 1 | 68.942 | 64.394 | −15.351 | 0.00 | 51.10 | A | C |
| ATOM | 26 | OE1 | GLU | A | 1 | 68.516 | 63.562 | −16.178 | 0.00 | 51.29 | A | O |
| ATOM | 27 | OE2 | GLU | A | 1 | 68.395 | 65.500 | −15.155 | 0.00 | 51.61 | A | O |
| ATOM | 28 | N | MET | A | 2 | 70.685 | 60.855 | −14.125 | 1.00 | 37.18 | A | N |
| ATOM | 29 | CA | MET | A | 2 | 70.525 | 60.001 | −12.942 | 1.00 | 32.72 | A | C |
| ATOM | 30 | C | MET | A | 2 | 70.875 | 58.531 | −13.154 | 1.00 | 29.50 | A | C |
| ATOM | 31 | O | MET | A | 2 | 71.014 | 57.787 | −12.183 | 1.00 | 29.19 | A | O |
| ATOM | 32 | CB | MET | A | 2 | 69.099 | 60.111 | −12.415 | 1.00 | 30.14 | A | C |
| ATOM | 33 | CG | MET | A | 2 | 68.733 | 61.514 | −12.005 | 1.00 | 34.84 | A | C |
| ATOM | 34 | SD | MET | A | 2 | 67.103 | 61.723 | −11.322 | 1.00 | 36.26 | A | S |
| ATOM | 35 | CE | MET | A | 2 | 66.607 | 63.243 | −12.134 | 1.00 | 40.05 | A | C |
| ATOM | 36 | N | VAL | A | 3 | 71.008 | 58.079 | −14.396 | 1.00 | 28.21 | A | N |
| ATOM | 37 | CA | VAL | A | 3 | 71.291 | 56.669 | −14.611 | 1.00 | 29.18 | A | C |
| ATOM | 38 | C | VAL | A | 3 | 72.690 | 56.364 | −14.085 | 1.00 | 27.28 | A | C |
| ATOM | 39 | O | VAL | A | 3 | 73.622 | 57.149 | −14.298 | 1.00 | 28.33 | A | O |
| ATOM | 40 | CB | VAL | A | 3 | 71.137 | 56.248 | −16.094 | 1.00 | 32.19 | A | C |
| ATOM | 41 | CG1 | VAL | A | 3 | 71.649 | 54.826 | −16.299 | 1.00 | 30.92 | A | C |
| ATOM | 42 | CG2 | VAL | A | 3 | 69.667 | 56.353 | −16.525 | 1.00 | 32.71 | A | C |
| ATOM | 43 | N | ASP | A | 4 | 72.803 | 55.254 | −13.359 | 1.00 | 28.19 | A | N |
| ATOM | 44 | CA | ASP | A | 4 | 74.066 | 54.739 | −12.825 | 1.00 | 29.50 | A | C |
| ATOM | 45 | C | ASP | A | 4 | 74.600 | 55.632 | −11.703 | 1.00 | 27.86 | A | C |
| ATOM | 46 | O | ASP | A | 4 | 75.797 | 55.682 | −11.454 | 1.00 | 28.77 | A | O |
| ATOM | 47 | CB | ASP | A | 4 | 75.107 | 54.575 | −13.940 | 1.00 | 32.06 | A | C |
| ATOM | 48 | CG | ASP | A | 4 | 76.254 | 53.655 | −13.553 | 1.00 | 37.52 | A | C |
| ATOM | 49 | OD1 | ASP | A | 4 | 76.029 | 52.572 | −12.945 | 1.00 | 38.24 | A | O |
| ATOM | 50 | OD2 | ASP | A | 4 | 77.438 | 53.952 | −13.829 | 1.00 | 45.15 | A | O |
| ATOM | 51 | N | ASN | A | 5 | 73.694 | 56.308 | −11.015 | 1.00 | 24.98 | A | N |
| ATOM | 52 | CA | ASN | A | 5 | 74.062 | 57.172 | −9.876 | 1.00 | 18.95 | A | C |
| ATOM | 53 | C | ASN | A | 5 | 74.270 | 56.415 | −8.544 | 1.00 | 22.40 | A | C |
| ATOM | 54 | O | ASN | A | 5 | 74.564 | 57.045 | −7.515 | 1.00 | 21.31 | A | O |
| ATOM | 55 | CB | ASN | A | 5 | 73.064 | 58.329 | −9.718 | 1.00 | 21.03 | A | C |
| ATOM | 56 | CG | ASN | A | 5 | 71.677 | 57.870 | −9.366 | 1.00 | 16.73 | A | C |
| ATOM | 57 | OD1 | ASN | A | 5 | 71.424 | 56.673 | −9.325 | 1.00 | 19.74 | A | O |
| ATOM | 58 | ND2 | ASN | A | 5 | 70.801 | 58.808 | −9.035 | 1.00 | 21.06 | A | N |
| ATOM | 59 | N | LEU | A | 6 | 74.099 | 55.098 | −8.562 | 1.00 | 15.94 | A | N |
| ATOM | 60 | CA | LEU | A | 6 | 74.323 | 54.236 | −7.397 | 1.00 | 16.57 | A | C |
| ATOM | 61 | C | LEU | A | 6 | 75.531 | 53.321 | −7.510 | 1.00 | 21.72 | A | C |
| ATOM | 62 | O | LEU | A | 6 | 75.855 | 52.780 | −8.581 | 1.00 | 21.55 | A | O |
| ATOM | 63 | CB | LEU | A | 6 | 73.109 | 53.352 | −7.078 | 1.00 | 18.17 | A | C |
| ATOM | 64 | CG | LEU | A | 6 | 71.707 | 53.957 | −6.866 | 1.00 | 19.32 | A | C |
| ATOM | 65 | CD1 | LEU | A | 6 | 70.695 | 52.916 | −6.521 | 1.00 | 17.46 | A | C |
| ATOM | 66 | CD2 | LEU | A | 6 | 71.748 | 54.997 | −5.797 | 1.00 | 21.42 | A | C |
| ATOM | 67 | N | ARG | A | 7 | 76.173 | 53.126 | −6.364 | 1.00 | 21.10 | A | N |
| ATOM | 68 | CA | ARG | A | 7 | 77.333 | 52.266 | −6.230 | 1.00 | 23.84 | A | C |
| ATOM | 69 | C | ARG | A | 7 | 77.237 | 51.485 | −4.939 | 1.00 | 25.78 | A | C |
| ATOM | 70 | O | ARG | A | 7 | 76.424 | 51.808 | −4.059 | 1.00 | 21.54 | A | O |
| ATOM | 71 | CB | ARG | A | 7 | 78.610 | 53.103 | −6.226 | 1.00 | 26.25 | A | C |
| ATOM | 72 | CG | ARG | A | 7 | 78.992 | 53.658 | −7.583 | 1.00 | 30.55 | A | C |
| ATOM | 73 | CD | ARG | A | 7 | 80.135 | 54.652 | −7.549 | 1.00 | 37.65 | A | C |
| ATOM | 74 | NE | ARG | A | 7 | 80.063 | 55.407 | −8.932 | 0.00 | 40.50 | A | N |
| ATOM | 75 | CZ | ARG | A | 7 | 80.997 | 56.306 | −9.222 | 0.00 | 41.92 | A | C |
| ATOM | 76 | NH1 | ARG | A | 7 | 80.991 | 56.911 | −10.402 | 0.00 | 42.93 | A | N |
| ATOM | 77 | NH2 | ARG | A | 7 | 81.937 | 56.601 | −8.335 | 0.00 | 42.80 | A | N |
| ATOM | 78 | N | GLY | A | 8 | 78.091 | 50.479 | −4.799 | 1.00 | 26.16 | A | N |
| ATOM | 79 | CA | GLY | A | 8 | 78.086 | 49.663 | −3.598 | 1.00 | 29.54 | A | C |
| ATOM | 80 | C | GLY | A | 8 | 79.032 | 48.490 | −3.639 | 1.00 | 31.18 | A | C |
| ATOM | 81 | O | GLY | A | 8 | 79.790 | 48.325 | −4.591 | 1.00 | 33.68 | A | O |
| ATOM | 82 | N | LYS | A | 9 | 78.986 | 47.685 | −2.587 | 1.00 | 34.88 | A | N |
| ATOM | 83 | CA | LYS | A | 9 | 79.643 | 48.390 | −2.578 | 1.00 | 36.27 | A | C |
| ATOM | 84 | C | LYS | A | 9 | 78.625 | 45.337 | −2.169 | 1.00 | 37.50 | A | C |
| ATOM | 85 | O | LYS | A | 9 | 77.771 | 45.576 | −1.316 | 1.00 | 32.87 | A | O |
| ATOM | 86 | CB | LYS | A | 9 | 80.861 | 46.396 | −1.649 | 1.00 | 39.66 | A | C |
| ATOM | 87 | CG | LYS | A | 9 | 81.975 | 47.324 | −2.120 | 1.00 | 45.29 | A | C |
| ATOM | 88 | CD | LYS | A | 9 | 83.346 | 46.635 | −2.207 | 1.00 | 50.21 | A | C |
| ATOM | 89 | CE | LYS | A | 9 | 84.382 | 47.543 | −2.887 | 1.00 | 52.01 | A | C |
| ATOM | 90 | NZ | LYS | A | 9 | 85.408 | 48.085 | −1.943 | 1.00 | 53.23 | A | N |
| ATOM | 91 | N | SER | A | 10 | 78.708 | 44.172 | −2.805 | 1.00 | 38.65 | A | N |
| ATOM | 92 | CA | SER | A | 10 | 77.807 | 43.063 | −2.525 | 1.00 | 39.77 | A | C |
| ATOM | 93 | C | SER | A | 10 | 77.658 | 42.852 | −1.026 | 1.00 | 38.92 | A | C |
| ATOM | 94 | O | SER | A | 10 | 78.658 | 42.718 | −0.316 | 1.00 | 38.89 | A | O |
| ATOM | 95 | CB | SER | A | 10 | 78.336 | 41.776 | −3.172 | 1.00 | 41.88 | A | C |
| ATOM | 96 | OG | SER | A | 10 | 77.485 | 40.680 | −2.879 | 1.00 | 44.59 | A | O |
| ATOM | 97 | N | GLY | A | 11 | 76.410 | 42.857 | −0.556 | 1.00 | 36.41 | A | N |
| ATOM | 98 | CA | GLY | A | 11 | 76.097 | 42.627 | 0.843 | 1.00 | 35.71 | A | C |
| ATOM | 99 | C | GLY | A | 11 | 76.076 | 43.859 | 1.738 | 1.00 | 35.38 | A | C |
| ATOM | 100 | O | GLY | A | 11 | 75.631 | 43.757 | 2.886 | 1.00 | 37.81 | A | O |
| ATOM | 101 | N | GLN | A | 12 | 76.519 | 45.005 | 1.213 | 1.00 | 34.18 | A | N |
| ATOM | 102 | CA | GLN | A | 12 | 76.732 | 46.234 | 1.999 | 1.00 | 35.64 | A | C |
| ATOM | 103 | C | GLN | A | 12 | 75.861 | 47.409 | 1.536 | 1.00 | 35.07 | A | C |
| ATOM | 104 | O | GLN | A | 12 | 76.148 | 48.558 | 1.881 | 1.00 | 36.40 | A | O |
| ATOM | 105 | CB | GLN | A | 12 | 78.196 | 46.693 | 1.913 | 1.00 | 37.52 | A | C |
| ATOM | 106 | CG | GLN | A | 12 | 79.230 | 45.703 | 2.437 | 1.00 | 42.55 | A | C |
| ATOM | 107 | CD | GLN | A | 12 | 80.653 | 46.267 | 2.465 | 1.00 | 40.98 | A | C |
| ATOM | 108 | OE1 | GLN | A | 12 | 81.562 | 45.623 | 2.984 | 1.00 | 49.77 | A | O |
| ATOM | 109 | NE2 | GLN | A | 12 | 80.846 | 47.450 | 1.904 | 1.00 | 50.11 | A | N |
| ATOM | 110 | N | GLY | A | 13 | 74.824 | 47.132 | 0.749 | 1.00 | 30.97 | A | N |
| ATOM | 111 | CA | GLY | A | 13 | 73.887 | 48.163 | 0.331 | 1.00 | 27.55 | A | C |
| ATOM | 112 | C | GLY | A | 13 | 74.366 | 49.021 | −0.820 | 1.00 | 25.65 | A | C |
| ATOM | 113 | O | GLY | A | 13 | 75.491 | 48.904 | −1.289 | 1.00 | 26.10 | A | O |
| ATOM | 114 | N | TYR | A | 14 | 73.477 | 49.892 | −1.275 | 1.00 | 17.01 | A | N |
| ATOM | 115 | CA | TYR | A | 14 | 73.738 | 50.794 | −2.395 | 1.00 | 17.38 | A | C |
| ATOM | 116 | C | TYR | A | 14 | 73.722 | 52.218 | −1.880 | 1.00 | 16.80 | A | C |
| ATOM | 117 | O | TYR | A | 14 | 72.851 | 52.561 | −1.072 | 1.00 | 17.47 | A | O |
| ATOM | 118 | CB | TYR | A | 14 | 72.635 | 50.663 | −3.446 | 1.00 | 18.29 | A | C |
| ATOM | 119 | CG | TYR | A | 14 | 72.651 | 49.339 | −4.162 | 1.00 | 21.45 | A | C |
| ATOM | 120 | CD1 | TYR | A | 14 | 72.134 | 48.194 | −3.574 | 1.00 | 20.72 | A | C |
| ATOM | 121 | CD2 | TYR | A | 14 | 73.201 | 49.239 | −5.434 | 1.00 | 21.04 | A | C |
| ATOM | 122 | CE1 | TYR | A | 14 | 72.164 | 46.981 | −4.246 | 1.00 | 20.87 | A | C |
| ATOM | 123 | CE2 | TYR | A | 14 | 73.233 | 48.043 | −6.101 | 1.00 | 23.36 | A | C |
| ATOM | 124 | CZ | TYR | A | 14 | 72.723 | 46.935 | −5.522 | 1.00 | 24.50 | A | C |
| ATOM | 125 | OH | TYR | A | 14 | 72.758 | 45.757 | −6.229 | 1.00 | 27.32 | A | O |
| ATOM | 126 | N | TYR | A | 15 | 74.636 | 53.044 | −2.387 | 1.00 | 18.15 | A | N |
| ATOM | 127 | CA | TYR | A | 15 | 74.727 | 54.431 | −1.976 | 1.00 | 15.54 | A | C |
| ATOM | 128 | C | TYR | A | 15 | 74.734 | 55.415 | −3.133 | 1.00 | 16.89 | A | C |
| ATOM | 129 | O | TYR | A | 15 | 75.171 | 55.108 | −4.243 | 1.00 | 17.87 | A | O |
| ATOM | 130 | CB | TYR | A | 15 | 75.951 | 54.666 | −1.064 | 1.00 | 16.46 | A | C |
| ATOM | 131 | CG | TYR | A | 15 | 77.308 | 54.342 | −1.685 | 1.00 | 15.58 | A | C |
| ATOM | 132 | CD1 | TYR | A | 15 | 77.966 | 55.246 | −2.501 | 1.00 | 19.48 | A | C |
| ATOM | 133 | CD2 | TYR | A | 15 | 77.919 | 53.139 | −1.411 | 1.00 | 19.60 | A | C |
| ATOM | 134 | CE1 | TYR | A | 15 | 79.201 | 54.956 | −3.034 | 1.00 | 21.95 | A | C |
| ATOM | 135 | CE2 | TYR | A | 15 | 79.165 | 52.838 | −1.926 | 1.00 | 23.26 | A | C |
| ATOM | 136 | CZ | TYR | A | 15 | 79.787 | 53.734 | −2.739 | 1.00 | 21.80 | A | C |
| ATOM | 137 | OH | TYR | A | 15 | 81.006 | 53.396 | −3.255 | 1.00 | 26.17 | A | O |
| ATOM | 138 | N | VAL | A | 16 | 74.279 | 56.620 | −2.823 | 1.00 | 17.50 | A | N |
| ATOM | 139 | CA | VAL | A | 16 | 74.197 | 57.728 | −3.760 | 1.00 | 19.34 | A | C |
| ATOM | 140 | C | VAL | A | 16 | 75.077 | 58.862 | −3.212 | 1.00 | 20.35 | A | C |
| ATOM | 141 | O | VAL | A | 16 | 75.165 | 59.056 | −1.995 | 1.00 | 29.27 | A | O |
| ATOM | 142 | CB | VAL | A | 16 | 72.715 | 58.201 | −3.936 | 1.00 | 18.58 | A | C |
| ATOM | 143 | CG1 | VAL | A | 16 | 72.177 | 58.911 | −2.680 | 1.00 | 18.67 | A | C |
| ATOM | 144 | CG2 | VAL | A | 16 | 72.554 | 59.101 | −5.172 | 1.00 | 21.03 | A | C |
| ATOM | 145 | N | GLU | A | 17 | 75.715 | 59.608 | −4.101 | 1.00 | 20.07 | A | N |
| ATOM | 146 | CA | GLU | A | 17 | 76.401 | 60.838 | −3.706 | 1.00 | 22.44 | A | C |
| ATOM | 147 | C | GLU | A | 17 | 75.398 | 61.943 | −3.372 | 1.00 | 22.83 | A | C |
| ATOM | 148 | O | GLU | A | 17 | 74.419 | 62.145 | −4.091 | 1.00 | 20.94 | A | O |
| ATOM | 149 | CB | GLU | A | 17 | 77.360 | 61.298 | −4.810 | 1.00 | 23.72 | A | C |
| ATOM | 150 | CG | GLU | A | 17 | 78.246 | 62.482 | −4.416 | 1.00 | 28.53 | A | C |
| ATOM | 151 | CD | GLU | A | 17 | 79.065 | 63.024 | −5.580 | 1.00 | 36.53 | A | C |
| ATOM | 152 | OE1 | GLU | A | 17 | 78.956 | 64.228 | −5.878 | 1.00 | 39.02 | A | O |
| ATOM | 153 | OE2 | GLU | A | 17 | 79.820 | 62.249 | −6.201 | 1.00 | 41.99 | A | O |
| ATOM | 154 | N | MET | A | 18 | 75.616 | 62.632 | −2.249 | 1.00 | 18.64 | A | N |
| ATOM | 155 | CA | MET | A | 18 | 74.824 | 63.788 | −1.849 | 1.00 | 18.78 | A | C |
| ATOM | 156 | C | MET | A | 18 | 75.744 | 64.904 | −1.365 | 1.00 | 24.24 | A | C |
| ATOM | 157 | O | MET | A | 18 | 76.919 | 64.671 | −1.079 | 1.00 | 23.12 | A | O |
| ATOM | 158 | CB | MET | A | 18 | 73.866 | 63.427 | −0.717 | 1.00 | 20.09 | A | C |
| ATOM | 159 | CG | MET | A | 18 | 72.884 | 62.284 | −1.064 | 1.00 | 17.91 | A | C |
| ATOM | 160 | SD | MET | A | 18 | 71.685 | 61.911 | 0.240 | 1.00 | 20.92 | A | S |
| ATOM | 161 | CE | MET | A | 18 | 70.491 | 63.197 | −0.005 | 1.00 | 21.35 | A | C |
| ATOM | 162 | N | THR | A | 19 | 75.229 | 66.121 | −1.313 | 1.00 | 24.86 | A | N |
| ATOM | 163 | CA | THR | A | 19 | 75.966 | 67.206 | −0.661 | 1.00 | 26.57 | A | C |
| ATOM | 164 | C | THR | A | 19 | 75.122 | 67.794 | 0.443 | 1.00 | 24.45 | A | C |
| ATOM | 165 | O | THR | A | 19 | 73.904 | 67.861 | 0.341 | 1.00 | 23.60 | A | O |
| ATOM | 166 | CB | THR | A | 19 | 76.392 | 68.292 | −1.665 | 1.00 | 28.59 | A | C |
| ATOM | 167 | OG1 | THR | A | 19 | 75.236 | 68.833 | −2.311 | 1.00 | 32.78 | A | O |
| ATOM | 168 | CG2 | THR | A | 19 | 77.235 | 67.712 | −2.775 | 1.00 | 28.11 | A | C |
| ATOM | 169 | N | VAL | A | 20 | 75.775 | 68.213 | 1.531 | 1.00 | 25.61 | A | N |
| ATOM | 170 | CA | VAL | A | 20 | 75.078 | 68.836 | 2.643 | 1.00 | 22.00 | A | C |
| ATOM | 171 | C | VAL | A | 20 | 75.826 | 70.130 | 2.995 | 1.00 | 21.90 | A | C |
| ATOM | 172 | O | VAL | A | 20 | 77.040 | 70.183 | 2.841 | 1.00 | 23.44 | A | O |
| ATOM | 173 | CB | VAL | A | 20 | 75.011 | 67.902 | 3.848 | 1.00 | 25.28 | A | C |
| ATOM | 174 | CG1 | VAL | A | 20 | 74.361 | 68.579 | 5.033 | 1.00 | 30.83 | A | C |
| ATOM | 175 | CG2 | VAL | A | 20 | 74.245 | 66.611 | 3.495 | 1.00 | 25.14 | A | C |
| ATOM | 176 | N | GLY | A | 21 | 75.077 | 71.146 | 3.422 | 1.00 | 25.14 | A | N |
| ATOM | 177 | CA | GLY | A | 21 | 75.623 | 72.434 | 3.837 | 1.00 | 27.79 | A | C |
| ATOM | 178 | C | GLY | A | 21 | 76.015 | 73.417 | 2.752 | 1.00 | 26.88 | A | C |
| ATOM | 179 | O | GLY | A | 21 | 75.906 | 73.137 | 1.551 | 1.00 | 27.40 | A | O |
| ATOM | 180 | N | SER | A | 22 | 76.466 | 74.594 | 3.202 | 1.00 | 28.28 | A | N |
| ATOM | 181 | CA | SER | A | 22 | 76.976 | 75.657 | 2.330 | 1.00 | 29.16 | A | C |
| ATOM | 182 | C | SER | A | 22 | 78.298 | 76.173 | 2.919 | 1.00 | 28.62 | A | C |
| ATOM | 183 | O | SER | A | 22 | 78.308 | 76.639 | 4.049 | 1.00 | 29.95 | A | O |
| ATOM | 184 | CB | SER | A | 22 | 75.983 | 76.815 | 2.238 | 1.00 | 29.69 | A | C |
| ATOM | 185 | OG | SER | A | 22 | 74.675 | 76.366 | 1.925 | 1.00 | 29.77 | A | O |
| ATOM | 186 | N | PRO | A | 23 | 79.407 | 76.052 | 2.198 | 1.00 | 28.22 | A | N |
| ATOM | 187 | CA | PRO | A | 23 | 79.461 | 75.401 | 0.884 | 1.00 | 30.78 | A | C |
| ATOM | 188 | C | PRO | A | 23 | 79.227 | 73.886 | 0.976 | 1.00 | 29.87 | A | C |
| ATOM | 189 | O | PRO | A | 23 | 79.338 | 73.300 | 2.063 | 1.00 | 25.45 | A | O |
| ATOM | 190 | CB | PRO | A | 23 | 80.875 | 75.693 | 0.407 | 1.00 | 31.63 | A | C |
| ATOM | 191 | CG | PRO | A | 23 | 81.664 | 75.968 | 1.651 | 1.00 | 29.94 | A | C |
| ATOM | 192 | CD | PRO | A | 23 | 80.727 | 76.545 | 2.629 | 1.00 | 33.02 | A | C |
| ATOM | 193 | N | PRO | A | 24 | 78.894 | 73.258 | −0.145 | 1.00 | 30.31 | A | N |
| ATOM | 194 | CA | PRO | A | 24 | 78.559 | 71.821 | −0.139 | 1.00 | 26.63 | A | C |
| ATOM | 195 | C | PRO | A | 24 | 79.673 | 70.857 | 0.304 | 1.00 | 25.38 | A | C |
| ATOM | 196 | O | PRO | A | 24 | 80.807 | 70.925 | −0.155 | 1.00 | 25.17 | A | O |
| ATOM | 197 | CB | PRO | A | 24 | 78.141 | 71.536 | −1.593 | 1.00 | 28.22 | A | C |
| ATOM | 198 | CG | PRO | A | 24 | 78.576 | 72.715 | −2.410 | 1.00 | 32.40 | A | C |
| ATOM | 199 | CD | PRO | A | 24 | 78.778 | 73.874 | −1.484 | 1.00 | 33.13 | A | C |
| ATOM | 200 | N | GLN | A | 25 | 79.292 | 69.920 | 1.169 | 1.00 | 24.26 | A | N |
| ATOM | 201 | CA | GLN | A | 25 | 80.144 | 68.839 | 1.620 | 1.00 | 23.05 | A | C |
| ATOM | 202 | C | GLN | A | 25 | 79.617 | 67.576 | 0.992 | 1.00 | 19.90 | A | C |
| ATOM | 203 | O | GLN | A | 25 | 78.470 | 67.220 | 1.220 | 1.00 | 20.87 | A | O |
| ATOM | 204 | CB | GLN | A | 25 | 80.075 | 68.728 | 3.127 | 1.00 | 20.92 | A | C |
| ATOM | 205 | CG | GLN | A | 25 | 80.581 | 69.995 | 3.817 | 1.00 | 25.92 | A | C |
| ATOM | 206 | CD | GLN | A | 25 | 80.491 | 69.911 | 5.317 | 1.00 | 24.91 | A | C |
| ATOM | 207 | OE1 | GLN | A | 25 | 80.742 | 68.850 | 5.894 | 1.00 | 21.17 | A | O |
| ATOM | 208 | NE2 | GLN | A | 25 | 80.153 | 71.021 | 5.957 | 1.00 | 26.06 | A | N |
| ATOM | 209 | N | THR | A | 26 | 80.439 | 66.926 | 0.187 | 1.00 | 23.72 | A | N |
| ATOM | 210 | CA | THR | A | 26 | 80.041 | 65.699 | −0.495 | 1.00 | 23.00 | A | C |
| ATOM | 211 | C | THR | A | 26 | 80.141 | 64.498 | 0.435 | 1.00 | 22.59 | A | C |
| ATOM | 212 | O | THR | A | 26 | 81.151 | 64.310 | 1.103 | 1.00 | 23.44 | A | O |
| ATOM | 213 | CB | THR | A | 26 | 80.943 | 65.456 | −1.685 | 1.00 | 24.91 | A | C |
| ATOM | 214 | OG1 | THR | A | 26 | 80.891 | 66.588 | −2.566 | 1.00 | 31.54 | A | O |
| ATOM | 215 | CG2 | THR | A | 26 | 80.428 | 64.292 | −2.537 | 1.00 | 25.28 | A | C |
| ATOM | 216 | N | LEU | A | 27 | 79.107 | 63.666 | 0.430 | 1.00 | 19.15 | A | N |
| ATOM | 217 | CA | LEU | A | 27 | 79.093 | 62.431 | 1.198 | 1.00 | 18.03 | A | C |
| ATOM | 218 | C | LEU | A | 27 | 78.394 | 61.329 | 0.375 | 1.00 | 22.50 | A | C |
| ATOM | 219 | O | LEU | A | 27 | 77.511 | 61.636 | −0.415 | 1.00 | 25.14 | A | O |
| ATOM | 220 | CB | LEU | A | 27 | 78.310 | 62.637 | 2.488 | 1.00 | 18.41 | A | C |
| ATOM | 221 | CG | LEU | A | 27 | 78.805 | 63.740 | 3.447 | 1.00 | 23.17 | A | C |
| ATOM | 222 | CD1 | LEU | A | 27 | 77.737 | 64.155 | 4.429 | 1.00 | 28.47 | A | C |
| ATOM | 223 | CD2 | LEU | A | 27 | 80.040 | 63.300 | 4.174 | 1.00 | 22.35 | A | C |
| ATOM | 224 | N | ASN | A | 28 | 78.804 | 60.075 | 0.562 | 1.00 | 19.63 | A | N |
| ATOM | 225 | CA | ASN | A | 28 | 78.097 | 58.926 | −0.013 | 1.00 | 18.44 | A | C |
| ATOM | 226 | C | ASN | A | 28 | 77.098 | 58.404 | 0.985 | 1.00 | 17.41 | A | C |
| ATOM | 227 | O | ASN | A | 28 | 77.467 | 58.130 | 2.122 | 1.00 | 15.99 | A | O |
| ATOM | 228 | CB | ASN | A | 28 | 79.059 | 57.817 | −0.346 | 1.00 | 17.43 | A | C |
| ATOM | 229 | CG | ASN | A | 28 | 79.868 | 58.114 | −1.556 | 1.00 | 22.09 | A | C |
| ATOM | 230 | OD1 | ASN | A | 28 | 79.407 | 58.837 | −2.434 | 1.00 | 21.00 | A | O |
| ATOM | 231 | ND2 | ASN | A | 28 | 81.084 | 57.573 | −1.622 | 1.00 | 22.09 | A | N |
| ATOM | 232 | N | ILE | A | 29 | 75.848 | 58.222 | 0.566 | 1.00 | 13.33 | A | N |
| ATOM | 233 | CA | ILE | A | 29 | 74.741 | 57.964 | 1.501 | 1.00 | 15.06 | A | C |
| ATOM | 234 | C | ILE | A | 29 | 73.969 | 56.724 | 1.072 | 1.00 | 15.98 | A | C |
| ATOM | 235 | O | ILE | A | 29 | 73.495 | 56.628 | −0.071 | 1.00 | 16.00 | A | O |
| ATOM | 236 | CB | ILE | A | 29 | 73.777 | 59.164 | 1.569 | 1.00 | 17.19 | A | C |
| ATOM | 237 | CG1 | ILE | A | 29 | 74.533 | 60.443 | 1.960 | 1.00 | 16.84 | A | C |
| ATOM | 238 | CG2 | ILE | A | 29 | 72.625 | 58.876 | 2.579 | 1.00 | 15.77 | A | C |
| ATOM | 239 | CD1 | ILE | A | 29 | 75.147 | 60.409 | 3.359 | 1.00 | 18.72 | A | C |
| ATOM | 240 | N | LEU | A | 30 | 73.829 | 55.787 | 1.997 | 1.00 | 15.17 | A | N |
| ATOM | 241 | CA | LEU | A | 30 | 73.110 | 54.541 | 1.743 | 1.00 | 16.63 | A | C |
| ATOM | 242 | C | LEU | A | 30 | 71.623 | 54.825 | 1.455 | 1.00 | 17.89 | A | C |
| ATOM | 243 | O | LEU | A | 30 | 71.000 | 55.542 | 2.186 | 1.00 | 17.80 | A | O |
| ATOM | 244 | CB | LEU | A | 30 | 73.251 | 53.629 | 2.964 | 1.00 | 14.92 | A | C |
| ATOM | 245 | CG | LEU | A | 30 | 72.441 | 52.335 | 2.947 | 1.00 | 24.85 | A | C |
| ATOM | 246 | CD1 | LEU | A | 30 | 73.456 | 51.336 | 1.962 | 0.00 | 19.90 | A | C |
| ATOM | 247 | CD2 | LEU | A | 30 | 72.418 | 51.625 | 4.210 | 0.00 | 19.96 | A | C |
| ATOM | 248 | N | VAL | A | 31 | 71.059 | 54.224 | 0.405 | 1.00 | 15.67 | A | N |
| ATOM | 249 | CA | VAL | A | 31 | 69.656 | 54.390 | 0.066 | 1.00 | 17.96 | A | C |
| ATOM | 250 | C | VAL | A | 31 | 68.865 | 53.269 | 0.715 | 1.00 | 18.65 | A | C |
| ATOM | 251 | O | VAL | A | 31 | 69.101 | 52.060 | 0.440 | 1.00 | 21.01 | A | O |
| ATOM | 252 | CB | VAL | A | 31 | 69.461 | 54.358 | −1.471 | 1.00 | 21.10 | A | C |
| ATOM | 253 | CG1 | VAL | A | 31 | 67.991 | 54.309 | −1.806 | 1.00 | 23.22 | A | C |
| ATOM | 254 | CG2 | VAL | A | 31 | 70.102 | 55.554 | −2.073 | 1.00 | 19.69 | A | C |
| ATOM | 255 | N | ASP | A | 32 | 67.936 | 53.656 | 1.591 | 1.00 | 18.25 | A | N |
| ATOM | 256 | CA | ASP | A | 32 | 67.221 | 52.712 | 2.456 | 1.00 | 20.14 | A | C |
| ATOM | 257 | C | ASP | A | 32 | 65.712 | 52.942 | 2.457 | 1.00 | 18.89 | A | C |
| ATOM | 258 | O | ASP | A | 32 | 65.217 | 53.839 | 3.144 | 1.00 | 18.73 | A | O |
| ATOM | 259 | CB | ASP | A | 32 | 67.748 | 52.832 | 3.905 | 1.00 | 20.81 | A | C |
| ATOM | 260 | CG | ASP | A | 32 | 67.163 | 51.747 | 4.850 | 1.00 | 27.29 | A | C |
| ATOM | 261 | OD1 | ASP | A | 32 | 66.652 | 50.729 | 4.345 | 1.00 | 28.02 | A | O |
| ATOM | 262 | OD2 | ASP | A | 32 | 67.178 | 51.817 | 6.113 | 1.00 | 29.94 | A | O |
| ATOM | 263 | N | THR | A | 33 | 64.947 | 52.108 | 1.735 | 1.00 | 15.71 | A | N |
| ATOM | 264 | CA | THR | A | 33 | 63.500 | 52.284 | 1.753 | 1.00 | 16.65 | A | C |
| ATOM | 265 | C | THR | A | 33 | 62.839 | 51.643 | 2.958 | 1.00 | 18.62 | A | C |
| ATOM | 266 | O | THR | A | 33 | 61.627 | 51.707 | 3.086 | 1.00 | 19.27 | A | O |
| ATOM | 267 | CB | THR | A | 33 | 62.855 | 51.726 | 0.459 | 1.00 | 17.78 | A | C |
| ATOM | 268 | OG1 | THR | A | 33 | 63.088 | 50.330 | 0.395 | 1.00 | 17.76 | A | O |
| ATOM | 269 | CG2 | THR | A | 33 | 63.526 | 52.289 | −0.756 | 1.00 | 20.47 | A | C |
| ATOM | 270 | N | GLY | A | 34 | 63.645 | 51.078 | 3.854 | 1.00 | 19.46 | A | N |
| ATOM | 271 | CA | GLY | A | 34 | 63.137 | 50.457 | 5.065 | 1.00 | 22.82 | A | C |
| ATOM | 272 | C | GLY | A | 34 | 63.251 | 51.314 | 6.315 | 1.00 | 24.98 | A | C |
| ATOM | 273 | O | GLY | A | 34 | 63.033 | 50.830 | 7.434 | 1.00 | 24.60 | A | O |
| ATOM | 274 | N | SER | A | 35 | 63.601 | 52.578 | 6.130 | 1.00 | 18.89 | A | N |
| ATOM | 275 | CA | SER | A | 35 | 63.672 | 53.543 | 7.231 | 1.00 | 21.21 | A | C |
| ATOM | 276 | C | SER | A | 35 | 63.376 | 54.978 | 6.749 | 1.00 | 18.57 | A | C |
| ATOM | 277 | O | SER | A | 35 | 63.245 | 55.229 | 5.535 | 1.00 | 21.32 | A | O |
| ATOM | 278 | CB | SER | A | 35 | 65.045 | 53.420 | 7.880 | 1.00 | 21.69 | A | C |
| ATOM | 279 | OG | SER | A | 35 | 66.063 | 53.982 | 7.078 | 1.00 | 20.28 | A | O |
| ATOM | 280 | N | SER | A | 36 | 63.253 | 55.940 | 7.678 | 1.00 | 18.30 | A | N |
| ATOM | 281 | CA | SER | A | 36 | 62.727 | 57.267 | 7.347 | 1.00 | 20.36 | A | C |
| ATOM | 282 | C | SER | A | 36 | 63.545 | 58.455 | 7.889 | 1.00 | 21.41 | A | C |
| ATOM | 283 | O | SER | A | 36 | 63.101 | 59.594 | 7.809 | 1.00 | 19.92 | A | O |
| ATOM | 284 | CB | SER | A | 36 | 61.267 | 57.375 | 7.824 | 1.00 | 25.82 | A | C |
| ATOM | 285 | OG | SER | A | 36 | 60.485 | 56.344 | 7.230 | 1.00 | 25.30 | A | O |
| ATOM | 286 | N | ASN | A | 37 | 64.748 | 58.181 | 8.396 | 1.00 | 19.59 | A | N |
| ATOM | 287 | CA | ASN | A | 37 | 65.676 | 59.222 | 8.853 | 1.00 | 20.44 | A | C |
| ATOM | 288 | C | ASN | A | 37 | 66.852 | 59.444 | 7.907 | 1.00 | 17.40 | A | C |
| ATOM | 289 | O | ASN | A | 37 | 67.426 | 58.484 | 7.386 | 1.00 | 17.40 | A | O |
| ATOM | 290 | CB | ASN | A | 37 | 66.262 | 58.847 | 10.225 | 1.00 | 19.75 | A | C |
| ATOM | 291 | CG | ASN | A | 37 | 65.330 | 59.162 | 11.365 | 1.00 | 25.09 | A | C |
| ATOM | 292 | OD1 | ASN | A | 37 | 65.323 | 60.288 | 11.888 | 1.00 | 26.01 | A | O |
| ATOM | 293 | ND2 | ASN | A | 37 | 64.555 | 58.177 | 11.776 | 1.00 | 21.61 | A | N |
| ATOM | 294 | N | PHE | A | 38 | 67.217 | 60.704 | 7.697 | 1.00 | 18.60 | A | N |
| ATOM | 295 | CA | PHE | A | 38 | 68.450 | 61.064 | 7.013 | 1.00 | 17.76 | A | C |
| ATOM | 296 | C | PHE | A | 38 | 69.494 | 61.330 | 8.089 | 1.00 | 17.46 | A | C |
| ATOM | 297 | O | PHE | A | 38 | 69.356 | 62.288 | 8.837 | 1.00 | 18.26 | A | O |
| ATOM | 298 | CB | PHE | A | 38 | 68.236 | 62.307 | 6.143 | 1.00 | 17.46 | A | C |
| ATOM | 299 | CG | PHE | A | 38 | 69.466 | 62.776 | 5.366 | 1.00 | 18.60 | A | C |
| ATOM | 300 | CD1 | PHE | A | 38 | 70.391 | 61.896 | 4.828 | 1.00 | 17.37 | A | C |
| ATOM | 301 | CD2 | PHE | A | 38 | 69.657 | 64.124 | 5.127 | 1.00 | 24.93 | A | C |
| ATOM | 302 | CE1 | PHE | A | 38 | 71.488 | 62.350 | 4.104 | 1.00 | 19.65 | A | C |
| ATOM | 303 | CE2 | PHE | A | 38 | 70.747 | 64.586 | 4.384 | 1.00 | 19.49 | A | C |
| ATOM | 304 | CZ | PHE | A | 38 | 71.669 | 63.701 | 3.881 | 1.00 | 23.24 | A | C |
| ATOM | 305 | N | ALA | A | 39 | 70.467 | 60.430 | 8.224 | 1.00 | 18.71 | A | N |
| ATOM | 306 | CA | ALA | A | 39 | 71.480 | 60.508 | 9.272 | 1.00 | 18.80 | A | C |
| ATOM | 307 | C | ALA | A | 39 | 72.866 | 60.348 | 8.667 | 1.00 | 20.90 | A | C |
| ATOM | 308 | O | ALA | A | 39 | 73.104 | 59.439 | 7.862 | 1.00 | 20.32 | A | O |
| ATOM | 309 | CB | ALA | A | 39 | 71.225 | 59.457 | 10.334 | 1.00 | 17.93 | A | C |
| ATOM | 310 | N | VAL | A | 40 | 73.792 | 61.223 | 9.058 | 1.00 | 19.20 | A | N |
| ATOM | 311 | CA | VAL | A | 40 | 75.145 | 61.189 | 8.526 | 1.00 | 18.03 | A | C |
| ATOM | 312 | C | VAL | A | 40 | 76.193 | 61.242 | 9.640 | 1.00 | 18.42 | A | C |
| ATOM | 313 | O | VAL | A | 40 | 76.027 | 61.985 | 10.580 | 1.00 | 15.83 | A | O |
| ATOM | 314 | CB | VAL | A | 40 | 75.398 | 62.372 | 7.587 | 1.00 | 19.32 | A | C |
| ATOM | 315 | CG1 | VAL | A | 40 | 74.430 | 62.354 | 6.382 | 1.00 | 24.72 | A | C |
| ATOM | 316 | CG2 | VAL | A | 40 | 75.304 | 63.711 | 8.319 | 1.00 | 25.33 | A | C |
| ATOM | 317 | N | GLY | A | 41 | 77.272 | 60.490 | 9.488 | 1.00 | 18.41 | A | N |
| ATOM | 318 | CA | GLY | A | 41 | 78.444 | 60.626 | 10.354 | 1.00 | 13.03 | A | C |
| ATOM | 319 | C | GLY | A | 41 | 78.921 | 62.049 | 10.463 | 1.00 | 16.57 | A | C |
| ATOM | 320 | O | GLY | A | 41 | 78.986 | 62.780 | 9.486 | 1.00 | 16.35 | A | O |
| ATOM | 321 | N | ALA | A | 42 | 79.186 | 62.482 | 11.688 | 1.00 | 18.46 | A | N |
| ATOM | 322 | CA | ALA | A | 42 | 79.513 | 63.880 | 11.952 | 1.00 | 16.09 | A | C |
| ATOM | 323 | C | ALA | A | 42 | 80.745 | 63.987 | 12.843 | 1.00 | 21.94 | A | C |
| ATOM | 324 | O | ALA | A | 42 | 81.068 | 65.059 | 13.334 | 1.00 | 21.99 | A | O |
| ATOM | 325 | CB | ALA | A | 42 | 78.326 | 64.558 | 12.613 | 1.00 | 19.21 | A | C |
| ATOM | 326 | N | ALA | A | 43 | 81.444 | 62.873 | 12.985 | 1.00 | 17.43 | A | N |
| ATOM | 327 | CA | ALA | A | 43 | 82.584 | 62.752 | 13.899 | 1.00 | 19.03 | A | C |
| ATOM | 328 | C | ALA | A | 43 | 83.590 | 61.822 | 13.222 | 1.00 | 22.11 | A | C |
| ATOM | 329 | O | ALA | A | 43 | 83.186 | 60.977 | 12.414 | 1.00 | 18.84 | A | O |
| ATOM | 330 | CB | ALA | A | 43 | 82.131 | 62.185 | 15.216 | 1.00 | 20.66 | A | C |
| ATOM | 331 | N | PRO | A | 44 | 84.880 | 61.964 | 13.530 | 1.00 | 21.75 | A | N |
| ATOM | 332 | CA | PRO | A | 44 | 85.928 | 61.128 | 12.903 | 1.00 | 22.99 | A | C |
| ATOM | 333 | C | PRO | A | 44 | 86.039 | 59.692 | 13.422 | 1.00 | 21.03 | A | C |
| ATOM | 334 | O | PRO | A | 44 | 87.044 | 59.283 | 13.989 | 1.00 | 22.42 | A | O |
| ATOM | 335 | CB | PRO | A | 44 | 87.204 | 61.930 | 13.173 | 1.00 | 23.97 | A | C |
| ATOM | 336 | CG | PRO | A | 44 | 86.923 | 62.655 | 14.467 | 1.00 | 21.28 | A | C |
| ATOM | 337 | CD | PRO | A | 44 | 85.466 | 63.000 | 14.406 | 1.00 | 22.65 | A | C |
| ATOM | 338 | N | HIS | A | 45 | 85.004 | 58.904 | 13.175 | 1.00 | 19.15 | A | N |
| ATOM | 339 | CA | HIS | A | 45 | 85.011 | 57.491 | 13.493 | 1.00 | 19.87 | A | C |
| ATOM | 340 | C | HIS | A | 45 | 86.074 | 56.884 | 12.559 | 1.00 | 23.49 | A | C |
| ATOM | 341 | O | HIS | A | 45 | 86.161 | 57.279 | 11.408 | 1.00 | 18.76 | A | O |
| ATOM | 342 | CB | HIS | A | 45 | 83.600 | 56.898 | 13.231 | 1.00 | 20.18 | A | C |
| ATOM | 343 | CG | HIS | A | 45 | 83.499 | 55.426 | 13.491 | 1.00 | 20.56 | A | C |
| ATOM | 344 | ND1 | HIS | A | 45 | 82.921 | 54.900 | 14.628 | 1.00 | 27.21 | A | N |
| ATOM | 345 | CD2 | HIS | A | 45 | 83.911 | 54.369 | 12.753 | 1.00 | 20.97 | A | C |
| ATOM | 346 | CE1 | HIS | A | 45 | 82.989 | 53.579 | 14.577 | 1.00 | 20.15 | A | C |
| ATOM | 347 | NE2 | HIS | A | 45 | 83.572 | 53.234 | 13.443 | 1.00 | 26.79 | A | N |
| ATOM | 348 | N | PRO | A | 46 | 86.900 | 55.958 | 13.039 | 1.00 | 23.59 | A | N |
| ATOM | 349 | CA | PRO | A | 46 | 87.999 | 55.418 | 12.221 | 1.00 | 26.27 | A | C |
| ATOM | 350 | C | PRO | A | 46 | 87.618 | 54.722 | 10.881 | 1.00 | 23.39 | A | C |
| ATOM | 351 | O | PRO | A | 46 | 88.449 | 54.679 | 9.975 | 1.00 | 27.08 | A | O |
| ATOM | 352 | CB | PRO | A | 46 | 88.677 | 54.416 | 13.175 | 1.00 | 24.42 | A | C |
| ATOM | 353 | CG | PRO | A | 46 | 87.621 | 54.034 | 14.147 | 1.00 | 27.39 | A | C |
| ATOM | 354 | CD | PRO | A | 46 | 86.863 | 55.335 | 14.378 | 1.00 | 25.05 | A | C |
| ATOM | 355 | N | PHE | A | 47 | 86.410 | 54.192 | 10.783 | 1.00 | 25.26 | A | N |
| ATOM | 356 | CA | PHE | A | 47 | 85.924 | 53.538 | 9.560 | 1.00 | 25.03 | A | C |
| ATOM | 357 | C | PHE | A | 47 | 85.523 | 54.517 | 8.446 | 1.00 | 22.84 | A | C |
| ATOM | 358 | O | PHE | A | 47 | 85.309 | 54.084 | 7.325 | 1.00 | 25.36 | A | O |
| ATOM | 359 | CB | PHE | A | 47 | 84.678 | 52.671 | 9.832 | 1.00 | 27.84 | A | C |
| ATOM | 360 | CG | PHE | A | 47 | 84.888 | 51.503 | 10.769 | 1.00 | 32.30 | A | C |
| ATOM | 361 | CD1 | PHE | A | 47 | 86.141 | 51.176 | 11.282 | 1.00 | 36.05 | A | C |
| ATOM | 362 | CD2 | PHE | A | 47 | 83.794 | 50.722 | 11.134 | 1.00 | 35.59 | A | C |
| ATOM | 363 | CE1 | PHE | A | 47 | 86.297 | 50.098 | 12.133 | 1.00 | 32.80 | A | C |
| ATOM | 364 | CE2 | PHE | A | 47 | 83.945 | 49.635 | 12.004 | 1.00 | 36.20 | A | C |
| ATOM | 365 | CZ | PHE | A | 47 | 85.197 | 49.326 | 12.489 | 1.00 | 37.31 | A | C |
| ATOM | 366 | N | LEU | A | 48 | 85.377 | 55.804 | 8.761 | 1.00 | 19.13 | A | N |
| ATOM | 367 | CA | LEU | A | 48 | 84.818 | 56.789 | 7.835 | 1.00 | 18.71 | A | C |
| ATOM | 368 | C | LEU | A | 48 | 85.829 | 57.499 | 6.963 | 1.00 | 22.04 | A | C |
| ATOM | 369 | O | LEU | A | 48 | 86.798 | 58.086 | 7.451 | 1.00 | 22.43 | A | O |
| ATOM | 370 | CB | LEU | A | 48 | 84.019 | 57.848 | 8.602 | 1.00 | 17.69 | A | C |
| ATOM | 371 | CG | LEU | A | 48 | 82.797 | 57.361 | 9.367 | 1.00 | 14.97 | A | C |
| ATOM | 372 | CD1 | LEU | A | 48 | 82.068 | 58.567 | 9.926 | 1.00 | 18.29 | A | C |
| ATOM | 373 | CD2 | LEU | A | 48 | 81.839 | 56.567 | 8.517 | 1.00 | 19.80 | A | C |
| ATOM | 374 | N | HIS | A | 49 | 85.553 | 57.517 | 5.666 | 1.00 | 19.90 | A | N |
| ATOM | 375 | CA | HIS | A | 49 | 86.310 | 58.348 | 4.715 | 1.00 | 23.16 | A | C |
| ATOM | 376 | C | HIS | A | 49 | 86.115 | 59.862 | 4.903 | 1.00 | 23.74 | A | C |
| ATOM | 377 | O | HIS | A | 49 | 87.033 | 60.658 | 4.676 | 1.00 | 24.96 | A | O |
| ATOM | 378 | CB | HIS | A | 49 | 85.901 | 58.027 | 3.277 | 1.00 | 24.78 | A | C |
| ATOM | 379 | CG | HIS | A | 49 | 86.253 | 56.648 | 2.822 | 1.00 | 18.81 | A | C |
| ATOM | 380 | ND1 | HIS | A | 49 | 87.368 | 56.386 | 2.054 | 1.00 | 23.64 | A | N |
| ATOM | 381 | CD2 | HIS | A | 49 | 85.623 | 55.463 | 2.989 | 1.00 | 17.53 | A | C |
| ATOM | 382 | CE1 | HIS | A | 49 | 87.408 | 55.095 | 1.779 | 1.00 | 20.49 | A | C |
| ATOM | 383 | NE2 | HIS | A | 49 | 86.361 | 54.512 | 2.331 | 1.00 | 25.00 | A | N |
| ATOM | 384 | N | ARG | A | 50 | 84.900 | 60.274 | 5.255 | 1.00 | 23.13 | A | N |
| ATOM | 385 | CA | ARG | A | 50 | 84.603 | 61.682 | 5.496 | 1.00 | 24.92 | A | C |
| ATOM | 386 | C | ARG | A | 50 | 83.387 | 61.768 | 6.398 | 1.00 | 22.50 | A | C |
| ATOM | 387 | O | ARG | A | 50 | 82.761 | 60.763 | 6.692 | 1.00 | 20.11 | A | O |
| ATOM | 388 | CB | ARG | A | 50 | 84.335 | 62.435 | 4.200 | 1.00 | 31.00 | A | C |
| ATOM | 389 | CG | ARG | A | 50 | 84.028 | 61.549 | 3.065 | 1.00 | 30.52 | A | C |
| ATOM | 390 | CD | ARG | A | 50 | 83.871 | 62.231 | 1.758 | 1.00 | 33.45 | A | C |
| ATOM | 391 | NE | ARG | A | 50 | 83.103 | 61.374 | 0.862 | 1.00 | 35.30 | A | N |
| ATOM | 392 | CZ | ARG | A | 50 | 82.912 | 61.613 | −0.430 | 1.00 | 41.98 | A | C |
| ATOM | 393 | NH1 | ARG | A | 50 | 83.440 | 62.692 | −1.000 | 1.00 | 41.62 | A | N |
| ATOM | 394 | NH2 | ARG | A | 50 | 82.188 | 60.765 | −1.159 | 1.00 | 41.63 | A | N |
| ATOM | 395 | N | TYR | A | 51 | 83.097 | 62.978 | 6.868 | 1.00 | 19.69 | A | N |
| ATOM | 396 | CA | TYR | A | 51 | 81.968 | 63.193 | 7.727 | 1.00 | 19.01 | A | C |
| ATOM | 397 | C | TYR | A | 51 | 81.513 | 64.641 | 7.644 | 1.00 | 17.45 | A | C |
| ATOM | 398 | O | TYR | A | 51 | 82.257 | 65.509 | 7.198 | 1.00 | 19.82 | A | O |
| ATOM | 399 | CB | TYR | A | 51 | 82.305 | 62.792 | 9.175 | 1.00 | 17.00 | A | C |
| ATOM | 400 | CG | TYR | A | 51 | 83.594 | 63.414 | 9.694 | 1.00 | 19.81 | A | C |
| ATOM | 401 | CD1 | TYR | A | 51 | 84.807 | 62.799 | 9.494 | 1.00 | 22.49 | A | C |
| ATOM | 402 | CD2 | TYR | A | 51 | 83.574 | 64.625 | 10.391 | 1.00 | 27.51 | A | C |
| ATOM | 403 | CE1 | TYR | A | 51 | 85.996 | 63.363 | 9.962 | 1.00 | 29.01 | A | C |
| ATOM | 404 | CE2 | TYR | A | 51 | 84.755 | 65.198 | 10.853 | 1.00 | 22.34 | A | C |
| ATOM | 405 | CZ | TYR | A | 51 | 85.959 | 64.561 | 10.639 | 1.00 | 26.38 | A | C |
| ATOM | 406 | OH | TYR | A | 51 | 87.153 | 65.103 | 11.102 | 1.00 | 27.75 | A | O |
| ATOM | 407 | N | TYR | A | 52 | 80.267 | 64.861 | 8.039 | 1.00 | 16.76 | A | N |
| ATOM | 408 | CA | TYR | A | 52 | 79.630 | 66.167 | 8.044 | 1.00 | 15.41 | A | C |
| ATOM | 409 | C | TYR | A | 52 | 80.251 | 67.057 | 9.094 | 1.00 | 18.19 | A | C |
| ATOM | 410 | O | TYR | A | 52 | 80.252 | 66.703 | 10.268 | 1.00 | 18.86 | A | O |
| ATOM | 411 | CB | TYR | A | 52 | 78.163 | 65.968 | 8.360 | 1.00 | 16.96 | A | C |
| ATOM | 412 | CG | TYR | A | 52 | 77.241 | 67.158 | 8.365 | 1.00 | 17.78 | A | C |
| ATOM | 413 | CD1 | TYR | A | 52 | 77.491 | 68.311 | 7.617 | 1.00 | 19.54 | A | C |
| ATOM | 414 | CD2 | TYR | A | 52 | 76.057 | 67.095 | 9.075 | 1.00 | 20.48 | A | C |
| ATOM | 415 | CE1 | TYR | A | 52 | 76.608 | 69.378 | 7.664 | 1.00 | 17.41 | A | C |
| ATOM | 416 | CE2 | TYR | A | 52 | 75.160 | 68.137 | 9.089 | 1.00 | 21.75 | A | C |
| ATOM | 417 | CZ | TYR | A | 52 | 75.443 | 69.280 | 8.373 | 1.00 | 20.07 | A | C |
| ATOM | 418 | OH | TYR | A | 52 | 74.507 | 70.291 | 8.424 | 1.00 | 24.27 | A | O |
| ATOM | 419 | N | GLN | A | 53 | 80.748 | 68.214 | 8.671 | 1.00 | 21.06 | A | N |
| ATOM | 420 | CA | GLN | A | 53 | 81.372 | 69.186 | 9.580 | 1.00 | 22.83 | A | C |
| ATOM | 421 | C | GLN | A | 53 | 80.474 | 70.420 | 9.662 | 1.00 | 18.33 | A | C |
| ATOM | 422 | O | GLN | A | 53 | 80.601 | 71.340 | 8.878 | 1.00 | 23.76 | A | O |
| ATOM | 423 | CB | GLN | A | 53 | 82.779 | 69.535 | 9.079 | 1.00 | 22.30 | A | C |
| ATOM | 424 | CG | GLN | A | 53 | 83.750 | 68.353 | 9.108 | 1.00 | 24.84 | A | C |
| ATOM | 425 | CD | GLN | A | 53 | 85.187 | 68.690 | 8.695 | 1.00 | 31.20 | A | C |
| ATOM | 426 | OE1 | GLN | A | 53 | 85.490 | 68.915 | 7.504 | 1.00 | 32.31 | A | O |
| ATOM | 427 | NE2 | GLN | A | 53 | 86.080 | 68.696 | 9.671 | 1.00 | 27.07 | A | N |
| ATOM | 428 | N | ARG | A | 54 | 79.537 | 70.385 | 10.597 | 1.00 | 20.86 | A | N |
| ATOM | 429 | CA | ARG | A | 54 | 78.545 | 71.442 | 10.758 | 1.00 | 21.52 | A | C |
| ATOM | 430 | C | ARG | A | 54 | 79.164 | 72.827 | 10.939 | 1.00 | 25.15 | A | C |
| ATOM | 431 | O | ARG | A | 54 | 78.568 | 73.828 | 10.536 | 1.00 | 26.20 | A | O |
| ATOM | 432 | CB | ARG | A | 54 | 77.629 | 71.138 | 11.918 | 1.00 | 21.46 | A | C |
| ATOM | 433 | CG | ARG | A | 54 | 76.652 | 69.995 | 11.655 | 1.00 | 22.36 | A | C |
| ATOM | 434 | CD | ARG | A | 54 | 75.989 | 69.437 | 12.869 | 1.00 | 24.51 | A | C |
| ATOM | 435 | NE | ARG | A | 54 | 76.919 | 68.779 | 13.780 | 1.00 | 20.24 | A | N |
| ATOM | 436 | CZ | ARG | A | 54 | 76.609 | 68.376 | 14.997 | 1.00 | 23.34 | A | C |
| ATOM | 437 | NH1 | ARG | A | 54 | 75.389 | 68.574 | 15.485 | 1.00 | 26.99 | A | N |
| ATOM | 438 | NH2 | ARG | A | 54 | 77.534 | 67.786 | 15.739 | 1.00 | 21.22 | A | N |
| ATOM | 439 | N | GLN | A | 55 | 80.362 | 72.880 | 11.523 | 1.00 | 25.18 | A | N |
| ATOM | 440 | CA | GLN | A | 55 | 81.055 | 74.153 | 11.741 | 1.00 | 25.49 | A | C |
| ATOM | 441 | C | GLN | A | 55 | 81.403 | 74.886 | 10.453 | 1.00 | 27.22 | A | C |
| ATOM | 442 | O | GLN | A | 55 | 81.623 | 76.106 | 10.471 | 1.00 | 31.96 | A | O |
| ATOM | 443 | CB | GLN | A | 55 | 82.342 | 73.951 | 12.586 | 1.00 | 25.44 | A | C |
| ATOM | 444 | CG | GLN | A | 55 | 83.508 | 73.285 | 11.866 | 1.00 | 26.87 | A | C |
| ATOM | 445 | CD | GLN | A | 55 | 83.607 | 71.787 | 12.100 | 1.00 | 22.47 | A | C |
| ATOM | 446 | OE1 | GLN | A | 55 | 84.649 | 71.186 | 11.858 | 1.00 | 28.14 | A | O |
| ATOM | 447 | NE2 | GLN | A | 55 | 82.531 | 71.192 | 12.526 | 1.00 | 19.06 | A | N |
| ATOM | 448 | N | LEU | A | 56 | 81.478 | 74.148 | 9.347 | 1.00 | 26.29 | A | N |
| ATOM | 449 | CA | LEU | A | 56 | 81.846 | 74.711 | 8.055 | 1.00 | 26.09 | A | C |
| ATOM | 450 | C | LEU | A | 56 | 80.646 | 75.193 | 7.224 | 1.00 | 28.01 | A | C |
| ATOM | 451 | O | LEU | A | 56 | 80.835 | 75.716 | 6.131 | 1.00 | 30.64 | A | O |
| ATOM | 452 | CB | LEU | A | 56 | 82.667 | 73.703 | 7.251 | 1.00 | 28.42 | A | C |
| ATOM | 453 | CG | LEU | A | 56 | 83.966 | 73.147 | 7.849 | 1.00 | 29.81 | A | C |
| ATOM | 454 | CD1 | LEU | A | 56 | 84.685 | 72.309 | 6.814 | 1.00 | 33.56 | A | C |
| ATOM | 455 | CD2 | LEU | A | 56 | 84.896 | 74.243 | 8.364 | 1.00 | 28.02 | A | C |
| ATOM | 456 | N | SER | A | 57 | 79.432 | 75.055 | 7.760 | 1.00 | 27.95 | A | N |
| ATOM | 457 | CA | SER | A | 57 | 78.199 | 75.322 | 7.009 | 1.00 | 27.26 | A | C |
| ATOM | 458 | C | SER | A | 57 | 77.432 | 76.528 | 7.548 | 1.00 | 26.45 | A | C |
| ATOM | 459 | O | SER | A | 57 | 76.970 | 76.523 | 8.701 | 1.00 | 27.40 | A | O |
| ATOM | 460 | CB | SER | A | 57 | 77.287 | 74.086 | 7.037 | 1.00 | 27.30 | A | C |
| ATOM | 461 | OG | SER | A | 57 | 76.004 | 74.353 | 6.482 | 1.00 | 24.82 | A | O |
| ATOM | 462 | N | SER | A | 58 | 77.250 | 77.541 | 6.704 | 1.00 | 31.30 | A | N |
| ATOM | 463 | CA | SER | A | 58 | 76.540 | 78.753 | 7.112 | 1.00 | 33.18 | A | C |
| ATOM | 464 | C | SER | A | 58 | 75.049 | 78.502 | 7.294 | 1.00 | 33.96 | A | C |
| ATOM | 465 | O | SER | A | 58 | 74.367 | 79.198 | 8.059 | 1.00 | 31.39 | A | O |
| ATOM | 466 | CB | SER | A | 58 | 76.761 | 79.879 | 6.097 | 1.00 | 35.14 | A | C |
| ATOM | 467 | OG | SER | A | 58 | 76.449 | 79.481 | 4.769 | 1.00 | 35.98 | A | O |
| ATOM | 468 | N | THR | A | 59 | 74.552 | 77.473 | 6.608 | 1.00 | 31.44 | A | N |
| ATOM | 469 | CA | THR | A | 59 | 73.128 | 77.222 | 6.528 | 1.00 | 28.82 | A | C |
| ATOM | 470 | C | THR | A | 59 | 72.637 | 76.209 | 7.545 | 1.00 | 27.75 | A | C |
| ATOM | 471 | O | THR | A | 59 | 71.431 | 75.989 | 7.648 | 1.00 | 26.38 | A | O |
| ATOM | 472 | CB | THR | A | 59 | 72.745 | 76.825 | 5.079 | 1.00 | 30.74 | A | C |
| ATOM | 473 | OG1 | THR | A | 59 | 73.712 | 75.937 | 4.512 | 1.00 | 26.79 | A | O |
| ATOM | 474 | CG2 | THR | A | 59 | 72.851 | 78.040 | 4.175 | 1.00 | 31.50 | A | C |
| ATOM | 475 | N | TYR | A | 60 | 73.559 | 75.630 | 8.325 | 1.00 | 25.76 | A | N |
| ATOM | 476 | CA | TYR | A | 60 | 73.204 | 74.716 | 9.405 | 1.00 | 27.01 | A | C |
| ATOM | 477 | C | TYR | A | 60 | 72.359 | 75.391 | 10.487 | 1.00 | 30.17 | A | C |
| ATOM | 478 | O | TYR | A | 60 | 72.671 | 76.504 | 10.908 | 1.00 | 32.85 | A | O |
| ATOM | 479 | CB | TYR | A | 60 | 74.475 | 74.108 | 10.024 | 1.00 | 29.24 | A | C |
| ATOM | 480 | CG | TYR | A | 60 | 74.208 | 73.401 | 11.319 | 1.00 | 32.58 | A | C |
| ATOM | 481 | CD1 | TYR | A | 60 | 73.616 | 72.137 | 11.341 | 1.00 | 33.45 | A | C |
| ATOM | 482 | CD2 | TYR | A | 60 | 74.507 | 74.016 | 12.539 | 1.00 | 35.22 | A | C |
| ATOM | 483 | CE1 | TYR | A | 60 | 73.344 | 71.495 | 12.545 | 1.00 | 34.91 | A | C |
| ATOM | 484 | CE2 | TYR | A | 60 | 74.242 | 73.384 | 13.741 | 1.00 | 35.99 | A | C |
| ATOM | 485 | CZ | TYR | A | 60 | 73.661 | 72.128 | 13.739 | 1.00 | 36.24 | A | C |
| ATOM | 486 | OH | TYR | A | 60 | 73.406 | 71.510 | 14.936 | 1.00 | 40.70 | A | O |
| ATOM | 487 | N | ARG | A | 61 | 71.302 | 74.710 | 10.934 | 1.00 | 29.78 | A | N |
| ATOM | 488 | CA | ARG | A | 61 | 70.489 | 75.137 | 12.074 | 1.00 | 32.29 | A | C |
| ATOM | 489 | C | ARG | A | 61 | 70.289 | 73.992 | 13.056 | 1.00 | 35.05 | A | C |
| ATOM | 490 | O | ARG | A | 61 | 69.781 | 72.931 | 12.695 | 1.00 | 33.45 | A | O |
| ATOM | 491 | CB | ARG | A | 61 | 69.113 | 75.638 | 11.635 | 1.00 | 34.98 | A | C |
| ATOM | 492 | CG | ARG | A | 61 | 69.146 | 76.790 | 10.663 | 1.00 | 33.55 | A | C |
| ATOM | 493 | CD | ARG | A | 61 | 67.756 | 77.209 | 10.187 | 1.00 | 39.45 | A | C |
| ATOM | 494 | NE | ARG | A | 61 | 67.802 | 78.053 | 8.991 | 1.00 | 43.50 | A | N |
| ATOM | 495 | CZ | ARG | A | 61 | 66.737 | 78.400 | 8.267 | 1.00 | 43.32 | A | C |
| ATOM | 496 | NH1 | ARG | A | 61 | 65.517 | 77.969 | 8.591 | 1.00 | 43.64 | A | N |
| ATOM | 497 | NH2 | ARG | A | 61 | 66.896 | 79.173 | 7.201 | 1.00 | 43.55 | A | N |
| ATOM | 498 | N | ASP | A | 62 | 70.681 | 74.222 | 14.302 | 1.00 | 32.81 | A | N |
| ATOM | 499 | CA | ASP | A | 62 | 70.488 | 73.277 | 15.385 | 1.00 | 34.32 | A | C |
| ATOM | 500 | C | ASP | A | 62 | 69.019 | 73.222 | 15.812 | 1.00 | 35.83 | A | C |
| ATOM | 501 | O | ASP | A | 62 | 68.368 | 74.257 | 15.972 | 1.00 | 37.43 | A | O |
| ATOM | 502 | CB | ASP | A | 62 | 71.385 | 73.703 | 16.561 | 1.00 | 36.21 | A | C |
| ATOM | 503 | CG | ASP | A | 62 | 71.724 | 72.567 | 17.509 | 1.00 | 37.73 | A | C |
| ATOM | 504 | OD1 | ASP | A | 62 | 71.078 | 71.513 | 17.462 | 1.00 | 39.38 | A | O |
| ATOM | 505 | OD2 | ASP | A | 62 | 72.632 | 72.654 | 18.366 | 1.00 | 38.06 | A | O |
| ATOM | 506 | N | LEU | A | 63 | 68.504 | 72.009 | 16.000 | 1.00 | 32.04 | A | N |
| ATOM | 507 | CA | LEU | A | 63 | 67.151 | 71.799 | 16.496 | 1.00 | 33.21 | A | C |
| ATOM | 508 | C | LEU | A | 63 | 67.155 | 71.580 | 18.003 | 1.00 | 31.37 | A | C |
| ATOM | 509 | O | LEU | A | 63 | 66.108 | 71.522 | 18.621 | 1.00 | 33.62 | A | O |
| ATOM | 510 | CB | LEU | A | 63 | 66.489 | 70.603 | 15.793 | 1.00 | 32.30 | A | C |
| ATOM | 511 | CG | LEU | A | 63 | 65.919 | 70.957 | 14.417 | 1.00 | 37.47 | A | C |
| ATOM | 512 | CD1 | LEU | A | 63 | 65.566 | 69.688 | 13.604 | 1.00 | 37.52 | A | C |
| ATOM | 513 | CD2 | LEU | A | 63 | 64.696 | 71.880 | 14.549 | 1.00 | 37.36 | A | C |
| ATOM | 514 | N | ARG | A | 64 | 68.345 | 71.460 | 18.580 | 1.00 | 34.84 | A | N |
| ATOM | 515 | CA | ARG | A | 64 | 68.514 | 71.279 | 20.012 | 1.00 | 34.85 | A | C |
| ATOM | 516 | C | ARG | A | 64 | 67.687 | 70.109 | 20.516 | 1.00 | 37.89 | A | C |
| ATOM | 517 | O | ARG | A | 64 | 66.925 | 70.220 | 21.474 | 1.00 | 37.04 | A | O |
| ATOM | 518 | CB | ARG | A | 64 | 68.180 | 72.583 | 20.753 | 1.00 | 37.97 | A | C |
| ATOM | 519 | CG | ARG | A | 64 | 68.865 | 73.821 | 20.152 | 1.00 | 37.97 | A | C |
| ATOM | 520 | CD | ARG | A | 64 | 68.726 | 75.089 | 21.000 | 1.00 | 41.38 | A | C |
| ATOM | 521 | NE | ARG | A | 64 | 69.447 | 74.699 | 22.367 | 0.00 | 47.96 | A | N |
| ATOM | 522 | CZ | ARG | A | 64 | 69.722 | 75.629 | 23.275 | 0.00 | 49.03 | A | C |
| ATOM | 523 | NH1 | ARG | A | 64 | 69.491 | 76.907 | 23.009 | 0.00 | 49.64 | A | N |
| ATOM | 524 | NH2 | ARG | A | 64 | 70.226 | 75.281 | 24.451 | 0.00 | 49.89 | A | N |
| ATOM | 525 | N | LYS | A | 65 | 67.844 | 68.973 | 19.843 | 1.00 | 34.71 | A | N |
| ATOM | 526 | CA | LYS | A | 65 | 67.212 | 67.732 | 20.266 | 1.00 | 35.06 | A | C |
| ATOM | 527 | C | LYS | A | 65 | 68.076 | 66.577 | 19.771 | 1.00 | 30.42 | A | C |
| ATOM | 528 | O | LYS | A | 65 | 68.655 | 66.665 | 18.695 | 1.00 | 31.69 | A | O |
| ATOM | 529 | CB | LYS | A | 65 | 65.801 | 67.642 | 19.676 | 1.00 | 39.80 | A | C |
| ATOM | 530 | CG | LYS | A | 65 | 64.967 | 66.448 | 20.138 | 1.00 | 43.42 | A | C |
| ATOM | 531 | CD | LYS | A | 65 | 63.513 | 66.564 | 19.672 | 1.00 | 47.97 | A | C |
| ATOM | 532 | CE | LYS | A | 65 | 62.653 | 65.440 | 20.263 | 1.00 | 50.01 | A | C |
| ATOM | 533 | NZ | LYS | A | 65 | 61.233 | 65.463 | 19.797 | 1.00 | 51.34 | A | N |
| ATOM | 534 | N | GLY | A | 66 | 68.190 | 65.522 | 20.565 | 1.00 | 31.22 | A | N |
| ATOM | 535 | CA | GLY | A | 66 | 68.910 | 64.339 | 20.149 | 1.00 | 31.55 | A | C |
| ATOM | 536 | C | GLY | A | 66 | 67.996 | 63.249 | 19.616 | 1.00 | 32.06 | A | C |
| ATOM | 537 | O | GLY | A | 66 | 66.772 | 63.399 | 19.632 | 1.00 | 33.71 | A | O |
| ATOM | 538 | N | VAL | A | 67 | 68.617 | 62.153 | 19.163 | 1.00 | 30.61 | A | N |
| ATOM | 539 | CA | VAL | A | 67 | 67.927 | 60.946 | 18.675 | 1.00 | 32.04 | A | C |
| ATOM | 540 | C | VAL | A | 67 | 68.756 | 59.693 | 18.978 | 1.00 | 32.39 | A | C |
| ATOM | 541 | O | VAL | A | 67 | 69.982 | 59.724 | 18.870 | 1.00 | 29.49 | A | O |
| ATOM | 542 | CB | VAL | A | 67 | 67.663 | 61.024 | 17.158 | 1.00 | 34.97 | A | C |
| ATOM | 543 | CG1 | VAL | A | 67 | 66.568 | 61.988 | 16.878 | 1.00 | 40.45 | A | C |
| ATOM | 544 | CG2 | VAL | A | 67 | 68.912 | 61.440 | 16.387 | 1.00 | 36.19 | A | C |
| ATOM | 545 | N | TYR | A | 68 | 68.108 | 58.602 | 19.384 | 1.00 | 32.50 | A | N |
| ATOM | 546 | CA | TYR | A | 68 | 68.817 | 57.361 | 19.709 | 1.00 | 36.46 | A | C |
| ATOM | 547 | C | TYR | A | 68 | 68.113 | 56.190 | 19.062 | 1.00 | 34.88 | A | C |
| ATOM | 548 | O | TYR | A | 68 | 66.962 | 55.916 | 19.383 | 1.00 | 36.97 | A | O |
| ATOM | 549 | CB | TYR | A | 68 | 68.902 | 57.148 | 21.229 | 1.00 | 36.07 | A | C |
| ATOM | 550 | CG | TYR | A | 68 | 69.801 | 55.993 | 21.670 | 1.00 | 41.81 | A | C |
| ATOM | 551 | CD1 | TYR | A | 68 | 69.460 | 54.665 | 21.395 | 1.00 | 43.38 | A | C |
| ATOM | 552 | CD2 | TYR | A | 68 | 70.981 | 56.226 | 22.379 | 1.00 | 44.20 | A | C |
| ATOM | 553 | CE1 | TYR | A | 68 | 70.274 | 53.605 | 21.798 | 1.00 | 43.39 | A | C |
| ATOM | 554 | CE2 | TYR | A | 68 | 71.805 | 55.167 | 22.789 | 1.00 | 44.55 | A | C |
| ATOM | 555 | CZ | TYR | A | 68 | 71.444 | 53.863 | 22.492 | 1.00 | 45.41 | A | C |
| ATOM | 556 | OH | TYR | A | 68 | 72.242 | 52.807 | 22.897 | 1.00 | 47.48 | A | O |
| ATOM | 557 | N | VAL | A | 69 | 68.826 | 55.477 | 18.196 | 1.00 | 33.48 | A | N |
| ATOM | 558 | CA | VAL | A | 69 | 68.249 | 54.404 | 17.376 | 1.08 | 34.57 | A | C |
| ATOM | 559 | C | VAL | A | 69 | 68.922 | 53.080 | 17.716 | 1.00 | 34.34 | A | C |
| ATOM | 560 | O | VAL | A | 69 | 69.996 | 52.793 | 17.192 | 1.00 | 28.53 | A | O |
| ATOM | 561 | CB | VAL | A | 69 | 68.440 | 54.691 | 15.866 | 1.00 | 35.13 | A | C |
| ATOM | 562 | CG1 | VAL | A | 69 | 67.944 | 53.526 | 15.002 | 1.00 | 38.45 | A | C |
| ATOM | 563 | CG2 | VAL | A | 69 | 67.754 | 56.000 | 15.484 | 1.00 | 36.74 | A | C |
| ATOM | 564 | N | PRO | A | 70 | 68.319 | 52.269 | 18.588 | 1.00 | 39.88 | A | N |
| ATOM | 565 | CA | PRO | A | 70 | 68.846 | 50.922 | 18.830 | 1.00 | 43.50 | A | C |
| ATOM | 566 | C | PRO | A | 70 | 68.577 | 50.028 | 17.629 | 1.00 | 47.11 | A | C |
| ATOM | 567 | O | PRO | A | 70 | 67.551 | 50.175 | 16.960 | 1.00 | 41.77 | A | O |
| ATOM | 568 | CB | PRO | A | 70 | 68.097 | 50.428 | 20.077 | 1.00 | 44.42 | A | C |
| ATOM | 569 | CG | PRO | A | 70 | 67.031 | 51.423 | 20.368 | 1.00 | 43.58 | A | C |
| ATOM | 570 | CD | PRO | A | 70 | 67.125 | 52.554 | 19.397 | 1.00 | 42.11 | A | C |
| ATOM | 571 | N | TYR | A | 71 | 69.527 | 49.140 | 17.367 | 1.00 | 51.98 | A | N |
| ATOM | 572 | CA | TYR | A | 71 | 69.474 | 48.179 | 16.276 | 1.00 | 56.73 | A | C |
| ATOM | 573 | C | TYR | A | 71 | 69.683 | 46.796 | 16.908 | 1.00 | 58.39 | A | C |
| ATOM | 574 | O | TYR | A | 71 | 69.428 | 46.618 | 18.105 | 1.00 | 57.75 | A | O |
| ATOM | 575 | CB | TYR | A | 71 | 70.558 | 48.519 | 15.229 | 1.00 | 57.66 | A | C |
| ATOM | 576 | CG | TYR | A | 71 | 70.091 | 49.405 | 14.090 | 1.00 | 59.91 | A | C |
| ATOM | 577 | CD1 | TYR | A | 71 | 70.760 | 50.591 | 13.779 | 1.00 | 60.36 | A | C |
| ATOM | 578 | CD2 | TYR | A | 71 | 68.995 | 49.049 | 13.304 | 1.00 | 61.59 | A | C |
| ATOM | 579 | CE1 | TYR | A | 71 | 70.334 | 51.408 | 12.725 | 1.00 | 60.84 | A | C |
| ATOM | 580 | CE2 | TYR | A | 71 | 68.568 | 49.857 | 12.249 | 1.00 | 62.07 | A | C |
| ATOM | 581 | CZ | TYR | A | 71 | 69.241 | 51.035 | 11.966 | 1.00 | 63.27 | A | C |
| ATOM | 582 | OH | TYR | A | 71 | 68.818 | 51.840 | 10.924 | 1.00 | 64.04 | A | O |
| ATOM | 583 | N | THR | A | 72 | 70.147 | 45.832 | 16.114 | 1.00 | 61.01 | A | N |
| ATOM | 584 | CA | THR | A | 72 | 70.319 | 44.444 | 16.556 | 1.00 | 60.90 | A | C |
| ATOM | 585 | C | THR | A | 72 | 71.093 | 44.294 | 17.877 | 1.00 | 59.74 | A | C |
| ATOM | 586 | O | THR | A | 72 | 70.491 | 44.060 | 18.931 | 1.00 | 58.04 | A | O |
| ATOM | 587 | CB | THR | A | 72 | 70.993 | 43.609 | 15.431 | 1.00 | 62.06 | A | C |
| ATOM | 588 | OG1 | THR | A | 72 | 72.170 | 44.276 | 14.951 | 1.00 | 61.28 | A | O |
| ATOM | 589 | CG2 | THR | A | 72 | 70.090 | 43.514 | 14.196 | 1.00 | 63.15 | A | C |
| ATOM | 590 | N | GLN | A | 73 | 72.418 | 44.402 | 17.800 | 1.00 | 57.85 | A | N |
| ATOM | 591 | CA | GLN | A | 73 | 73.287 | 44.461 | 18.971 | 1.00 | 57.41 | A | C |
| ATOM | 592 | C | GLN | A | 73 | 74.155 | 45.726 | 18.850 | 1.00 | 54.83 | A | C |
| ATOM | 593 | O | GLN | A | 73 | 75.303 | 45.747 | 19.299 | 1.00 | 57.07 | A | O |
| ATOM | 594 | CB | GLN | A | 73 | 74.153 | 43.194 | 19.060 | 1.00 | 58.83 | A | C |
| ATOM | 595 | CG | GLN | A | 73 | 73.865 | 42.294 | 20.273 | 1.00 | 60.65 | A | C |
| ATOM | 596 | CD | GLN | A | 73 | 74.720 | 42.630 | 21.504 | 1.00 | 63.27 | A | C |
| ATOM | 597 | OE1 | GLN | A | 73 | 75.959 | 42.582 | 21.450 | 1.00 | 61.11 | A | O |
| ATOM | 598 | NE2 | GLN | A | 73 | 74.058 | 42.943 | 22.619 | 1.00 | 61.34 | A | N |
| ATOM | 599 | N | GLY | A | 74 | 73.591 | 46.763 | 18.223 | 1.00 | 48.16 | A | N |
| ATOM | 600 | CA | GLY | A | 74 | 74.262 | 48.041 | 18.020 | 1.00 | 42.89 | A | C |
| ATOM | 601 | C | GLY | A | 74 | 73.290 | 49.214 | 18.016 | 1.00 | 38.35 | A | C |
| ATOM | 602 | O | GLY | A | 74 | 72.224 | 49.115 | 18.625 | 1.00 | 39.09 | A | O |
| ATOM | 603 | N | LYS | A | 75 | 73.656 | 50.320 | 17.360 | 1.00 | 32.71 | A | N |
| ATOM | 604 | CA | LYS | A | 75 | 72.844 | 51.554 | 17.362 | 1.00 | 31.39 | A | C |
| ATOM | 605 | C | LYS | A | 75 | 73.525 | 52.762 | 16.664 | 1.00 | 24.85 | A | C |
| ATOM | 606 | O | LYS | A | 75 | 74.685 | 52.698 | 16.338 | 1.00 | 21.55 | A | O |
| ATOM | 607 | CB | LYS | A | 75 | 72.483 | 51.946 | 18.800 | 1.00 | 34.61 | A | C |
| ATOM | 608 | CG | LYS | A | 75 | 73.667 | 52.144 | 19.731 | 1.00 | 39.32 | A | C |
| ATOM | 609 | CD | LYS | A | 75 | 74.545 | 53.318 | 19.299 | 1.00 | 39.96 | A | C |
| ATOM | 610 | CE | LYS | A | 75 | 75.034 | 54.144 | 20.451 | 1.00 | 40.91 | A | C |
| ATOM | 611 | NZ | LYS | A | 75 | 74.297 | 55.407 | 20.464 | 1.00 | 45.34 | A | N |
| ATOM | 612 | N | TRP | A | 76 | 72.782 | 53.843 | 16.434 | 1.00 | 22.44 | A | N |
| ATOM | 613 | CA | TRP | A | 76 | 73.372 | 55.173 | 16.224 | 1.00 | 25.02 | A | C |
| ATOM | 614 | C | TRP | A | 76 | 72.594 | 56.201 | 17.012 | 1.00 | 23.16 | A | C |
| ATOM | 615 | O | TRP | A | 76 | 71.429 | 56.007 | 17.353 | 1.00 | 21.34 | A | O |
| ATOM | 616 | CB | TRP | A | 76 | 73.512 | 55.570 | 14.732 | 1.00 | 24.36 | A | C |
| ATOM | 617 | CG | TRP | A | 76 | 72.243 | 55.752 | 13.957 | 1.00 | 25.79 | A | C |
| ATOM | 618 | CD1 | TRP | A | 76 | 71.643 | 54.833 | 13.136 | 1.00 | 26.34 | A | C |
| ATOM | 619 | CD2 | TRP | A | 76 | 71.424 | 56.932 | 13.896 | 1.00 | 21.29 | A | C |
| ATOM | 620 | NE1 | TRP | A | 76 | 70.491 | 55.364 | 12.595 | 1.00 | 27.23 | A | N |
| ATOM | 621 | CE2 | TRP | A | 76 | 70.348 | 56.656 | 13.030 | 1.00 | 25.91 | A | C |
| ATOM | 622 | CE3 | TRP | A | 76 | 71.497 | 58.202 | 14.479 | 1.00 | 24.01 | A | C |
| ATOM | 623 | CZ2 | TRP | A | 76 | 69.349 | 57.595 | 12.752 | 1.00 | 26.87 | A | C |
| ATOM | 624 | CZ3 | TRP | A | 76 | 70.512 | 59.124 | 14.202 | 1.00 | 26.34 | A | C |
| ATOM | 625 | CH2 | TRP | A | 76 | 69.448 | 58.818 | 13.345 | 1.00 | 25.94 | A | C |
| ATOM | 626 | N | GLU | A | 77 | 73.291 | 57.271 | 17.354 | 1.00 | 22.20 | A | N |
| ATOM | 627 | CA | GLU | A | 77 | 72.753 | 58.327 | 18.164 | 1.00 | 24.84 | A | C |
| ATOM | 628 | C | GLU | A | 77 | 73.255 | 59.632 | 17.575 | 1.00 | 22.78 | A | C |
| ATOM | 629 | O | GLU | A | 77 | 74.386 | 59.723 | 17.089 | 1.00 | 19.61 | A | O |
| ATOM | 630 | CB | GLU | A | 77 | 73.214 | 58.140 | 19.621 | 1.00 | 28.88 | A | C |
| ATOM | 631 | CG | GLU | A | 77 | 72.959 | 59.331 | 20.529 | 1.00 | 35.35 | A | C |
| ATOM | 632 | CD | GLU | A | 77 | 73.323 | 59.057 | 21.980 | 0.50 | 36.38 | A | C |
| ATOM | 633 | OE1 | GLU | A | 77 | 74.397 | 58.470 | 22.222 | 0.50 | 42.18 | A | O |
| ATOM | 634 | OE2 | GLU | A | 77 | 72.536 | 59.431 | 22.878 | 0.50 | 39.02 | A | O |
| ATOM | 635 | N | GLY | A | 78 | 72.418 | 60.651 | 17.573 | 1.00 | 24.09 | A | N |
| ATOM | 636 | CA | GLY | A | 78 | 72.811 | 61.883 | 16.933 | 1.00 | 25.68 | A | C |
| ATOM | 637 | C | GLY | A | 78 | 72.160 | 63.134 | 17.453 | 1.00 | 25.43 | A | C |
| ATOM | 638 | O | GLY | A | 78 | 71.328 | 63.116 | 18.350 | 1.00 | 27.93 | A | O |
| ATOM | 639 | N | GLU | A | 79 | 72.579 | 64.234 | 16.861 | 1.00 | 23.79 | A | N |
| ATOM | 640 | CA | GLU | A | 79 | 72.078 | 65.542 | 17.187 | 1.00 | 23.88 | A | C |
| ATOM | 641 | C | GLU | A | 79 | 71.283 | 65.981 | 15.979 | 1.00 | 22.32 | A | C |
| ATOM | 642 | O | GLU | A | 79 | 71.800 | 65.979 | 14.875 | 1.00 | 26.62 | A | O |
| ATOM | 643 | CB | GLU | A | 79 | 73.255 | 66.487 | 17.457 | 1.00 | 23.99 | A | C |
| ATOM | 644 | CG | GLU | A | 79 | 74.109 | 66.052 | 18.641 | 1.00 | 29.60 | A | C |
| ATOM | 645 | CD | GLU | A | 79 | 75.420 | 66.826 | 18.790 | 1.00 | 33.85 | A | C |
| ATOM | 646 | OE1 | GLU | A | 79 | 76.205 | 66.467 | 19.685 | 1.00 | 34.63 | A | O |
| ATOM | 647 | OE2 | GLU | A | 79 | 75.670 | 67.782 | 18.030 | 1.00 | 37.21 | A | O |
| ATOM | 648 | N | LEU | A | 80 | 70.017 | 66.338 | 16.180 | 1.00 | 22.69 | A | N |
| ATOM | 649 | CA | LEU | A | 80 | 69.184 | 66.809 | 15.075 | 1.00 | 24.78 | A | C |
| ATOM | 650 | C | LEU | A | 80 | 69.419 | 68.267 | 14.685 | 1.00 | 26.56 | A | C |
| ATOM | 651 | O | LEU | A | 80 | 69.596 | 69.139 | 15.528 | 1.00 | 26.06 | A | O |
| ATOM | 652 | CB | LEU | A | 80 | 67.704 | 66.617 | 15.403 | 1.00 | 26.07 | A | C |
| ATOM | 653 | CG | LEU | A | 80 | 67.233 | 65.168 | 15.432 | 1.00 | 31.35 | A | C |
| ATOM | 654 | CD1 | LEU | A | 80 | 65.863 | 65.082 | 16.077 | 1.00 | 28.71 | A | C |
| ATOM | 655 | CD2 | LEU | A | 80 | 67.212 | 64.609 | 14.015 | 1.00 | 32.32 | A | C |
| ATOM | 656 | N | GLY | A | 81 | 69.390 | 68.525 | 13.383 | 1.00 | 23.46 | A | N |
| ATOM | 657 | CA | GLY | A | 81 | 69.500 | 69.861 | 12.822 | 1.00 | 22.46 | A | C |
| ATOM | 658 | C | GLY | A | 81 | 68.854 | 69.916 | 11.448 | 1.00 | 26.63 | A | C |
| ATOM | 659 | O | GLY | A | 81 | 68.308 | 68.927 | 11.002 | 1.00 | 22.44 | A | O |
| ATOM | 660 | N | THR | A | 82 | 68.884 | 71.065 | 10.787 | 1.00 | 26.31 | A | N |
| ATOM | 661 | CA | THR | A | 82 | 68.530 | 71.138 | 9.369 | 1.00 | 28.52 | A | C |
| ATOM | 662 | C | THR | A | 82 | 69.634 | 71.813 | 8.631 | 1.00 | 25.22 | A | C |
| ATOM | 663 | O | THR | A | 82 | 70.436 | 72.529 | 9.225 | 1.00 | 27.82 | A | O |
| ATOM | 664 | CB | THR | A | 82 | 67.190 | 71.888 | 9.127 | 1.00 | 29.47 | A | C |
| ATOM | 665 | OG1 | THR | A | 82 | 67.310 | 73.253 | 9.554 | 1.00 | 27.90 | A | O |
| ATOM | 666 | CG2 | THR | A | 62 | 66.069 | 71.306 | 9.972 | 1.00 | 30.70 | A | C |
| ATOM | 667 | N | ASP | A | 83 | 69.704 | 71.567 | 7.326 | 1.00 | 24.75 | A | N |
| ATOM | 668 | CA | ASP | A | 83 | 70.679 | 72.180 | 6.447 | 1.00 | 22.11 | A | C |
| ATOM | 669 | C | ASP | A | 83 | 70.241 | 71.993 | 5.009 | 1.00 | 24.09 | A | C |
| ATOM | 670 | O | ASP | A | 83 | 69.261 | 71.285 | 4.741 | 1.00 | 26.17 | A | O |
| ATOM | 671 | CB | ASP | A | 83 | 72.075 | 71.559 | 6.652 | 1.00 | 24.10 | A | C |
| ATOM | 672 | CG | ASP | A | 83 | 73.213 | 72.542 | 6.376 | 1.00 | 26.19 | A | C |
| ATOM | 673 | OD1 | ASP | A | 83 | 73.067 | 73.513 | 5.580 | 1.00 | 27.95 | A | O |
| ATOM | 674 | OD2 | ASP | A | 83 | 74.328 | 72.409 | 6.924 | 1.00 | 25.64 | A | O |
| ATOM | 675 | N | LEU | A | 84 | 70.973 | 72.591 | 4.081 | 1.00 | 26.89 | A | N |
| ATOM | 676 | CA | LEU | A | 84 | 70.641 | 72.502 | 2.658 | 1.00 | 27.35 | A | C |
| ATOM | 677 | C | LEU | A | 84 | 71.224 | 71.225 | 2.078 | 1.00 | 28.51 | A | C |
| ATOM | 678 | O | LEU | A | 84 | 72.398 | 70.936 | 2.266 | 1.00 | 25.25 | A | O |
| ATOM | 679 | CB | LEU | A | 84 | 71.193 | 73.717 | 1.915 | 1.00 | 29.63 | A | C |
| ATOM | 680 | CG | LEU | A | 84 | 70.550 | 75.047 | 2.345 | 1.00 | 31.38 | A | C |
| ATOM | 681 | CD1 | LEU | A | 84 | 71.025 | 76.228 | 1.501 | 1.00 | 30.45 | A | C |
| ATOM | 682 | CD2 | LEU | A | 84 | 69.027 | 74.949 | 2.301 | 1.00 | 30.98 | A | C |
| ATOM | 683 | N | VAL | A | 85 | 70.392 | 70.465 | 1.373 | 1.00 | 25.49 | A | N |
| ATOM | 684 | CA | VAL | A | 85 | 70.790 | 69.203 | 0.768 | 1.00 | 28.08 | A | C |
| ATOM | 685 | C | VAL | A | 85 | 70.523 | 69.177 | −0.737 | 1.00 | 27.65 | A | C |
| ATOM | 686 | O | VAL | A | 85 | 69.511 | 69.686 | −1.213 | 1.00 | 27.43 | A | O |
| ATOM | 687 | CB | VAL | A | 85 | 70.063 | 68.028 | 1.439 | 1.00 | 27.18 | A | C |
| ATOM | 688 | CG1 | VAL | A | 85 | 70.564 | 66.696 | 0.875 | 1.00 | 27.97 | A | C |
| ATOM | 689 | CG2 | VAL | A | 85 | 70.273 | 68.084 | 2.950 | 1.00 | 29.93 | A | C |
| ATOM | 690 | N | SER | A | 86 | 71.451 | 68.587 | −1.472 | 1.00 | 28.67 | A | N |
| ATOM | 691 | CA | SER | A | 86 | 71.331 | 68.409 | −2.913 | 1.00 | 30.97 | A | C |
| ATOM | 692 | C | SER | A | 86 | 71.823 | 67.015 | −3.293 | 1.00 | 31.51 | A | C |
| ATOM | 693 | O | SER | A | 86 | 72.512 | 66.354 | −2.509 | 1.00 | 25.69 | A | O |
| ATOM | 694 | CB | SER | A | 86 | 72.138 | 69.485 | −3.642 | 1.00 | 33.70 | A | C |
| ATOM | 695 | OG | SER | A | 86 | 71.607 | 69.737 | −4.930 | 1.00 | 42.01 | A | O |
| ATOM | 696 | N | ILE | A | 87 | 71.459 | 66.563 | −4.494 | 1.00 | 24.97 | A | N |
| ATOM | 697 | CA | ILE | A | 87 | 71.895 | 65.277 | −5.006 | 1.00 | 25.75 | A | C |
| ATOM | 698 | C | ILE | A | 87 | 72.489 | 65.559 | −6.384 | 1.00 | 29.06 | A | C |
| ATOM | 699 | O | ILE | A | 87 | 71.737 | 65.734 | −7.354 | 1.00 | 27.25 | A | O |
| ATOM | 700 | CB | ILE | A | 87 | 70.713 | 64.275 | −5.094 | 1.00 | 25.77 | A | C |
| ATOM | 701 | CG1 | ILE | A | 87 | 70.062 | 64.090 | −3.729 | 1.00 | 26.43 | A | C |
| ATOM | 702 | CG2 | ILE | A | 87 | 71.187 | 62.939 | −5.631 | 1.00 | 23.43 | A | C |
| ATOM | 703 | CD1 | ILE | A | 87 | 68.758 | 63.332 | −3.747 | 1.00 | 29.21 | A | C |
| ATOM | 704 | N | PRO | A | 88 | 73.817 | 65.654 | −6.453 | 1.00 | 29.18 | A | N |
| ATOM | 705 | CA | PRO | A | 88 | 74.531 | 66.013 | −7.689 | 1.00 | 30.76 | A | C |
| ATOM | 706 | C | PRO | A | 88 | 74.063 | 65.286 | −8.956 | 1.00 | 32.20 | A | C |
| ATOM | 707 | O | PRO | A | 88 | 73.924 | 65.938 | −9.987 | 1.00 | 33.45 | A | O |
| ATOM | 708 | CB | PRO | A | 88 | 75.971 | 65.632 | −7.358 | 1.00 | 31.55 | A | C |
| ATOM | 709 | CG | PRO | A | 88 | 76.067 | 65.895 | −5.896 | 1.00 | 30.46 | A | C |
| ATOM | 710 | CD | PRO | A | 88 | 74.762 | 65.455 | −5.339 | 1.00 | 28.40 | A | C |
| ATOM | 711 | N | HIS | A | 89 | 73.857 | 63.972 | −8.872 | 1.00 | 27.36 | A | N |
| ATOM | 712 | CA | HIS | A | 89 | 73.332 | 63.162 | −9.978 | 1.00 | 28.26 | A | C |
| ATOM | 713 | C | HIS | A | 89 | 71.871 | 62.815 | −9.715 | 1.00 | 29.29 | A | C |
| ATOM | 714 | O | HIS | A | 89 | 71.449 | 61.661 | −9.847 | 1.00 | 28.09 | A | O |
| ATOM | 715 | CB | HIS | A | 89 | 74.173 | 61.907 | −10.160 | 1.00 | 28.17 | A | C |
| ATOM | 716 | CG | HIS | A | 89 | 75.632 | 62.184 | −10.362 | 1.00 | 38.05 | A | C |
| ATOM | 717 | ND1 | HIS | A | 89 | 76.120 | 62.833 | −11.478 | 1.00 | 41.06 | A | N |
| ATOM | 718 | CD2 | HIS | A | 89 | 76.708 | 61.905 | −9.588 | 1.00 | 38.74 | A | C |
| ATOM | 719 | CE1 | HIS | A | 89 | 77.435 | 62.933 | −11.384 | 1.00 | 40.63 | A | C |
| ATOM | 720 | NE2 | HIS | A | 89 | 77.817 | 62.376 | −10.248 | 1.00 | 41.19 | A | N |
| ATOM | 721 | N | GLY | A | 90 | 71.120 | 63.846 | −9.334 | 1.00 | 32.50 | A | N |
| ATOM | 722 | CA | GLY | A | 90 | 69.696 | 63.769 | −9.051 | 1.00 | 31.86 | A | C |
| ATOM | 723 | C | GLY | A | 90 | 69.005 | 64.963 | −9.686 | 1.00 | 30.26 | A | C |
| ATOM | 724 | O | GLY | A | 90 | 69.524 | 65.524 | −10.644 | 1.00 | 31.12 | A | O |
| ATOM | 725 | N | PRO | A | 91 | 67.861 | 65.382 | −9.158 | 1.00 | 32.37 | A | N |
| ATOM | 726 | CA | PRO | A | 91 | 67.175 | 66.565 | −9.691 | 1.00 | 34.88 | A | C |
| ATOM | 727 | C | PRO | A | 91 | 67.987 | 67.835 | −9.410 | 1.00 | 39.91 | A | C |
| ATOM | 728 | O | PRO | A | 91 | 68.764 | 67.852 | −8.458 | 1.00 | 38.58 | A | O |
| ATOM | 729 | CB | PRO | A | 91 | 65.837 | 66.579 | −8.937 | 1.00 | 35.23 | A | C |
| ATOM | 730 | CG | PRO | A | 91 | 66.049 | 65.738 | −7.711 | 1.00 | 34.49 | A | C |
| ATOM | 731 | CD | PRO | A | 91 | 67.164 | 64.807 | −7.994 | 1.00 | 33.82 | A | C |
| ATOM | 732 | N | ASN | A | 92 | 67.809 | 68.863 | −10.238 | 1.00 | 43.06 | A | N |
| ATOM | 733 | CA | ASN | A | 92 | 68.496 | 70.152 | −10.086 | 1.00 | 45.05 | A | C |
| ATOM | 734 | C | ASN | A | 92 | 67.841 | 71.060 | −9.034 | 1.00 | 44.59 | A | C |
| ATOM | 735 | O | ASN | A | 92 | 67.368 | 72.156 | −9.337 | 1.00 | 44.08 | A | O |
| ATOM | 736 | CB | ASN | A | 92 | 68.546 | 70.860 | −11.441 | 1.00 | 47.21 | A | C |
| ATOM | 737 | CG | ASN | A | 92 | 69.438 | 72.079 | −11.431 | 1.00 | 50.71 | A | C |
| ATOM | 738 | OD1 | ASN | A | 92 | 70.604 | 72.003 | −11.044 | 1.00 | 52.68 | A | O |
| ATOM | 739 | ND2 | ASN | A | 92 | 68.895 | 73.217 | −11.863 | 1.00 | 52.24 | A | N |
| ATOM | 740 | N | VAL | A | 93 | 67.830 | 70.592 | −7.789 | 1.00 | 41.78 | A | N |
| ATOM | 741 | CA | VAL | A | 93 | 67.205 | 71.310 | −6.691 | 1.00 | 36.70 | A | C |
| ATOM | 742 | C | VAL | A | 93 | 68.043 | 71.217 | −5.428 | 1.00 | 35.54 | A | C |
| ATOM | 743 | O | VAL | A | 93 | 68.907 | 70.353 | −5.304 | 1.00 | 36.77 | A | O |
| ATOM | 744 | CB | VAL | A | 93 | 65.794 | 70.772 | −6.374 | 1.00 | 38.91 | A | C |
| ATOM | 745 | CG1 | VAL | A | 93 | 64.868 | 70.960 | −7.573 | 1.00 | 37.74 | A | C |
| ATOM | 746 | CG2 | VAL | A | 93 | 65.848 | 69.310 | −5.921 | 1.00 | 37.34 | A | C |
| ATOM | 747 | N | THR | A | 94 | 67.772 | 72.139 | −4.513 | 1.00 | 33.85 | A | N |
| ATOM | 748 | CA | THR | A | 94 | 68.320 | 72.119 | −3.178 | 1.00 | 35.85 | A | C |
| ATOM | 749 | C | THR | A | 94 | 67.170 | 72.293 | −2.216 | 1.00 | 36.41 | A | C |
| ATOM | 750 | O | THR | A | 94 | 66.283 | 73.119 | −2.443 | 1.00 | 38.29 | A | O |
| ATOM | 751 | CB | THR | A | 94 | 69.327 | 73.252 | −3.009 | 1.00 | 37.46 | A | C |
| ATOM | 752 | OG1 | THR | A | 94 | 70.459 | 73.016 | −3.855 | 1.00 | 37.95 | A | O |
| ATOM | 753 | CG2 | THR | A | 94 | 69.910 | 73.256 | −1.599 | 1.00 | 39.22 | A | C |
| ATOM | 754 | N | VAL | A | 95 | 67.162 | 71.515 | −1.143 | 1.00 | 32.79 | A | N |
| ATOM | 755 | CA | VAL | A | 95 | 66.110 | 71.652 | −0.155 | 1.00 | 32.68 | A | C |
| ATOM | 756 | C | VAL | A | 95 | 66.660 | 71.686 | 1.261 | 1.00 | 30.49 | A | C |
| ATOM | 757 | O | VAL | A | 95 | 67.762 | 71.240 | 1.499 | 1.00 | 31.56 | A | O |
| ATOM | 758 | CB | VAL | A | 95 | 65.071 | 70.544 | −0.291 | 1.00 | 36.55 | A | C |
| ATOM | 759 | CG1 | VAL | A | 95 | 64.479 | 70.568 | −1.709 | 1.00 | 38.67 | A | C |
| ATOM | 760 | CG2 | VAL | A | 95 | 65.663 | 69.183 | 0.025 | 1.00 | 33.09 | A | C |
| ATOM | 761 | N | ARG | A | 96 | 65.883 | 72.244 | 2.181 | 1.00 | 30.99 | A | N |
| ATOM | 762 | CA | ARG | A | 96 | 66.212 | 72.215 | 3.597 | 1.00 | 29.56 | A | C |
| ATOM | 763 | C | ARG | A | 96 | 65.620 | 70.957 | 4.208 | 1.00 | 28.73 | A | C |
| ATOM | 764 | O | ARG | A | 96 | 64.402 | 70.809 | 4.302 | 1.00 | 30.19 | A | O |
| ATOM | 765 | CB | ARG | A | 96 | 65.686 | 73.459 | 4.320 | 1.00 | 33.02 | A | C |
| ATOM | 766 | CG | ARG | A | 96 | 65.976 | 73.474 | 5.835 | 1.00 | 36.80 | A | C |
| ATOM | 767 | CD | ARG | A | 96 | 65.954 | 74.863 | 6.457 | 1.00 | 38.14 | A | C |
| ATOM | 768 | NE | ARG | A | 96 | 67.041 | 75.677 | 5.929 | 1.00 | 37.92 | A | N |
| ATOM | 769 | CZ | ARG | A | 96 | 68.265 | 75.747 | 6.442 | 1.00 | 37.97 | A | C |
| ATOM | 770 | NH1 | ARG | A | 96 | 68.600 | 75.050 | 7.524 | 1.00 | 38.44 | A | N |
| ATOM | 771 | NH2 | ARG | A | 96 | 69.160 | 76.512 | 5.846 | 1.00 | 33.62 | A | N |
| ATOM | 772 | N | ALA | A | 97 | 66.503 | 70.048 | 4.606 | 1.00 | 24.74 | A | N |
| ATOM | 773 | CA | ALA | A | 97 | 66.126 | 68.764 | 5.167 | 1.00 | 27.21 | A | C |
| ATOM | 774 | C | ALA | A | 97 | 66.541 | 68.668 | 6.614 | 1.00 | 22.38 | A | C |
| ATOM | 775 | O | ALA | A | 97 | 67.523 | 69.278 | 7.026 | 1.00 | 23.68 | A | O |
| ATOM | 776 | CB | ALA | A | 97 | 66.801 | 67.648 | 4.380 | 1.00 | 24.80 | A | C |
| ATOM | 777 | N | ASN | A | 98 | 65.796 | 67.884 | 7.378 | 1.00 | 21.67 | A | N |
| ATOM | 778 | CA | ASN | A | 98 | 66.281 | 67.388 | 8.644 | 1.00 | 22.81 | A | C |
| ATOM | 779 | C | ASN | A | 98 | 67.502 | 66.503 | 8.409 | 1.00 | 25.29 | A | C |
| ATOM | 780 | O | ASN | A | 98 | 67.538 | 65.738 | 7.451 | 1.00 | 21.43 | A | O |
| ATOM | 781 | CB | ASN | A | 98 | 65.184 | 66.605 | 9.351 | 1.00 | 23.87 | A | C |
| ATOM | 782 | CG | ASN | A | 98 | 64.033 | 67.503 | 9.805 | 1.00 | 31.55 | A | C |
| ATOM | 783 | OD1 | ASN | A | 98 | 64.257 | 68.532 | 10.448 | 1.00 | 28.77 | A | O |
| ATOM | 784 | ND2 | ASN | A | 98 | 62.801 | 67.115 | 9.469 | 1.00 | 29.01 | A | N |
| ATOM | 785 | N | ILE | A | 99 | 68.517 | 66.652 | 9.255 | 1.00 | 23.47 | A | N |
| ATOM | 786 | CA | ILE | A | 99 | 69.693 | 65.781 | 9.240 | 1.00 | 21.95 | A | C |
| ATOM | 787 | C | ILE | A | 99 | 70.048 | 65.437 | 10.685 | 1.00 | 19.63 | A | C |
| ATOM | 788 | O | ILE | A | 99 | 70.186 | 66.339 | 11.529 | 1.00 | 24.71 | A | O |
| ATOM | 789 | CB | ILE | A | 99 | 70.902 | 66.475 | 8.586 | 1.00 | 22.78 | A | C |
| ATOM | 790 | CG1 | ILE | A | 99 | 70.571 | 66.968 | 7.184 | 1.00 | 19.57 | A | C |
| ATOM | 791 | CG2 | ILE | A | 99 | 72.076 | 65.544 | 8.527 | 1.00 | 25.77 | A | C |
| ATOM | 792 | CD1 | ILE | A | 99 | 71.663 | 67.806 | 6.568 | 1.00 | 26.04 | A | C |
| ATOM | 793 | N | ALA | A | 100 | 70.167 | 64.149 | 10.968 | 1.00 | 17.47 | A | N |
| ATOM | 794 | CA | ALA | A | 100 | 70.721 | 63.657 | 12.223 | 1.00 | 17.42 | A | C |
| ATOM | 795 | C | ALA | A | 100 | 72.245 | 63.532 | 12.075 | 1.00 | 21.54 | A | C |
| ATOM | 796 | O | ALA | A | 100 | 72.742 | 62.697 | 11.325 | 1.00 | 18.72 | A | O |
| ATOM | 797 | CB | ALA | A | 100 | 70.116 | 62.345 | 12.607 | 1.00 | 21.16 | A | C |
| ATOM | 798 | N | ALA | A | 101 | 72.981 | 64.369 | 12.804 | 1.00 | 18.65 | A | N |
| ATOM | 799 | CA | ALA | A | 101 | 74.436 | 64.308 | 12.819 | 1.00 | 19.52 | A | C |
| ATOM | 800 | C | ALA | A | 101 | 74.849 | 63.244 | 13.813 | 1.00 | 19.24 | A | C |
| ATOM | 801 | O | ALA | A | 101 | 74.595 | 63.358 | 15.017 | 1.00 | 22.30 | A | O |
| ATOM | 802 | CB | ALA | A | 101 | 75.052 | 65.702 | 13.163 | 1.00 | 21.40 | A | C |
| ATOM | 803 | N | ILE | A | 102 | 75.398 | 62.150 | 13.311 | 1.00 | 15.90 | A | N |
| ATOM | 804 | CA | ILE | A | 102 | 75.660 | 60.973 | 14.129 | 1.00 | 17.94 | A | C |
| ATOM | 805 | C | ILE | A | 102 | 76.952 | 61.245 | 14.892 | 1.00 | 19.52 | A | C |
| ATOM | 806 | O | ILE | A | 102 | 77.978 | 61.511 | 14.288 | 1.00 | 19.99 | A | O |
| ATOM | 807 | CB | ILE | A | 102 | 75.842 | 59.690 | 13.277 | 1.00 | 15.21 | A | C |
| ATOM | 808 | CG1 | ILE | A | 102 | 74.554 | 59.374 | 12.505 | 1.00 | 16.99 | A | C |
| ATOM | 809 | CG2 | ILE | A | 102 | 76.224 | 58.519 | 14.178 | 1.00 | 18.39 | A | C |
| ATOM | 810 | CD1 | ILE | A | 102 | 74.673 | 58.276 | 11.472 | 1.00 | 19.74 | A | C |
| ATOM | 811 | N | THR | A | 103 | 76.866 | 61.146 | 16.212 | 1.00 | 21.46 | A | N |
| ATOM | 812 | CA | THR | A | 103 | 77.982 | 61.450 | 17.114 | 1.00 | 25.21 | A | C |
| ATOM | 813 | C | THR | A | 103 | 78.451 | 60.245 | 17.925 | 1.00 | 26.42 | A | C |
| ATOM | 814 | O | THR | A | 103 | 79.504 | 60.296 | 18.556 | 1.00 | 27.83 | A | O |
| ATOM | 815 | CB | THR | A | 103 | 77.556 | 62.579 | 18.073 | 1.00 | 24.52 | A | C |
| ATOM | 816 | OG1 | THR | A | 103 | 76.344 | 62.216 | 18.746 | 1.00 | 26.84 | A | O |
| ATOM | 817 | CG2 | THR | A | 103 | 77.183 | 63.831 | 17.317 | 1.00 | 25.79 | A | C |
| ATOM | 818 | N | GLU | A | 104 | 77.668 | 59.168 | 17.934 | 1.00 | 23.45 | A | N |
| ATOM | 819 | CA | GLU | A | 104 | 78.061 | 57.917 | 18.576 | 1.00 | 19.81 | A | C |
| ATOM | 820 | C | GLU | A | 104 | 77.351 | 56.767 | 17.877 | 1.00 | 21.65 | A | C |
| ATOM | 821 | O | GLU | A | 104 | 76.208 | 56.921 | 17.465 | 1.00 | 21.87 | A | O |
| ATOM | 822 | CB | GLU | A | 104 | 77.725 | 57.928 | 20.088 | 1.00 | 28.17 | A | C |
| ATOM | 823 | CG | GLU | A | 104 | 78.291 | 56.737 | 20.854 | 1.00 | 33.07 | A | C |
| ATOM | 824 | CD | GLU | A | 104 | 77.964 | 56.726 | 22.350 | 1.00 | 42.35 | A | C |
| ATOM | 825 | OE1 | GLU | A | 104 | 77.594 | 57.785 | 22.928 | 1.00 | 48.99 | A | O |
| ATOM | 826 | OE2 | GLU | A | 104 | 78.089 | 55.637 | 22.961 | 1.00 | 51.21 | A | O |
| ATOM | 827 | N | SER | A | 105 | 78.043 | 55.649 | 17.693 | 1.00 | 19.13 | A | N |
| ATOM | 828 | CA | SER | A | 105 | 77.446 | 54.481 | 17.026 | 1.00 | 20.88 | A | C |
| ATOM | 829 | C | SER | A | 105 | 78.126 | 53.167 | 17.421 | 1.00 | 24.82 | A | C |
| ATOM | 830 | O | SER | A | 105 | 79.260 | 53.151 | 17.929 | 1.00 | 23.75 | A | O |
| ATOM | 831 | CB | SER | A | 105 | 77.440 | 54.676 | 15.490 | 1.00 | 18.17 | A | C |
| ATOM | 832 | OG | SER | A | 105 | 78.758 | 54.663 | 15.012 | 1.00 | 21.83 | A | O |
| ATOM | 833 | N | ASP | A | 106 | 77.400 | 52.072 | 17.214 | 1.00 | 22.55 | A | N |
| ATOM | 834 | CA | ASP | A | 106 | 77.913 | 50.733 | 17.411 | 1.00 | 24.69 | A | C |
| ATOM | 835 | C | ASP | A | 106 | 77.315 | 49.839 | 16.312 | 1.00 | 23.68 | A | C |
| ATOM | 836 | O | ASP | A | 106 | 76.094 | 49.837 | 16.093 | 1.00 | 22.98 | A | O |
| ATOM | 837 | CB | ASP | A | 106 | 77.556 | 50.196 | 18.792 | 1.00 | 29.23 | A | C |
| ATOM | 838 | CG | ASP | A | 106 | 77.998 | 48.751 | 18.973 | 0.50 | 30.82 | A | C |
| ATOM | 839 | OD1 | ASP | A | 106 | 79.136 | 48.520 | 19.419 | 0.50 | 35.18 | A | O |
| ATOM | 840 | OD2 | ASP | A | 106 | 77.279 | 47.781 | 18.668 | 0.50 | 34.57 | A | O |
| ATOM | 841 | N | LYS | A | 107 | 78.190 | 49.123 | 15.618 | 1.00 | 25.54 | A | N |
| ATOM | 842 | CA | LYS | A | 107 | 77.820 | 48.161 | 14.572 | 1.00 | 22.36 | A | C |
| ATOM | 843 | C | LYS | A | 107 | 76.966 | 48.753 | 13.446 | 1.00 | 25.69 | A | C |
| ATOM | 844 | O | LYS | A | 107 | 76.176 | 48.054 | 12.825 | 1.00 | 22.57 | A | O |
| ATOM | 845 | CB | LYS | A | 107 | 77.139 | 46.935 | 15.195 | 1.00 | 27.43 | A | C |
| ATOM | 846 | CG | LYS | A | 107 | 78.066 | 46.130 | 16.101 | 1.00 | 32.24 | A | C |
| ATOM | 847 | CD | LYS | A | 107 | 77.314 | 45.034 | 16.835 | 1.00 | 33.50 | A | C |
| ATOM | 848 | CE | LYS | A | 107 | 78.004 | 44.328 | 17.899 | 0.00 | 31.63 | A | C |
| ATOM | 849 | NZ | LYS | A | 107 | 79.348 | 43.882 | 17.435 | 0.00 | 31.79 | A | N |
| ATOM | 850 | N | PHE | A | 108 | 77.151 | 50.043 | 13.187 | 1.00 | 22.67 | A | N |
| ATOM | 851 | CA | PHE | A | 108 | 76.412 | 50.770 | 12.161 | 1.00 | 20.97 | A | C |
| ATOM | 852 | C | PHE | A | 108 | 77.306 | 50.946 | 10.954 | 1.00 | 19.92 | A | C |
| ATOM | 853 | O | PHE | A | 108 | 77.016 | 50.416 | 9.875 | 1.00 | 21.69 | A | O |
| ATOM | 854 | CB | PHE | A | 108 | 75.946 | 52.125 | 12.691 | 1.00 | 19.57 | A | C |
| ATOM | 855 | CG | PHE | A | 108 | 75.153 | 52.921 | 11.701 | 1.00 | 20.66 | A | C |
| ATOM | 856 | CD1 | PHE | A | 108 | 73.870 | 52.520 | 11.338 | 1.00 | 25.16 | A | C |
| ATOM | 857 | CD2 | PHE | A | 108 | 75.688 | 54.053 | 11.107 | 1.00 | 22.61 | A | C |
| ATOM | 858 | CE1 | PHE | A | 108 | 73.139 | 53.250 | 10.405 | 1.00 | 25.50 | A | C |
| ATOM | 859 | CE2 | PHE | A | 108 | 74.963 | 54.790 | 10.190 | 1.00 | 22.84 | A | C |
| ATOM | 860 | CZ | PHE | A | 108 | 73.677 | 54.381 | 9.832 | 1.00 | 26.62 | A | C |
| ATOM | 861 | N | PHE | A | 109 | 78.401 | 51.682 | 11.129 | 1.00 | 20.35 | A | N |
| ATOM | 862 | CA | PHE | A | 109 | 79.372 | 51.887 | 10.044 | 1.00 | 20.96 | A | C |
| ATOM | 863 | C | PHE | A | 109 | 80.123 | 50.581 | 9.813 | 1.00 | 19.74 | A | C |
| ATOM | 864 | O | PHE | A | 109 | 80.361 | 49.824 | 10.769 | 1.00 | 24.70 | A | O |
| ATOM | 865 | CB | PHE | A | 109 | 80.325 | 53.065 | 10.348 | 1.00 | 19.85 | A | C |
| ATOM | 866 | CG | PHE | A | 109 | 79.617 | 54.398 | 10.489 | 1.00 | 16.19 | A | C |
| ATOM | 867 | CD1 | PHE | A | 109 | 78.862 | 54.897 | 9.435 | 1.00 | 22.18 | A | C |
| ATOM | 868 | CD2 | PHE | A | 109 | 79.726 | 55.162 | 11.633 | 1.00 | 22.85 | A | C |
| ATOM | 869 | CE1 | PHE | A | 109 | 78.197 | 56.107 | 9.532 | 1.00 | 21.10 | A | C |
| ATOM | 870 | CE2 | PHE | A | 109 | 79.066 | 56.377 | 11.728 | 1.00 | 21.43 | A | C |
| ATOM | 871 | CZ | PHE | A | 109 | 78.284 | 56.841 | 10.663 | 1.00 | 22.40 | A | C |
| ATOM | 872 | N | ILE | A | 110 | 80.460 | 50.285 | 8.556 | 1.00 | 24.38 | A | N |
| ATOM | 873 | CA | ILE | A | 110 | 81.176 | 49.060 | 8.204 | 1.00 | 23.73 | A | C |
| ATOM | 874 | C | ILE | A | 110 | 82.627 | 49.382 | 7.863 | 1.00 | 25.17 | A | C |
| ATOM | 875 | O | ILE | A | 110 | 82.917 | 50.295 | 7.077 | 1.00 | 21.65 | A | O |
| ATOM | 876 | CB | ILE | A | 110 | 80.510 | 48.364 | 6.998 | 1.00 | 23.43 | A | C |
| ATOM | 877 | CG1 | ILE | A | 110 | 79.073 | 47.944 | 7.330 | 1.00 | 26.13 | A | C |
| ATOM | 878 | CG2 | ILE | A | 110 | 81.354 | 47.171 | 6.511 | 1.00 | 27.63 | A | C |
| ATOM | 879 | CD1 | ILE | A | 110 | 78.262 | 47.542 | 6.104 | 1.00 | 29.01 | A | C |
| ATOM | 880 | N | ASN | A | 111 | 83.535 | 48.616 | 8.453 | 1.00 | 24.01 | A | N |
| ATOM | 881 | CA | ASN | A | 111 | 84.958 | 48.786 | 8.213 | 1.00 | 25.66 | A | C |
| ATOM | 882 | C | ASN | A | 111 | 85.302 | 48.367 | 6.782 | 1.00 | 21.62 | A | C |
| ATOM | 883 | O | ASN | A | 111 | 85.122 | 47.210 | 6.395 | 1.00 | 24.50 | A | O |
| ATOM | 884 | CB | ASN | A | 111 | 85.762 | 47.950 | 9.219 | 1.00 | 26.77 | A | C |
| ATOM | 885 | CG | ASN | A | 111 | 87.239 | 48.324 | 9.252 | 1.00 | 30.39 | A | C |
| ATOM | 886 | OD1 | ASN | A | 111 | 87.614 | 49.478 | 9.012 | 1.00 | 29.76 | A | O |
| ATOM | 887 | ND2 | ASN | A | 111 | 88.081 | 47.348 | 9.588 | 1.00 | 28.98 | A | N |
| ATOM | 888 | N | GLY | A | 112 | 85.815 | 49.310 | 6.008 | 1.00 | 21.53 | A | N |
| ATOM | 889 | CA | GLY | A | 112 | 86.127 | 49.082 | 4.604 | 1.00 | 26.83 | A | C |
| ATOM | 890 | C | GLY | A | 112 | 85.073 | 49.602 | 3.630 | 1.00 | 27.54 | A | C |
| ATOM | 891 | O | GLY | A | 112 | 85.274 | 49.562 | 2.419 | 1.00 | 26.87 | A | O |
| ATOM | 892 | N | SER | A | 113 | 83.950 | 50.086 | 4.145 | 1.00 | 28.16 | A | N |
| ATOM | 893 | CA | SER | A | 113 | 82.869 | 50.607 | 3.301 | 1.00 | 23.29 | A | C |
| ATOM | 894 | C | SER | A | 113 | 83.152 | 52.034 | 2.864 | 1.00 | 22.88 | A | C |
| ATOM | 895 | O | SER | A | 113 | 83.981 | 52.730 | 3.462 | 1.00 | 22.23 | A | O |
| ATOM | 896 | CB | SER | A | 113 | 81.537 | 50.544 | 4.053 | 1.00 | 26.77 | A | C |
| ATOM | 897 | OG | SER | A | 113 | 81.450 | 51.622 | 4.968 | 1.00 | 32.46 | A | O |
| ATOM | 898 | N | ASN | A | 114 | 82.451 | 52.469 | 1.818 | 1.00 | 19.83 | A | N |
| ATOM | 899 | CA | ASN | A | 114 | 82.632 | 53.785 | 1.195 | 1.00 | 20.70 | A | C |
| ATOM | 900 | C | ASN | A | 114 | 81.400 | 54.686 | 1.349 | 1.00 | 17.94 | A | C |
| ATOM | 901 | O | ASN | A | 114 | 81.228 | 55.627 | 0.596 | 1.00 | 20.08 | A | O |
| ATOM | 902 | CB | ASN | A | 114 | 82.973 | 53.574 | −0.303 | 1.00 | 20.54 | A | C |
| ATOM | 903 | CG | ASN | A | 114 | 83.533 | 54.827 | −1.004 | 1.00 | 26.09 | A | C |
| ATOM | 904 | OD1 | ASN | A | 114 | 83.189 | 55.100 | −2.165 | 1.00 | 29.37 | A | O |
| ATOM | 905 | ND2 | ASN | A | 114 | 84.441 | 55.540 | −0.348 | 1.00 | 22.18 | A | N |
| ATOM | 906 | N | TRP | A | 115 | 80.558 | 54.414 | 2.354 | 1.00 | 16.89 | A | N |
| ATOM | 907 | CA | TRP | A | 115 | 79.453 | 55.295 | 2.658 | 1.00 | 16.46 | A | C |
| ATOM | 908 | C | TRP | A | 115 | 79.548 | 55.772 | 4.100 | 1.00 | 18.12 | A | C |
| ATOM | 909 | O | TRP | A | 115 | 80.184 | 55.126 | 4.943 | 1.00 | 20.65 | A | O |
| ATOM | 910 | CB | TRP | A | 115 | 78.093 | 54.631 | 2.393 | 1.00 | 18.60 | A | C |
| ATOM | 911 | CG | TRP | A | 115 | 77.869 | 53.335 | 3.061 | 1.00 | 18.81 | A | C |
| ATOM | 912 | CD1 | TRP | A | 115 | 78.058 | 52.098 | 2.520 | 1.00 | 27.02 | A | C |
| ATOM | 913 | CD2 | TRP | A | 115 | 77.372 | 53.109 | 4.403 | 1.00 | 19.85 | A | C |
| ATOM | 914 | NE1 | TRP | A | 115 | 77.734 | 51.123 | 3.434 | 1.00 | 28.07 | A | N |
| ATOM | 915 | CE2 | TRP | A | 115 | 77.311 | 51.716 | 4.597 | 1.00 | 28.04 | A | C |
| ATOM | 916 | CE3 | TRP | A | 115 | 76.983 | 53.943 | 5.453 | 1.00 | 21.36 | A | C |
| ATOM | 917 | CZ2 | TRP | A | 115 | 76.877 | 51.142 | 5.799 | 1.00 | 27.30 | A | C |
| ATOM | 918 | CZ3 | TRP | A | 115 | 76.544 | 53.371 | 6.643 | 1.00 | 22.49 | A | C |
| ATOM | 919 | CH2 | TRP | A | 115 | 76.510 | 51.996 | 6.808 | 1.00 | 24.66 | A | C |
| ATOM | 920 | N | GLU | A | 116 | 78.910 | 56.905 | 4.345 | 1.00 | 18.09 | A | N |
| ATOM | 921 | CA | GLU | A | 116 | 79.049 | 57.666 | 5.584 | 1.00 | 18.37 | A | C |
| ATOM | 922 | C | GLU | A | 116 | 77.726 | 58.110 | 6.220 | 1.00 | 21.39 | A | C |
| ATOM | 923 | O | GLU | A | 116 | 77.719 | 58.866 | 7.185 | 1.00 | 19.69 | A | O |
| ATOM | 924 | CB | GLU | A | 116 | 79.891 | 58.924 | 5.282 | 1.00 | 21.09 | A | C |
| ATOM | 925 | CG | GLU | A | 116 | 81.298 | 58.664 | 4.834 | 1.00 | 30.57 | A | C |
| ATOM | 926 | CD | GLU | A | 116 | 81.495 | 58.683 | 3.331 | 1.00 | 19.12 | A | C |
| ATOM | 927 | OE1 | GLU | A | 116 | 80.945 | 59.571 | 2.609 | 1.00 | 25.47 | A | O |
| ATOM | 928 | OE2 | GLU | A | 116 | 82.237 | 57.811 | 2.889 | 1.00 | 30.78 | A | O |
| ATOM | 929 | N | GLY | A | 117 | 76.601 | 57.670 | 5.680 | 1.00 | 15.48 | A | N |
| ATOM | 930 | CA | GLY | A | 117 | 75.302 | 58.008 | 6.218 | 1.00 | 14.95 | A | C |
| ATOM | 931 | C | GLY | A | 117 | 74.221 | 57.194 | 5.523 | 1.00 | 17.22 | A | C |
| ATOM | 932 | O | GLY | A | 117 | 74.517 | 56.329 | 4.686 | 1.00 | 15.79 | A | O |
| ATOM | 933 | N | ILE | A | 118 | 72.980 | 57.475 | 5.888 | 1.00 | 18.20 | A | N |
| ATOM | 934 | CA | ILE | A | 118 | 71.810 | 56.721 | 5.455 | 1.00 | 12.85 | A | C |
| ATOM | 935 | C | ILE | A | 118 | 70.668 | 57.692 | 5.108 | 1.00 | 15.45 | A | C |
| ATOM | 936 | O | ILE | A | 118 | 70.426 | 58.691 | 5.805 | 1.00 | 15.49 | A | O |
| ATOM | 937 | CB | ILE | A | 118 | 71.401 | 55.687 | 6.518 | 1.00 | 16.49 | A | C |
| ATOM | 938 | CG1 | ILE | A | 118 | 70.260 | 54.788 | 6.018 | 1.00 | 20.60 | A | C |
| ATOM | 939 | CG2 | ILE | A | 118 | 70.977 | 56.368 | 7.820 | 1.00 | 18.54 | A | C |
| ATOM | 940 | CD1 | ILE | A | 118 | 69.959 | 53.672 | 6.975 | 1.00 | 22.49 | A | C |
| ATOM | 941 | N | LEU | A | 119 | 69.973 | 57.386 | 4.012 | 1.00 | 16.51 | A | N |
| ATOM | 942 | CA | LEU | A | 119 | 68.850 | 58.180 | 3.520 | 1.00 | 17.34 | A | C |
| ATOM | 943 | C | LEU | A | 119 | 67.605 | 57.332 | 3.631 | 1.00 | 17.57 | A | C |
| ATOM | 944 | O | LEU | A | 119 | 67.370 | 56.426 | 2.823 | 1.00 | 17.07 | A | O |
| ATOM | 945 | CB | LEU | A | 119 | 69.061 | 58.614 | 2.073 | 1.00 | 16.12 | A | C |
| ATOM | 946 | CG | LEU | A | 119 | 67.954 | 59.469 | 1.461 | 1.00 | 20.50 | A | C |
| ATOM | 947 | CD1 | LEU | A | 119 | 67.744 | 60.734 | 2.237 | 1.00 | 21.51 | A | C |
| ATOM | 948 | CD2 | LEU | A | 119 | 68.286 | 59.797 | 0.034 | 1.00 | 22.00 | A | C |
| ATOM | 949 | N | GLY | A | 120 | 66.817 | 57.600 | 4.659 | 1.00 | 15.26 | A | N |
| ATOM | 950 | CA | GLY | A | 120 | 65.590 | 56.864 | 4.892 | 1.00 | 16.17 | A | C |
| ATOM | 951 | C | GLY | A | 120 | 64.506 | 57.419 | 3.975 | 1.00 | 16.03 | A | C |
| ATOM | 952 | O | GLY | A | 120 | 64.131 | 58.593 | 4.102 | 1.00 | 20.14 | A | O |
| ATOM | 953 | N | LEU | A | 121 | 64.011 | 56.582 | 3.064 | 1.00 | 15.98 | A | N |
| ATOM | 954 | CA | LEU | A | 121 | 63.037 | 57.010 | 2.038 | 1.00 | 16.93 | A | C |
| ATOM | 955 | C | LEU | A | 121 | 61.586 | 56.616 | 2.330 | 1.00 | 18.71 | A | C |
| ATOM | 956 | O | LEU | A | 121 | 60.683 | 56.874 | 1.530 | 1.00 | 20.26 | A | O |
| ATOM | 957 | CB | LEU | A | 121 | 63.460 | 56.449 | 0.682 | 1.00 | 16.32 | A | C |
| ATOM | 958 | CG | LEU | A | 121 | 64.699 | 57.128 | 0.084 | 1.00 | 18.18 | A | C |
| ATOM | 959 | CD1 | LEU | A | 121 | 65.208 | 56.418 | −1.167 | 1.00 | 17.74 | A | C |
| ATOM | 960 | CD2 | LEU | A | 121 | 64.505 | 58.626 | −0.230 | 1.00 | 19.83 | A | C |
| ATOM | 961 | N | ALA | A | 122 | 61.377 | 55.931 | 3.440 | 1.00 | 17.96 | A | N |
| ATOM | 962 | CA | ALA | A | 122 | 60.037 | 55.568 | 3.916 | 1.00 | 19.62 | A | C |
| ATOM | 963 | C | ALA | A | 122 | 59.307 | 56.740 | 4.589 | 1.00 | 24.01 | A | C |
| ATOM | 964 | O | ALA | A | 122 | 59.734 | 57.890 | 4.476 | 1.00 | 24.71 | A | O |
| ATOM | 965 | CB | ALA | A | 122 | 60.130 | 54.361 | 4.829 | 1.00 | 20.17 | A | C |
| ATOM | 966 | N | TYR | A | 123 | 58.185 | 56.447 | 5.256 | 1.00 | 23.30 | A | N |
| ATOM | 967 | CA | TYR | A | 123 | 57.265 | 57.473 | 5.703 | 1.00 | 25.93 | A | C |
| ATOM | 968 | C | TYR | A | 123 | 57.492 | 57.894 | 7.163 | 1.00 | 23.41 | A | C |
| ATOM | 969 | O | TYR | A | 123 | 58.146 | 57.192 | 7.931 | 1.00 | 25.28 | A | O |
| ATOM | 970 | CB | TYR | A | 123 | 55.836 | 56.968 | 5.559 | 1.00 | 25.54 | A | C |
| ATOM | 971 | CG | TYR | A | 123 | 55.441 | 56.697 | 4.129 | 1.00 | 24.32 | A | C |
| ATOM | 972 | CD1 | TYR | A | 123 | 55.015 | 57.724 | 3.310 | 1.00 | 25.79 | A | C |
| ATOM | 973 | CD2 | TYR | A | 123 | 55.491 | 55.421 | 3.609 | 1.00 | 26.50 | A | C |
| ATOM | 974 | CE1 | TYR | A | 123 | 54.622 | 57.486 | 1.998 | 1.00 | 28.42 | A | C |
| ATOM | 975 | CE2 | TYR | A | 123 | 55.120 | 55.171 | 2.293 | 1.00 | 27.28 | A | C |
| ATOM | 976 | CZ | TYR | A | 123 | 54.678 | 56.195 | 1.501 | 1.00 | 25.25 | A | C |
| ATOM | 977 | OH | TYR | A | 123 | 54.315 | 55.950 | 0.184 | 1.00 | 26.89 | A | O |
| ATOM | 978 | N | ALA | A | 124 | 56.879 | 59.014 | 7.519 | 1.00 | 29.24 | A | N |
| ATOM | 979 | CA | ALA | A | 124 | 57.082 | 59.664 | 8.820 | 1.00 | 30.24 | A | C |
| ATOM | 980 | C | ALA | A | 124 | 56.708 | 58.812 | 10.018 | 1.00 | 35.27 | A | C |
| ATOM | 981 | O | ALA | A | 124 | 57.302 | 58.953 | 11.091 | 1.00 | 33.99 | A | O |
| ATOM | 982 | CB | ALA | A | 124 | 56.356 | 60.972 | 8.858 | 1.00 | 31.62 | A | C |
| ATOM | 983 | N | GLU | A | 125 | 55.754 | 57.903 | 9.834 | 1.00 | 34.28 | A | N |
| ATOM | 984 | CA | GLU | A | 125 | 55.295 | 57.003 | 10.894 | 1.00 | 37.92 | A | C |
| ATOM | 985 | C | GLU | A | 125 | 56.415 | 56.274 | 11.647 | 1.00 | 37.65 | A | C |
| ATOM | 986 | O | GLU | A | 125 | 56.299 | 56.030 | 12.853 | 1.00 | 38.82 | A | O |
| ATOM | 987 | CB | GLU | A | 125 | 54.330 | 55.968 | 10.293 | 1.00 | 40.24 | A | C |
| ATOM | 988 | CG | GLU | A | 125 | 53.444 | 55.252 | 11.295 | 1.00 | 45.50 | A | C |
| ATOM | 989 | CD | GLU | A | 125 | 52.121 | 55.962 | 11.496 | 1.00 | 52.07 | A | C |
| ATOM | 990 | OE1 | GLU | A | 125 | 52.131 | 57.123 | 11.977 | 1.00 | 54.94 | A | O |
| ATOM | 991 | OE2 | GLU | A | 125 | 51.075 | 55.364 | 11.163 | 1.00 | 57.37 | A | O |
| ATOM | 992 | N | ILE | A | 126 | 57.491 | 55.918 | 10.941 | 1.00 | 31.63 | A | N |
| ATOM | 993 | CA | ILE | A | 126 | 58.585 | 55.155 | 11.525 | 1.00 | 28.26 | A | C |
| ATOM | 994 | C | ILE | A | 126 | 59.866 | 55.991 | 11.687 | 1.00 | 26.21 | A | C |
| ATOM | 995 | O | ILE | A | 126 | 60.920 | 55.440 | 11.948 | 1.00 | 28.24 | A | O |
| ATOM | 996 | CB | ILE | A | 126 | 58.878 | 53.883 | 10.690 | 1.00 | 31.52 | A | C |
| ATOM | 997 | CG1 | ILE | A | 126 | 59.197 | 54.235 | 9.234 | 1.00 | 28.75 | A | C |
| ATOM | 998 | CG2 | ILE | A | 126 | 57.699 | 52.908 | 10.764 | 1.00 | 31.40 | A | C |
| ATOM | 999 | CD1 | ILE | A | 126 | 59.677 | 53.053 | 8.429 | 1.00 | 29.82 | A | C |
| ATOM | 1000 | N | ALA | A | 127 | 59.751 | 57.298 | 11.493 | 1.00 | 26.38 | A | N |
| ATOM | 1001 | CA | ALA | A | 127 | 60.844 | 58.222 | 11.762 | 1.00 | 28.14 | A | C |
| ATOM | 1002 | C | ALA | A | 127 | 61.072 | 58.286 | 13.267 | 1.00 | 30.57 | A | C |
| ATOM | 1003 | O | ALA | A | 127 | 60.139 | 58.129 | 14.056 | 1.00 | 27.34 | A | O |
| ATOM | 1004 | CB | ALA | A | 127 | 60.516 | 59.588 | 11.228 | 1.00 | 26.20 | A | C |
| ATOM | 1005 | N | ARG | A | 128 | 62.323 | 58.479 | 13.650 | 1.00 | 32.29 | A | N |
| ATOM | 1006 | CA | ARG | A | 128 | 62.686 | 58.711 | 15.042 | 1.00 | 32.33 | A | C |
| ATOM | 1007 | C | ARG | A | 128 | 63.110 | 60.172 | 15.214 | 1.00 | 32.76 | A | C |
| ATOM | 1008 | O | ARG | A | 128 | 63.673 | 60.773 | 14.288 | 1.00 | 28.12 | A | O |
| ATOM | 1009 | CB | ARG | A | 128 | 63.775 | 57.748 | 15.468 | 1.00 | 33.84 | A | C |
| ATOM | 1010 | CG | ARG | A | 128 | 63.268 | 56.329 | 15.638 | 1.00 | 39.19 | A | C |
| ATOM | 1011 | CD | ARG | A | 128 | 64.006 | 55.302 | 14.843 | 1.00 | 43.32 | A | C |
| ATOM | 1012 | NE | ARG | A | 128 | 63.338 | 54.007 | 14.915 | 1.00 | 49.88 | A | N |
| ATOM | 1013 | CZ | ARG | A | 128 | 63.811 | 52.881 | 14.384 | 1.00 | 49.47 | A | C |
| ATOM | 1014 | NH1 | ARG | A | 128 | 63.115 | 51.757 | 14.508 | 1.00 | 52.74 | A | N |
| ATOM | 1015 | NH2 | ARG | A | 128 | 64.968 | 52.865 | 13.731 | 1.00 | 50.48 | A | N |
| ATOM | 1016 | N | PRO | A | 129 | 62.816 | 60.791 | 16.364 | 1.00 | 31.47 | A | N |
| ATOM | 1017 | CA | PRO | A | 129 | 62.218 | 60.159 | 17.553 | 1.00 | 34.71 | A | C |
| ATOM | 1018 | C | PRO | A | 129 | 60.705 | 59.942 | 17.479 | 1.00 | 34.15 | A | C |
| ATOM | 1019 | O | PRO | A | 129 | 60.172 | 59.122 | 18.229 | 1.00 | 37.59 | A | O |
| ATOM | 1020 | CB | PRO | A | 129 | 62.498 | 61.176 | 18.670 | 1.00 | 32.03 | A | C |
| ATOM | 1021 | CG | PRO | A | 129 | 62.887 | 62.461 | 18.005 | 1.00 | 33.32 | A | C |
| ATOM | 1022 | CD | PRO | A | 129 | 63.036 | 62.232 | 16.548 | 1.00 | 34.13 | A | C |
| ATOM | 1023 | N | ASP | A | 130 | 60.031 | 60.701 | 16.626 | 1.00 | 34.80 | A | N |
| ATOM | 1024 | CA | ASP | A | 130 | 58.604 | 60.519 | 16.390 | 1.00 | 38.13 | A | C |
| ATOM | 1025 | C | ASP | A | 130 | 58.234 | 60.967 | 14.976 | 1.00 | 36.50 | A | C |
| ATOM | 1026 | O | ASP | A | 130 | 59.075 | 61.471 | 14.227 | 1.00 | 36.70 | A | O |
| ATOM | 1027 | CB | ASP | A | 130 | 57.779 | 61.280 | 17.450 | 1.00 | 39.51 | A | C |
| ATOM | 1028 | CG | ASP | A | 130 | 58.154 | 62.756 | 17.558 | 1.00 | 44.22 | A | C |
| ATOM | 1029 | OD1 | ASP | A | 130 | 58.795 | 63.139 | 18.571 | 1.00 | 51.20 | A | O |
| ATOM | 1030 | OD2 | ASP | A | 130 | 57.839 | 63.614 | 16.705 | 1.00 | 44.98 | A | O |
| ATOM | 1031 | N | ASP | A | 131 | 56.963 | 60.814 | 14.623 | 1.00 | 38.18 | A | N |
| ATOM | 1032 | CA | ASP | A | 131 | 56.511 | 61.090 | 13.261 | 1.00 | 38.88 | A | C |
| ATOM | 1033 | C | ASP | A | 131 | 56.397 | 62.569 | 12.911 | 1.00 | 36.53 | A | C |
| ATOM | 1034 | O | ASP | A | 131 | 55.943 | 62.905 | 11.827 | 1.00 | 35.45 | A | O |
| ATOM | 1035 | CB | ASP | A | 131 | 55.191 | 60.346 | 12.950 | 1.00 | 39.38 | A | C |
| ATOM | 1036 | CG | ASP | A | 131 | 54.010 | 60.844 | 13.771 | 1.00 | 41.70 | A | C |
| ATOM | 1037 | OD1 | ASP | A | 131 | 54.067 | 61.976 | 14.296 | 1.00 | 42.89 | A | O |
| ATOM | 1038 | OD2 | ASP | A | 131 | 52.970 | 60.165 | 13.935 | 1.00 | 42.85 | A | O |
| ATOM | 1039 | N | SER | A | 132 | 56.801 | 63.462 | 13.815 | 1.00 | 37.22 | A | N |
| ATOM | 1040 | CA | SER | A | 132 | 56.825 | 64.891 | 13.495 | 1.00 | 34.53 | A | C |
| ATOM | 1041 | C | SER | A | 132 | 58.138 | 65.313 | 12.811 | 1.00 | 32.93 | A | C |
| ATOM | 1042 | O | SER | A | 132 | 58.242 | 66.415 | 12.301 | 1.00 | 31.50 | A | O |
| ATOM | 1043 | CB | SER | A | 132 | 56.569 | 65.733 | 14.753 | 1.00 | 36.86 | A | C |
| ATOM | 1044 | OG | SER | A | 132 | 57.784 | 66.236 | 15.282 | 1.00 | 41.97 | A | O |
| ATOM | 1045 | N | LEU | A | 133 | 59.142 | 64.442 | 12.800 | 1.00 | 33.79 | A | N |
| ATOM | 1046 | CA | LEU | A | 133 | 60.371 | 64.730 | 12.053 | 1.00 | 32.04 | A | C |
| ATOM | 1047 | C | LEU | A | 133 | 60.174 | 64.308 | 10.601 | 1.00 | 30.10 | A | C |
| ATOM | 1048 | O | LEU | A | 133 | 60.179 | 63.117 | 10.279 | 1.00 | 31.31 | A | O |
| ATOM | 1049 | CB | LEU | A | 133 | 61.586 | 64.035 | 12.652 | 1.00 | 30.30 | A | C |
| ATOM | 1050 | CG | LEU | A | 133 | 62.901 | 64.622 | 12.116 | 1.00 | 31.05 | A | C |
| ATOM | 1051 | CD1 | LEU | A | 133 | 63.289 | 65.900 | 12.891 | 1.00 | 30.64 | A | C |
| ATOM | 1052 | CD2 | LEU | A | 133 | 64.000 | 63.606 | 12.180 | 1.00 | 26.23 | A | C |
| ATOM | 1053 | N | GLU | A | 134 | 60.028 | 65.294 | 9.734 | 1.00 | 32.24 | A | N |
| ATOM | 1054 | CA | GLU | A | 134 | 59.630 | 65.044 | 8.362 | 1.00 | 30.89 | A | C |
| ATOM | 1055 | C | GLU | A | 134 | 60.812 | 64.398 | 7.611 | 1.00 | 30.52 | A | C |
| ATOM | 1056 | O | GLU | A | 134 | 61.938 | 64.919 | 7.650 | 1.00 | 27.23 | A | O |
| ATOM | 1057 | CB | GLU | A | 134 | 59.088 | 66.332 | 7.709 | 1.00 | 35.75 | A | C |
| ATOM | 1058 | CG | GLU | A | 134 | 59.723 | 66.804 | 6.414 | 1.00 | 41.00 | A | C |
| ATOM | 1059 | CD | GLU | A | 134 | 59.016 | 68.022 | 5.819 | 1.00 | 43.25 | A | C |
| ATOM | 1060 | OE1 | GLU | A | 134 | 59.719 | 68.930 | 5.302 | 1.00 | 45.72 | A | O |
| ATOM | 1061 | OE2 | GLU | A | 134 | 57.763 | 68.096 | 5.869 | 1.00 | 48.16 | A | O |
| ATOM | 1062 | N | PRO | A | 135 | 60.566 | 63.249 | 6.975 | 1.00 | 25.18 | A | N |
| ATOM | 1063 | CA | PRO | A | 135 | 61.581 | 62.606 | 6.119 | 1.00 | 24.30 | A | C |
| ATOM | 1064 | C | PRO | A | 135 | 62.039 | 63.453 | 4.958 | 1.00 | 19.94 | A | C |
| ATOM | 1065 | O | PRO | A | 135 | 61.337 | 64.319 | 4.481 | 1.00 | 23.86 | A | O |
| ATOM | 1066 | CB | PRO | A | 135 | 60.847 | 61.379 | 5.579 | 1.00 | 22.83 | A | C |
| ATOM | 1067 | CG | PRO | A | 135 | 59.796 | 61.109 | 6.573 | 1.00 | 25.70 | A | C |
| ATOM | 1068 | CD | PRO | A | 135 | 59.328 | 62.450 | 7.020 | 1.00 | 24.61 | A | C |
| ATOM | 1069 | N | PHE | A | 136 | 63.243 | 63.160 | 4.474 | 1.00 | 19.75 | A | N |
| ATOM | 1070 | CA | PHE | A | 136 | 63.850 | 63.848 | 3.367 | 1.00 | 20.77 | A | C |
| ATOM | 1071 | C | PHE | A | 136 | 62.945 | 64.000 | 2.166 | 1.00 | 25.10 | A | C |
| ATOM | 1072 | O | PHE | A | 136 | 62.798 | 65.099 | 1.632 | 1.00 | 23.63 | A | O |
| ATOM | 1073 | CB | PHE | A | 136 | 65.094 | 63.106 | 2.886 | 1.00 | 21.20 | A | C |
| ATOM | 1074 | CG | PHE | A | 136 | 65.704 | 63.716 | 1.669 | 1.00 | 19.23 | A | C |
| ATOM | 1075 | CD1 | PHE | A | 136 | 66.414 | 64.905 | 1.758 | 1.00 | 26.30 | A | C |
| ATOM | 1076 | CD2 | PHE | A | 136 | 65.522 | 63.144 | 0.421 | 1.00 | 23.32 | A | C |
| ATOM | 1077 | CE1 | PHE | A | 136 | 66.962 | 65.494 | 0.626 | 1.00 | 25.62 | A | C |
| ATOM | 1078 | CE2 | PHE | A | 136 | 66.078 | 63.727 | −0.719 | 1.00 | 23.61 | A | C |
| ATOM | 1079 | CZ | PHE | A | 136 | 66.787 | 64.903 | −0.615 | 1.00 | 29.48 | A | C |
| ATOM | 1080 | N | PHE | A | 137 | 62.402 | 62.886 | 1.694 | 1.00 | 21.94 | A | N |
| ATOM | 1081 | CA | PHE | A | 137 | 61.655 | 62.903 | 0.444 | 1.00 | 20.25 | A | C |
| ATOM | 1082 | C | PHE | A | 137 | 60.396 | 63.749 | 0.582 | 1.00 | 21.90 | A | C |
| ATOM | 1083 | O | PHE | A | 137 | 59.966 | 64.370 | −0.379 | 1.00 | 24.14 | A | O |
| ATOM | 1084 | CB | PHE | A | 137 | 61.271 | 61.509 | −0.026 | 1.00 | 18.67 | A | C |
| ATOM | 1085 | CG | PHE | A | 137 | 61.039 | 61.440 | −1.491 | 1.00 | 20.54 | A | C |
| ATOM | 1086 | CD1 | PHE | A | 137 | 62.099 | 61.302 | −2.361 | 1.00 | 23.22 | A | C |
| ATOM | 1087 | CD2 | PHE | A | 137 | 59.757 | 61.511 | −2.003 | 1.00 | 22.93 | A | C |
| ATOM | 1088 | CE1 | PHE | A | 137 | 61.900 | 61.241 | −3.721 | 1.00 | 27.68 | A | C |
| ATOM | 1089 | CE2 | PHE | A | 137 | 59.551 | 61.462 | −3.374 | 1.00 | 20.33 | A | C |
| ATOM | 1090 | CZ | PHE | A | 137 | 60.616 | 61.334 | −4.232 | 1.00 | 22.33 | A | C |
| ATOM | 1091 | N | ASP | A | 138 | 59.814 | 63.750 | 1.775 | 1.00 | 22.45 | A | N |
| ATOM | 1092 | CA | ASP | A | 138 | 58.649 | 64.582 | 2.081 | 1.00 | 25.79 | A | C |
| ATOM | 1093 | C | ASP | A | 138 | 59.020 | 66.055 | 1.966 | 1.00 | 27.43 | A | C |
| ATOM | 1094 | O | ASP | A | 138 | 58.296 | 66.825 | 1.326 | 1.00 | 30.80 | A | O |
| ATOM | 1095 | CB | ASP | A | 138 | 58.124 | 64.309 | 3.479 | 1.00 | 27.52 | A | C |
| ATOM | 1096 | CG | ASP | A | 138 | 57.419 | 62.982 | 3.596 | 1.00 | 33.12 | A | C |
| ATOM | 1097 | OD1 | ASP | A | 138 | 56.177 | 63.001 | 3.674 | 1.00 | 41.90 | A | O |
| ATOM | 1098 | OD2 | ASP | A | 138 | 58.004 | 61.870 | 3.644 | 1.00 | 36.84 | A | O |
| ATOM | 1099 | N | SER | A | 139 | 60.141 | 66.452 | 2.573 | 1.00 | 25.82 | A | N |
| ATOM | 1100 | CA | SER | A | 139 | 60.662 | 67.825 | 2.393 | 1.00 | 26.28 | A | C |
| ATOM | 1101 | C | SER | A | 139 | 60.957 | 68.169 | 0.940 | 1.00 | 26.44 | A | C |
| ATOM | 1102 | O | SER | A | 139 | 60.719 | 69.293 | 0.496 | 1.00 | 28.87 | A | O |
| ATOM | 1103 | CB | SER | A | 139 | 61.951 | 68.049 | 3.204 | 1.00 | 21.06 | A | C |
| ATOM | 1104 | OG | SER | A | 139 | 61.769 | 67.663 | 4.541 | 1.00 | 25.91 | A | O |
| ATOM | 1105 | N | LEU | A | 140 | 61.493 | 67.212 | 0.188 | 1.00 | 24.71 | A | N |
| ATOM | 1106 | CA | LEU | A | 140 | 61.816 | 67.443 | −1.209 | 1.00 | 24.32 | A | C |
| ATOM | 1107 | C | LEU | A | 140 | 60.547 | 67.732 | −2.019 | 1.00 | 26.78 | A | C |
| ATOM | 1108 | O | LEU | A | 140 | 60.555 | 68.649 | −2.851 | 1.00 | 28.93 | A | O |
| ATOM | 1109 | CB | LEU | A | 140 | 62.555 | 66.253 | −1.819 | 1.00 | 25.33 | A | C |
| ATOM | 1110 | CG | LEU | A | 140 | 62.797 | 66.212 | −3.332 | 1.00 | 27.48 | A | C |
| ATOM | 1111 | CD1 | LEU | A | 140 | 63.903 | 67.142 | −3.762 | 1.00 | 30.78 | A | C |
| ATOM | 1112 | CD2 | LEU | A | 140 | 63.124 | 64.795 | −3.764 | 1.00 | 32.40 | A | C |
| ATOM | 1113 | N | VAL | A | 141 | 59.482 | 66.964 | −1.774 | 1.00 | 27.82 | A | N |
| ATOM | 1114 | CA | VAL | A | 141 | 58.236 | 67.121 | −2.539 | 1.00 | 27.51 | A | C |
| ATOM | 1115 | C | VAL | A | 141 | 57.548 | 68.440 | −2.159 | 1.00 | 33.11 | A | C |
| ATOM | 1116 | O | VAL | A | 141 | 57.054 | 69.159 | −3.024 | 1.00 | 34.42 | A | O |
| ATOM | 1117 | CB | VAL | A | 141 | 57.268 | 65.953 | −2.319 | 1.00 | 30.35 | A | C |
| ATOM | 1118 | CG1 | VAL | A | 141 | 55.923 | 66.224 | −2.980 | 1.00 | 31.75 | A | C |
| ATOM | 1119 | CG2 | VAL | A | 141 | 57.849 | 64.666 | −2.885 | 1.00 | 30.79 | A | C |
| ATOM | 1120 | N | LYS | A | 142 | 57.541 | 68.747 | −0.868 | 1.00 | 32.37 | A | N |
| ATOM | 1121 | CA | LYS | A | 142 | 56.876 | 69.931 | −0.338 | 1.00 | 37.06 | A | C |
| ATOM | 1122 | C | LYS | A | 142 | 57.527 | 71.211 | −0.828 | 1.00 | 34.32 | A | C |
| ATOM | 1123 | O | LYS | A | 142 | 56.826 | 72.191 | −1.091 | 1.00 | 35.77 | A | O |
| ATOM | 1124 | CB | LYS | A | 142 | 56.876 | 69.897 | 1.187 | 1.00 | 37.65 | A | C |
| ATOM | 1125 | CG | LYS | A | 142 | 56.135 | 71.055 | 1.850 | 1.00 | 44.63 | A | C |
| ATOM | 1126 | CD | LYS | A | 142 | 55.689 | 70.702 | 3.264 | 1.00 | 46.28 | A | C |
| ATOM | 1127 | CE | LYS | A | 142 | 54.644 | 71.684 | 3.779 | 1.00 | 49.93 | A | C |
| ATOM | 1128 | NZ | LYS | A | 142 | 54.400 | 71.364 | 5.250 | 0.00 | 45.29 | A | N |
| ATOM | 1129 | N | GLN | A | 143 | 58.848 | 71.196 | −0.999 | 1.00 | 30.71 | A | N |
| ATOM | 1130 | CA | GLN | A | 143 | 59.602 | 72.415 | −1.260 | 1.00 | 32.98 | A | C |
| ATOM | 1131 | C | GLN | A | 143 | 59.948 | 72.655 | −2.726 | 1.00 | 31.39 | A | C |
| ATOM | 1132 | O | GLN | A | 143 | 60.393 | 73.754 | −3.071 | 1.00 | 35.65 | A | O |
| ATOM | 1133 | CB | GLN | A | 143 | 60.900 | 72.429 | −0.443 | 1.00 | 29.67 | A | C |
| ATOM | 1134 | CG | GLN | A | 143 | 60.712 | 72.505 | 1.045 | 1.00 | 29.86 | A | C |
| ATOM | 1135 | CD | GLN | A | 143 | 62.033 | 72.359 | 1.785 | 1.00 | 22.99 | A | C |
| ATOM | 1136 | OE1 | GLN | A | 143 | 62.072 | 71.774 | 2.871 | 1.00 | 32.84 | A | O |
| ATOM | 1137 | NE2 | GLN | A | 143 | 63.100 | 72.879 | 1.202 | 1.00 | 24.10 | A | N |
| ATOM | 1138 | N | THR | A | 144 | 59.767 | 71.650 | −3.588 | 1.00 | 30.44 | A | N |
| ATOM | 1139 | CA | THR | A | 144 | 60.095 | 71.786 | −5.011 | 1.00 | 31.63 | A | C |
| ATOM | 1140 | C | THR | A | 144 | 58.950 | 71.268 | −5.887 | 1.00 | 32.37 | A | C |
| ATOM | 1141 | O | THR | A | 144 | 57.910 | 70.882 | −5.368 | 1.00 | 35.67 | A | O |
| ATOM | 1142 | CB | THR | A | 144 | 61.405 | 71.032 | −5.365 | 1.00 | 35.08 | A | C |
| ATOM | 1143 | OG1 | THR | A | 144 | 61.169 | 69.613 | −5.395 | 1.00 | 34.32 | A | O |
| ATOM | 1144 | CG2 | THR | A | 144 | 62.458 | 71.221 | −4.298 | 1.00 | 34.68 | A | C |
| ATOM | 1145 | N | HIS | A | 145 | 59.165 | 71.247 | −7.203 | 1.00 | 36.14 | A | N |
| ATOM | 1146 | CA | HIS | A | 145 | 58.193 | 70.682 | −8.155 | 1.00 | 39.38 | A | C |
| ATOM | 1147 | C | HIS | A | 145 | 58.512 | 69.232 | −8.562 | 1.00 | 37.43 | A | C |
| ATOM | 1148 | O | HIS | A | 145 | 57.961 | 68.715 | −9.544 | 1.00 | 33.33 | A | O |
| ATOM | 1149 | CB | HIS | A | 145 | 58.097 | 71.563 | −9.409 | 1.00 | 43.05 | A | C |
| ATOM | 1150 | CG | HIS | A | 145 | 57.493 | 72.910 | −9.154 | 1.00 | 47.15 | A | C |
| ATOM | 1151 | ND1 | HIS | A | 145 | 56.200 | 73.072 | −8.703 | 1.00 | 50.46 | A | N |
| ATOM | 1152 | CD2 | HIS | A | 145 | 58.006 | 74.157 | −9.284 | 1.00 | 49.69 | A | C |
| ATOM | 1153 | CE1 | HIS | A | 145 | 55.941 | 74.361 | −8.570 | 1.00 | 51.63 | A | C |
| ATOM | 1154 | NE2 | HIS | A | 145 | 57.021 | 75.041 | −8.913 | 1.00 | 51.86 | A | N |
| ATOM | 1155 | N | VAL | A | 146 | 59.379 | 68.565 | −7.798 | 1.00 | 33.76 | A | N |
| ATOM | 1156 | CA | VAL | A | 146 | 59.705 | 67.163 | −8.059 | 1.00 | 29.14 | A | C |
| ATOM | 1157 | C | VAL | A | 146 | 58.472 | 66.301 | −7.774 | 1.00 | 22.43 | A | C |
| ATOM | 1158 | O | VAL | A | 146 | 57.885 | 66.398 | −6.697 | 1.00 | 24.83 | A | O |
| ATOM | 1159 | CB | VAL | A | 146 | 60.921 | 66.695 | −7.206 | 1.00 | 27.14 | A | C |
| ATOM | 1160 | CG1 | VAL | A | 146 | 61.151 | 65.185 | −7.339 | 1.00 | 27.28 | A | C |
| ATOM | 1161 | CG2 | VAL | A | 146 | 62.178 | 67.468 | −7.626 | 1.00 | 26.85 | A | C |
| ATOM | 1162 | N | PRO | A | 147 | 58.045 | 65.483 | −8.744 | 1.00 | 26.48 | A | N |
| ATOM | 1163 | CA | PRO | A | 147 | 56.864 | 64.637 | −8.557 | 1.00 | 26.08 | A | C |
| ATOM | 1164 | C | PRO | A | 147 | 57.049 | 63.662 | −7.403 | 1.00 | 22.97 | A | C |
| ATOM | 1165 | O | PRO | A | 147 | 58.185 | 63.217 | −7.166 | 1.00 | 27.41 | A | O |
| ATOM | 1166 | CB | PRO | A | 147 | 56.749 | 63.885 | −9.882 | 1.00 | 25.30 | A | C |
| ATOM | 1167 | CG | PRO | A | 147 | 57.462 | 64.717 | −10.865 | 1.00 | 27.21 | A | C |
| ATOM | 1168 | CD | PRO | A | 147 | 58.636 | 65.310 | −10.089 | 1.00 | 25.24 | A | C |
| ATOM | 1169 | N | ASN | A | 148 | 55.963 | 63.339 | −6.711 | 1.00 | 21.69 | A | N |
| ATOM | 1170 | CA | ASN | A | 148 | 56.014 | 62.466 | −5.551 | 1.00 | 21.05 | A | C |
| ATOM | 1171 | C | ASN | A | 148 | 56.167 | 60.969 | −5.908 | 1.00 | 21.77 | A | C |
| ATOM | 1172 | O | ASN | A | 148 | 55.305 | 60.152 | −5.607 | 1.00 | 21.62 | A | O |
| ATOM | 1173 | CB | ASN | A | 148 | 54.797 | 62.717 | −4.670 | 1.00 | 23.86 | A | C |
| ATOM | 1174 | CG | ASN | A | 148 | 54.878 | 62.024 | −3.338 | 1.00 | 22.10 | A | C |
| ATOM | 1175 | OD1 | ASN | A | 148 | 55.967 | 61.652 | −2.885 | 1.00 | 20.69 | A | O |
| ATOM | 1176 | ND2 | ASN | A | 148 | 53.716 | 61.795 | −2.710 | 1.00 | 25.43 | A | N |
| ATOM | 1177 | N | LEU | A | 149 | 57.291 | 60.629 | −6.524 | 1.00 | 20.43 | A | N |
| ATOM | 1178 | CA | LEU | A | 149 | 57.666 | 59.252 | −6.775 | 1.00 | 22.50 | A | C |
| ATOM | 1179 | C | LEU | A | 149 | 59.152 | 59.110 | −7.020 | 1.00 | 21.20 | A | C |
| ATOM | 1180 | O | LEU | A | 149 | 59.838 | 60.073 | −7.389 | 1.00 | 19.54 | A | O |
| ATOM | 1181 | CB | LEU | A | 149 | 56.859 | 58.654 | −7.927 | 1.00 | 25.03 | A | C |
| ATOM | 1182 | CG | LEU | A | 149 | 57.349 | 58.789 | −9.346 | 1.00 | 28.60 | A | C |
| ATOM | 1183 | CD1 | LEU | A | 149 | 56.502 | 57.899 | −10.267 | 1.00 | 30.70 | A | C |
| ATOM | 1184 | CD2 | LEU | A | 149 | 57.237 | 60.237 | −9.725 | 1.00 | 30.68 | A | C |
| ATOM | 1185 | N | PHE | A | 150 | 59.678 | 57.919 | −6.745 | 1.00 | 18.27 | A | N |
| ATOM | 1186 | CA | PHE | A | 150 | 61.044 | 57.586 | −7.149 | 1.00 | 18.21 | A | C |
| ATOM | 1187 | C | PHE | A | 150 | 61.116 | 56.104 | −7.566 | 1.00 | 17.26 | A | C |
| ATOM | 1188 | O | PHE | A | 150 | 60.229 | 55.324 | −7.235 | 1.00 | 17.41 | A | O |
| ATOM | 1189 | CB | PHE | A | 150 | 62.054 | 57.925 | −6.045 | 1.00 | 16.20 | A | C |
| ATOM | 1190 | CG | PHE | A | 150 | 61.904 | 57.072 | −4.808 | 1.00 | 15.43 | A | C |
| ATOM | 1191 | CD1 | PHE | A | 150 | 61.042 | 57.450 | −3.805 | 1.00 | 18.11 | A | C |
| ATOM | 1192 | CD2 | PHE | A | 150 | 62.614 | 55.885 | −4.681 | 1.00 | 17.12 | A | C |
| ATOM | 1193 | CE1 | PHE | A | 150 | 60.883 | 56.655 | −2.694 | 1.00 | 16.60 | A | C |
| ATOM | 1194 | CE2 | PHE | A | 150 | 62.477 | 55.092 | −3.564 | 1.00 | 16.66 | A | C |
| ATOM | 1195 | CZ | PHE | A | 150 | 61.588 | 55.468 | −2.576 | 1.00 | 18.98 | A | C |
| ATOM | 1196 | N | SER | A | 151 | 62.143 | 55.741 | −8.320 | 1.00 | 15.58 | A | N |
| ATOM | 1197 | CA | SER | A | 151 | 62.353 | 54.360 | −8.764 | 1.00 | 13.42 | A | C |
| ATOM | 1198 | C | SER | A | 151 | 63.779 | 53.904 | −8.612 | 1.00 | 15.43 | A | C |
| ATOM | 1199 | O | SER | A | 151 | 64.717 | 54.708 | −8.638 | 1.00 | 17.96 | A | O |
| ATOM | 1200 | CB | SER | A | 151 | 61.880 | 54.171 | −10.200 | 1.00 | 18.86 | A | C |
| ATOM | 1201 | OG | SER | A | 151 | 62.440 | 55.169 | −11.021 | 1.00 | 19.88 | A | O |
| ATOM | 1202 | N | LEU | A | 152 | 63.932 | 52.603 | −8.401 | 1.00 | 15.64 | A | N |
| ATOM | 1203 | CA | LEU | A | 152 | 65.213 | 51.992 | −8.105 | 1.00 | 16.25 | A | C |
| ATOM | 1204 | C | LEU | A | 152 | 65.456 | 50.817 | −9.015 | 1.00 | 17.29 | A | C |
| ATOM | 1205 | O | LEU | A | 152 | 64.596 | 49.925 | −9.143 | 1.09 | 17.99 | A | O |
| ATOM | 1206 | CB | LEU | A | 152 | 65.248 | 51.493 | −6.650 | 1.00 | 16.35 | A | C |
| ATOM | 1207 | CG | LEU | A | 152 | 65.317 | 52.590 | −5.590 | 1.00 | 18.65 | A | C |
| ATOM | 1208 | CD1 | LEU | A | 152 | 65.177 | 51.994 | −4.208 | 1.00 | 19.71 | A | C |
| ATOM | 1209 | CD2 | LEU | A | 152 | 66.585 | 53.418 | −5.725 | 1.00 | 19.60 | A | C |
| ATOM | 1210 | N | GLN | A | 153 | 66.618 | 50.820 | −9.646 | 1.00 | 19.70 | A | N |
| ATOM | 1211 | CA | GLN | A | 153 | 67.115 | 49.692 | −10.419 | 1.00 | 19.09 | A | C |
| ATOM | 1212 | C | GLN | A | 153 | 68.422 | 49.296 | −9.747 | 1.00 | 17.61 | A | C |
| ATOM | 1213 | O | GLN | A | 153 | 69.438 | 49.964 | −9.921 | 1.00 | 22.26 | A | O |
| ATOM | 1214 | CB | GLN | A | 153 | 67.368 | 50.078 | −11.883 | 1.00 | 23.44 | A | C |
| ATOM | 1215 | CG | GLN | A | 153 | 67.771 | 48.873 | −12.721 | 1.00 | 24.58 | A | C |
| ATOM | 1216 | CD | GLN | A | 153 | 68.573 | 49.194 | −13.957 | 1.00 | 26.89 | A | C |
| ATOM | 1217 | OE1 | GLN | A | 153 | 69.610 | 49.863 | −13.895 | 1.00 | 32.38 | A | O |
| ATOM | 1218 | NE2 | GLN | A | 153 | 68.116 | 48.681 | −15.089 | 1.00 | 27.76 | A | N |
| ATOM | 1219 | N | LEU | A | 154 | 68.392 | 48.247 | −8.941 | 1.00 | 17.74 | A | N |
| ATOM | 1220 | CA | LEU | A | 154 | 69.618 | 47.726 | −8.329 | 1.00 | 21.30 | A | C |
| ATOM | 1221 | C | LEU | A | 154 | 70.186 | 46.576 | −9.166 | 1.00 | 28.90 | A | C |
| ATOM | 1222 | O | LEU | A | 154 | 69.479 | 45.609 | −9.464 | 1.00 | 29.97 | A | O |
| ATOM | 1223 | CB | LEU | A | 154 | 69.339 | 47.276 | −6.898 | 1.00 | 21.73 | A | C |
| ATOM | 1224 | CG | LEU | A | 154 | 68.556 | 48.277 | −6.046 | 1.00 | 22.21 | A | C |
| ATOM | 1225 | CD1 | LEU | A | 154 | 68.239 | 47.712 | −4.686 | 1.00 | 25.89 | A | C |
| ATOM | 1226 | CD2 | LEU | A | 154 | 69.266 | 49.619 | −5.888 | 1.00 | 22.60 | A | C |
| ATOM | 1227 | N | CYS | A | 155 | 71.461 | 46.678 | −9.537 | 1.00 | 32.67 | A | N |
| ATOM | 1228 | CA | CYS | A | 155 | 72.096 | 45.709 | −10.442 | 1.00 | 36.54 | A | C |
| ATOM | 1229 | C | CYS | A | 155 | 73.103 | 44.805 | −9.720 | 1.00 | 41.47 | A | C |
| ATOM | 1230 | O | CYS | A | 155 | 72.719 | 43.815 | −9.116 | 1.00 | 43.64 | A | O |
| ATOM | 1231 | CB | CYS | A | 155 | 72.744 | 46.440 | −11.616 | 1.09 | 36.93 | A | C |
| ATOM | 1232 | SG | CYS | A | 155 | 71.580 | 47.462 | −12.528 | 1.00 | 37.02 | A | S |
| ATOM | 1233 | N | GLY | A | 156 | 74.389 | 45.122 | −9.802 | 1.00 | 49.66 | A | N |
| ATOM | 1234 | CA | GLY | A | 156 | 75.416 | 44.312 | −9.170 | 1.00 | 52.13 | A | C |
| ATOM | 1235 | C | GLY | A | 156 | 75.784 | 43.035 | −9.897 | 1.00 | 54.64 | A | C |
| ATOM | 1226 | O | GLY | A | 156 | 75.586 | 41.937 | −9.372 | 1.00 | 55.03 | A | O |
| ATOM | 1237 | N | ALA | A | 157 | 76.323 | 43.196 | −11.106 | 1.00 | 58.96 | A | N |
| ATOM | 1238 | CA | ALA | A | 157 | 76.935 | 42.104 | −11.872 | 1.00 | 59.70 | A | C |
| ATOM | 1239 | C | ALA | A | 157 | 76.142 | 40.799 | −11.808 | 1.00 | 61.26 | A | C |
| ATOM | 1240 | O | ALA | A | 157 | 76.543 | 39.845 | −11.131 | 1.00 | 63.37 | A | O |
| ATOM | 1241 | CB | ALA | A | 157 | 78.377 | 41.881 | −11.396 | 1.00 | 61.07 | A | C |
| ATOM | 1242 | N | ALA | A | 168 | 81.887 | 41.703 | −5.577 | 1.00 | 52.10 | A | N |
| ATOM | 1243 | CA | ALA | A | 168 | 82.673 | 42.857 | −6.011 | 1.00 | 51.66 | A | C |
| ATOM | 1244 | C | ALA | A | 168 | 81.807 | 44.132 | −6.026 | 1.00 | 49.66 | A | C |
| ATOM | 1245 | O | ALA | A | 168 | 80.833 | 44.234 | −5.270 | 1.00 | 47.62 | A | O |
| ATOM | 1246 | CB | ALA | A | 168 | 83.302 | 42.585 | −7.389 | 1.00 | 52.14 | A | C |
| ATOM | 1247 | N | SER | A | 169 | 82.169 | 45.100 | −6.865 | 1.00 | 48.50 | A | N |
| ATOM | 1248 | CA | SER | A | 169 | 81.455 | 46.373 | −6.933 | 1.00 | 47.11 | A | C |
| ATOM | 1249 | C | SER | A | 169 | 80.128 | 46.241 | −7.693 | 1.00 | 45.49 | A | C |
| ATOM | 1250 | O | SER | A | 169 | 80.102 | 45.793 | −8.833 | 1.00 | 42.21 | A | O |
| ATOM | 1251 | CB | SER | A | 169 | 82.336 | 47.432 | −7.596 | 1.00 | 48.06 | A | C |
| ATOM | 1252 | OG | SER | A | 169 | 81.625 | 48.637 | −7.812 | 1.00 | 53.03 | A | O |
| ATOM | 1253 | N | VAL | A | 170 | 79.036 | 46.648 | −7.048 | 1.00 | 40.36 | A | N |
| ATOM | 1254 | CA | VAL | A | 170 | 77.714 | 46.662 | −7.675 | 1.00 | 36.88 | A | C |
| ATOM | 1255 | C | VAL | A | 170 | 77.329 | 48.074 | −8.121 | 1.00 | 33.45 | A | C |
| ATOM | 1256 | O | VAL | A | 170 | 77.980 | 49.050 | −7.751 | 1.00 | 27.09 | A | O |
| ATOM | 1257 | CB | VAL | A | 170 | 76.636 | 46.100 | −6.714 | 1.00 | 35.68 | A | C |
| ATOM | 1258 | CG1 | VAL | A | 170 | 76.978 | 44.662 | −6.301 | 1.00 | 38.27 | A | C |
| ATOM | 1259 | CG2 | VAL | A | 170 | 76.471 | 46.986 | −5.476 | 1.00 | 36.83 | A | C |
| ATOM | 1260 | N | GLY | A | 171 | 76.256 | 48.174 | −8.905 | 1.00 | 30.02 | A | N |
| ATOM | 1261 | CA | GLY | A | 171 | 75.760 | 49.457 | −9.360 | 1.00 | 28.27 | A | C |
| ATOM | 1262 | C | GLV | A | 171 | 74.250 | 49.511 | −9.521 | 1.00 | 23.99 | A | C |
| ATOM | 1263 | O | GLY | A | 171 | 73.567 | 48.502 | −9.456 | 1.00 | 30.07 | A | O |
| ATOM | 1264 | N | GLY | A | 172 | 73.748 | 50.704 | −9.785 | 1.00 | 22.93 | A | N |
| ATOM | 1265 | CA | GLY | A | 172 | 72.321 | 50.912 | −9.960 | 1.00 | 24.79 | A | C |
| ATOM | 1266 | C | GLY | A | 172 | 71.921 | 52.328 | −10.318 | 1.00 | 21.72 | A | C |
| ATOM | 1267 | O | GLY | A | 172 | 72.755 | 53.177 | −10.586 | 1.00 | 20.97 | A | O |
| ATOM | 1268 | N | SER | A | 173 | 70.615 | 52.576 | −10.323 | 1.00 | 20.55 | A | N |
| ATOM | 1269 | CA | SER | A | 173 | 70.056 | 53.881 | −10.618 | 1.00 | 19.75 | A | C |
| ATOM | 1270 | C | SER | A | 173 | 68.934 | 54.169 | −9.642 | 1.00 | 17.92 | A | C |
| ATOM | 1271 | O | SER | A | 173 | 68.098 | 53.318 | −9.396 | 1.00 | 19.13 | A | O |
| ATOM | 1272 | CB | SER | A | 173 | 69.490 | 53.959 | −12.045 | 1.00 | 20.34 | A | C |
| ATOM | 1273 | OG | SER | A | 173 | 70.498 | 53.718 | −13.025 | 1.00 | 23.31 | A | O |
| ATOM | 1274 | N | MET | A | 174 | 68.935 | 55.371 | −9.085 | 1.09 | 19.22 | A | N |
| ATOM | 1275 | CA | MET | A | 174 | 67.794 | 55.904 | −8.368 | 1.00 | 19.42 | A | C |
| ATOM | 1276 | C | MET | A | 174 | 67.284 | 57.099 | −9.164 | 1.00 | 20.07 | A | C |
| ATOM | 1277 | O | MET | A | 174 | 67.936 | 58.150 | −9.226 | 1.00 | 18.63 | A | O |
| ATOM | 1278 | CB | MET | A | 174 | 68.156 | 56.332 | −6.953 | 1.00 | 19.01 | A | C |
| ATOM | 1279 | CG | MET | A | 174 | 66.982 | 56.914 | −6.230 | 1.00 | 22.45 | A | C |
| ATOM | 1280 | SD | MET | A | 174 | 67.349 | 57.388 | −4.532 | 1.00 | 24.52 | A | S |
| ATOM | 1281 | CE | MET | A | 174 | 68.659 | 58.440 | −4.766 | 1.00 | 27.89 | A | C |
| ATOM | 1282 | N | ILE | A | 175 | 66.135 | 56.904 | −9.818 | 1.00 | 21.08 | A | N |
| ATOM | 1283 | CA | ILE | A | 175 | 65.469 | 57.972 | −10.548 | 1.00 | 16.99 | A | C |
| ATOM | 1284 | C | ILE | A | 175 | 64.484 | 58.690 | −9.642 | 1.00 | 16.84 | A | C |
| ATOM | 1285 | O | ILE | A | 175 | 63.468 | 58.119 | −9.242 | 1.00 | 19.54 | A | O |
| ATOM | 1286 | CB | ILE | A | 175 | 64.740 | 57.415 | −11.800 | 1.00 | 22.71 | A | C |
| ATOM | 1287 | CG1 | ILE | A | 175 | 65.645 | 56.492 | −12.632 | 1.00 | 21.64 | A | C |
| ATOM | 1288 | CG2 | ILE | A | 175 | 64.160 | 58.559 | −12.633 | 1.00 | 22.47 | A | C |
| ATOM | 1289 | CD1 | ILE | A | 175 | 66.942 | 57.124 | −13.192 | 1.00 | 21.34 | A | C |
| ATOM | 1290 | N | ILE | A | 176 | 64.820 | 59.929 | −9.276 | 1.00 | 22.11 | A | N |
| ATOM | 1291 | CA | ILE | A | 176 | 64.012 | 60.750 | −8.396 | 1.00 | 22.51 | A | C |
| ATOM | 1292 | C | ILE | A | 176 | 63.045 | 61.581 | −9.230 | 1.00 | 23.28 | A | C |
| ATOM | 1293 | O | ILE | A | 176 | 63.464 | 62.397 | −10.056 | 1.00 | 27.99 | A | O |
| ATOM | 1294 | CB | ILE | A | 176 | 64.908 | 61.703 | −7.567 | 1.00 | 24.59 | A | C |
| ATOM | 1295 | CG1 | ILE | A | 176 | 65.914 | 60.917 | −6.718 | 1.00 | 27.72 | A | C |
| ATOM | 1296 | CG2 | ILE | A | 176 | 64.059 | 62.601 | −6.699 | 1.00 | 26.83 | A | C |
| ATOM | 1297 | CD1 | ILE | A | 176 | 65.274 | 60.104 | −5.618 | 1.00 | 30.97 | A | C |
| ATOM | 1298 | N | GLY | A | 177 | 61.762 | 61.359 | −9.003 | 1.00 | 23.04 | A | N |
| ATOM | 1299 | CA | GLY | A | 177 | 60.711 | 62.169 | −9.578 | 1.00 | 23.58 | A | C |
| ATOM | 1300 | C | GLY | A | 177 | 60.114 | 61.540 | −10.810 | 1.00 | 27.69 | A | C |
| ATOM | 1301 | O | GLY | A | 177 | 59.224 | 62.125 | −11.428 | 1.00 | 29.57 | A | O |
| ATOM | 1302 | N | GLY | A | 178 | 60.561 | 60.340 | −11.160 | 1.00 | 24.14 | A | N |
| ATOM | 1303 | CA | GLY | A | 178 | 60.023 | 59.706 | −12.342 | 1.00 | 26.72 | A | C |
| ATOM | 1304 | C | GLY | A | 178 | 60.460 | 58.295 | −12.586 | 1.00 | 27.37 | A | C |
| ATOM | 1305 | O | GLY | A | 178 | 61.017 | 57.610 | −11.712 | 1.00 | 24.67 | A | O |
| ATOM | 1306 | N | ILE | A | 179 | 60.153 | 57.861 | −13.800 | 1.00 | 24.35 | A | N |
| ATOM | 1307 | CA | ILE | A | 179 | 60.475 | 56.541 | −14.300 | 1.00 | 28.10 | A | C |
| ATOM | 1308 | C | ILE | A | 179 | 61.277 | 56.703 | −15.591 | 1.00 | 30.00 | A | C |
| ATOM | 1309 | O | ILE | A | 179 | 61.031 | 57.640 | −16.367 | 1.00 | 29.66 | A | O |
| ATOM | 1310 | CB | ILE | A | 179 | 59.174 | 55.751 | −14.552 | 1.00 | 28.71 | A | C |
| ATOM | 1311 | CG1 | ILE | A | 179 | 58.240 | 55.833 | −13.314 | 1.00 | 31.09 | A | C |
| ATOM | 1312 | CG2 | ILE | A | 179 | 59.480 | 54.319 | −14.890 | 1.00 | 32.54 | A | C |
| ATOM | 1313 | CD1 | ILE | A | 179 | 56.941 | 55.086 | −13.456 | 1.00 | 35.50 | A | C |
| ATOM | 1314 | N | ASP | A | 180 | 62.241 | 55.806 | −15.795 | 1.00 | 29.78 | A | N |
| ATOM | 1315 | CA | ASP | A | 180 | 63.094 | 55.783 | −16.983 | 1.00 | 32.20 | A | C |
| ATOM | 1316 | C | ASP | A | 180 | 62.895 | 54.450 | −17.703 | 1.00 | 30.45 | A | C |
| ATOM | 1317 | O | ASP | A | 180 | 63.345 | 53.399 | −17.240 | 1.00 | 27.79 | A | O |
| ATOM | 1318 | CB | ASP | A | 180 | 64.566 | 55.955 | −16.576 | 1.00 | 31.37 | A | C |
| ATOM | 1319 | CG | ASP | A | 180 | 65.488 | 56.151 | −17.759 | 1.00 | 37.38 | A | C |
| ATOM | 1320 | OD1 | ASP | A | 180 | 65.155 | 55.692 | −18.868 | 1.00 | 40.09 | A | O |
| ATOM | 1321 | OD2 | ASP | A | 180 | 66.577 | 56.746 | −17.670 | 1.00 | 37.04 | A | O |
| ATOM | 1322 | N | HIS | A | 181 | 62.235 | 54.504 | −18.856 | 1.00 | 34.60 | A | N |
| ATOM | 1323 | CA | HIS | A | 181 | 61.829 | 53.298 | −19.592 | 1.00 | 34.97 | A | C |
| ATOM | 1324 | C | HIS | A | 181 | 62.986 | 52.434 | −20.108 | 1.00 | 35.24 | A | C |
| ATOM | 1325 | O | HIS | A | 181 | 62.825 | 51.229 | −20.323 | 1.00 | 34.58 | A | O |
| ATOM | 1326 | CB | HIS | A | 181 | 60.868 | 53.695 | −20.721 | 1.00 | 38.80 | A | C |
| ATOM | 1327 | CG | HIS | A | 181 | 59.662 | 54.442 | −20.233 | 0.50 | 38.66 | A | C |
| ATOM | 1328 | ND1 | HIS | A | 181 | 58.846 | 53.959 | −19.234 | 0.50 | 38.35 | A | N |
| ATOM | 1329 | CD2 | HIS | A | 181 | 59.158 | 55.649 | −20.580 | 0.50 | 40.41 | A | C |
| ATOM | 1330 | CE1 | HIS | A | 181 | 57.880 | 54.828 | −18.998 | 0.50 | 40.15 | A | C |
| ATOM | 1331 | NE2 | HIS | A | 181 | 58.045 | 55.863 | −19.803 | 0.50 | 40.15 | A | N |
| ATOM | 1332 | N | SER | A | 182 | 64.167 | 53.026 | −20.244 | 1.00 | 33.98 | A | N |
| ATOM | 1333 | CA | SER | A | 182 | 65.369 | 52.269 | −20.589 | 1.00 | 34.19 | A | C |
| ATOM | 1334 | C | SER | A | 182 | 65.834 | 51.304 | −19.484 | 1.00 | 31.16 | A | C |
| ATOM | 1335 | O | SER | A | 182 | 66.638 | 50.418 | −19.736 | 1.00 | 29.95 | A | O |
| ATOM | 1336 | CB | SER | A | 182 | 66.507 | 53.230 | −20.966 | 1.00 | 36.56 | A | C |
| ATOM | 1337 | OG | SER | A | 182 | 66.853 | 54.088 | −19.886 | 1.00 | 39.80 | A | O |
| ATOM | 1338 | N | LEU | A | 183 | 65.318 | 51.458 | −18.261 | 1.00 | 28.19 | A | N |
| ATOM | 1339 | CA | LEU | A | 183 | 65.719 | 50.607 | −17.156 | 1.00 | 26.88 | A | C |
| ATOM | 1340 | C | LEU | A | 183 | 64.920 | 49.310 | −17.012 | 1.00 | 26.28 | A | C |
| ATOM | 1341 | O | LEU | A | 183 | 65.267 | 48.479 | −16.178 | 1.00 | 21.62 | A | O |
| ATOM | 1342 | CB | LEU | A | 183 | 65.646 | 51.397 | −15.838 | 1.00 | 26.71 | A | C |
| ATOM | 1343 | CG | LEU | A | 183 | 66.557 | 52.620 | −15.805 | 1.00 | 30.09 | A | C |
| ATOM | 1344 | CD1 | LEU | A | 183 | 66.413 | 53.360 | −14.479 | 1.00 | 28.34 | A | C |
| ATOM | 1345 | CD2 | LEU | A | 183 | 67.997 | 52.185 | −16.027 | 1.00 | 33.43 | A | C |
| ATOM | 1346 | N | TYR | A | 184 | 63.865 | 49.130 | −17.822 | 1.00 | 26.39 | A | N |
| ATOM | 1347 | CA | TYR | A | 184 | 63.038 | 47.935 | −17.710 | 1.00 | 22.78 | A | C |
| ATOM | 1348 | C | TYR | A | 184 | 62.486 | 47.453 | −19.040 | 1.00 | 22.69 | A | C |
| ATOM | 1349 | O | TYR | A | 184 | 62.380 | 48.227 | −19.990 | 1.00 | 23.92 | A | O |
| ATOM | 1350 | CB | TYR | A | 184 | 61.856 | 48.135 | −16.742 | 1.00 | 22.73 | A | C |
| ATOM | 1351 | CG | TYR | A | 184 | 60.726 | 49.040 | −17.193 | 1.00 | 20.79 | A | C |
| ATOM | 1352 | CD1 | TYR | A | 184 | 59.540 | 48.523 | −17.735 | 1.00 | 21.72 | A | C |
| ATOM | 1353 | CD2 | TYR | A | 184 | 60.812 | 50.410 | −17.035 | 1.00 | 23.49 | A | C |
| ATOM | 1354 | CE1 | TYR | A | 184 | 58.500 | 49.357 | −18.123 | 1.00 | 23.85 | A | C |
| ATOM | 1355 | CE2 | TYR | A | 184 | 59.776 | 51.250 | −17.395 | 1.00 | 21.72 | A | C |
| ATOM | 1356 | CZ | TYR | A | 184 | 58.616 | 50.718 | −17.949 | 1.00 | 23.96 | A | C |
| ATOM | 1357 | OH | TYR | A | 184 | 57.603 | 51.567 | −18.306 | 1.00 | 27.89 | A | O |
| ATOM | 1358 | N | THR | A | 185 | 62.082 | 46.190 | −19.048 | 1.00 | 26.60 | A | N |
| ATOM | 1359 | CA | THR | A | 185 | 61.397 | 45.590 | −20.194 | 1.00 | 23.12 | A | C |
| ATOM | 1360 | C | THR | A | 185 | 60.012 | 45.120 | −19.777 | 1.00 | 26.64 | A | C |
| ATOM | 1361 | O | THR | A | 185 | 59.754 | 44.849 | −18.608 | 1.00 | 24.65 | A | O |
| ATOM | 1362 | CB | THR | A | 185 | 62.215 | 44.414 | −20.801 | 1.00 | 25.79 | A | C |
| ATOM | 1363 | OG1 | THR | A | 185 | 62.261 | 43.299 | −19.906 | 1.00 | 28.94 | A | O |
| ATOM | 1364 | CG2 | THR | A | 185 | 63.702 | 44.791 | −20.980 | 1.00 | 32.36 | A | C |
| ATOM | 1365 | N | GLY | A | 186 | 59.127 | 44.998 | −20.762 | 1.00 | 30.22 | A | N |
| ATOM | 1366 | CA | GLY | A | 186 | 57.765 | 44.602 | −20.489 | 1.00 | 28.03 | A | C |
| ATOM | 1367 | C | GLY | A | 186 | 57.019 | 45.726 | −19.805 | 1.00 | 24.17 | A | C |
| ATOM | 1368 | O | GLY | A | 186 | 57.380 | 46.894 | −19.927 | 1.00 | 29.37 | A | O |
| ATOM | 1369 | N | SER | A | 187 | 55.952 | 45.365 | −19.102 | 1.00 | 23.81 | A | N |
| ATOM | 1370 | CA | SER | A | 187 | 55.062 | 46.328 | −18.488 | 1.00 | 21.71 | A | C |
| ATOM | 1371 | C | SER | A | 187 | 55.311 | 46.342 | −16.996 | 1.00 | 19.66 | A | C |
| ATOM | 1372 | O | SER | A | 187 | 55.732 | 45.342 | −16.426 | 1.00 | 20.42 | A | O |
| ATOM | 1373 | CB | SER | A | 187 | 53.601 | 45.940 | −18.750 | 1.00 | 23.20 | A | C |
| ATOM | 1374 | OG | SER | A | 187 | 52.695 | 46.740 | −18.000 | 1.00 | 25.16 | A | O |
| ATOM | 1375 | N | LEU | A | 188 | 55.046 | 47.493 | −16.390 | 1.00 | 18.89 | A | N |
| ATOM | 1376 | CA | LEU | A | 188 | 54.965 | 47.608 | −14.928 | 1.00 | 17.77 | A | C |
| ATOM | 1377 | C | LEU | A | 188 | 53.629 | 47.054 | −14.466 | 1.00 | 19.39 | A | C |
| ATOM | 1378 | O | LEU | A | 188 | 52.601 | 47.299 | −15.082 | 1.00 | 21.63 | A | O |
| ATOM | 1379 | CB | LEU | A | 188 | 55.067 | 49.054 | −14.470 | 1.00 | 18.74 | A | C |
| ATOM | 1380 | CG | LEU | A | 188 | 56.433 | 49.736 | −14.522 | 1.00 | 18.45 | A | C |
| ATOM | 1381 | CD1 | LEU | A | 188 | 56.311 | 51.222 | −14.556 | 1.00 | 20.69 | A | C |
| ATOM | 1382 | CD2 | LEU | A | 188 | 57.295 | 49.273 | −13.305 | 1.00 | 19.48 | A | C |
| ATOM | 1383 | N | TRP | A | 189 | 53.670 | 46.295 | −13.384 | 1.00 | 13.99 | A | N |
| ATOM | 1384 | CA | TRP | A | 189 | 52.524 | 45.838 | −12.633 | 1.00 | 15.66 | A | C |
| ATOM | 1385 | C | TRP | A | 189 | 52.595 | 46.453 | −11.245 | 1.00 | 16.26 | A | C |
| ATOM | 1386 | O | TRP | A | 189 | 53.650 | 46.442 | −10.633 | 1.00 | 17.41 | A | O |
| ATOM | 1387 | CB | TRP | A | 189 | 52.542 | 44.325 | −12.516 | 1.00 | 15.88 | A | C |
| ATOM | 1388 | CG | TRP | A | 189 | 52.121 | 43.681 | −13.817 | 1.00 | 18.59 | A | C |
| ATOM | 1389 | CD1 | TRP | A | 189 | 52.916 | 43.461 | −14.898 | 1.00 | 21.85 | A | C |
| ATOM | 1390 | CD2 | TRP | A | 189 | 50.800 | 43.262 | −14.200 | 1.00 | 18.24 | A | C |
| ATOM | 1391 | NE1 | TRP | A | 189 | 52.189 | 42.888 | −15.919 | 1.00 | 23.05 | A | N |
| ATOM | 1392 | CE2 | TRP | A | 189 | 50.885 | 42.772 | −15.522 | 1.00 | 19.78 | A | C |
| ATOM | 1393 | CE3 | TRP | A | 189 | 49.552 | 43.264 | −13.570 | 1.00 | 15.96 | A | C |
| ATOM | 1394 | CZ2 | TRP | A | 189 | 49.777 | 42.292 | −16.224 | 1.00 | 20.18 | A | C |
| ATOM | 1395 | CZ3 | TRP | A | 189 | 48.436 | 42.778 | −14.274 | 1.00 | 17.80 | A | C |
| ATOM | 1396 | CH2 | TRP | A | 189 | 48.570 | 42.298 | −15.590 | 1.00 | 16.72 | A | C |
| ATOM | 1397 | N | TYR | A | 190 | 51.467 | 46.920 | −10.739 | 1.00 | 14.59 | A | N |
| ATOM | 1398 | CA | TYR | A | 190 | 51.425 | 47.625 | −9.453 | 1.00 | 14.85 | A | C |
| ATOM | 1399 | C | TYR | A | 190 | 50.631 | 46.901 | −8.353 | 1.00 | 17.95 | A | C |
| ATOM | 1400 | O | TYR | A | 190 | 49.564 | 46.289 | −8.586 | 1.00 | 13.30 | A | O |
| ATOM | 1401 | CB | TYR | A | 190 | 50.864 | 49.021 | −9.636 | 1.00 | 13.88 | A | C |
| ATOM | 1402 | CG | TYR | A | 190 | 51.635 | 49.973 | −10.515 | 1.00 | 15.59 | A | C |
| ATOM | 1403 | CD1 | TYR | A | 190 | 52.573 | 50.842 | −9.977 | 1.00 | 15.38 | A | C |
| ATOM | 1404 | CD2 | TYR | A | 190 | 51.339 | 50.092 | −11.866 | 1.00 | 17.42 | A | C |
| ATOM | 1405 | CE1 | TYR | A | 190 | 53.237 | 51.770 | −10.760 | 1.00 | 17.56 | A | C |
| ATOM | 1406 | CE2 | TYR | A | 190 | 52.018 | 51.038 | −12.685 | 1.00 | 14.93 | A | C |
| ATOM | 1407 | CZ | TYR | A | 190 | 52.954 | 51.873 | −12.107 | 1.00 | 18.74 | A | C |
| ATOM | 1408 | OH | TYR | A | 190 | 53.638 | 52.785 | −12.865 | 1.00 | 17.23 | A | O |
| ATOM | 1409 | N | THR | A | 191 | 51.182 | 46.980 | −7.139 | 1.00 | 16.74 | A | N |
| ATOM | 1410 | CA | THR | A | 191 | 50.568 | 46.429 | −5.953 | 1.00 | 15.64 | A | C |
| ATOM | 1411 | C | THR | A | 191 | 50.304 | 47.626 | −5.008 | 1.00 | 17.89 | A | C |
| ATOM | 1412 | O | THR | A | 191 | 51.106 | 48.544 | −4.975 | 1.00 | 16.52 | A | O |
| ATOM | 1413 | CB | THR | A | 191 | 51.520 | 45.392 | −5.357 | 1.00 | 16.95 | A | C |
| ATOM | 1414 | OG1 | THR | A | 191 | 50.861 | 44.672 | −4.325 | 1.00 | 19.32 | A | O |
| ATOM | 1415 | CG2 | THR | A | 191 | 52.768 | 46.057 | −4.680 | 1.00 | 16.37 | A | C |
| ATOM | 1416 | N | PRO | A | 192 | 49.168 | 47.686 | −4.309 | 1.00 | 20.12 | A | N |
| ATOM | 1417 | CA | PRO | A | 192 | 48.944 | 48.801 | −3.365 | 1.00 | 21.23 | A | C |
| ATOM | 1418 | C | PRO | A | 192 | 49.911 | 48.871 | −2.178 | 1.00 | 17.60 | A | C |
| ATOM | 1419 | O | PRO | A | 192 | 50.370 | 47.856 | −1.643 | 1.00 | 24.36 | A | O |
| ATOM | 1420 | CB | PRO | A | 192 | 47.504 | 48.585 | −2.876 | 1.00 | 21.11 | A | C |
| ATOM | 1421 | CG | PRO | A | 192 | 46.881 | 47.748 | −3.955 | 1.00 | 22.26 | A | C |
| ATOM | 1422 | CD | PRO | A | 192 | 47.980 | 46.828 | −4.424 | 1.00 | 23.51 | A | C |
| ATOM | 1423 | N | ILE | A | 193 | 50.235 | 50.099 | −1.797 | 1.00 | 21.72 | A | N |
| ATOM | 1424 | CA | ILE | A | 193 | 50.881 | 50.339 | −0.515 | 1.00 | 22.71 | A | C |
| ATOM | 1425 | C | ILE | A | 193 | 49.758 | 50.230 | 0.508 | 1.00 | 23.63 | A | C |
| ATOM | 1426 | O | ILE | A | 193 | 48.881 | 51.079 | 0.568 | 1.00 | 29.68 | A | O |
| ATOM | 1427 | CB | ILE | A | 193 | 51.550 | 51.713 | −0.453 | 1.00 | 24.36 | A | C |
| ATOM | 1428 | CG1 | ILE | A | 193 | 52.730 | 51.781 | −1.438 | 1.00 | 24.14 | A | C |
| ATOM | 1429 | CG2 | ILE | A | 193 | 52.036 | 51.993 | 0.987 | 1.00 | 24.12 | A | C |
| ATOM | 1430 | CD1 | ILE | A | 193 | 53.313 | 53.171 | −1.629 | 1.00 | 22.49 | A | C |
| ATOM | 1431 | N | ARG | A | 194 | 49.764 | 49.145 | 1.257 | 1.00 | 29.07 | A | N |
| ATOM | 1432 | CA | ARG | A | 194 | 48.696 | 48.887 | 2.199 | 1.00 | 32.57 | A | C |
| ATOM | 1433 | C | ARG | A | 194 | 48.547 | 50.005 | 3.219 | 1.00 | 33.92 | A | C |
| ATOM | 1434 | O | ARG | A | 194 | 47.446 | 50.517 | 3.435 | 1.00 | 36.50 | A | O |
| ATOM | 1435 | CB | ARG | A | 194 | 48.940 | 47.592 | 2.930 | 1.00 | 31.65 | A | C |
| ATOM | 1436 | CG | ARG | A | 194 | 47.768 | 47.245 | 3.797 | 1.00 | 32.32 | A | C |
| ATOM | 1437 | CD | ARG | A | 194 | 48.031 | 46.137 | 4.719 | 1.00 | 34.05 | A | C |
| ATOM | 1438 | NE | ARG | A | 194 | 46.832 | 45.833 | 5.482 | 1.00 | 37.69 | A | N |
| ATOM | 1439 | CZ | ARG | A | 194 | 46.774 | 44.914 | 6.424 | 1.00 | 43.49 | A | C |
| ATOM | 1440 | NH1 | ARG | A | 194 | 47.853 | 44.211 | 6.726 | 1.00 | 45.66 | A | N |
| ATOM | 1441 | NH2 | ARG | A | 194 | 45.636 | 44.700 | 7.079 | 1.00 | 44.35 | A | N |
| ATOM | 1442 | N | ARG | A | 195 | 49.668 | 50.353 | 3.839 | 1.00 | 35.28 | A | N |
| ATOM | 1443 | CA | ARG | A | 195 | 49.740 | 51.436 | 4.817 | 1.00 | 35.11 | A | C |
| ATOM | 1444 | C | ARG | A | 195 | 51.059 | 52.190 | 4.631 | 1.00 | 32.34 | A | C |
| ATOM | 1445 | O | ARG | A | 195 | 52.089 | 51.576 | 4.401 | 1.00 | 28.84 | A | O |
| ATOM | 1446 | CB | ARG | A | 195 | 49.683 | 50.857 | 6.226 | 1.00 | 35.59 | A | C |
| ATOM | 1447 | CG | ARG | A | 195 | 49.645 | 51.910 | 7.339 | 1.00 | 40.30 | A | C |
| ATOM | 1448 | CD | ARG | A | 195 | 48.907 | 51.460 | 8.591 | 1.00 | 43.51 | A | C |
| ATOM | 1449 | NE | ARG | A | 195 | 49.734 | 50.619 | 9.458 | 1.00 | 44.69 | A | N |
| ATOM | 1450 | CZ | ARG | A | 195 | 50.582 | 51.069 | 10.387 | 1.00 | 46.19 | A | C |
| ATOM | 1451 | NH1 | ARG | A | 195 | 50.753 | 52.371 | 10.585 | 1.00 | 46.87 | A | N |
| ATOM | 1452 | NH2 | ARG | A | 195 | 51.274 | 50.201 | 11.124 | 1.00 | 46.12 | A | N |
| ATOM | 1453 | N | GLU | A | 196 | 51.016 | 53.508 | 4.766 | 1.00 | 33.16 | A | N |
| ATOM | 1454 | CA | GLU | A | 196 | 52.167 | 54.353 | 4.500 | 1.00 | 34.04 | A | C |
| ATOM | 1455 | C | GLU | A | 196 | 52.963 | 54.554 | 5.774 | 1.00 | 31.83 | A | C |
| ATOM | 1456 | O | GLU | A | 196 | 52.790 | 55.566 | 6.460 | 1.00 | 35.74 | A | O |
| ATOM | 1457 | CB | GLU | A | 196 | 51.728 | 55.699 | 3.953 | 1.00 | 33.34 | A | C |
| ATOM | 1458 | CG | GLU | A | 196 | 50.986 | 55.643 | 2.624 | 1.00 | 38.79 | A | C |
| ATOM | 1459 | CD | GLU | A | 196 | 50.230 | 56.927 | 2.341 | 1.00 | 42.74 | A | C |
| ATOM | 1460 | OE1 | GLU | A | 196 | 49.199 | 57.182 | 3.009 | 1.00 | 47.60 | A | O |
| ATOM | 1461 | OE2 | GLU | A | 196 | 50.661 | 57.688 | 1.450 | 1.00 | 42.51 | A | O |
| ATOM | 1462 | N | TRP | A | 197 | 53.805 | 53.567 | 6.075 | 1.00 | 29.99 | A | N |
| ATOM | 1463 | CA | TRP | A | 197 | 54.773 | 53.629 | 7.184 | 1.00 | 32.01 | A | C |
| ATOM | 1464 | C | TRP | A | 197 | 56.104 | 53.059 | 6.668 | 1.00 | 29.04 | A | C |
| ATOM | 1465 | O | TRP | A | 197 | 56.938 | 53.829 | 6.210 | 1.00 | 30.54 | A | O |
| ATOM | 1466 | CB | TRP | A | 197 | 54.229 | 52.970 | 8.474 | 1.00 | 31.92 | A | C |
| ATOM | 1467 | CG | TRP | A | 197 | 53.800 | 51.538 | 8.412 | 1.00 | 36.08 | A | C |
| ATOM | 1468 | CD1 | TRP | A | 197 | 53.091 | 50.926 | 7.418 | 1.00 | 36.38 | A | C |
| ATOM | 1469 | CD2 | TRP | A | 197 | 54.023 | 50.532 | 9.414 | 1.00 | 40.65 | A | C |
| ATOM | 1470 | NE1 | TRP | A | 197 | 52.887 | 49.605 | 7.726 | 1.00 | 40.70 | A | N |
| ATOM | 1471 | CE2 | TRP | A | 197 | 53.446 | 49.337 | 8.948 | 1.00 | 41.77 | A | C |
| ATOM | 1472 | CE3 | TRP | A | 197 | 54.672 | 50.518 | 10.658 | 1.00 | 41.22 | A | C |
| ATOM | 1473 | CZ2 | TRP | A | 197 | 53.486 | 48.146 | 9.680 | 1.00 | 43.13 | A | C |
| ATOM | 1474 | CZ3 | TRP | A | 197 | 54.720 | 49.337 | 11.381 | 1.00 | 40.93 | A | C |
| ATOM | 1475 | CH2 | TRP | A | 197 | 54.129 | 48.166 | 10.891 | 1.00 | 42.69 | A | C |
| ATOM | 1476 | N | TYR | A | 198 | 56.303 | 51.746 | 6.699 | 1.00 | 24.59 | A | N |
| ATOM | 1477 | CA | TYR | A | 198 | 57.184 | 51.077 | 5.740 | 1.00 | 25.69 | A | C |
| ATOM | 1478 | C | TYR | A | 198 | 56.456 | 51.094 | 4.391 | 1.00 | 22.71 | A | C |
| ATOM | 1479 | O | TYR | A | 198 | 55.317 | 51.519 | 4.305 | 1.00 | 25.15 | A | O |
| ATOM | 1480 | CB | TYR | A | 198 | 57.455 | 49.620 | 6.110 | 1.00 | 28.07 | A | C |
| ATOM | 1481 | CG | TYR | A | 198 | 58.137 | 49.394 | 7.453 | 1.00 | 32.69 | A | C |
| ATOM | 1482 | CD1 | TYR | A | 198 | 59.514 | 49.273 | 7.541 | 1.00 | 36.04 | A | C |
| ATOM | 1483 | CD2 | TYR | A | 198 | 57.393 | 49.289 | 8.627 | 1.00 | 35.45 | A | C |
| ATOM | 1484 | CE1 | TYR | A | 198 | 60.146 | 49.054 | 8.769 | 1.00 | 35.82 | A | C |
| ATOM | 1485 | CE2 | TYR | A | 198 | 58.015 | 49.079 | 9.865 | 1.00 | 36.80 | A | C |
| ATOM | 1486 | CZ | TYR | A | 198 | 59.385 | 48.962 | 9.927 | 1.00 | 38.75 | A | C |
| ATOM | 1487 | OH | TYR | A | 198 | 60.007 | 48.744 | 11.143 | 1.00 | 38.81 | A | O |
| ATOM | 1488 | N | TYR | A | 199 | 57.142 | 50.654 | 3.347 | 1.00 | 21.11 | A | N |
| ATOM | 1489 | CA | TYR | A | 199 | 56.497 | 50.364 | 2.048 | 1.00 | 21.41 | A | C |
| ATOM | 1490 | C | TYR | A | 199 | 55.866 | 48.991 | 2.152 | 1.00 | 18.46 | A | C |
| ATOM | 1491 | O | TYR | A | 199 | 56.471 | 47.969 | 1.784 | 1.00 | 18.42 | A | O |
| ATOM | 1492 | CB | TYR | A | 199 | 57.521 | 50.484 | 0.914 | 1.00 | 21.15 | A | C |
| ATOM | 1493 | CG | TYR | A | 199 | 57.861 | 51.927 | 0.640 | 1.00 | 18.31 | A | C |
| ATOM | 1494 | CD1 | TYR | A | 199 | 56.965 | 52.770 | −0.020 | 1.00 | 17.27 | A | C |
| ATOM | 1495 | CD2 | TYR | A | 199 | 59.078 | 52.478 | 1.059 | 1.00 | 17.06 | A | C |
| ATOM | 1496 | CE1 | TYR | A | 199 | 57.275 | 54.106 | −0.239 | 1.00 | 18.14 | A | C |
| ATOM | 1497 | CE2 | TYR | A | 199 | 59.394 | 53.799 | 0.816 | 1.00 | 14.53 | A | C |
| ATOM | 1498 | CZ | TYR | A | 199 | 58.510 | 54.608 | 0.178 | 1.00 | 17.63 | A | C |
| ATOM | 1499 | OH | TYR | A | 199 | 58.822 | 55.911 | −0.024 | 1.00 | 17.71 | A | O |
| ATOM | 1500 | N | GLU | A | 200 | 54.664 | 48.979 | 2.742 | 1.00 | 21.57 | A | N |
| ATOM | 1501 | CA | GLU | A | 200 | 54.004 | 47.722 | 3.098 | 1.00 | 22.77 | A | C |
| ATOM | 1502 | C | GLU | A | 200 | 53.206 | 47.182 | 1.904 | 1.00 | 16.75 | A | C |
| ATOM | 1503 | O | GLU | A | 200 | 52.457 | 47.916 | 1.322 | 1.00 | 24.39 | A | O |
| ATOM | 1504 | CB | GLU | A | 200 | 53.030 | 47.909 | 4.260 | 1.00 | 24.90 | A | C |
| ATOM | 1505 | CG | GLU | A | 200 | 52.680 | 46.604 | 4.946 | 1.00 | 27.85 | A | C |
| ATOM | 1506 | CD | GLU | A | 200 | 51.514 | 46.716 | 5.919 | 1.00 | 28.16 | A | C |
| ATOM | 1507 | OE1 | GLU | A | 200 | 51.081 | 47.840 | 6.278 | 1.00 | 35.16 | A | O |
| ATOM | 1508 | OE2 | GLU | A | 200 | 50.987 | 45.649 | 6.300 | 1.00 | 34.56 | A | O |
| ATOM | 1509 | N | VAL | A | 201 | 53.386 | 45.920 | 1.596 | 1.00 | 22.87 | A | N |
| ATOM | 1510 | CA | VAL | A | 201 | 52.595 | 45.240 | 0.554 | 1.00 | 21.15 | A | C |
| ATOM | 1511 | C | VAL | A | 201 | 52.057 | 43.892 | 1.045 | 1.00 | 26.77 | A | C |
| ATOM | 1512 | O | VAL | A | 201 | 52.462 | 43.402 | 2.103 | 1.00 | 26.68 | A | O |
| ATOM | 1513 | CB | VAL | A | 201 | 53.455 | 44.997 | −0.684 | 1.00 | 22.52 | A | C |
| ATOM | 1514 | CG1 | VAL | A | 201 | 54.012 | 46.314 | −1.198 | 1.00 | 22.34 | A | C |
| ATOM | 1515 | CG2 | VAL | A | 201 | 54.593 | 43.991 | −0.400 | 1.00 | 23.70 | A | C |
| ATOM | 1516 | N | ILE | A | 202 | 51.187 | 43.262 | 0.248 | 1.00 | 20.41 | A | N |
| ATOM | 1517 | CA | ILE | A | 202 | 50.579 | 41.984 | 0.632 | 1.00 | 24.08 | A | C |
| ATOM | 1518 | C | ILE | A | 262 | 50.901 | 40.908 | −0.404 | 1.00 | 23.18 | A | C |
| ATOM | 1519 | O | ILE | A | 202 | 50.569 | 41.064 | −1.572 | 1.00 | 21.92 | A | O |
| ATOM | 1520 | CB | ILE | A | 202 | 49.041 | 42.119 | 0.801 | 1.00 | 24.91 | A | C |
| ATOM | 1521 | CG1 | ILE | A | 202 | 48.697 | 43.058 | 1.967 | 1.00 | 28.97 | A | C |
| ATOM | 1522 | CG2 | ILE | A | 202 | 48.410 | 40.742 | 1.042 | 1.00 | 26.55 | A | C |
| ATOM | 1523 | CD1 | ILE | A | 202 | 47.237 | 43.384 | 2.081 | 1.00 | 28.35 | A | C |
| ATOM | 1524 | N | ILE | A | 203 | 51.552 | 39.836 | 0.037 | 1.00 | 20.58 | A | N |
| ATOM | 1525 | CA | ILE | A | 203 | 51.806 | 38.642 | −0.749 | 1.00 | 23.06 | A | C |
| ATOM | 1526 | C | ILE | A | 203 | 50.600 | 37.712 | −0.588 | 1.00 | 23.74 | A | C |
| ATOM | 1527 | O | ILE | A | 203 | 50.113 | 37.480 | 0.521 | 1.00 | 25.10 | A | O |
| ATOM | 1528 | CB | ILE | A | 203 | 53.097 | 37.940 | −0.293 | 1.00 | 24.14 | A | C |
| ATOM | 1529 | CG1 | ILE | A | 203 | 54.310 | 38.807 | −0.656 | 1.00 | 25.32 | A | C |
| ATOM | 1530 | CG2 | ILE | A | 203 | 53.196 | 36.561 | −0.924 | 1.00 | 23.21 | A | C |
| ATOM | 1531 | CD1 | ILE | A | 203 | 55.655 | 38.280 | −0.193 | 1.00 | 30.24 | A | C |
| ATOM | 1532 | N | VAL | A | 204 | 50.065 | 37.249 | −1.705 | 1.00 | 24.79 | A | N |
| ATOM | 1533 | CA | VAL | A | 204 | 48.827 | 36.465 | −1.685 | 1.00 | 22.84 | A | C |
| ATOM | 1534 | C | VAL | A | 204 | 49.050 | 34.992 | −2.011 | 1.00 | 27.15 | A | C |
| ATOM | 1535 | O | VAL | A | 204 | 48.192 | 34.158 | −1.721 | 1.00 | 28.57 | A | O |
| ATOM | 1536 | CB | VAL | A | 204 | 47.764 | 37.091 | −2.640 | 1.00 | 21.80 | A | C |
| ATOM | 1537 | CG1 | VAL | A | 204 | 47.505 | 38.524 | −2.253 | 1.00 | 20.60 | A | C |
| ATOM | 1538 | CG2 | VAL | A | 204 | 48.210 | 36.970 | −4.075 | 1.00 | 24.51 | A | C |
| ATOM | 1539 | N | ARG | A | 205 | 50.191 | 34.678 | −2.612 | 1.00 | 24.48 | A | N |
| ATOM | 1540 | CA | ARG | A | 205 | 50.540 | 33.330 | −3.018 | 1.00 | 27.35 | A | C |
| ATOM | 1541 | C | ARG | A | 205 | 52.049 | 33.204 | −3.207 | 1.00 | 32.98 | A | C |
| ATOM | 1542 | O | ARG | A | 205 | 52.755 | 34.167 | −3.563 | 1.00 | 24.15 | A | O |
| ATOM | 1543 | CB | ARG | A | 205 | 49.815 | 32.977 | −4.325 | 1.00 | 30.35 | A | C |
| ATOM | 1544 | CG | ARG | A | 205 | 49.857 | 31.540 | −4.763 | 1.00 | 33.44 | A | C |
| ATOM | 1545 | CD | ARG | A | 205 | 49.122 | 31.314 | −6.095 | 1.00 | 36.40 | A | C |
| ATOM | 1546 | NE | ARG | A | 205 | 49.502 | 30.060 | −6.747 | 1.00 | 40.66 | A | N |
| ATOM | 1547 | CZ | ARG | A | 205 | 48.730 | 28.978 | −6.838 | 1.00 | 44.96 | A | C |
| ATOM | 1548 | NH1 | ARG | A | 205 | 47.510 | 28.961 | −6.312 | 1.00 | 49.55 | A | N |
| ATOM | 1549 | NH2 | ARG | A | 205 | 49.185 | 27.893 | −7.457 | 1.00 | 48.81 | A | N |
| ATOM | 1550 | N | VAL | A | 206 | 52.548 | 32.004 | −2.961 | 1.00 | 31.90 | A | N |
| ATOM | 1551 | CA | VAL | A | 206 | 53.955 | 31.713 | −3.133 | 1.00 | 31.00 | A | C |
| ATOM | 1552 | C | VAL | A | 206 | 54.077 | 30.348 | −3.747 | 1.00 | 36.19 | A | C |
| ATOM | 1553 | O | VAL | A | 206 | 53.523 | 29.380 | −3.227 | 1.00 | 37.64 | A | O |
| ATOM | 1554 | CB | VAL | A | 206 | 54.727 | 31.762 | −1.786 | 1.00 | 34.34 | A | C |
| ATOM | 1555 | CG1 | VAL | A | 206 | 56.208 | 31.653 | −2.026 | 1.00 | 38.60 | A | C |
| ATOM | 1556 | CG2 | VAL | A | 206 | 54.407 | 33.029 | −1.020 | 1.00 | 34.28 | A | C |
| ATOM | 1557 | N | GLU | A | 207 | 54.746 | 30.296 | −4.889 | 1.00 | 35.57 | A | N |
| ATOM | 1558 | CA | GLU | A | 207 | 55.129 | 29.059 | −5.525 | 1.00 | 40.01 | A | C |
| ATOM | 1559 | C | GLU | A | 207 | 56.630 | 28.880 | −5.382 | 1.00 | 43.14 | A | C |
| ATOM | 1560 | O | GLU | A | 207 | 57.371 | 29.858 | −5.263 | 1.00 | 38.43 | A | O |
| ATOM | 1561 | CB | GLU | A | 207 | 54.766 | 29.102 | −7.007 | 1.00 | 41.74 | A | C |
| ATOM | 1562 | CG | GLU | A | 207 | 53.270 | 29.123 | −7.274 | 1.00 | 41.66 | A | C |
| ATOM | 1563 | CD | GLU | A | 207 | 52.934 | 29.348 | −8.733 | 1.00 | 41.56 | A | C |
| ATOM | 1564 | OE1 | GLU | A | 207 | 53.854 | 29.540 | −9.548 | 1.00 | 40.42 | A | O |
| ATOM | 1565 | OE2 | GLU | A | 207 | 51.733 | 29.347 | −9.071 | 1.00 | 47.50 | A | O |
| ATOM | 1566 | N | ILE | A | 208 | 57.063 | 27.620 | −5.371 | 1.00 | 44.90 | A | N |
| ATOM | 1567 | CA | ILE | A | 208 | 58.462 | 27.260 | −5.592 | 1.00 | 48.28 | A | C |
| ATOM | 1568 | C | ILE | A | 208 | 58.488 | 26.248 | −6.737 | 1.00 | 50.10 | A | C |
| ATOM | 1569 | O | ILE | A | 208 | 57.926 | 25.155 | −6.628 | 1.00 | 48.21 | A | O |
| ATOM | 1570 | CB | ILE | A | 208 | 59.106 | 26.692 | −4.312 | 1.00 | 48.76 | A | C |
| ATOM | 1571 | CG1 | ILE | A | 208 | 59.185 | 27.770 | −3.227 | 1.00 | 50.92 | A | C |
| ATOM | 1572 | CG2 | ILE | A | 208 | 60.499 | 26.179 | −4.607 | 1.00 | 50.10 | A | C |
| ATOM | 1573 | CD1 | ILE | A | 208 | 59.221 | 27.225 | −1.835 | 1.00 | 52.20 | A | C |
| ATOM | 1574 | N | ASN | A | 209 | 59.102 | 26.647 | −7.846 | 1.00 | 51.40 | A | N |
| ATOM | 1575 | CA | ASN | A | 209 | 59.111 | 25.882 | −9.091 | 1.00 | 56.38 | A | C |
| ATOM | 1576 | C | ASN | A | 209 | 57.756 | 25.851 | −9.827 | 1.00 | 56.68 | A | C |
| ATOM | 1577 | O | ASN | A | 209 | 57.689 | 25.425 | −10.982 | 1.00 | 55.91 | A | O |
| ATOM | 1578 | CB | ASN | A | 209 | 59.636 | 24.456 | −8.847 | 1.00 | 58.04 | A | C |
| ATOM | 1579 | CG | ASN | A | 209 | 60.332 | 23.865 | −10.064 | 1.00 | 60.28 | A | C |
| ATOM | 1580 | OD1 | ASN | A | 209 | 60.251 | 22.656 | −10.316 | 1.00 | 65.04 | A | O |
| ATOM | 1581 | ND2 | ASN | A | 209 | 61.025 | 24.707 | −10.820 | 1.00 | 62.80 | A | N |
| ATOM | 1582 | N | GLY | A | 210 | 56.696 | 26.338 | −9.181 | 1.00 | 56.56 | A | N |
| ATOM | 1583 | CA | GLY | A | 210 | 55.345 | 26.202 | −9.699 | 1.00 | 56.62 | A | C |
| ATOM | 1584 | C | GLY | A | 210 | 54.374 | 25.584 | −8.708 | 1.00 | 57.30 | A | C |
| ATOM | 1585 | O | GLY | A | 210 | 53.169 | 25.564 | −8.959 | 1.00 | 58.65 | A | O |
| ATOM | 1586 | N | GLN | A | 211 | 54.885 | 25.094 | −7.582 | 1.00 | 56.04 | A | N |
| ATOM | 1587 | CA | GLN | A | 211 | 54.071 | 24.409 | −6.589 | 1.00 | 56.19 | A | C |
| ATOM | 1588 | C | GLN | A | 211 | 53.873 | 25.311 | −5.383 | 1.00 | 55.79 | A | C |
| ATOM | 1589 | O | GLN | A | 211 | 54.839 | 25.720 | −4.748 | 1.00 | 51.74 | A | O |
| ATOM | 1590 | CB | GLN | A | 211 | 54.761 | 23.112 | −6.168 | 1.00 | 58.40 | A | C |
| ATOM | 1591 | CG | GLN | A | 211 | 54.940 | 22.113 | −7.308 | 1.00 | 60.83 | A | C |
| ATOM | 1592 | CD | GLN | A | 211 | 55.915 | 20.988 | −6.973 | 1.00 | 64.24 | A | C |
| ATOM | 1593 | OE1 | GLN | A | 211 | 56.594 | 20.463 | −7.863 | 1.00 | 64.43 | A | O |
| ATOM | 1594 | NE2 | GLN | A | 211 | 55.983 | 20.614 | −5.694 | 1.00 | 66.64 | A | N |
| ATOM | 1595 | N | ASP | A | 212 | 52.628 | 25.626 | −5.048 | 1.00 | 54.01 | A | N |
| ATOM | 1596 | CA | ASP | A | 212 | 52.407 | 26.569 | −3.959 | 1.00 | 56.91 | A | C |
| ATOM | 1597 | C | ASP | A | 212 | 52.447 | 25.914 | −2.575 | 1.00 | 57.30 | A | C |
| ATOM | 1598 | O | ASP | A | 212 | 52.369 | 24.693 | −2.454 | 1.00 | 55.10 | A | O |
| ATOM | 1599 | CB | ASP | A | 212 | 51.157 | 27.428 | −4.209 | 1.00 | 56.98 | A | C |
| ATOM | 1600 | CG | ASP | A | 212 | 49.890 | 26.788 | −3.737 | 1.00 | 57.67 | A | C |
| ATOM | 1601 | OD1 | ASP | A | 212 | 49.566 | 25.680 | −4.212 | 1.00 | 57.46 | A | O |
| ATOM | 1602 | OD2 | ASP | A | 212 | 49.139 | 27.347 | −2.909 | 1.00 | 59.70 | A | O |
| ATOM | 1603 | N | LEU | A | 213 | 52.623 | 26.738 | −1.545 | 1.00 | 58.77 | A | N |
| ATOM | 1604 | CA | LEU | A | 213 | 52.860 | 26.263 | −0.176 | 1.00 | 60.89 | A | C |
| ATOM | 1605 | C | LEU | A | 213 | 51.546 | 26.024 | 0.566 | 1.00 | 61.05 | A | C |
| ATOM | 1606 | O | LEU | A | 213 | 51.518 | 25.356 | 1.596 | 1.00 | 58.81 | A | O |
| ATOM | 1607 | CB | LEU | A | 213 | 53.725 | 27.270 | 0.598 | 1.00 | 61.26 | A | C |
| ATOM | 1608 | CG | LEU | A | 213 | 55.223 | 27.312 | 0.263 | 1.00 | 62.51 | A | C |
| ATOM | 1609 | CD1 | LEU | A | 213 | 55.490 | 27.170 | −1.234 | 1.00 | 62.99 | A | C |
| ATOM | 1610 | CD2 | LEU | A | 213 | 55.857 | 28.602 | 0.793 | 1.00 | 63.51 | A | C |
| ATOM | 1611 | N | LYS | A | 214 | 50.470 | 26.602 | 0.039 | 1.00 | 62.93 | A | N |
| ATOM | 1612 | CA | LYS | A | 214 | 49.107 | 26.273 | 0.441 | 1.00 | 65.41 | A | C |
| ATOM | 1613 | C | LYS | A | 214 | 48.812 | 26.553 | 1.915 | 1.00 | 64.81 | A | C |
| ATOM | 1614 | O | LYS | A | 214 | 47.985 | 25.872 | 2.523 | 1.00 | 66.72 | A | O |
| ATOM | 1615 | CB | LYS | A | 214 | 48.781 | 24.808 | 0.083 | 1.00 | 66.77 | A | C |
| ATOM | 1616 | CG | LYS | A | 214 | 47.364 | 24.625 | −0.469 | 1.00 | 69.56 | A | C |
| ATOM | 1617 | CD | LYS | A | 214 | 46.931 | 23.158 | −0.553 | 1.00 | 70.85 | A | C |
| ATOM | 1618 | CE | LYS | A | 214 | 45.423 | 23.023 | −0.346 | 1.00 | 70.83 | A | C |
| ATOM | 1619 | NZ | LYS | A | 214 | 44.873 | 21.747 | −0.879 | 1.00 | 72.20 | A | N |
| ATOM | 1620 | N | MET | A | 215 | 49.465 | 27.568 | 2.480 | 1.00 | 62.76 | A | N |
| ATOM | 1621 | CA | MET | A | 215 | 49.187 | 27.959 | 3.862 | 1.00 | 61.10 | A | C |
| ATOM | 1622 | C | MET | A | 215 | 48.402 | 29.267 | 3.925 | 1.00 | 57.64 | A | C |
| ATOM | 1623 | O | MET | A | 215 | 48.495 | 30.102 | 3.024 | 1.00 | 57.95 | A | O |
| ATOM | 1624 | CB | MET | A | 215 | 50.474 | 28.035 | 4.701 | 1.00 | 62.37 | A | C |
| ATOM | 1625 | CG | MET | A | 215 | 51.555 | 28.957 | 4.190 | 1.00 | 63.11 | A | C |
| ATOM | 1626 | SD | MET | A | 215 | 53.067 | 28.833 | 5.203 | 1.00 | 62.67 | A | S |
| ATOM | 1627 | CE | MET | A | 215 | 54.211 | 28.376 | 3.978 | 1.00 | 62.72 | A | C |
| ATOM | 1628 | N | ASP | A | 216 | 47.599 | 29.420 | 4.976 | 1.00 | 53.34 | A | N |
| ATOM | 1629 | CA | ASP | A | 216 | 46.873 | 30.660 | 5.220 | 1.00 | 52.79 | A | C |
| ATOM | 1630 | C | ASP | A | 216 | 47.755 | 31.819 | 4.770 | 1.00 | 51.45 | A | C |
| ATOM | 1631 | O | ASP | A | 216 | 48.861 | 32.008 | 5.283 | 1.00 | 45.58 | A | O |
| ATOM | 1632 | CB | ASP | A | 216 | 46.517 | 30.792 | 6.705 | 1.00 | 53.42 | A | C |
| ATOM | 1633 | CG | ASP | A | 216 | 45.523 | 31.911 | 6.981 | 1.00 | 55.05 | A | C |
| ATOM | 1634 | OD1 | ASP | A | 216 | 45.109 | 32.639 | 6.045 | 1.00 | 53.37 | A | O |
| ATOM | 1635 | OD2 | ASP | A | 216 | 45.096 | 32.134 | 8.132 | 1.00 | 59.33 | A | O |
| ATOM | 1636 | N | CYS | A | 217 | 47.285 | 32.567 | 3.779 | 1.00 | 49.19 | A | N |
| ATOM | 1637 | CA | CYS | A | 217 | 48.119 | 33.601 | 3.172 | 1.00 | 49.39 | A | C |
| ATOM | 1638 | C | CYS | A | 217 | 48.345 | 34.770 | 4.144 | 1.00 | 48.78 | A | C |
| ATOM | 1639 | O | CYS | A | 217 | 49.274 | 35.554 | 3.966 | 1.00 | 49.38 | A | O |
| ATOM | 1640 | CB | CYS | A | 217 | 47.515 | 34.072 | 1.843 | 1.00 | 46.23 | A | C |
| ATOM | 1641 | SG | CYS | A | 217 | 45.917 | 34.862 | 2.016 | 1.00 | 48.31 | A | S |
| ATOM | 1642 | N | LYS | A | 218 | 47.489 | 34.869 | 5.166 | 1.00 | 48.17 | A | N |
| ATOM | 1643 | CA | LYS | A | 218 | 47.671 | 35.792 | 6.291 | 1.00 | 46.87 | A | C |
| ATOM | 1644 | C | LYS | A | 218 | 49.055 | 35.646 | 6.937 | 1.00 | 44.90 | A | C |
| ATOM | 1645 | O | LYS | A | 218 | 49.632 | 36.624 | 7.446 | 1.00 | 37.36 | A | O |
| ATOM | 1646 | CB | LYS | A | 218 | 46.585 | 35.551 | 7.345 | 1.00 | 48.51 | A | C |
| ATOM | 1647 | CG | LYS | A | 218 | 46.222 | 36.772 | 8.167 | 1.00 | 52.96 | A | C |
| ATOM | 1648 | CD | LYS | A | 218 | 44.760 | 36.731 | 8.616 | 1.00 | 54.70 | A | C |
| ATOM | 1649 | CE | LYS | A | 218 | 44.287 | 38.081 | 9.134 | 1.00 | 56.36 | A | C |
| ATOM | 1650 | NZ | LYS | A | 218 | 42.812 | 38.224 | 8.965 | 1.00 | 58.19 | A | N |
| ATOM | 1651 | N | GLU | A | 219 | 49.579 | 34.425 | 6.899 | 1.00 | 41.30 | A | N |
| ATOM | 1652 | CA | GLU | A | 219 | 50.894 | 34.118 | 7.460 | 1.00 | 41.57 | A | C |
| ATOM | 1653 | C | GLU | A | 219 | 52.043 | 34.739 | 6.674 | 1.00 | 41.40 | A | C |
| ATOM | 1654 | O | GLU | A | 219 | 53.112 | 35.003 | 7.245 | 1.00 | 38.39 | A | O |
| ATOM | 1655 | CB | GLU | A | 219 | 51.129 | 32.603 | 7.526 | 1.00 | 42.52 | A | C |
| ATOM | 1656 | CG | GLU | A | 219 | 50.072 | 31.806 | 8.284 | 1.00 | 43.99 | A | C |
| ATOM | 1657 | CD | GLU | A | 219 | 50.103 | 32.015 | 9.793 | 1.00 | 49.15 | A | C |
| ATOM | 1658 | OE1 | GLU | A | 219 | 49.422 | 31.235 | 10.503 | 1.00 | 49.54 | A | O |
| ATOM | 1659 | OE2 | GLU | A | 219 | 50.786 | 32.950 | 10.276 | 1.00 | 47.89 | A | O |
| ATOM | 1660 | N | TYR | A | 220 | 51.851 | 34.902 | 5.364 | 1.00 | 35.85 | A | N |
| ATOM | 1661 | CA | TYR | A | 220 | 52.867 | 35.487 | 4.479 | 1.00 | 35.18 | A | C |
| ATOM | 1662 | C | TYR | A | 220 | 53.164 | 36.913 | 4.830 | 1.00 | 28.13 | A | C |
| ATOM | 1663 | O | TYR | A | 220 | 54.185 | 37.465 | 4.445 | 1.00 | 34.25 | A | O |
| ATOM | 1664 | CB | TYR | A | 220 | 52.413 | 35.452 | 3.004 | 1.00 | 36.98 | A | C |
| ATOM | 1665 | CG | TYR | A | 220 | 52.256 | 34.069 | 2.389 | 1.00 | 37.36 | A | C |
| ATOM | 1666 | CD1 | TYR | A | 220 | 51.233 | 33.809 | 1.478 | 1.00 | 37.78 | A | C |
| ATOM | 1667 | CD2 | TYR | A | 220 | 53.132 | 33.026 | 2.702 | 1.00 | 40.58 | A | C |
| ATOM | 1668 | CE1 | TYR | A | 220 | 51.093 | 32.557 | 0.894 | 1.00 | 38.43 | A | C |
| ATOM | 1669 | CE2 | TYR | A | 220 | 52.996 | 31.775 | 2.136 | 1.00 | 40.55 | A | C |
| ATOM | 1670 | CZ | TYR | A | 220 | 51.962 | 31.539 | 1.233 | 1.00 | 41.11 | A | C |
| ATOM | 1671 | OH | TYR | A | 220 | 51.819 | 30.299 | 0.656 | 1.00 | 42.27 | A | O |
| ATOM | 1672 | N | ASN | A | 221 | 52.230 | 37.542 | 5.520 | 1.00 | 33.23 | A | N |
| ATOM | 1673 | CA | ASN | A | 221 | 52.322 | 38.933 | 5.865 | 1.00 | 35.09 | A | C |
| ATOM | 1674 | C | ASN | A | 221 | 52.201 | 39.102 | 7.363 | 1.00 | 31.87 | A | C |
| ATOM | 1675 | O | ASN | A | 221 | 51.682 | 40.126 | 7.831 | 1.00 | 33.51 | A | O |
| ATOM | 1676 | CB | ASN | A | 221 | 51.201 | 39.649 | 5.143 | 1.00 | 36.43 | A | C |
| ATOM | 1677 | CG | ASN | A | 221 | 51.102 | 39.213 | 3.695 | 1.00 | 37.85 | A | C |
| ATOM | 1678 | OD1 | ASN | A | 221 | 50.157 | 38.521 | 3.300 | 1.00 | 33.97 | A | O |
| ATOM | 1679 | ND2 | ASN | A | 221 | 52.119 | 39.561 | 2.910 | 1.00 | 28.28 | A | N |
| ATOM | 1680 | N | TYR | A | 222 | 52.668 | 38.088 | 8.091 | 1.00 | 39.46 | A | N |
| ATOM | 1681 | CA | TYR | A | 222 | 52.401 | 37.971 | 9.525 | 1.00 | 42.38 | A | C |
| ATOM | 1682 | C | TYR | A | 222 | 53.237 | 39.004 | 10.244 | 1.00 | 43.57 | A | C |
| ATOM | 1683 | O | TYR | A | 222 | 54.475 | 38.894 | 10.348 | 1.00 | 33.19 | A | O |
| ATOM | 1684 | CB | TYR | A | 222 | 52.673 | 36.559 | 10.071 | 1.00 | 45.87 | A | C |
| ATOM | 1685 | CG | TYR | A | 222 | 52.591 | 36.428 | 11.592 | 1.00 | 48.19 | A | C |
| ATOM | 1686 | CD1 | TYR | A | 222 | 51.870 | 37.337 | 12.382 | 1.00 | 51.26 | A | C |
| ATOM | 1687 | CD2 | TYR | A | 222 | 53.241 | 35.392 | 12.236 | 1.00 | 51.18 | A | C |
| ATOM | 1688 | CE1 | TYR | A | 222 | 51.815 | 37.204 | 13.774 | 1.00 | 52.32 | A | C |
| ATOM | 1689 | CE2 | TYR | A | 222 | 53.192 | 35.245 | 13.616 | 1.00 | 52.66 | A | C |
| ATOM | 1690 | CZ | TYR | A | 222 | 52.481 | 36.154 | 14.381 | 1.00 | 54.09 | A | C |
| ATOM | 1691 | OH | TYR | A | 222 | 52.436 | 36.004 | 15.751 | 1.00 | 56.15 | A | O |
| ATOM | 1692 | N | ASP | A | 223 | 52.486 | 39.968 | 10.768 | 1.00 | 46.73 | A | N |
| ATOM | 1693 | CA | ASP | A | 223 | 52.934 | 41.294 | 11.099 | 1.00 | 47.06 | A | C |
| ATOM | 1694 | C | ASP | A | 223 | 52.865 | 42.194 | 9.864 | 1.00 | 43.47 | A | C |
| ATOM | 1695 | O | ASP | A | 223 | 52.008 | 43.088 | 9.777 | 1.00 | 45.70 | A | O |
| ATOM | 1696 | CB | ASP | A | 223 | 54.339 | 41.289 | 11.693 | 1.00 | 50.64 | A | C |
| ATOM | 1697 | CG | ASP | A | 223 | 54.663 | 42.585 | 12.348 | 1.00 | 48.82 | A | C |
| ATOM | 1698 | OD1 | ASP | A | 223 | 54.041 | 42.879 | 13.392 | 1.00 | 56.19 | A | O |
| ATOM | 1699 | OD2 | ASP | A | 223 | 55.483 | 43.386 | 11.871 | 1.00 | 54.79 | A | O |
| ATOM | 1700 | N | LYS | A | 224 | 53.745 | 41.920 | 8.908 | 1.00 | 43.90 | A | N |
| ATOM | 1701 | CA | LYS | A | 224 | 54.128 | 42.889 | 7.872 | 1.00 | 43.50 | A | C |
| ATOM | 1702 | C | LYS | A | 224 | 54.720 | 42.221 | 6.651 | 1.00 | 38.65 | A | C |
| ATOM | 1703 | O | LYS | A | 224 | 55.321 | 41.177 | 6.749 | 1.00 | 37.31 | A | O |
| ATOM | 1704 | CB | LYS | A | 224 | 55.234 | 43.791 | 8.425 | 1.00 | 44.84 | A | C |
| ATOM | 1705 | CG | LYS | A | 224 | 54.814 | 45.182 | 8.824 | 1.00 | 49.52 | A | C |
| ATOM | 1706 | CD | LYS | A | 224 | 56.030 | 46.109 | 8.874 | 1.00 | 50.93 | A | C |
| ATOM | 1707 | CE | LYS | A | 224 | 56.970 | 45.783 | 10.029 | 1.00 | 52.43 | A | C |
| ATOM | 1708 | NZ | LYS | A | 224 | 58.303 | 45.329 | 9.550 | 1.00 | 53.74 | A | N |
| ATOM | 1709 | N | SER | A | 225 | 54.605 | 42.855 | 5.487 | 1.00 | 34.67 | A | N |
| ATOM | 1710 | CA | SER | A | 225 | 55.428 | 42.463 | 4.347 | 1.00 | 29.36 | A | C |
| ATOM | 1711 | C | SER | A | 225 | 55.923 | 43.766 | 3.727 | 1.00 | 23.18 | A | C |
| ATOM | 1712 | O | SER | A | 225 | 55.109 | 44.637 | 3.491 | 1.00 | 24.20 | A | O |
| ATOM | 1713 | CB | SER | A | 225 | 54.634 | 41.642 | 3.319 | 1.00 | 33.54 | A | C |
| ATOM | 1714 | OG | SER | A | 225 | 54.443 | 40.303 | 3.737 | 1.00 | 31.33 | A | O |
| ATOM | 1715 | N | ILE | A | 226 | 57.235 | 43.914 | 3.514 | 1.00 | 24.65 | A | N |
| ATOM | 1716 | CA | ILE | A | 226 | 57.814 | 45.209 | 3.080 | 1.00 | 24.70 | A | C |
| ATOM | 1717 | C | ILE | A | 226 | 58.879 | 45.132 | 1.973 | 1.00 | 20.50 | A | C |
| ATOM | 1718 | O | ILE | A | 226 | 59.568 | 44.138 | 1.762 | 1.00 | 22.18 | A | O |
| ATOM | 1719 | CB | ILE | A | 226 | 58.378 | 46.052 | 4.318 | 1.00 | 26.96 | A | C |
| ATOM | 1720 | CG1 | ILE | A | 226 | 59.715 | 45.495 | 4.804 | 1.00 | 29.94 | A | C |
| ATOM | 1721 | CG2 | ILE | A | 226 | 57.350 | 46.153 | 5.417 | 1.00 | 29.80 | A | C |
| ATOM | 1722 | CD1 | ILE | A | 226 | 60.392 | 46.347 | 5.918 | 1.00 | 30.64 | A | C |
| ATOM | 1723 | N | VAL | A | 227 | 59.046 | 46.229 | 1.249 | 1.00 | 21.76 | A | N |
| ATOM | 1724 | CA | VAL | A | 227 | 60.073 | 46.314 | 0.221 | 1.00 | 20.70 | A | C |
| ATOM | 1725 | C | VAL | A | 227 | 61.187 | 47.177 | 0.820 | 1.00 | 22.68 | A | C |
| ATOM | 1726 | O | VAL | A | 227 | 60.949 | 48.339 | 1.114 | 1.00 | 25.66 | A | O |
| ATOM | 1727 | CB | VAL | A | 227 | 59.486 | 46.982 | −1.036 | 1.00 | 22.09 | A | C |
| ATOM | 1728 | CG1 | VAL | A | 227 | 60.466 | 46.919 | −2.217 | 1.00 | 22.41 | A | C |
| ATOM | 1729 | CG2 | VAL | A | 227 | 58.172 | 46.322 | −1.385 | 1.00 | 22.10 | A | C |
| ATOM | 1730 | N | ASP | A | 228 | 62.377 | 46.598 | 1.025 | 1.00 | 25.94 | A | N |
| ATOM | 1731 | CA | ASP | A | 228 | 63.488 | 47.250 | 1.750 | 1.00 | 26.69 | A | C |
| ATOM | 1732 | C | ASP | A | 228 | 64.859 | 47.181 | 1.053 | 1.00 | 25.68 | A | C |
| ATOM | 1733 | O | ASP | A | 228 | 65.552 | 46.160 | 1.089 | 1.00 | 28.14 | A | O |
| ATOM | 1734 | CB | ASP | A | 228 | 63.610 | 46.628 | 3.151 | 1.00 | 30.80 | A | C |
| ATOM | 1735 | CG | ASP | A | 228 | 64.507 | 47.435 | 4.073 | 1.00 | 36.12 | A | C |
| ATOM | 1736 | OD1 | ASP | A | 228 | 65.240 | 48.316 | 3.575 | 1.00 | 32.68 | A | O |
| ATOM | 1737 | OD2 | ASP | A | 228 | 64.544 | 47.261 | 5.315 | 1.00 | 43.13 | A | O |
| ATOM | 1738 | N | SER | A | 229 | 65.273 | 48.283 | 0.448 | 1.00 | 20.40 | A | N |
| ATOM | 1739 | CA | SER | A | 229 | 66.534 | 48.352 | 0.261 | 1.00 | 19.83 | A | C |
| ATOM | 1740 | C | SER | A | 229 | 67.766 | 48.269 | 0.668 | 1.00 | 20.00 | A | C |
| ATOM | 1741 | O | SER | A | 229 | 68.848 | 47.998 | 0.202 | 1.00 | 20.19 | A | O |
| ATOM | 1742 | CB | SER | A | 229 | 66.608 | 49.641 | −1.084 | 1.00 | 19.70 | A | C |
| ATOM | 1743 | OG | SER | A | 229 | 66.651 | 50.793 | −0.239 | 1.00 | 20.04 | A | O |
| ATOM | 1744 | N | GLY | A | 230 | 67.582 | 48.539 | 1.955 | 1.00 | 24.02 | A | N |
| ATOM | 1745 | CA | GLY | A | 230 | 68.666 | 48.429 | 2.928 | 1.00 | 30.23 | A | C |
| ATOM | 1746 | C | GLY | A | 230 | 69.016 | 46.983 | 3.270 | 1.00 | 32.91 | A | C |
| ATOM | 1747 | O | GLY | A | 230 | 70.179 | 46.641 | 3.517 | 1.00 | 37.27 | A | O |
| ATOM | 1748 | N | THR | A | 231 | 67.998 | 46.129 | 3.290 | 1.00 | 35.70 | A | N |
| ATOM | 1749 | CA | THR | A | 231 | 68.157 | 44.736 | 3.700 | 1.00 | 36.19 | A | C |
| ATOM | 1750 | C | THR | A | 231 | 68.647 | 43.943 | 2.502 | 1.00 | 36.17 | A | C |
| ATOM | 1751 | O | THR | A | 231 | 68.125 | 44.098 | 1.392 | 1.00 | 36.88 | A | O |
| ATOM | 1752 | CB | THR | A | 231 | 66.811 | 44.203 | 4.216 | 1.00 | 36.06 | A | C |
| ATOM | 1753 | OG1 | THR | A | 231 | 66.371 | 44.988 | 5.333 | 1.00 | 41.33 | A | O |
| ATOM | 1754 | CG2 | THR | A | 231 | 66.931 | 42.806 | 4.770 | 1.00 | 39.41 | A | C |
| ATOM | 1755 | N | THR | A | 232 | 69.676 | 43.117 | 2.701 | 1.00 | 34.96 | A | N |
| ATOM | 1756 | CA | THR | A | 232 | 70.213 | 42.299 | 1.624 | 1.00 | 31.73 | A | C |
| ATOM | 1757 | C | THR | A | 232 | 69.299 | 41.111 | 1.311 | 1.00 | 35.19 | A | C |
| ATOM | 1758 | O | THR | A | 232 | 69.149 | 40.754 | 0.150 | 1.00 | 34.98 | A | O |
| ATOM | 1759 | CB | THR | A | 232 | 71.612 | 41.737 | 1.968 | 1.00 | 36.41 | A | C |
| ATOM | 1760 | OG1 | THR | A | 232 | 72.502 | 42.796 | 2.338 | 1.00 | 33.05 | A | O |
| ATOM | 1761 | CG2 | THR | A | 232 | 72.274 | 41.136 | 0.735 | 1.00 | 36.22 | A | C |
| ATOM | 1762 | N | ASN | A | 233 | 68.735 | 40.502 | 2.352 | 1.00 | 38.19 | A | N |
| ATOM | 1763 | CA | ASN | A | 233 | 68.029 | 39.230 | 2.211 | 1.00 | 41.17 | A | C |
| ATOM | 1764 | C | ASN | A | 233 | 66.520 | 39.365 | 2.052 | 1.00 | 40.23 | A | C |
| ATOM | 1765 | O | ASN | A | 233 | 65.922 | 40.416 | 2.327 | 1.00 | 39.76 | A | O |
| ATOM | 1766 | CB | ASN | A | 233 | 68.307 | 38.323 | 3.420 | 1.00 | 43.40 | A | C |
| ATOM | 1767 | CG | ASN | A | 233 | 69.767 | 37.864 | 3.503 | 1.00 | 44.47 | A | C |
| ATOM | 1768 | OD1 | ASN | A | 233 | 70.293 | 37.678 | 4.593 | 1.00 | 53.39 | A | O |
| ATOM | 1769 | ND2 | ASN | A | 233 | 70.409 | 37.667 | 2.360 | 1.00 | 47.21 | A | N |
| ATOM | 1770 | N | LEU | A | 234 | 65.927 | 38.259 | 1.613 | 1.00 | 39.45 | A | N |
| ATOM | 1771 | CA | LEU | A | 234 | 64.497 | 38.025 | 1.667 | 1.00 | 35.83 | A | C |
| ATOM | 1772 | C | LEU | A | 234 | 64.178 | 37.466 | 3.035 | 1.00 | 31.21 | A | C |
| ATOM | 1773 | O | LEU | A | 234 | 64.504 | 36.342 | 3.319 | 1.00 | 35.53 | A | O |
| ATOM | 1774 | CB | LEU | A | 234 | 64.119 | 37.022 | 0.562 | 1.00 | 41.31 | A | C |
| ATOM | 1775 | CG | LEU | A | 234 | 62.727 | 36.992 | −0.082 | 1.00 | 42.04 | A | C |
| ATOM | 1776 | CD1 | LEU | A | 234 | 62.447 | 35.596 | −0.613 | 1.00 | 45.40 | A | C |
| ATOM | 1777 | CD2 | LEU | A | 234 | 61.630 | 37.429 | 0.851 | 1.00 | 42.45 | A | C |
| ATOM | 1778 | N | ARG | A | 235 | 63.564 | 38.263 | 3.906 | 1.00 | 39.51 | A | N |
| ATOM | 1779 | CA | ARG | A | 235 | 63.200 | 37.811 | 5.254 | 1.00 | 37.00 | A | C |
| ATOM | 1780 | C | ARG | A | 235 | 61.737 | 37.315 | 5.290 | 1.00 | 38.00 | A | C |
| ATOM | 1781 | O | ARG | A | 235 | 60.863 | 37.918 | 4.699 | 1.00 | 31.41 | A | O |
| ATOM | 1782 | CB | ARG | A | 235 | 63.434 | 38.930 | 6.278 | 1.00 | 40.50 | A | C |
| ATOM | 1783 | CG | ARG | A | 235 | 64.843 | 39.557 | 6.210 | 1.00 | 43.67 | A | C |
| ATOM | 1784 | CD | ARG | A | 235 | 65.208 | 40.478 | 7.378 | 1.00 | 46.74 | A | C |
| ATOM | 1785 | NE | ARG | A | 235 | 65.177 | 39.774 | 8.659 | 1.00 | 49.76 | A | N |
| ATOM | 1786 | CZ | ARG | A | 235 | 64.729 | 40.272 | 9.823 | 1.00 | 52.94 | A | C |
| ATOM | 1787 | NH1 | ARG | A | 235 | 64.272 | 41.522 | 9.918 | 1.00 | 51.18 | A | N |
| ATOM | 1788 | NH2 | ARG | A | 235 | 64.743 | 39.503 | 10.914 | 1.00 | 51.36 | A | N |
| ATOM | 1789 | N | LEU | A | 236 | 61.473 | 36.226 | 6.008 | 1.00 | 37.36 | A | N |
| ATOM | 1790 | CA | LEU | A | 236 | 60.156 | 35.571 | 5.992 | 1.00 | 37.98 | A | C |
| ATOM | 1791 | C | LEU | A | 236 | 59.691 | 35.254 | 7.408 | 1.00 | 39.27 | A | C |
| ATOM | 1792 | O | LEU | A | 236 | 60.503 | 34.847 | 8.230 | 1.00 | 37.29 | A | O |
| ATOM | 1793 | CB | LEU | A | 236 | 60.238 | 34.271 | 5.210 | 1.00 | 37.66 | A | C |
| ATOM | 1794 | CG | LEU | A | 236 | 60.689 | 34.363 | 3.745 | 1.00 | 36.72 | A | C |
| ATOM | 1795 | CD1 | LEU | A | 236 | 60.713 | 32.994 | 3.135 | 1.00 | 34.34 | A | C |
| ATOM | 1796 | CD2 | LEU | A | 236 | 59.784 | 35.269 | 2.922 | 1.00 | 37.91 | A | C |
| ATOM | 1797 | N | PRO | A | 237 | 58.399 | 35.408 | 7.719 | 1.00 | 37.12 | A | N |
| ATOM | 1798 | CA | PRO | A | 237 | 57.939 | 35.039 | 9.061 | 1.00 | 35.72 | A | C |
| ATOM | 1799 | C | PRO | A | 237 | 58.324 | 33.600 | 9.304 | 1.00 | 36.04 | A | C |
| ATOM | 1800 | O | PRO | A | 237 | 58.364 | 32.870 | 8.326 | 1.00 | 31.43 | A | O |
| ATOM | 1801 | CB | PRO | A | 237 | 56.425 | 35.222 | 8.985 | 1.00 | 36.23 | A | C |
| ATOM | 1802 | CG | PRO | A | 237 | 56.243 | 36.263 | 7.917 | 1.00 | 36.72 | A | C |
| ATOM | 1803 | CD | PRO | A | 237 | 57.297 | 35.918 | 6.887 | 1.00 | 37.44 | A | C |
| ATOM | 1804 | N | LYS | A | 238 | 58.647 | 33.213 | 10.538 | 1.00 | 34.49 | A | N |
| ATOM | 1805 | CA | LYS | A | 238 | 59.098 | 31.839 | 10.805 | 1.00 | 34.56 | A | C |
| ATOM | 1806 | C | LYS | A | 238 | 58.322 | 30.780 | 10.051 | 1.00 | 32.78 | A | C |
| ATOM | 1807 | O | LYS | A | 238 | 58.908 | 29.860 | 9.452 | 1.00 | 32.03 | A | O |
| ATOM | 1808 | CB | LYS | A | 238 | 58.881 | 31.433 | 12.243 | 1.09 | 34.24 | A | C |
| ATOM | 1809 | CG | LYS | A | 238 | 59.299 | 32.363 | 13.281 | 1.00 | 30.75 | A | C |
| ATOM | 1810 | CD | LYS | A | 238 | 58.868 | 31.768 | 14.539 | 1.00 | 5.80 | A | C |
| ATOM | 1811 | CE | LYS | A | 238 | 57.493 | 31.846 | 14.975 | 1.00 | 27.32 | A | C |
| ATOM | 1812 | NZ | LYS | A | 238 | 57.007 | 31.123 | 16.239 | 1.00 | 32.55 | A | N |
| ATOM | 1813 | N | LYS | A | 239 | 56.998 | 30.879 | 10.142 | 1.00 | 28.36 | A | N |
| ATOM | 1814 | CA | LYS | A | 239 | 56.149 | 29.747 | 9.735 | 1.00 | 37.49 | A | C |
| ATOM | 1815 | C | LYS | A | 239 | 56.292 | 29.508 | 8.233 | 1.00 | 35.49 | A | C |
| ATOM | 1816 | O | LYS | A | 239 | 56.310 | 28.360 | 7.762 | 1.00 | 30.89 | A | O |
| ATOM | 1817 | CB | LYS | A | 239 | 54.675 | 29.990 | 10.108 | 1.00 | 39.29 | A | C |
| ATOM | 1818 | CG | LYS | A | 239 | 54.110 | 28.997 | 11.108 | 1.00 | 46.03 | A | C |
| ATOM | 1819 | CD | LYS | A | 239 | 52.700 | 29.406 | 11.566 | 1.00 | 48.69 | A | C |
| ATOM | 1820 | CE | LYS | A | 239 | 51.634 | 28.404 | 11.117 | 1.00 | 50.81 | A | C |
| ATOM | 1821 | NZ | LYS | A | 239 | 50.243 | 28.846 | 11.463 | 1.00 | 49.91 | A | N |
| ATOM | 1822 | N | VAL | A | 240 | 56.411 | 30.614 | 7.497 | 1.00 | 32.65 | A | N |
| ATOM | 1823 | CA | VAL | A | 240 | 56.599 | 30.569 | 6.057 | 1.00 | 32.95 | A | C |
| ATOM | 1824 | C | VAL | A | 240 | 58.018 | 30.172 | 5.684 | 1.00 | 28.03 | A | C |
| ATOM | 1825 | O | VAL | A | 240 | 58.201 | 29.484 | 4.704 | 1.00 | 26.73 | A | O |
| ATOM | 1826 | CB | VAL | A | 240 | 56.323 | 31.928 | 5.390 | 1.00 | 28.86 | A | C |
| ATOM | 1827 | CG1 | VAL | A | 240 | 56.411 | 31.801 | 3.908 | 1.00 | 29.02 | A | C |
| ATOM | 1828 | CG2 | VAL | A | 240 | 54.963 | 32.517 | 5.818 | 1.00 | 33.40 | A | C |
| ATOM | 1829 | N | PHE | A | 241 | 59.019 | 30.655 | 6.430 | 1.00 | 31.69 | A | N |
| ATOM | 1830 | CA | PHE | A | 241 | 60.402 | 30.267 | 6.159 | 1.00 | 34.11 | A | C |
| ATOM | 1831 | C | PHE | A | 241 | 60.559 | 28.745 | 6.338 | 1.00 | 32.98 | A | C |
| ATOM | 1832 | O | PHE | A | 241 | 61.100 | 28.029 | 5.470 | 1.00 | 29.40 | A | O |
| ATOM | 1833 | CB | PHE | A | 241 | 61.360 | 30.997 | 7.095 | 1.00 | 35.69 | A | C |
| ATOM | 1834 | CG | PHE | A | 241 | 62.748 | 30.459 | 7.057 | 1.00 | 39.37 | A | C |
| ATOM | 1835 | CD1 | PHE | A | 241 | 63.601 | 30.785 | 6.012 | 1.00 | 42.58 | A | C |
| ATOM | 1836 | CD2 | PHE | A | 241 | 63.197 | 29.583 | 8.047 | 1.00 | 42.39 | A | C |
| ATOM | 1837 | CE1 | PHE | A | 241 | 64.895 | 30.272 | 5.968 | 1.00 | 43.06 | A | C |
| ATOM | 1838 | CE2 | PHE | A | 241 | 64.479 | 29.073 | 8.007 | 1.00 | 38.87 | A | C |
| ATOM | 1839 | CZ | PHE | A | 241 | 65.329 | 29.419 | 6.964 | 1.00 | 43.23 | A | C |
| ATOM | 1840 | N | GLU | A | 242 | 60.075 | 28.249 | 7.468 | 1.00 | 32.78 | A | N |
| ATOM | 1841 | CA | GLU | A | 242 | 60.011 | 26.800 | 7.696 | 1.00 | 36.60 | A | C |
| ATOM | 1842 | C | GLU | A | 242 | 59.505 | 26.025 | 6.487 | 1.00 | 33.79 | A | C |
| ATOM | 1843 | O | GLU | A | 242 | 60.160 | 25.083 | 6.053 | 1.00 | 35.48 | A | O |
| ATOM | 1844 | CB | GLU | A | 242 | 59.123 | 26.473 | 8.899 | 1.00 | 37.43 | A | C |
| ATOM | 1845 | CG | GLU | A | 242 | 59.830 | 26.686 | 10.217 | 1.00 | 43.87 | A | C |
| ATOM | 1846 | CD | GLU | A | 242 | 60.878 | 25.635 | 10.508 | 1.00 | 45.01 | A | C |
| ATOM | 1847 | OE1 | GLU | A | 242 | 61.759 | 25.906 | 11.358 | 1.00 | 45.07 | A | O |
| ATOM | 1848 | OE2 | GLU | A | 242 | 60.818 | 24.545 | 9.888 | 1.00 | 52.08 | A | O |
| ATOM | 1849 | N | ALA | A | 243 | 58.358 | 26.437 | 5.942 | 1.00 | 34.61 | A | N |
| ATOM | 1850 | CA | ALA | A | 243 | 57.752 | 25.723 | 4.804 | 1.00 | 36.67 | A | C |
| ATOM | 1851 | C | ALA | A | 243 | 58.531 | 25.984 | 3.523 | 1.00 | 35.54 | A | C |
| ATOM | 1852 | O | ALA | A | 243 | 58.735 | 25.093 | 2.706 | 1.00 | 30.69 | A | O |
| ATOM | 1853 | CB | ALA | A | 243 | 56.307 | 26.138 | 4.615 | 1.00 | 36.74 | A | C |
| ATOM | 1854 | N | ALA | A | 244 | 58.961 | 27.231 | 3.375 | 1.00 | 36.29 | A | N |
| ATOM | 1855 | CA | ALA | A | 244 | 59.717 | 27.682 | 2.224 | 1.00 | 36.28 | A | C |
| ATOM | 1856 | C | ALA | A | 244 | 60.970 | 26.841 | 2.063 | 1.00 | 36.59 | A | C |
| ATOM | 1857 | O | ALA | A | 244 | 61.133 | 26.184 | 1.058 | 1.00 | 35.35 | A | O |
| ATOM | 1858 | CB | ALA | A | 244 | 60.073 | 29.142 | 2.383 | 1.00 | 34.54 | A | C |
| ATOM | 1859 | N | VAL | A | 245 | 61.853 | 26.884 | 3.064 | 1.00 | 38.90 | A | N |
| ATOM | 1860 | CA | VAL | A | 245 | 63.002 | 25.982 | 3.143 | 1.00 | 42.56 | A | C |
| ATOM | 1861 | C | VAL | A | 245 | 62.658 | 24.500 | 2.899 | 1.00 | 40.13 | A | C |
| ATOM | 1862 | O | VAL | A | 245 | 63.341 | 23.835 | 2.114 | 1.00 | 40.38 | A | O |
| ATOM | 1863 | CB | VAL | A | 245 | 63.742 | 26.130 | 4.515 | 1.00 | 43.37 | A | C |
| ATOM | 1864 | CG1 | VAL | A | 245 | 64.651 | 24.918 | 4.821 | 1.09 | 47.23 | A | C |
| ATOM | 1865 | CG2 | VAL | A | 245 | 64.541 | 27.420 | 4.534 | 1.00 | 44.13 | A | C |
| ATOM | 1866 | N | LYS | A | 246 | 61.627 | 23.974 | 3.556 | 1.00 | 39.54 | A | N |
| ATOM | 1867 | CA | LYS | A | 246 | 61.270 | 22.556 | 3.352 | 1.00 | 44.44 | A | C |
| ATOM | 1868 | C | LYS | A | 246 | 61.172 | 22.215 | 1.859 | 1.00 | 43.12 | A | C |
| ATOM | 1869 | O | LYS | A | 246 | 61.745 | 21.233 | 1.407 | 1.00 | 40.51 | A | O |
| ATOM | 1870 | CB | LYS | A | 246 | 59.965 | 22.180 | 4.068 | 1.00 | 46.80 | A | C |
| ATOM | 1871 | CG | LYS | A | 246 | 59.575 | 20.695 | 3.924 | 1.00 | 50.38 | A | C |
| ATOM | 1872 | CD | LYS | A | 246 | 58.263 | 20.380 | 4.653 | 1.00 | 52.29 | A | C |
| ATOM | 1873 | CE | LYS | A | 246 | 57.530 | 19.182 | 4.042 | 1.00 | 54.52 | A | C |
| ATOM | 1874 | NZ | LYS | A | 246 | 58.452 | 18.072 | 3.656 | 1.00 | 53.80 | A | N |
| ATOM | 1875 | N | SER | A | 247 | 60.473 | 23.051 | 1.097 | 1.00 | 44.68 | A | N |
| ATOM | 1876 | CA | SER | A | 247 | 60.282 | 22.809 | −0.337 | 1.00 | 45.24 | A | C |
| ATOM | 1877 | C | SER | A | 247 | 61.505 | 23.062 | −1.226 | 1.00 | 43.18 | A | C |
| ATOM | 1878 | O | SER | A | 247 | 61.653 | 22.423 | −2.258 | 1.00 | 38.59 | A | O |
| ATOM | 1879 | CB | SER | A | 247 | 59.126 | 23.654 | −0.869 | 1.00 | 45.97 | A | C |
| ATOM | 1880 | OG | SER | A | 247 | 59.035 | 23.478 | −2.266 | 1.00 | 42.87 | A | O |
| ATOM | 1881 | N | ILE | A | 248 | 62.345 | 24.027 | −0.861 | 1.00 | 47.83 | A | N |
| ATOM | 1882 | CA | ILE | A | 248 | 63.534 | 24.348 | −1.658 | 1.00 | 50.81 | A | C |
| ATOM | 1883 | C | ILE | A | 248 | 64.570 | 23.241 | −1.475 | 1.00 | 52.35 | A | C |
| ATOM | 1884 | O | ILE | A | 248 | 65.200 | 22.787 | −2.440 | 1.00 | 48.20 | A | O |
| ATOM | 1885 | CB | ILE | A | 248 | 64.116 | 25.716 | −1.260 | 1.00 | 51.35 | A | C |
| ATOM | 1886 | CG1 | ILE | A | 248 | 63.101 | 26.823 | −1.548 | 1.00 | 50.95 | A | C |
| ATOM | 1887 | CG2 | ILE | A | 248 | 65.428 | 25.983 | −2.015 | 1.00 | 51.10 | A | C |
| ATOM | 1888 | CD1 | ILE | A | 248 | 63.447 | 28.154 | −0.913 | 1.00 | 51.52 | A | C |
| ATOM | 1889 | N | LYS | A | 249 | 64.725 | 22.814 | −0.227 | 1.00 | 53.72 | A | N |
| ATOM | 1890 | CA | LYS | A | 249 | 65.451 | 21.585 | 0.108 | 1.00 | 59.31 | A | C |
| ATOM | 1891 | C | LYS | A | 249 | 65.021 | 20.377 | −0.745 | 1.00 | 61.00 | A | C |
| ATOM | 1892 | O | LYS | A | 249 | 65.871 | 19.634 | −1.233 | 1.00 | 58.84 | A | O |
| ATOM | 1893 | CB | LYS | A | 249 | 65.260 | 21.257 | 1.598 | 1.00 | 61.17 | A | C |
| ATOM | 1894 | CG | LYS | A | 249 | 66.419 | 20.522 | 2.240 | 1.00 | 63.43 | A | C |
| ATOM | 1895 | CD | LYS | A | 249 | 66.187 | 20.317 | 3.740 | 1.00 | 65.86 | A | C |
| ATOM | 1896 | CE | LYS | A | 249 | 66.299 | 21.620 | 4.530 | 1.00 | 67.19 | A | C |
| ATOM | 1897 | NZ | LYS | A | 249 | 66.791 | 21.417 | 5.929 | 1.00 | 68.30 | A | N |
| ATOM | 1898 | N | ALA | A | 250 | 63.711 | 20.207 | −0.942 | 1.00 | 63.51 | A | N |
| ATOM | 1899 | CA | ALA | A | 250 | 63.160 | 19.006 | −1.589 | 1.00 | 66.01 | A | C |
| ATOM | 1900 | C | ALA | A | 250 | 63.564 | 18.839 | −3.059 | 1.00 | 67.43 | A | C |
| ATOM | 1901 | O | ALA | A | 250 | 64.214 | 17.861 | −3.408 | 1.00 | 67.59 | A | O |
| ATOM | 1902 | CB | ALA | A | 250 | 61.635 | 18.974 | −1.455 | 1.00 | 65.47 | A | C |
| ATOM | 1903 | N | ALA | A | 251 | 63.185 | 19.783 | −3.917 | 1.00 | 69.93 | A | N |
| ATOM | 1904 | CA | ALA | A | 251 | 63.539 | 19.694 | −5.342 | 1.00 | 70.60 | A | C |
| ATOM | 1905 | C | ALA | A | 251 | 64.985 | 20.123 | −5.633 | 1.00 | 70.50 | A | C |
| ATOM | 1906 | O | ALA | A | 251 | 65.364 | 20.268 | −6.794 | 1.00 | 69.29 | A | O |
| ATOM | 1907 | CB | ALA | A | 251 | 62.547 | 20.488 | −6.212 | 1.00 | 70.96 | A | C |
| ATOM | 1908 | N | SER | A | 252 | 65.778 | 20.338 | −4.582 | 1.00 | 70.95 | A | N |
| ATOM | 1909 | CA | SER | A | 252 | 67.213 | 20.562 | −4.718 | 1.00 | 72.23 | A | C |
| ATOM | 1910 | C | SER | A | 252 | 68.016 | 19.483 | −3.985 | 1.00 | 73.46 | A | C |
| ATOM | 1911 | O | SER | A | 252 | 69.189 | 19.680 | −3.661 | 1.00 | 71.37 | A | O |
| ATOM | 1912 | CB | SER | A | 252 | 67.582 | 21.951 | −4.189 | 1.00 | 72.68 | A | C |
| ATOM | 1913 | OG | SER | A | 252 | 67.505 | 21.999 | −2.775 | 1.00 | 73.18 | A | O |
| ATOM | 1914 | N | SER | A | 253 | 67.389 | 18.332 | −3.756 | 1.00 | 75.44 | A | N |
| ATOM | 1915 | CA | SER | A | 253 | 68.011 | 17.239 | −3.011 | 1.00 | 77.44 | A | C |
| ATOM | 1916 | C | SER | A | 253 | 69.079 | 16.491 | −3.819 | 1.00 | 79.67 | A | C |
| ATOM | 1917 | O | SER | A | 253 | 69.783 | 15.645 | −3.263 | 1.00 | 79.72 | A | O |
| ATOM | 1918 | CB | SER | A | 253 | 66.944 | 16.250 | −2.532 | 1.00 | 77.46 | A | C |
| ATOM | 1919 | OG | SER | A | 253 | 66.037 | 16.870 | −1.637 | 1.00 | 76.30 | A | O |
| ATOM | 1920 | N | THR | A | 254 | 69.196 | 16.799 | −5.116 | 1.00 | 81.88 | A | N |
| ATOM | 1921 | CA | THR | A | 254 | 70.232 | 16.215 | −5.983 | 1.00 | 83.80 | A | C |
| ATOM | 1922 | C | THR | A | 254 | 71.624 | 16.245 | −5.334 | 1.00 | 85.34 | A | C |
| ATOM | 1923 | O | THR | A | 254 | 72.423 | 15.330 | −5.538 | 1.00 | 85.86 | A | O |
| ATOM | 1924 | CB | THR | A | 254 | 70.270 | 16.936 | −7.360 | 1.00 | 83.52 | A | C |
| ATOM | 1925 | OG1 | THR | A | 254 | 68.992 | 16.851 | −7.999 | 1.00 | 83.21 | A | O |
| ATOM | 1926 | CG2 | THR | A | 254 | 71.205 | 16.228 | −8.342 | 1.00 | 83.62 | A | C |
| ATOM | 1927 | N | GLU | A | 255 | 71.909 | 17.296 | −4.565 | 1.00 | 86.72 | A | N |
| ATOM | 1928 | CA | GLU | A | 255 | 73.121 | 17.354 | −3.746 | 1.00 | 87.75 | A | C |
| ATOM | 1929 | C | GLU | A | 255 | 72.785 | 17.714 | −2.302 | 1.00 | 88.51 | A | C |
| ATOM | 1930 | O | GLU | A | 255 | 72.017 | 18.643 | −2.048 | 1.00 | 89.05 | A | O |
| ATOM | 1931 | CB | GLU | A | 255 | 74.103 | 18.379 | −4.307 | 1.00 | 87.94 | A | C |
| ATOM | 1932 | CG | GLU | A | 255 | 74.553 | 18.101 | −5.731 | 1.00 | 88.21 | A | C |
| ATOM | 1933 | CD | GLU | A | 255 | 75.403 | 19.222 | −6.297 | 1.00 | 88.85 | A | C |
| ATOM | 1934 | OE1 | GLU | A | 255 | 76.162 | 19.847 | −5.521 | 1.00 | 88.18 | A | O |
| ATOM | 1935 | OE2 | GLU | A | 255 | 75.308 | 19.478 | −7.518 | 1.00 | 89.16 | A | O |
| ATOM | 1936 | N | LYS | A | 256 | 73.367 | 16.973 | −1.361 | 1.00 | 89.37 | A | N |
| ATOM | 1937 | CA | LYS | A | 256 | 73.181 | 17.233 | 0.066 | 1.00 | 89.70 | A | C |
| ATOM | 1938 | C | LYS | A | 256 | 74.060 | 18.406 | 0.515 | 1.00 | 89.43 | A | C |
| ATOM | 1939 | O | LYS | A | 256 | 75.211 | 18.519 | 0.090 | 1.00 | 90.96 | A | O |
| ATOM | 1940 | CB | LYS | A | 256 | 73.524 | 15.976 | 0.878 | 1.00 | 89.79 | A | C |
| ATOM | 1941 | CG | LYS | A | 256 | 73.344 | 16.118 | 2.390 | 1.00 | 89.80 | A | C |
| ATOM | 1942 | CD | LYS | A | 256 | 73.488 | 14.778 | 3.106 | 1.00 | 89.76 | A | C |
| ATOM | 1943 | CE | LYS | A | 256 | 74.916 | 14.250 | 3.037 | 1.00 | 89.66 | A | C |
| ATOM | 1944 | NZ | LYS | A | 256 | 75.135 | 13.099 | 3.955 | 1.00 | 89.59 | A | N |
| ATOM | 1945 | N | PHE | A | 257 | 73.509 | 19.278 | 1.359 | 1.00 | 88.33 | A | N |
| ATOM | 1946 | CA | PHE | A | 257 | 74.277 | 20.358 | 1.988 | 1.00 | 87.80 | A | C |
| ATOM | 1947 | C | PHE | A | 257 | 73.957 | 20.434 | 3.486 | 1.00 | 85.90 | A | C |
| ATOM | 1948 | O | PHE | A | 257 | 72.901 | 19.963 | 3.916 | 1.00 | 84.62 | A | O |
| ATOM | 1949 | CB | PHE | A | 257 | 73.977 | 21.698 | 1.307 | 1.00 | 88.75 | A | C |
| ATOM | 1950 | CG | PHE | A | 257 | 74.158 | 21.672 | −0.188 | 1.00 | 90.18 | A | C |
| ATOM | 1951 | CD1 | PHE | A | 257 | 73.128 | 22.071 | −1.035 | 1.00 | 90.73 | A | C |
| ATOM | 1952 | CD2 | PHE | A | 257 | 75.358 | 21.243 | −0.747 | 1.00 | 90.65 | A | C |
| ATOM | 1953 | CE1 | PHE | A | 257 | 73.295 | 22.043 | −2.417 | 1.00 | 91.50 | A | C |
| ATOM | 1954 | CE2 | PHE | A | 257 | 75.530 | 21.208 | −2.125 | 1.00 | 91.35 | A | C |
| ATOM | 1955 | CZ | PHE | A | 257 | 74.499 | 21.611 | −2.961 | 1.00 | 91.69 | A | C |
| ATOM | 1956 | N | PRO | A | 258 | 74.857 | 21.019 | 4.282 | 1.00 | 84.41 | A | N |
| ATOM | 1957 | CA | PRO | A | 258 | 74.671 | 21.079 | 5.743 | 1.00 | 83.94 | A | C |
| ATOM | 1958 | C | PRO | A | 258 | 73.334 | 21.697 | 6.182 | 1.00 | 83.10 | A | C |
| ATOM | 1959 | O | PRO | A | 258 | 72.764 | 22.519 | 5.459 | 1.00 | 82.64 | A | O |
| ATOM | 1960 | CB | PRO | A | 258 | 75.840 | 21.957 | 6.218 | 1.00 | 83.97 | A | C |
| ATOM | 1961 | CG | PRO | A | 258 | 76.862 | 21.878 | 5.141 | 1.00 | 84.54 | A | C |
| ATOM | 1962 | CD | PRO | A | 258 | 76.116 | 21.664 | 3.861 | 1.00 | 84.44 | A | C |
| ATOM | 1963 | N | ASP | A | 259 | 72.852 | 21.302 | 7.360 | 1.00 | 81.28 | A | N |
| ATOM | 1964 | CA | ASP | A | 259 | 71.608 | 21.847 | 7.916 | 1.00 | 79.36 | A | C |
| ATOM | 1965 | C | ASP | A | 259 | 71.767 | 23.327 | 8.300 | 1.00 | 76.80 | A | C |
| ATOM | 1966 | O | ASP | A | 259 | 70.804 | 24.097 | 8.228 | 1.00 | 75.77 | A | O |
| ATOM | 1967 | CB | ASP | A | 259 | 71.140 | 21.025 | 9.133 | 1.00 | 80.43 | A | C |
| ATOM | 1968 | CG | ASP | A | 259 | 69.749 | 20.420 | 8.947 | 1.00 | 81.62 | A | C |
| ATOM | 1969 | OD1 | ASP | A | 259 | 69.433 | 19.944 | 7.832 | 1.00 | 81.91 | A | O |
| ATOM | 1970 | OD2 | ASP | A | 259 | 68.906 | 20.364 | 9.870 | 1.00 | 82.81 | A | O |
| ATOM | 1971 | N | GLY | A | 260 | 72.981 | 23.716 | 8.694 | 1.00 | 72.15 | A | N |
| ATOM | 1972 | CA | GLY | A | 260 | 73.280 | 25.095 | 9.051 | 1.00 | 68.23 | A | C |
| ATOM | 1973 | C | GLY | A | 260 | 73.394 | 26.055 | 7.873 | 1.00 | 65.01 | A | C |
| ATOM | 1974 | O | GLY | A | 260 | 73.306 | 27.266 | 8.055 | 1.00 | 62.06 | A | O |
| ATOM | 1975 | N | PHE | A | 261 | 73.601 | 25.529 | 6.670 | 1.00 | 61.00 | A | N |
| ATOM | 1976 | CA | PHE | A | 261 | 73.582 | 26.350 | 5.456 | 1.00 | 59.12 | A | C |
| ATOM | 1977 | C | PHE | A | 261 | 72.247 | 27.101 | 5.311 | 1.00 | 57.30 | A | C |
| ATOM | 1978 | O | PHE | A | 261 | 72.217 | 28.296 | 5.013 | 1.00 | 47.15 | A | O |
| ATOM | 1979 | CB | PHE | A | 261 | 73.833 | 25.475 | 4.222 | 1.00 | 59.54 | A | C |
| ATOM | 1980 | CG | PHE | A | 261 | 73.504 | 26.148 | 2.920 | 1.00 | 58.56 | A | C |
| ATOM | 1981 | CD1 | PHE | A | 261 | 74.289 | 27.187 | 2.447 | 1.00 | 57.89 | A | C |
| ATOM | 1982 | CD2 | PHE | A | 261 | 72.415 | 25.741 | 2.169 | 1.00 | 58.88 | A | C |
| ATOM | 1983 | CE1 | PHE | A | 261 | 73.996 | 27.807 | 1.259 | 1.00 | 56.77 | A | C |
| ATOM | 1984 | CE2 | PHE | A | 261 | 72.118 | 26.359 | 0.972 | 1.00 | 58.83 | A | C |
| ATOM | 1985 | CZ | PHE | A | 261 | 72.912 | 27.393 | 0.516 | 1.00 | 58.32 | A | C |
| ATOM | 1986 | N | TRP | A | 262 | 71.151 | 26.389 | 5.557 | 1.00 | 56.79 | A | N |
| ATOM | 1987 | CA | TRP | A | 262 | 69.813 | 26.949 | 5.396 | 1.00 | 58.03 | A | C |
| ATOM | 1988 | C | TRP | A | 262 | 69.475 | 27.995 | 6.467 | 1.00 | 58.02 | A | C |
| ATOM | 1989 | O | TRP | A | 262 | 68.525 | 28.760 | 6.296 | 1.00 | 57.45 | A | O |
| ATOM | 1990 | CB | TRP | A | 262 | 68.759 | 25.832 | 5.406 | 1.00 | 59.10 | A | C |
| ATOM | 1991 | CG | TRP | A | 262 | 69.026 | 24.721 | 4.432 | 1.00 | 60.40 | A | C |
| ATOM | 1992 | CD1 | TRP | A | 262 | 69.372 | 23.430 | 4.730 | 1.00 | 61.96 | A | C |
| ATOM | 1993 | CD2 | TRP | A | 262 | 68.974 | 24.800 | 3.003 | 1.00 | 61.91 | A | C |
| ATOM | 1994 | NE1 | TRP | A | 262 | 69.535 | 22.704 | 3.574 | 1.00 | 60.97 | A | N |
| ATOM | 1995 | CE2 | TRP | A | 262 | 69.298 | 23.520 | 2.498 | 1.00 | 61.58 | A | C |
| ATOM | 1996 | CE3 | TRP | A | 262 | 68.688 | 25.824 | 2.092 | 1.00 | 62.89 | A | C |
| ATOM | 1997 | CZ2 | TRP | A | 262 | 69.343 | 23.240 | 1.132 | 1.00 | 62.27 | A | C |
| ATOM | 1998 | CZ3 | TRP | A | 262 | 68.729 | 25.543 | 0.731 | 1.00 | 63.90 | A | C |
| ATOM | 1999 | CH2 | TRP | A | 262 | 69.057 | 24.260 | 0.267 | 1.00 | 63.92 | A | C |
| ATOM | 2000 | N | LEU | A | 263 | 70.236 | 28.025 | 7.563 | 1.00 | 56.89 | A | N |
| ATOM | 2001 | CA | LEU | A | 263 | 70.040 | 29.026 | 8.626 | 1.00 | 57.55 | A | C |
| ATOM | 2002 | C | LEU | A | 263 | 71.038 | 30.204 | 8.576 | 1.00 | 55.97 | A | C |
| ATOM | 2003 | O | LEU | A | 263 | 71.084 | 31.020 | 9.501 | 1.00 | 51.83 | A | O |
| ATOM | 2004 | CB | LEU | A | 263 | 70.102 | 28.355 | 10.010 | 1.00 | 58.30 | A | C |
| ATOM | 2005 | CG | LEU | A | 263 | 68.913 | 27.494 | 10.470 | 1.00 | 61.17 | A | C |
| ATOM | 2006 | CD1 | LEU | A | 263 | 67.569 | 28.209 | 10.298 | 1.00 | 62.02 | A | C |
| ATOM | 2007 | CD2 | LEU | A | 263 | 68.900 | 26.160 | 9.759 | 1.00 | 61.49 | A | C |
| ATOM | 2008 | N | GLY | A | 264 | 71.821 | 30.300 | 7.501 | 1.00 | 54.23 | A | N |
| ATOM | 2009 | CA | GLY | A | 264 | 72.793 | 31.378 | 7.352 | 1.00 | 55.51 | A | C |
| ATOM | 2010 | C | GLY | A | 264 | 73.964 | 31.272 | 8.318 | 1.00 | 53.62 | A | C |
| ATOM | 2011 | O | GLY | A | 264 | 74.657 | 32.259 | 8.582 | 1.00 | 52.01 | A | O |
| ATOM | 2012 | N | GLU | A | 265 | 74.180 | 30.057 | 8.822 | 1.00 | 52.02 | A | N |
| ATOM | 2013 | CA | GLU | A | 265 | 75.202 | 29.749 | 9.818 | 1.00 | 52.34 | A | C |
| ATOM | 2014 | C | GLU | A | 265 | 76.498 | 29.185 | 9.216 | 1.00 | 51.88 | A | C |
| ATOM | 2015 | O | GLU | A | 265 | 77.521 | 29.092 | 9.902 | 1.00 | 50.86 | A | O |
| ATOM | 2016 | CB | GLU | A | 265 | 74.620 | 28.757 | 10.823 | 1.00 | 53.23 | A | C |
| ATOM | 2017 | CG | GLU | A | 265 | 73.484 | 29.351 | 11.651 | 1.00 | 56.42 | A | C |
| ATOM | 2018 | CD | GLU | A | 265 | 72.811 | 28.342 | 12.572 | 1.00 | 59.67 | A | C |
| ATOM | 2019 | OE1 | GLU | A | 265 | 73.053 | 27.121 | 12.415 | 1.00 | 62.41 | A | O |
| ATOM | 2020 | OE2 | GLU | A | 265 | 72.029 | 28.777 | 13.451 | 1.00 | 58.43 | A | O |
| ATOM | 2021 | N | GLN | A | 266 | 76.444 | 28.781 | 7.951 | 1.00 | 48.71 | A | N |
| ATOM | 2022 | CA | GLN | A | 266 | 77.650 | 28.438 | 7.197 | 1.00 | 50.93 | A | C |
| ATOM | 2023 | C | GLN | A | 266 | 77.436 | 28.743 | 5.726 | 1.00 | 50.91 | A | C |
| ATOM | 2024 | O | GLN | A | 266 | 76.300 | 28.790 | 5.262 | 1.00 | 48.81 | A | O |
| ATOM | 2025 | CB | GLN | A | 266 | 77.919 | 26.957 | 7.385 | 1.00 | 51.13 | A | C |
| ATOM | 2026 | CG | GLN | A | 266 | 79.152 | 26.489 | 6.603 | 0.00 | 20.00 | A | C |
| ATOM | 2027 | CD | GLN | A | 266 | 79.377 | 25.016 | 6.849 | 0.00 | 20.00 | A | C |
| ATOM | 2028 | OE1 | GLN | A | 266 | 79.286 | 24.174 | 5.970 | 0.00 | 20.00 | A | O |
| ATOM | 2029 | NE2 | GLN | A | 266 | 79.677 | 24.718 | 8.129 | 0.00 | 20.00 | A | N |
| ATOM | 2030 | N | LEU | A | 267 | 78.523 | 28.947 | 4.988 | 1.00 | 52.35 | A | N |
| ATOM | 2031 | CA | LEU | A | 267 | 78.414 | 29.197 | 3.555 | 1.00 | 55.74 | A | C |
| ATOM | 2032 | C | LEU | A | 267 | 78.794 | 27.955 | 2.759 | 1.00 | 55.94 | A | C |
| ATOM | 2033 | O | LEU | A | 267 | 79.623 | 27.161 | 3.189 | 1.00 | 55.10 | A | O |
| ATOM | 2034 | CB | LEU | A | 267 | 79.228 | 30.427 | 3.117 | 1.00 | 57.95 | A | C |
| ATOM | 2035 | CG | LEU | A | 267 | 80.592 | 30.763 | 3.719 | 1.00 | 59.40 | A | C |
| ATOM | 2036 | CD1 | LEU | A | 267 | 81.667 | 29.797 | 3.226 | 1.00 | 61.73 | A | C |
| ATQM | 2037 | CD2 | LEU | A | 267 | 80.966 | 32.199 | 3.379 | 1.00 | 59.25 | A | C |
| ATOM | 2038 | N | VAL | A | 268 | 78.141 | 27.778 | 1.614 | 1.00 | 59.08 | A | N |
| ATOM | 2039 | CA | VAL | A | 268 | 78.394 | 26.635 | 0.734 | 1.00 | 63.27 | A | C |
| ATOM | 2040 | C | VAL | A | 268 | 79.437 | 27.028 | −0.317 | 1.00 | 65.28 | A | C |
| ATOM | 2041 | O | VAL | A | 268 | 79.581 | 28.206 | −0.628 | 1.00 | 65.73 | A | O |
| ATOM | 2042 | CB | VAL | A | 268 | 77.072 | 26.103 | 0.084 | 1.00 | 63.60 | A | C |
| ATOM | 2043 | CG1 | VAL | A | 268 | 76.461 | 27.114 | −0.900 | 1.00 | 64.14 | A | C |
| ATOM | 2044 | CG2 | VAL | A | 268 | 77.302 | 24.759 | −0.593 | 1.00 | 63.65 | A | C |
| ATOM | 2045 | N | CYS | A | 269 | 80.182 | 26.050 | −0.830 | 1.00 | 67.59 | A | N |
| ATOM | 2046 | CA | CYS | A | 269 | 81.255 | 26.315 | −1.794 | 1.00 | 70.85 | A | C |
| ATOM | 2047 | C | CYS | A | 269 | 81.288 | 25.302 | −2.943 | 1.00 | 71.91 | A | C |
| ATOM | 2048 | O | CYS | A | 269 | 81.068 | 24.106 | −2.740 | 1.00 | 70.92 | A | O |
| ATOM | 2049 | CB | CYS | A | 269 | 82.618 | 26.330 | −1.086 | 1.00 | 71.01 | A | C |
| ATOM | 2050 | SG | CYS | A | 269 | 82.804 | 27.634 | 0.160 | 1.00 | 73.46 | A | S |
| ATOM | 2051 | N | TRP | A | 270 | 81.560 | 25.804 | −4.147 | 1.00 | 73.59 | A | N |
| ATQM | 2052 | CA | TRP | A | 270 | 81.768 | 24.975 | −5.335 | 1.00 | 74.68 | A | C |
| ATOM | 2053 | C | TRP | A | 270 | 83.036 | 25.412 | −6.065 | 1.00 | 75.66 | A | C |
| ATOM | 2054 | O | TRP | A | 270 | 83.455 | 26.563 | −5.952 | 1.00 | 74.31 | A | O |
| ATOM | 2055 | CB | TRP | A | 270 | 80.584 | 25.108 | −6.289 | 1.00 | 75.03 | A | C |
| ATOM | 2056 | CG | TRP | A | 270 | 79.330 | 24.451 | −5.812 | 1.00 | 74.79 | A | C |
| ATOM | 2057 | CD1 | TRP | A | 270 | 79.034 | 23.118 | −5.848 | 1.00 | 74.58 | A | C |
| ATOM | 2058 | CD2 | TRP | A | 270 | 78.191 | 25.099 | −5.239 | 1.00 | 74.13 | A | C |
| ATOM | 2059 | NE1 | TRP | A | 270 | 77.781 | 22.898 | −5.329 | 1.00 | 74.78 | A | N |
| ATOM | 2060 | CE2 | TRP | A | 270 | 77.241 | 24.098 | −4.946 | 1.00 | 74.47 | A | C |
| ATOM | 2061 | CE3 | TRP | A | 270 | 77.875 | 26.431 | −4.939 | 1.00 | 73.49 | A | C |
| ATOM | 2062 | CZ2 | TRP | A | 270 | 76.000 | 24.386 | −4.372 | 1.00 | 74.22 | A | C |
| ATOM | 2063 | CZ3 | TRP | A | 270 | 76.649 | 26.715 | −4.371 | 1.00 | 73.73 | A | C |
| ATOM | 2064 | CH2 | TRP | A | 270 | 75.727 | 25.696 | −4.088 | 1.00 | 73.99 | A | C |
| ATOM | 2065 | N | GLN | A | 271 | 83.633 | 24.497 | −6.827 | 1.00 | 77.31 | A | N |
| ATOM | 2066 | CA | GLN | A | 271 | 84.835 | 24.808 | −7.603 | 1.00 | 78.64 | A | C |
| ATOM | 2067 | C | GLN | A | 271 | 84.559 | 25.956 | −8.579 | 1.00 | 79.98 | A | C |
| ATOM | 2068 | O | GLN | A | 271 | 83.424 | 26.136 | −9.025 | 1.00 | 79.95 | A | O |
| ATOM | 2069 | CB | GLN | A | 271 | 85.233 | 23.565 | −8.378 | 1.00 | 78.38 | A | C |
| ATOM | 2070 | CG | GLN | A | 271 | 85.694 | 22.427 | −7.461 | 0.00 | 20.00 | A | C |
| ATOM | 2071 | CD | GLN | A | 271 | 86.108 | 21.239 | −8.297 | 0.00 | 20.00 | A | C |
| ATOM | 2072 | OE1 | GLN | A | 271 | 86.517 | 20.197 | −7.812 | 0.00 | 20.00 | A | O |
| ATOM | 2073 | NE2 | GLN | A | 271 | 85.995 | 21.443 | −9.624 | 0.00 | 20.00 | A | N |
| ATOM | 2074 | N | ALA | A | 272 | 85.596 | 26.733 | −8.891 | 1.00 | 81.27 | A | N |
| ATOM | 2075 | CA | ALA | A | 272 | 85.467 | 27.927 | −9.736 | 1.00 | 82.23 | A | C |
| ATOM | 2076 | C | ALA | A | 272 | 84.604 | 27.706 | −10.989 | 1.00 | 82.74 | A | C |
| ATOM | 2077 | O | ALA | A | 272 | 85.006 | 27.012 | −11.926 | 1.00 | 82.97 | A | O |
| ATOM | 2078 | CB | ALA | A | 272 | 86.850 | 28.443 | −10.131 | 1.00 | 82.17 | A | C |
| ATOM | 2079 | N | GLY | A | 273 | 83.408 | 28.289 | −10.977 | 1.00 | 83.80 | A | N |
| ATOM | 2080 | CA | GLY | A | 273 | 82.523 | 28.301 | −12.132 | 1.00 | 84.66 | A | C |
| ATOM | 2081 | C | GLY | A | 273 | 81.761 | 27.008 | −12.373 | 1.00 | 84.93 | A | C |
| ATOM | 2082 | O | GLY | A | 273 | 81.383 | 26.720 | −13.511 | 1.00 | 86.15 | A | O |
| ATOM | 2083 | N | THR | A | 274 | 81.509 | 26.250 | −11.305 | 1.00 | 84.63 | A | N |
| ATOM | 2084 | CA | THR | A | 274 | 80.861 | 24.940 | −11.396 | 1.00 | 84.75 | A | C |
| ATOM | 2085 | C | THR | A | 274 | 79.570 | 24.921 | −10.581 | 1.00 | 84.35 | A | C |
| ATOM | 2086 | O | THR | A | 274 | 79.158 | 23.880 | −10.064 | 1.00 | 82.91 | A | O |
| ATOM | 2087 | CB | THR | A | 274 | 81.812 | 23.828 | −10.895 | 1.00 | 85.61 | A | C |
| ATOM | 2088 | OG1 | THR | A | 274 | 82.135 | 24.043 | −9.514 | 1.00 | 85.37 | A | O |
| ATOM | 2089 | CG2 | THR | A | 274 | 83.162 | 23.879 | −11.618 | 1.00 | 86.24 | A | C |
| ATOM | 2090 | N | THR | A | 275 | 78.929 | 26.079 | −10.480 | 1.00 | 84.51 | A | N |
| ATOM | 2091 | CA | THR | A | 275 | 77.719 | 26.213 | −9.688 | 1.00 | 85.22 | A | C |
| ATOM | 2092 | C | THR | A | 275 | 76.535 | 25.616 | −10.451 | 1.00 | 84.97 | A | C |
| ATOM | 2093 | O | THR | A | 275 | 76.311 | 25.959 | −11.614 | 1.00 | 83.74 | A | O |
| ATOM | 2094 | CB | THR | A | 275 | 77.450 | 27.690 | −9.370 | 1.00 | 85.63 | A | C |
| ATOM | 2095 | OG1 | THR | A | 275 | 78.649 | 28.313 | −8.889 | 1.00 | 87.20 | A | O |
| ATOM | 2096 | CG2 | THR | A | 275 | 76.472 | 27.827 | −8.210 | 1.00 | 86.40 | A | C |
| ATOM | 2097 | N | PRO | A | 276 | 75.790 | 24.718 | −9.808 | 1.00 | 84.86 | A | N |
| ATOM | 2098 | CA | PRO | A | 276 | 74.591 | 24.138 | −10.420 | 1.00 | 84.84 | A | C |
| ATOM | 2099 | C | PRO | A | 276 | 73.386 | 25.087 | −10.330 | 1.00 | 84.60 | A | C |
| ATOM | 2100 | O | PRO | A | 276 | 72.759 | 25.213 | −9.270 | 1.00 | 83.40 | A | O |
| ATOM | 2101 | CB | PRO | A | 276 | 74.363 | 22.864 | −9.599 | 1.00 | 85.26 | A | C |
| ATOM | 2102 | CG | PRO | A | 276 | 74.945 | 23.160 | −8.247 | 1.00 | 84.89 | A | C |
| ATOM | 2103 | CD | PRO | A | 276 | 76.033 | 24.178 | −8.456 | 1.00 | 84.87 | A | C |
| ATOM | 2104 | N | TRP | A | 277 | 73.084 | 25.757 | −11.441 | 1.00 | 83.87 | A | N |
| ATOM | 2105 | CA | TRP | A | 277 | 71.945 | 26.671 | −11.512 | 1.00 | 82.59 | A | C |
| ATOM | 2106 | C | TRP | A | 277 | 70.645 | 25.914 | −11.259 | 1.00 | 78.91 | A | C |
| ATOM | 2107 | O | TRP | A | 277 | 69.868 | 26.253 | −10.361 | 1.00 | 76.37 | A | O |
| ATOM | 2108 | CB | TRP | A | 277 | 71.852 | 27.327 | −12.903 | 1.00 | 84.62 | A | C |
| ATOM | 2109 | CG | TRP | A | 277 | 72.863 | 28.426 | −13.258 | 1.00 | 86.23 | A | C |
| ATOM | 2110 | CD1 | TRP | A | 277 | 73.299 | 28.743 | −14.520 | 1.00 | 86.71 | A | C |
| ATOM | 2111 | CD2 | TRP | A | 277 | 73.518 | 29.358 | −12.371 | 1.00 | 86.55 | A | C |
| ATOM | 2112 | NE1 | TRP | A | 277 | 74.186 | 29.792 | −14.473 | 1.00 | 86.87 | A | N |
| ATOM | 2113 | CE2 | TRP | A | 277 | 74.340 | 30.190 | −13.171 | 1.00 | 87.19 | A | C |
| ATOM | 2114 | CE3 | TRP | A | 277 | 73.503 | 29.573 | −10.982 | 1.00 | 86.48 | A | C |
| ATOM | 2115 | CZ2 | TRP | A | 277 | 75.129 | 31.211 | −12.632 | 1.00 | 87.46 | A | C |
| ATOM | 2116 | CZ3 | TRP | A | 277 | 74.291 | 30.587 | −10.449 | 1.00 | 86.65 | A | C |
| ATOM | 2117 | CH2 | TRP | A | 277 | 75.092 | 31.392 | −11.273 | 1.00 | 87.15 | A | C |
| ATOM | 2118 | N | ASN | A | 278 | 70.450 | 24.864 | −12.052 | 1.00 | 75.16 | A | N |
| ATOM | 2119 | CA | ASN | A | 278 | 69.149 | 24.232 | −12.228 | 1.00 | 71.96 | A | C |
| ATOM | 2120 | C | ARN | A | 278 | 68.678 | 23.332 | −11.094 | 1.00 | 68.53 | A | C |
| ATOM | 2121 | O | ASN | A | 278 | 67.516 | 22.952 | −11.057 | 1.00 | 65.41 | A | O |
| ATOM | 2122 | CB | ASN | A | 278 | 69.145 | 23.456 | −13.544 | 1.00 | 71.99 | A | C |
| ATOM | 2123 | CG | ASN | A | 278 | 69.124 | 24.372 | −14.749 | 1.00 | 73.02 | A | C |
| ATOM | 2124 | OD1 | ASN | A | 278 | 68.090 | 24.528 | −15.397 | 1.00 | 75.67 | A | O |
| ATOM | 2125 | ND2 | ASN | A | 278 | 70.261 | 25.002 | −15.043 | 1.00 | 71.03 | A | N |
| ATOM | 2126 | N | ILE | A | 279 | 69.567 | 23.002 | −10.164 | 1.00 | 67.52 | A | N |
| ATOM | 2127 | CA | ILE | A | 279 | 69.188 | 22.191 | −9.006 | 1.00 | 66.60 | A | C |
| ATOM | 2128 | C | ILE | A | 279 | 68.277 | 22.978 | −8.046 | 1.00 | 63.12 | A | C |
| ATOM | 2129 | O | ILE | A | 279 | 67.509 | 22.382 | −7.293 | 1.00 | 61.43 | A | O |
| ATOM | 2130 | CB | ILE | A | 279 | 70.451 | 21.673 | −8.266 | 1.00 | 69.14 | A | C |
| ATOM | 2131 | CG1 | ILE | A | 279 | 71.311 | 20.801 | −9.196 | 1.00 | 70.33 | A | C |
| ATOM | 2132 | CG2 | ILE | A | 279 | 70.069 | 20.890 | −7.004 | 1.00 | 70.79 | A | C |
| ATOM | 2133 | CD1 | ILE | A | 279 | 70.601 | 19.582 | −9.773 | 1.00 | 71.75 | A | C |
| ATOM | 2134 | N | PHE | A | 280 | 68.364 | 24.310 | −8.089 | 1.00 | 58.16 | A | N |
| ATOM | 2135 | CA | PHE | A | 280 | 67.577 | 25.184 | −7.215 | 1.00 | 54.73 | A | C |
| ATOM | 2136 | C | PHE | A | 280 | 66.327 | 25.723 | −7.929 | 1.00 | 48.44 | A | C |
| ATOM | 2137 | O | PHE | A | 280 | 66.404 | 26.160 | −9.065 | 1.00 | 42.63 | A | O |
| ATOM | 2138 | CB | PHE | A | 280 | 68.440 | 26.352 | −6.737 | 1.00 | 54.07 | A | C |
| ATOM | 2139 | CG | PHE | A | 280 | 69.641 | 25.934 | −5.950 | 1.00 | 56.01 | A | C |
| ATOM | 2140 | CD1 | PHE | A | 280 | 70.860 | 25.735 | −6.582 | 1.00 | 56.28 | A | C |
| ATOM | 2141 | CD2 | PHE | A | 280 | 69.554 | 25.741 | −4.578 | 1.00 | 56.86 | A | C |
| ATOM | 2142 | CE1 | PHE | A | 280 | 71.975 | 25.351 | −5.861 | 1.00 | 58.79 | A | C |
| ATOM | 2143 | CE2 | PHE | A | 280 | 70.663 | 25.351 | −3.846 | 1.00 | 58.48 | A | C |
| ATOM | 2144 | CZ | PHE | A | 280 | 71.880 | 25.158 | −4.487 | 1.00 | 58.24 | A | C |
| ATOM | 2145 | N | PRO | A | 281 | 65.183 | 25.713 | −7.253 | 1.00 | 46.45 | A | N |
| ATOM | 2146 | CA | PRO | A | 281 | 63.933 | 26.153 | −7.873 | 1.00 | 45.34 | A | C |
| ATOM | 2147 | C | PRO | A | 281 | 63.830 | 27.670 | −7.930 | 1.00 | 46.32 | A | C |
| ATOM | 2148 | O | PRO | A | 281 | 64.540 | 28.377 | −7.209 | 1.00 | 44.85 | A | O |
| ATOM | 2149 | CB | PRO | A | 281 | 62.875 | 25.625 | −6.911 | 1.00 | 47.02 | A | C |
| ATOM | 2159 | CG | PRO | A | 281 | 63.552 | 25.707 | −5.574 | 1.00 | 48.68 | A | C |
| ATOM | 2151 | CD | PRO | A | 281 | 64.985 | 25.312 | −5.845 | 1.00 | 47.62 | A | C |
| ATOM | 2152 | N | VAL | A | 282 | 62.942 | 28.158 | −8.783 | 1.00 | 44.02 | A | N |
| ATOM | 2153 | CA | VAL | A | 282 | 62.594 | 29.567 | −8.785 | 1.00 | 44.12 | A | C |
| ATOM | 2154 | C | VAL | A | 282 | 61.625 | 29.811 | −7.650 | 1.00 | 44.35 | A | C |
| ATOM | 2155 | O | VAL | A | 282 | 60.976 | 28.886 | −7.174 | 1.00 | 43.30 | A | O |
| ATOM | 2156 | CB | VAL | A | 282 | 61.977 | 30.021 | −10.119 | 1.00 | 44.10 | A | C |
| ATOM | 2157 | CG1 | VAL | A | 282 | 62.930 | 29.754 | −11.259 | 1.00 | 40.34 | A | C |
| ATOM | 2158 | CG2 | VAL | A | 282 | 60.621 | 29.359 | −10.368 | 1.00 | 48.29 | A | C |
| ATOM | 2159 | N | ILE | A | 283 | 61.555 | 31.048 | −7.186 | 1.00 | 40.14 | A | N |
| ATOM | 2160 | CA | ILE | A | 283 | 60.502 | 31.421 | −6.273 | 1.00 | 42.52 | A | C |
| ATOM | 2161 | C | ILE | A | 283 | 59.656 | 32.479 | −6.970 | 1.00 | 39.13 | A | C |
| ATOM | 2162 | O | ILE | A | 283 | 60.171 | 33.297 | −7.743 | 1.00 | 35.09 | A | O |
| ATOM | 2163 | CB | ILE | A | 283 | 61.043 | 31.847 | −4.878 | 1.00 | 43.69 | A | C |
| ATOM | 2164 | CG1 | ILE | A | 283 | 59.925 | 32.432 | −4.023 | 1.00 | 46.75 | A | C |
| ATOM | 2165 | CG2 | ILE | A | 283 | 62.174 | 32.818 | −4.984 | 1.00 | 47.57 | A | C |
| ATOM | 2166 | CD1 | ILE | A | 283 | 60.216 | 32.406 | −2.547 | 1.00 | 47.98 | A | C |
| ATOM | 2167 | N | SER | A | 284 | 58.350 | 32.359 | −6.758 | 1.00 | 35.81 | A | N |
| ATOM | 2168 | CA | SER | A | 284 | 57.354 | 33.282 | −7.292 | 1.00 | 33.91 | A | C |
| ATOM | 2169 | C | SER | A | 284 | 56.555 | 33.814 | −6.121 | 1.00 | 32.23 | A | C |
| ATOM | 2170 | O | SER | A | 284 | 56.080 | 33.051 | −5.274 | 1.00 | 31.03 | A | O |
| ATOM | 2171 | CB | SER | A | 284 | 56.457 | 32.574 | −8.313 | 1.00 | 31.65 | A | C |
| ATOM | 2172 | OG | SER | A | 284 | 57.075 | 32.546 | −9.585 | 1.00 | 35.60 | A | O |
| ATOM | 2173 | N | LEU | A | 285 | 56.455 | 35.137 | −6.028 | 1.00 | 25.42 | A | N |
| ATOM | 2174 | CA | LEU | A | 285 | 55.588 | 35.760 | −5.069 | 1.00 | 24.59 | A | C |
| ATOM | 2175 | C | LEU | A | 285 | 54.478 | 36.397 | −5.890 | 1.00 | 21.13 | A | C |
| ATOM | 2176 | O | LEU | A | 285 | 54.770 | 37.108 | −6.839 | 1.00 | 19.53 | A | O |
| ATOM | 2177 | CB | LEU | A | 285 | 56.348 | 36.799 | −4.263 | 1.00 | 27.87 | A | C |
| ATOM | 2178 | CG | LEU | A | 285 | 57.674 | 36.297 | −3.682 | 1.00 | 27.04 | A | C |
| ATOM | 2179 | CD1 | LEU | A | 285 | 58.356 | 37.456 | −3.002 | 1.00 | 31.32 | A | C |
| ATOM | 2180 | CD2 | LEU | A | 285 | 57.428 | 35.159 | −2.702 | 1.00 | 29.67 | A | C |
| ATOM | 2181 | N | TYR | A | 286 | 53.233 | 36.065 | −5.587 | 1.00 | 22.23 | A | N |
| ATOM | 2182 | CA | TYR | A | 286 | 52.112 | 36.768 | −6.196 | 1.00 | 22.87 | A | C |
| ATOM | 2183 | C | TYR | A | 286 | 51.807 | 37.909 | −5.280 | 1.00 | 20.51 | A | C |
| ATOM | 2184 | O | TYR | A | 286 | 51.686 | 37.712 | −4.069 | 1.00 | 23.12 | A | O |
| ATOM | 2185 | CB | TYR | A | 286 | 50.871 | 35.898 | −6.336 | 1.00 | 18.84 | A | C |
| ATOM | 2186 | CG | TYR | A | 286 | 50.989 | 34.755 | −7.339 | 1.00 | 20.36 | A | C |
| ATOM | 2187 | CD1 | TYR | A | 286 | 51.857 | 33.691 | −7.125 | 1.00 | 27.97 | A | C |
| ATOM | 2188 | CD2 | TYR | A | 286 | 50.168 | 34.720 | −8.477 | 1.00 | 22.74 | A | C |
| ATOM | 2189 | CE1 | TYR | A | 286 | 51.937 | 32.641 | −8.024 | 1.00 | 28.14 | A | C |
| ATOM | 2190 | CE2 | TYR | A | 286 | 50.243 | 33.661 | −9.393 | 1.00 | 19.26 | A | C |
| ATOM | 2191 | CZ | TYR | A | 286 | 51.125 | 32.630 | −9.148 | 1.00 | 24.29 | A | C |
| ATOM | 2192 | OH | TYR | A | 286 | 51.198 | 31.595 | −10.033 | 1.00 | 23.89 | A | O |
| ATOM | 2193 | N | LEU | A | 287 | 51.672 | 39.113 | −5.849 | 1.00 | 17.57 | A | N |
| ATOM | 2194 | CA | LEU | A | 287 | 51.327 | 40.293 | −5.084 | 1.00 | 18.37 | A | C |
| ATOM | 2195 | C | LEU | A | 287 | 49.902 | 40.714 | −5.367 | 1.00 | 19.30 | A | C |
| ATOM | 2196 | O | LEU | A | 287 | 49.413 | 40.525 | −6.486 | 1.00 | 17.31 | A | O |
| ATOM | 2197 | CB | LEU | A | 287 | 52.291 | 41.429 | −5.435 | 1.00 | 16.99 | A | C |
| ATOM | 2198 | CG | LEU | A | 287 | 53.759 | 41.076 | −5.143 | 1.00 | 19.76 | A | C |
| ATOM | 2199 | CD1 | LEU | A | 287 | 54.689 | 42.131 | −5.672 | 1.00 | 21.23 | A | C |
| ATOM | 2200 | CD2 | LEU | A | 287 | 53.943 | 40.852 | −3.653 | 1.00 | 27.06 | A | C |
| ATOM | 2201 | N | MET | A | 288 | 49.250 | 41.310 | −4.369 | 1.00 | 17.49 | A | N |
| ATOM | 2202 | CA | MET | A | 288 | 47.906 | 41.869 | −4.524 | 1.00 | 14.88 | A | C |
| ATOM | 2203 | C | MET | A | 288 | 47.938 | 42.861 | −5.688 | 1.00 | 17.43 | A | C |
| ATOM | 2204 | O | MET | A | 288 | 48.833 | 43.675 | −5.798 | 1.00 | 16.72 | A | O |
| ATOM | 2205 | CB | MET | A | 288 | 47.471 | 42.597 | −3.242 | 1.00 | 19.44 | A | C |
| ATOM | 2206 | CG | MET | A | 288 | 46.150 | 43.315 | −3.360 | 1.00 | 21.18 | A | C |
| ATOM | 2207 | SD | MET | A | 288 | 45.656 | 44.123 | −1.787 | 1.00 | 27.75 | A | S |
| ATOM | 2208 | CE | MET | A | 288 | 45.045 | 42.809 | −0.930 | 1.00 | 26.84 | A | C |
| ATOM | 2209 | N | GLY | A | 289 | 46.961 | 42.792 | −6.574 | 1.00 | 15.66 | A | N |
| ATOM | 2210 | CA | GLY | A | 289 | 46.942 | 43.753 | −7.664 | 1.00 | 17.02 | A | C |
| ATOM | 2211 | C | GLY | A | 289 | 46.143 | 45.000 | −7.381 | 1.00 | 16.69 | A | C |
| ATOM | 2212 | O | GLY | A | 289 | 45.655 | 45.211 | −6.267 | 1.00 | 16.95 | A | O |
| ATOM | 2213 | N | GLU | A | 290 | 45.922 | 45.786 | −8.425 | 1.00 | 16.66 | A | N |
| ATOM | 2214 | CA | GLU | A | 290 | 45.190 | 47.057 | −8.298 | 1.00 | 17.93 | A | C |
| ATOM | 2215 | C | GLU | A | 290 | 43.656 | 46.888 | −8.251 | 1.00 | 18.47 | A | C |
| ATOM | 2216 | O | GLU | A | 290 | 42.944 | 47.782 | −7.800 | 1.00 | 20.92 | A | O |
| ATOM | 2217 | CB | GLU | A | 290 | 45.541 | 47.958 | −9.465 | 1.00 | 21.26 | A | C |
| ATOM | 2292 | O | THR | A | 299 | 61.614 | 36.337 | −11.139 | 1.00 | 28.84 | A | O |
| ATOM | 2293 | CB | THR | A | 299 | 59.291 | 34.640 | −11.560 | 1.00 | 27.03 | A | C |
| ATOM | 2294 | OG1 | THR | A | 299 | 57.902 | 34.287 | −11.572 | 1.00 | 32.46 | A | O |
| ATOM | 2295 | CG2 | THR | A | 299 | 60.001 | 33.283 | −11.620 | 1.00 | 28.77 | A | C |
| ATOM | 2296 | N | ILE | A | 300 | 61.803 | 35.268 | −9.175 | 1.00 | 31.26 | A | N |
| ATOM | 2297 | CA | ILE | A | 300 | 63.270 | 35.393 | −9.098 | 1.00 | 33.70 | A | C |
| ATOM | 2298 | C | ILE | A | 300 | 63.938 | 34.033 | −9.195 | 1.00 | 39.02 | A | C |
| ATOM | 2299 | O | ILE | A | 300 | 63.273 | 32.989 | −9.287 | 1.00 | 37.86 | A | O |
| ATOM | 2300 | CB | ILE | A | 300 | 63.729 | 36.107 | −7.793 | 1.00 | 35.95 | A | C |
| ATOM | 2301 | CG1 | ILE | A | 300 | 63.443 | 35.258 | −6.566 | 1.00 | 37.33 | A | C |
| ATOM | 2302 | CG2 | ILE | A | 300 | 63.049 | 37.457 | −7.639 | 1.00 | 35.67 | A | C |
| ATOM | 2303 | CD1 | ILE | A | 300 | 64.152 | 35.715 | −5.318 | 1.00 | 39.22 | A | C |
| ATOM | 2304 | N | LEU | A | 301 | 65.265 | 34.067 | −9.161 | 1.00 | 39.05 | A | N |
| ATOM | 2305 | CA | LEU | A | 301 | 66.093 | 32.901 | −9.421 | 1.00 | 37.54 | A | C |
| ATOM | 2306 | C | LEU | A | 301 | 67.013 | 32.602 | −8.255 | 1.00 | 38.40 | A | C |
| ATOM | 2307 | O | LEU | A | 301 | 67.182 | 33.428 | −7.371 | 1.00 | 36.53 | A | O |
| ATOM | 2308 | CB | LEU | A | 301 | 66.933 | 33.169 | −10.664 | 1.00 | 36.30 | A | C |
| ATOM | 2309 | CG | LEU | A | 301 | 66.126 | 33.447 | −11.937 | 1.00 | 34.74 | A | C |
| ATOM | 2310 | CD1 | LEU | A | 301 | 67.030 | 33.935 | −13.022 | 1.00 | 32.47 | A | C |
| ATOM | 2311 | CD2 | LEU | A | 301 | 65.387 | 32.196 | −12.430 | 1.00 | 39.84 | A | C |
| ATOM | 2312 | N | PRO | A | 302 | 67.619 | 31.420 | −8.264 | 1.00 | 39.73 | A | N |
| ATOM | 2313 | CA | PRO | A | 302 | 68.706 | 31.113 | −7.335 | 1.00 | 41.84 | A | C |
| ATOM | 2314 | C | PRO | A | 302 | 69.828 | 32.157 | −7.387 | 1.00 | 41.87 | A | C |
| ATOM | 2315 | O | PRO | A | 302 | 70.427 | 32.420 | −6.356 | 1.00 | 45.40 | A | O |
| ATOM | 2316 | CB | PRO | A | 302 | 69.208 | 29.759 | −7.834 | 1.00 | 41.99 | A | C |
| ATOM | 2317 | CG | PRO | A | 302 | 68.031 | 29.157 | −8.495 | 1.00 | 42.63 | A | C |
| ATOM | 2318 | CD | PRO | A | 302 | 67.321 | 30.285 | −9.150 | 1.00 | 40.17 | A | C |
| ATOM | 2319 | N | GLN | A | 303 | 70.094 | 32.759 | −8.546 | 1.00 | 40.21 | A | N |
| ATOM | 2320 | CA | GLN | A | 303 | 71.197 | 33.721 | −8.659 | 1.00 | 41.56 | A | C |
| ATOM | 2321 | C | GLN | A | 303 | 70.933 | 34.984 | −7.824 | 1.00 | 43.24 | A | C |
| ATOM | 2322 | O | GLN | A | 303 | 71.837 | 35.788 | −7.598 | 1.00 | 38.56 | A | O |
| ATOM | 2323 | CB | GLN | A | 303 | 71.523 | 34.117 | −10.119 | 1.00 | 44.66 | A | C |
| ATOM | 2324 | CG | GLN | A | 303 | 70.564 | 33.666 | −11.201 | 1.00 | 47.40 | A | C |
| ATOM | 2325 | CD | GLN | A | 303 | 70.716 | 32.201 | −11.545 | 1.00 | 50.63 | A | C |
| ATOM | 2326 | OE1 | GLN | A | 303 | 69.911 | 31.372 | −11.120 | 1.00 | 55.64 | A | O |
| ATOM | 2327 | NE2 | GLN | A | 303 | 71.742 | 31.877 | −12.332 | 1.00 | 51.86 | A | N |
| ATOM | 2328 | N | GLN | A | 304 | 69.689 | 35.145 | −7.382 | 1.00 | 41.50 | A | N |
| ATOM | 2329 | CA | GLN | A | 304 | 69.273 | 36.282 | −6.589 | 1.00 | 46.43 | A | C |
| ATOM | 2330 | C | GLN | A | 304 | 69.293 | 35.906 | −5.100 | 1.00 | 47.20 | A | C |
| ATOM | 2331 | O | GLN | A | 304 | 69.738 | 36.705 | −4.270 | 1.00 | 45.43 | A | O |
| ATOM | 2332 | CB | GLN | A | 304 | 67.871 | 36.735 | −7.046 | 1.00 | 49.88 | A | C |
| ATOM | 2333 | CG | GLN | A | 304 | 67.862 | 37.811 | −8.157 | 1.00 | 51.33 | A | C |
| ATOM | 2334 | CD | GLN | A | 304 | 68.273 | 37.315 | −9.548 | 1.00 | 54.63 | A | C |
| ATOM | 2335 | OE1 | GLN | A | 304 | 67.918 | 37.933 | −10.556 | 1.00 | 54.99 | A | O |
| ATOM | 2336 | NE2 | GLN | A | 304 | 69.031 | 36.224 | −9.607 | 1.00 | 57.60 | A | N |
| ATOM | 2337 | N | TYR | A | 305 | 68.838 | 34.694 | −4.760 | 1.00 | 47.73 | A | N |
| ATOM | 2338 | CA | TYR | A | 305 | 68.895 | 34.241 | −3.364 | 1.00 | 51.85 | A | C |
| ATOM | 2339 | C | TYR | A | 305 | 70.132 | 33.385 | −3.029 | 1.00 | 53.38 | A | C |
| ATOM | 2340 | O | TYR | A | 305 | 70.267 | 32.911 | −1.903 | 1.00 | 53.57 | A | O |
| ATOM | 2341 | CB | TYR | A | 305 | 67.573 | 33.577 | −2.910 | 1.00 | 52.81 | A | C |
| ATOM | 2342 | CG | TYR | A | 305 | 67.247 | 32.202 | −3.471 | 1.00 | 53.16 | A | C |
| ATOM | 2343 | CD1 | TYR | A | 305 | 67.828 | 31.052 | −2.943 | 1.00 | 52.42 | A | C |
| ATOM | 2344 | CD2 | TYR | A | 305 | 66.309 | 32.053 | −4.494 | 1.00 | 52.93 | A | C |
| ATOM | 2345 | CE1 | TYR | A | 305 | 67.515 | 29.789 | −3.446 | 1.00 | 53.23 | A | C |
| ATOM | 2346 | CE2 | TYR | A | 305 | 65.985 | 30.796 | −5.002 | 1.00 | 53.91 | A | C |
| ATOM | 2347 | CZ | TYR | A | 305 | 66.592 | 29.669 | −4.474 | 1.00 | 53.62 | A | C |
| ATOM | 2348 | OH | TYR | A | 305 | 66.272 | 28.425 | −4.971 | 1.00 | 53.84 | A | O |
| ATOM | 2349 | N | LEU | A | 306 | 71.033 | 33.228 | −3.997 | 1.00 | 55.03 | A | N |
| ATOM | 2350 | CA | LEU | A | 306 | 72.355 | 32.633 | −3.777 | 1.00 | 61.56 | A | C |
| ATOM | 2351 | C | LEU | A | 306 | 73.390 | 33.700 | −4.150 | 1.00 | 63.69 | A | C |
| ATOM | 2352 | O | LEU | A | 306 | 73.663 | 33.946 | −5.332 | 1.00 | 64.04 | A | O |
| ATOM | 2353 | CB | LEU | A | 306 | 72.559 | 31.361 | −4.610 | 1.00 | 62.70 | A | C |
| ATOM | 2354 | CG | LEU | A | 306 | 72.418 | 29.992 | −3.934 | 1.00 | 65.11 | A | C |
| ATOM | 2355 | CD1 | LEU | A | 306 | 71.256 | 29.932 | −2.950 | 1.00 | 65.92 | A | C |
| ATOM | 2356 | CD2 | LEU | A | 306 | 72.262 | 28.920 | −5.001 | 1.00 | 65.99 | A | C |
| ATOM | 2357 | N | ARG | A | 307 | 73.965 | 34.321 | −3.126 | 1.00 | 67.42 | A | N |
| ATOM | 2358 | CA | ARG | A | 307 | 74.753 | 35.546 | −3.285 | 1.00 | 69.54 | A | C |
| ATOM | 2359 | C | ARG | A | 307 | 76.226 | 35.241 | −3.045 | 1.00 | 70.36 | A | C |
| ATOM | 2360 | O | ARG | A | 307 | 76.568 | 34.726 | −1.981 | 1.00 | 69.95 | A | O |
| ATOM | 2361 | CB | ARG | A | 307 | 74.270 | 36.631 | −2.302 | 1.00 | 70.52 | A | C |
| ATOM | 2362 | CG | ARG | A | 307 | 73.942 | 36.126 | −0.885 | 1.00 | 71.76 | A | C |
| ATOM | 2363 | CD | ARG | A | 307 | 73.347 | 37.158 | 0.060 | 1.00 | 71.91 | A | C |
| ATOM | 2364 | NE | ARG | A | 307 | 74.308 | 37.604 | 1.064 | 1.00 | 71.58 | A | N |
| ATOM | 2365 | CZ | ARG | A | 307 | 75.160 | 38.609 | 0.900 | 1.00 | 72.31 | A | C |
| ATOM | 2440 | OD2 | ASP | A | 317 | 87.616 | 26.284 | −4.381 | 1.00 | 83.59 | A | O |
| ATOM | 2441 | N | ASP | A | 318 | 84.113 | 29.504 | −5.421 | 1.00 | 77.10 | A | N |
| ATOM | 2442 | CA | ASP | A | 318 | 82.894 | 30.315 | −5.525 | 1.00 | 76.86 | A | C |
| ATOM | 2443 | C | ASP | A | 318 | 81.909 | 29.982 | −4.398 | 1.00 | 75.04 | A | C |
| ATOM | 2444 | O | ASP | A | 318 | 81.025 | 29.137 | −4.565 | 1.00 | 76.20 | A | O |
| ATOM | 2445 | CB | ASP | A | 318 | 82.212 | 30.093 | −6.880 | 1.00 | 76.77 | A | C |
| ATOM | 2446 | CG | ASP | A | 318 | 83.043 | 30.590 | −8.044 | 1.00 | 77.69 | A | C |
| ATOM | 2447 | OD1 | ASP | A | 318 | 84.270 | 30.781 | −7.874 | 1.00 | 78.53 | A | O |
| ATOM | 2448 | OD2 | ASP | A | 318 | 82.550 | 30.811 | −9.170 | 1.00 | 77.17 | A | O |
| ATOM | 2449 | N | CYS | A | 319 | 82.065 | 30.653 | −3.259 | 1.00 | 73.01 | A | N |
| ATOM | 2450 | CA | CYS | A | 319 | 81.211 | 30.428 | −2.094 | 1.00 | 71.05 | A | C |
| ATOM | 2451 | C | CYS | A | 319 | 80.044 | 31.414 | −2.062 | 1.00 | 68.67 | A | C |
| ATOM | 2452 | O | CYS | A | 319 | 80.235 | 32.611 | −2.258 | 1.00 | 66.66 | A | O |
| ATOM | 2453 | CB | CYS | A | 319 | 82.026 | 30.554 | −0.803 | 1.00 | 71.92 | A | C |
| ATOM | 2454 | SG | CYS | A | 319 | 83.414 | 29.394 | −0.686 | 1.00 | 74.09 | A | S |
| ATOM | 2455 | N | TYR | A | 320 | 78.839 | 30.899 | −1.832 | 1.00 | 66.44 | A | N |
| ATOM | 2456 | CA | TYR | A | 320 | 77.645 | 31.728 | −1.691 | 1.00 | 64.22 | A | C |
| ATOM | 2457 | C | TYR | A | 320 | 77.013 | 31.498 | −0.320 | 1.00 | 62.66 | A | C |
| ATOM | 2458 | O | TYR | A | 320 | 77.332 | 30.523 | 0.365 | 1.00 | 61.41 | A | O |
| ATOM | 2459 | CB | TYR | A | 320 | 76.611 | 31.384 | −2.764 | 1.00 | 64.24 | A | C |
| ATOM | 2460 | CG | TYR | A | 320 | 77.108 | 31.323 | −4.198 | 1.00 | 64.74 | A | C |
| ATOM | 2461 | CD1 | TYR | A | 320 | 77.957 | 30.302 | −4.632 | 1.00 | 64.73 | A | C |
| ATOM | 2462 | CD2 | TYR | A | 320 | 76.682 | 32.260 | −5.139 | 1.00 | 64.78 | A | C |
| ATOM | 2463 | CE1 | TYR | A | 320 | 78.390 | 30.239 | −5.961 | 1.00 | 64.57 | A | C |
| ATOM | 2464 | CE2 | TYR | A | 320 | 77.106 | 32.202 | −6.465 | 1.00 | 64.30 | A | C |
| ATOM | 2465 | CZ | TYR | A | 320 | 77.957 | 31.193 | −6.871 | 1.00 | 64.35 | A | C |
| ATOM | 2466 | OH | TYR | A | 320 | 78.374 | 31.146 | −8.185 | 1.00 | 62.89 | A | O |
| ATOM | 2467 | N | LYS | A | 321 | 76.111 | 32.392 | 0.076 | 1.00 | 59.11 | A | N |
| ATOM | 2468 | CA | LYS | A | 321 | 75.320 | 32.189 | 1.286 | 1.00 | 58.36 | A | C |
| ATOM | 2469 | C | LYS | A | 321 | 73.816 | 32.265 | 0.996 | 1.00 | 55.24 | A | C |
| ATOM | 2470 | O | LYS | A | 321 | 73.353 | 33.065 | 0.181 | 1.00 | 47.87 | A | O |
| ATOM | 2471 | CB | LYS | A | 321 | 75.747 | 33.160 | 2.398 | 1.00 | 59.88 | A | C |
| ATOM | 2472 | CG | LYS | A | 321 | 74.853 | 34.371 | 2.629 | 1.00 | 62.72 | A | C |
| ATOM | 2473 | CD | LYS | A | 321 | 75.277 | 35.120 | 3.888 | 1.00 | 64.37 | A | C |
| ATOM | 2474 | CE | LYS | A | 321 | 74.699 | 34.483 | 5.145 | 1.00 | 64.91 | A | C |
| ATOM | 2475 | NZ | LYS | A | 321 | 74.999 | 35.289 | 6.363 | 1.00 | 66.23 | A | N |
| ATOM | 2476 | N | PHE | A | 322 | 73.062 | 31.401 | 1.664 | 1.00 | 51.88 | A | N |
| ATOM | 2477 | CA | PHE | A | 322 | 71.619 | 31.397 | 1.531 | 1.00 | 49.73 | A | C |
| ATOM | 2478 | C | PHE | A | 322 | 71.087 | 32.737 | 2.019 | 1.00 | 49.06 | A | C |
| ATOM | 2479 | O | PHE | A | 322 | 71.346 | 33.148 | 3.154 | 1.00 | 45.51 | A | O |
| ATOM | 2480 | CB | PHE | A | 322 | 70.999 | 30.235 | 2.321 | 1.00 | 49.10 | A | C |
| ATOM | 2481 | CG | PHE | A | 322 | 69.573 | 29.925 | 1.935 | 1.00 | 44.63 | A | C |
| ATOM | 2482 | CD1 | PHE | A | 322 | 68.563 | 29.921 | 2.894 | 1.00 | 45.73 | A | C |
| ATOM | 2483 | CD2 | PHE | A | 322 | 69.248 | 29.629 | 0.623 | 1.00 | 39.25 | A | C |
| ATOM | 2484 | CE1 | PHE | A | 322 | 67.252 | 29.634 | 2.539 | 1.00 | 44.70 | A | C |
| ATOM | 2485 | CE2 | PHE | A | 322 | 67.950 | 29.351 | 0.261 | 1.00 | 41.50 | A | C |
| ATOM | 2486 | CZ | PHE | A | 322 | 66.947 | 29.353 | 1.218 | 1.00 | 42.07 | A | C |
| ATOM | 2487 | N | ALA | A | 323 | 70.339 | 33.399 | 1.142 | 1.00 | 47.94 | A | N |
| ATOM | 2488 | CA | ALA | A | 323 | 69.901 | 34.771 | 1.336 | 1.00 | 46.61 | A | C |
| ATOM | 2489 | C | ALA | A | 323 | 68.426 | 34.839 | 1.712 | 1.00 | 44.07 | A | C |
| ATOM | 2490 | O | ALA | A | 323 | 67.809 | 35.887 | 1.548 | 1.00 | 33.93 | A | O |
| ATOM | 2491 | CB | ALA | A | 323 | 70.133 | 35.564 | 0.054 | 1.00 | 49.38 | A | C |
| ATOM | 2492 | N | ILE | A | 324 | 67.853 | 33.725 | 2.169 | 1.00 | 37.73 | A | N |
| ATOM | 2493 | CA | ILE | A | 324 | 66.520 | 33.746 | 2.739 | 1.00 | 38.21 | A | C |
| ATOM | 2494 | C | ILE | A | 324 | 66.643 | 33.442 | 4.214 | 1.00 | 34.73 | A | C |
| ATOM | 2495 | O | ILE | A | 324 | 67.442 | 32.611 | 4.619 | 1.00 | 36.43 | A | O |
| ATOM | 2496 | CB | ILE | A | 324 | 65.577 | 32.736 | 2.038 | 1.00 | 38.89 | A | C |
| ATOM | 2497 | CG1 | ILE | A | 324 | 65.714 | 32.862 | 0.518 | 1.00 | 38.34 | A | C |
| ATOM | 2498 | CG2 | ILE | A | 324 | 64.126 | 32.960 | 2.495 | 1.00 | 40.76 | A | C |
| ATOM | 2499 | CD1 | ILE | A | 324 | 64.684 | 32.110 | −0.277 | 1.00 | 41.65 | A | C |
| ATOM | 2500 | N | SER | A | 325 | 65.840 | 34.112 | 5.020 | 1.00 | 32.69 | A | N |
| ATOM | 2501 | CA | SER | A | 325 | 66.013 | 34.031 | 6.460 | 1.00 | 38.71 | A | C |
| ATOM | 2502 | C | SER | A | 325 | 64.722 | 34.407 | 7.139 | 1.00 | 39.64 | A | C |
| ATOM | 2503 | O | SER | A | 325 | 63.792 | 34.883 | 6.509 | 1.00 | 40.29 | A | O |
| ATOM | 2504 | CB | SER | A | 325 | 67.150 | 34.953 | 6.925 | 1.00 | 40.03 | A | C |
| ATOM | 2505 | OG | SER | A | 325 | 66.788 | 36.327 | 6.838 | 1.00 | 38.81 | A | O |
| ATOM | 2506 | N | GLN | A | 326 | 64.677 | 34.221 | 8.440 | 1.00 | 38.99 | A | N |
| ATOM | 2507 | CA | GLN | A | 326 | 63.414 | 34.244 | 9.134 | 1.00 | 39.09 | A | C |
| ATOM | 2508 | C | GLN | A | 326 | 63.294 | 35.561 | 9.885 | 1.00 | 36.76 | A | C |
| ATOM | 2509 | O | GLN | A | 326 | 64.259 | 36.297 | 10.004 | 1.00 | 39.83 | A | O |
| ATOM | 2510 | CB | GLN | A | 326 | 63.287 | 33.011 | 10.037 | 1.00 | 41.88 | A | C |
| ATOM | 2511 | CG | GLN | A | 326 | 64.481 | 32.024 | 9.957 | 1.00 | 45.10 | A | C |
| ATOM | 2512 | CD | GLN | A | 326 | 64.374 | 30.817 | 10.892 | 1.00 | 50.52 | A | C |
| ATOM | 2513 | OE1 | GLN | A | 326 | 65.393 | 30.286 | 11.322 | 1.00 | 51.97 | A | O |
| ATOM | 2514 | NE2 | GLN | A | 326 | 63.151 | 30.388 | 11.202 | 1.00 | 51.73 | A | N |
| ATOM | 2515 | N | SER | A | 327 | 62.108 | 35.878 | 10.374 | 1.00 | 30.92 | A | N |
| ATOM | 2516 | CA | SER | A | 327 | 61.904 | 37.188 | 10.988 | 1.00 | 35.17 | A | C |
| ATOM | 2517 | C | SER | A | 327 | 60.788 | 37.140 | 11.993 | 1.00 | 36.09 | A | C |
| ATOM | 2518 | O | SER | A | 327 | 59.978 | 36.208 | 12.018 | 1.00 | 38.75 | A | O |
| ATOM | 2519 | CB | SER | A | 327 | 61.578 | 38.259 | 9.921 | 1.00 | 35.42 | A | C |
| ATOM | 2520 | OG | SER | A | 327 | 60.882 | 39.380 | 10.482 | 1.00 | 33.54 | A | O |
| ATOM | 2521 | N | SER | A | 328 | 60.723 | 38.174 | 12.808 | 1.00 | 31.04 | A | N |
| ATOM | 2522 | CA | SER | A | 328 | 59.597 | 38.328 | 13.697 | 1.00 | 37.93 | A | C |
| ATOM | 2523 | C | SER | A | 328 | 58.868 | 39.654 | 13.520 | 1.00 | 39.07 | A | C |
| ATOM | 2524 | O | SER | A | 328 | 57.960 | 39.967 | 14.296 | 1.00 | 43.35 | A | O |
| ATOM | 2525 | CB | SER | A | 328 | 60.086 | 38.167 | 15.123 | 1.00 | 41.74 | A | C |
| ATOM | 2526 | OG | SER | A | 328 | 60.967 | 39.227 | 15.485 | 1.00 | 45.41 | A | O |
| ATOM | 2527 | N | THR | A | 329 | 59.257 | 40.409 | 12.492 | 1.00 | 37.31 | A | N |
| ATOM | 2528 | CA | THR | A | 329 | 58.675 | 41.715 | 12.186 | 1.00 | 40.83 | A | C |
| ATOM | 2529 | C | THR | A | 329 | 58.020 | 41.703 | 10.797 | 1.00 | 39.53 | A | C |
| ATOM | 2530 | O | THR | A | 329 | 57.814 | 42.771 | 10.218 | 1.00 | 37.06 | A | O |
| ATOM | 2531 | CB | THR | A | 329 | 59.770 | 42.814 | 12.193 | 1.00 | 42.95 | A | C |
| ATOM | 2532 | OG1 | THR | A | 329 | 60.831 | 42.455 | 11.287 | 1.00 | 41.26 | A | O |
| ATOM | 2533 | CG2 | THR | A | 329 | 60.441 | 42.933 | 13.558 | 1.00 | 44.90 | A | C |
| ATOM | 2534 | N | GLY | A | 330 | 57.724 | 40.510 | 10.270 | 1.00 | 37.04 | A | N |
| ATOM | 2535 | CA | GLY | A | 330 | 57.032 | 40.358 | 8.988 | 1.00 | 35.68 | A | C |
| ATOM | 2536 | C | GLY | A | 330 | 57.929 | 39.930 | 7.836 | 1.00 | 34.85 | A | C |
| ATOM | 2537 | O | GLY | A | 330 | 59.067 | 39.526 | 8.047 | 1.00 | 32.50 | A | O |
| ATOM | 2538 | N | THR | A | 331 | 57.398 | 39.974 | 6.605 | 1.00 | 33.39 | A | N |
| ATOM | 2539 | CA | THR | A | 331 | 58.207 | 39.709 | 5.419 | 1.00 | 28.89 | A | C |
| ATOM | 2540 | C | THR | A | 331 | 58.979 | 40.971 | 4.998 | 1.00 | 27.79 | A | C |
| ATOM | 2541 | O | THR | A | 331 | 58.496 | 42.086 | 5.175 | 1.00 | 33.40 | A | O |
| ATOM | 2542 | CB | THR | A | 331 | 57.320 | 39.267 | 4.249 | 1.00 | 30.99 | A | C |
| ATOM | 2543 | OG1 | THR | A | 331 | 56.695 | 38.020 | 4.561 | 1.00 | 30.88 | A | O |
| ATOM | 2544 | CG2 | THR | A | 331 | 58.157 | 38.989 | 2.983 | 1.00 | 33.73 | A | C |
| ATOM | 2545 | N | VAL | A | 332 | 60.177 | 40.764 | 4.470 | 1.00 | 22.23 | A | N |
| ATOM | 2546 | CA | VAL | A | 332 | 61.065 | 41.847 | 3.989 | 1.00 | 27.82 | A | C |
| ATOM | 2547 | C | VAL | A | 332 | 61.630 | 41.466 | 2.632 | 1.00 | 24.10 | A | C |
| ATOM | 2548 | O | VAL | A | 332 | 62.394 | 40.540 | 2.501 | 1.00 | 26.31 | A | O |
| ATOM | 2549 | CB | VAL | A | 332 | 62.274 | 42.139 | 4.935 | 1.00 | 28.52 | A | C |
| ATOM | 2550 | CG1 | VAL | A | 332 | 63.138 | 43.293 | 4.367 | 1.00 | 32.83 | A | C |
| ATOM | 2551 | CG2 | VAL | A | 332 | 61.801 | 42.468 | 6.329 | 1.00 | 30.87 | A | C |
| ATOM | 2552 | N | MET | A | 333 | 61.242 | 42.199 | 1.591 | 1.00 | 29.37 | A | N |
| ATOM | 2553 | CA | MET | A | 333 | 61.760 | 41.968 | 0.251 | 1.00 | 23.35 | A | C |
| ATOM | 2554 | C | MET | A | 333 | 63.012 | 42.813 | −0.015 | 1.00 | 26.09 | A | C |
| ATOM | 2555 | O | MET | A | 333 | 62.920 | 43.935 | −0.512 | 1.00 | 22.66 | A | O |
| ATOM | 2556 | CB | MET | A | 333 | 60.687 | 42.296 | −0.804 | 1.00 | 26.84 | A | C |
| ATOM | 2557 | CG | MET | A | 333 | 59.550 | 41.295 | −0.855 | 1.00 | 28.90 | A | C |
| ATOM | 2558 | SD | MET | A | 333 | 58.086 | 41.883 | −1.807 | 1.00 | 33.93 | A | S |
| ATOM | 2559 | CE | MET | A | 333 | 58.640 | 41.701 | −3.325 | 1.00 | 30.83 | A | C |
| ATOM | 2560 | N | GLY | A | 334 | 64.179 | 42.265 | 0.294 | 1.00 | 26.44 | A | N |
| ATOM | 2561 | CA | GLY | A | 334 | 65.428 | 43.015 | 0.190 | 1.00 | 28.29 | A | C |
| ATOM | 2562 | C | GLY | A | 334 | 66.044 | 43.002 | −1.185 | 1.00 | 28.85 | A | C |
| ATOM | 2563 | O | GLY | A | 334 | 65.370 | 42.791 | −2.185 | 1.00 | 28.04 | A | O |
| ATOM | 2564 | N | ALA | A | 335 | 67.350 | 43.220 | −1.243 | 1.00 | 29.46 | A | N |
| ATOM | 2565 | CA | ALA | A | 335 | 68.097 | 43.214 | −2.489 | 1.00 | 28.30 | A | C |
| ATOM | 2566 | C | ALA | A | 335 | 67.939 | 41.952 | −3.330 | 1.00 | 31.11 | A | C |
| ATOM | 2567 | O | ALA | A | 335 | 68.001 | 42.021 | −4.563 | 1.00 | 29.82 | A | O |
| ATOM | 2568 | CB | ALA | A | 335 | 69.578 | 43.470 | −2.206 | 1.00 | 34.20 | A | C |
| ATOM | 2569 | N | VAL | A | 336 | 67.738 | 40.805 | −2.671 | 1.00 | 31.66 | A | N |
| ATOM | 2570 | CA | VAL | A | 336 | 67.506 | 39.532 | −3.349 | 1.00 | 35.42 | A | C |
| ATOM | 2571 | C | VAL | A | 336 | 66.412 | 39.733 | −4.393 | 1.00 | 32.46 | A | C |
| ATOM | 2572 | O | VAL | A | 336 | 66.626 | 39.464 | −5.574 | 1.00 | 36.08 | A | O |
| ATOM | 2573 | CB | VAL | A | 336 | 67.096 | 38.405 | −2.341 | 1.00 | 38.14 | A | C |
| ATOM | 2574 | CG1 | VAL | A | 336 | 66.466 | 37.196 | −3.057 | 1.00 | 41.63 | A | C |
| ATOM | 2575 | CG2 | VAL | A | 336 | 68.294 | 37.960 | −1.518 | 1.00 | 42.38 | A | C |
| ATOM | 2576 | N | ILE | A | 337 | 65.271 | 40.248 | −3.944 | 1.00 | 32.57 | A | N |
| ATOM | 2577 | CA | ILE | A | 337 | 64.130 | 40.507 | −4.832 | 1.00 | 31.81 | A | C |
| ATOM | 2578 | C | ILE | A | 337 | 64.389 | 41.687 | −5.760 | 1.00 | 29.91 | A | C |
| ATOM | 2579 | O | ILE | A | 337 | 64.231 | 41.592 | −6.969 | 1.00 | 27.22 | A | O |
| ATOM | 2580 | CB | ILE | A | 337 | 62.835 | 40.731 | −4.005 | 1.00 | 34.05 | A | C |
| ATOM | 2581 | CG1 | ILE | A | 337 | 62.466 | 39.472 | −3.216 | 1.00 | 37.53 | A | C |
| ATOM | 2582 | CG2 | ILE | A | 337 | 61.668 | 41.174 | −4.903 | 1.00 | 34.31 | A | C |
| ATOM | 2583 | CD1 | ILE | A | 337 | 61.814 | 38.383 | −4.043 | 1.00 | 39.14 | A | C |
| ATOM | 2584 | N | MET | A | 338 | 64.813 | 42.816 | −5.202 | 1.00 | 28.42 | A | N |
| ATOM | 2585 | CA | MET | A | 338 | 64.909 | 44.038 | −5.998 | 1.00 | 27.14 | A | C |
| ATOM | 2586 | C | MET | A | 338 | 65.914 | 43.969 | −7.154 | 1.00 | 28.91 | A | C |
| ATOM | 2587 | O | MET | A | 338 | 65.753 | 44.650 | −8.166 | 1.00 | 28.30 | A | O |
| ATOM | 2588 | CB | MET | A | 338 | 65.195 | 45.214 | −5.067 | 1.00 | 25.93 | A | C |
| ATOM | 2589 | CG | MET | A | 338 | 64.083 | 45.457 | −4.082 | 1.00 | 27.03 | A | C |
| ATOM | 2590 | SD | MET | A | 338 | 64.367 | 46.907 | −3.076 | 1.00 | 25.07 | A | S |
| ATOM | 2591 | CE | MET | A | 338 | 64.174 | 48.235 | −4.312 | 1.00 | 20.81 | A | C |
| ATOM | 2592 | N | GLU | A | 339 | 66.954 | 43.142 | −7.040 | 1.00 | 29.81 | A | N |
| ATOM | 2593 | CA | GLU | A | 339 | 67.909 | 43.018 | −8.142 | 1.00 | 32.30 | A | C |
| ATOM | 2594 | C | GLU | A | 339 | 67.318 | 42.371 | −9.403 | 1.00 | 30.06 | A | C |
| ATOM | 2595 | O | GLU | A | 339 | 67.874 | 42.502 | −10.488 | 1.00 | 33.61 | A | O |
| ATOM | 2596 | CB | GLU | A | 339 | 69.174 | 42.269 | −7.704 | 1.00 | 33.90 | A | C |
| ATOM | 2597 | CG | GLU | A | 339 | 70.197 | 43.177 | −7.027 | 1.00 | 38.40 | A | C |
| ATOM | 2598 | CD | GLU | A | 339 | 71.139 | 42.424 | −6.107 | 1.00 | 42.07 | A | C |
| ATOM | 2599 | OE1 | GLU | A | 339 | 71.439 | 41.242 | −6.391 | 1.00 | 39.96 | A | O |
| ATOM | 2600 | OE2 | GLU | A | 339 | 71.570 | 43.015 | −5.095 | 1.00 | 43.98 | A | O |
| ATOM | 2601 | N | GLY | A | 340 | 66.187 | 41.687 | −9.259 | 1.00 | 30.73 | A | N |
| ATOM | 2602 | CA | GLY | A | 340 | 65.475 | 41.165 | −10.411 | 1.00 | 29.00 | A | C |
| ATOM | 2603 | C | GLY | A | 340 | 64.626 | 42.169 | −11.162 | 1.00 | 25.72 | A | C |
| ATOM | 2604 | O | GLY | A | 340 | 64.289 | 41.949 | −12.331 | 1.00 | 25.52 | A | O |
| ATOM | 2605 | N | PHE | A | 341 | 64.278 | 43.281 | −10.509 | 1.00 | 21.32 | A | N |
| ATOM | 2606 | CA | PHE | A | 341 | 63.243 | 44.173 | −11.017 | 1.00 | 17.05 | A | C |
| ATOM | 2607 | C | PHE | A | 341 | 63.561 | 45.656 | −10.971 | 1.00 | 19.54 | A | C |
| ATOM | 2608 | O | PHE | A | 341 | 64.379 | 46.111 | −10.174 | 1.00 | 20.37 | A | O |
| ATOM | 2609 | CB | PHE | A | 341 | 61.961 | 43.899 | −10.222 | 1.00 | 18.19 | A | C |
| ATOM | 2610 | CG | PHE | A | 341 | 61.630 | 42.440 | −10.137 | 1.00 | 20.96 | A | C |
| ATOM | 2611 | CD1 | PHE | A | 341 | 61.108 | 41.770 | −11.237 | 1.00 | 22.17 | A | C |
| ATOM | 2612 | CD2 | PHE | A | 341 | 61.910 | 41.717 | −8.998 | 1.00 | 23.21 | A | C |
| ATOM | 2613 | CE1 | PHE | A | 341 | 60.853 | 40.399 | −11.160 | 1.00 | 17.74 | A | C |
| ATOM | 2614 | CE2 | PHE | A | 341 | 61.650 | 40.351 | −8.939 | 1.00 | 23.34 | A | C |
| ATOM | 2615 | CZ | PHE | A | 341 | 61.134 | 39.705 | −10.012 | 1.00 | 24.23 | A | C |
| ATOM | 2616 | N | TYR | A | 342 | 62.952 | 46.413 | −11.875 | 1.00 | 17.67 | A | N |
| ATOM | 2617 | CA | TYR | A | 342 | 62.820 | 47.837 | −11.702 | 1.00 | 17.52 | A | C |
| ATOM | 2618 | C | TYR | A | 342 | 61.608 | 48.077 | −10.810 | 1.00 | 22.25 | A | C |
| ATOM | 2619 | O | TYR | A | 342 | 60.494 | 47.620 | −11.100 | 1.00 | 19.30 | A | O |
| ATOM | 2620 | CB | TYR | A | 342 | 62.656 | 48.485 | −13.040 | 1.00 | 17.92 | A | C |
| ATOM | 2621 | CG | TYR | A | 342 | 62.654 | 49.980 | −13.067 | 1.00 | 18.37 | A | C |
| ATOM | 2622 | CD1 | TYR | A | 342 | 63.668 | 50.730 | −12.467 | 1.00 | 19.68 | A | C |
| ATOM | 2623 | CD2 | TYR | A | 342 | 61.681 | 50.654 | −13.765 | 1.00 | 23.05 | A | C |
| ATOM | 2624 | CE1 | TYR | A | 342 | 63.684 | 52.115 | −12.562 | 1.00 | 22.84 | A | C |
| ATOM | 2625 | CE2 | TYR | A | 342 | 61.693 | 52.006 | −13.868 | 1.00 | 22.40 | A | C |
| ATOM | 2626 | CZ | TYR | A | 342 | 62.693 | 52.750 | −13.264 | 1.00 | 22.81 | A | C |
| ATOM | 2627 | OH | TYR | A | 342 | 62.667 | 54.131 | −13.385 | 1.00 | 26.20 | A | O |
| ATOM | 2628 | N | VAL | A | 343 | 61.840 | 48.777 | −9.705 | 1.00 | 15.05 | A | N |
| ATOM | 2629 | CA | VAL | A | 343 | 60.827 | 49.008 | −8.688 | 1.00 | 17.53 | A | C |
| ATOM | 2630 | C | VAL | A | 343 | 60.510 | 50.494 | −8.598 | 1.00 | 14.20 | A | C |
| ATOM | 2631 | O | VAL | A | 343 | 61.378 | 51.334 | −8.376 | 1.00 | 15.91 | A | O |
| ATOM | 2632 | CB | VAL | A | 343 | 61.259 | 48.442 | −7.305 | 1.00 | 15.41 | A | C |
| ATOM | 2633 | CG1 | VAL | A | 343 | 60.123 | 48.560 | −6.267 | 1.00 | 18.03 | A | C |
| ATOM | 2634 | CG2 | VAL | A | 343 | 61.704 | 47.022 | −7.473 | 1.00 | 18.07 | A | C |
| ATOM | 2635 | N | VAL | A | 344 | 59.231 | 50.791 | −8.767 | 1.00 | 11.66 | A | N |
| ATOM | 2636 | CA | VAL | A | 344 | 58.682 | 52.123 | −8.783 | 1.00 | 12.22 | A | C |
| ATOM | 2637 | C | VAL | A | 344 | 57.903 | 52.401 | −7.510 | 1.00 | 13.35 | A | C |
| ATOM | 2638 | O | VAL | A | 344 | 56.875 | 51.802 | −7.235 | 1.00 | 16.24 | A | O |
| ATOM | 2639 | CB | VAL | A | 344 | 57.774 | 52.308 | −10.027 | 1.00 | 15.89 | A | C |
| ATOM | 2640 | CG1 | VAL | A | 344 | 57.159 | 53.685 | −10.035 | 1.00 | 18.74 | A | C |
| ATOM | 2641 | CG2 | VAL | A | 344 | 58.587 | 52.106 | −11.280 | 1.00 | 18.50 | A | C |
| ATOM | 2642 | N | PHE | A | 345 | 58.418 | 53.322 | −6.713 | 1.00 | 16.40 | A | N |
| ATOM | 2643 | CA | PHE | A | 345 | 57.771 | 53.763 | −5.483 | 1.00 | 15.06 | A | C |
| ATOM | 2644 | C | PHE | A | 345 | 56.833 | 54.934 | −5.754 | 1.00 | 15.39 | A | C |
| ATOM | 2645 | O | PHE | A | 345 | 57.192 | 56.113 | −5.655 | 1.00 | 17.43 | A | O |
| ATOM | 2646 | CB | PHE | A | 345 | 58.846 | 54.062 | −4.416 | 1.00 | 17.40 | A | C |
| ATOM | 2647 | CG | PHE | A | 345 | 59.670 | 52.855 | −4.040 | 1.00 | 15.11 | A | C |
| ATOM | 2648 | CD1 | PHE | A | 345 | 60.702 | 52.386 | −4.863 | 1.00 | 13.07 | A | C |
| ATOM | 2649 | CD2 | PHE | A | 345 | 59.402 | 52.153 | −2.882 | 1.00 | 13.71 | A | C |
| ATOM | 2650 | CE1 | PHE | A | 345 | 61.446 | 51.242 | −4.510 | 1.00 | 15.84 | A | C |
| ATOM | 2651 | CE2 | PHE | A | 345 | 60.169 | 51.040 | −2.507 | 1.00 | 11.40 | A | C |
| ATOM | 2652 | CZ | PHE | A | 345 | 61.186 | 50.581 | −3.327 | 1.00 | 15.67 | A | C |
| ATOM | 2653 | N | ASP | A | 346 | 55.633 | 54.601 | −6.206 | 1.00 | 16.44 | A | N |
| ATOM | 2654 | CA | ASP | A | 346 | 54.671 | 55.593 | −6.672 | 1.00 | 15.35 | A | C |
| ATOM | 2655 | C | ASP | A | 346 | 53.855 | 56.101 | −5.495 | 1.00 | 16.03 | A | C |
| ATOM | 2656 | O | ASP | A | 346 | 52.711 | 55.700 | −5.254 | 1.00 | 19.50 | A | O |
| ATOM | 2657 | CB | ASP | A | 346 | 53.800 | 54.986 | −7.778 | 1.00 | 18.03 | A | C |
| ATOM | 2658 | CG | ASP | A | 346 | 52.872 | 55.995 | −8.420 | 1.00 | 25.99 | A | C |
| ATOM | 2659 | OD1 | ASP | A | 346 | 52.844 | 57.166 | −7.967 | 1.00 | 28.56 | A | O |
| ATOM | 2660 | OD2 | ASP | A | 346 | 52.120 | 55.680 | −9.382 | 1.00 | 23.57 | A | O |
| ATOM | 2661 | N | ARG | A | 347 | 54.491 | 56.978 | −4.725 | 1.00 | 17.55 | A | N |
| ATOM | 2662 | CA | ARG | A | 347 | 53.908 | 57.497 | −3.499 | 1.00 | 21.29 | A | C |
| ATOM | 2663 | C | ARG | A | 347 | 52.632 | 58.294 | −3.785 | 1.00 | 18.95 | A | C |
| ATOM | 2664 | O | ARG | A | 347 | 51.701 | 58.266 | −2.991 | 1.00 | 22.26 | A | O |
| ATOM | 2665 | CB | ARG | A | 347 | 54.932 | 58.369 | −2.765 | 1.00 | 19.24 | A | C |
| ATOM | 2666 | CG | ARG | A | 347 | 56.184 | 57.635 | −2.282 | 1.00 | 22.31 | A | C |
| ATOM | 2667 | CD | ARG | A | 347 | 57.359 | 58.594 | −2.059 | 1.00 | 23.46 | A | C |
| ATOM | 2668 | NE | ARG | A | 347 | 57.009 | 59.652 | −1.092 | 1.00 | 23.99 | A | N |
| ATOM | 2669 | CZ | ARG | A | 347 | 57.403 | 59.691 | 0.183 | 1.00 | 30.72 | A | C |
| ATOM | 2670 | NH1 | ARG | A | 347 | 57.031 | 60.700 | 0.959 | 1.00 | 32.59 | A | N |
| ATOM | 2671 | NH2 | ARG | A | 347 | 58.174 | 58.745 | 0.696 | 1.00 | 27.96 | A | N |
| ATOM | 2672 | N | ALA | A | 348 | 52.590 | 58.959 | −4.936 | 1.00 | 21.83 | A | N |
| ATOM | 2673 | CA | ALA | A | 348 | 51.439 | 59.780 | −5.327 | 1.00 | 24.64 | A | C |
| ATOM | 2674 | C | ALA | A | 348 | 50.148 | 58.953 | −5.399 | 1.00 | 28.25 | A | C |
| ATOM | 2675 | O | ALA | A | 348 | 49.056 | 59.426 | −5.028 | 1.00 | 24.96 | A | O |
| ATOM | 2676 | CB | ALA | A | 348 | 51.721 | 60.425 | −6.668 | 1.00 | 24.31 | A | C |
| ATOM | 2677 | N | ARG | A | 349 | 50.282 | 57.724 | −5.896 | 1.00 | 25.65 | A | N |
| ATOM | 2678 | CA | ARG | A | 349 | 49.151 | 56.806 | −6.029 | 1.00 | 25.84 | A | C |
| ATOM | 2679 | C | ARG | A | 349 | 49.168 | 55.627 | −5.077 | 1.00 | 25.58 | A | C |
| ATOM | 2680 | O | ARG | A | 349 | 48.460 | 54.653 | −5.319 | 1.00 | 25.28 | A | O |
| ATOM | 2681 | CB | ARG | A | 349 | 49.100 | 56.276 | −7.459 | 1.00 | 29.93 | A | C |
| ATOM | 2682 | CG | ARG | A | 349 | 49.176 | 57.344 | −8.488 | 1.00 | 33.60 | A | C |
| ATOM | 2683 | CD | ARG | A | 349 | 48.502 | 57.000 | −9.775 | 1.00 | 36.74 | A | C |
| ATOM | 2684 | NE | ARG | A | 349 | 48.827 | 58.016 | −10.763 | 1.00 | 42.71 | A | N |
| ATOM | 2685 | CZ | ARG | A | 349 | 48.278 | 59.227 | −10.814 | 1.00 | 48.30 | A | C |
| ATOM | 2686 | NH1 | ARG | A | 349 | 47.316 | 59.600 | −9.964 | 1.00 | 46.44 | A | N |
| ATOM | 2687 | NH2 | ARG | A | 349 | 48.686 | 60.072 | −11.751 | 1.00 | 50.26 | A | N |
| ATOM | 2688 | N | LYS | A | 350 | 49.954 | 55.721 | −3.989 | 1.00 | 23.32 | A | N |
| ATOM | 2689 | CA | LYS | A | 350 | 50.022 | 54.700 | −2.945 | 1.00 | 24.27 | A | C |
| ATOM | 2690 | C | LYS | A | 350 | 50.163 | 53.310 | −3.549 | 1.00 | 19.69 | A | C |
| ATOM | 2691 | O | LYS | A | 350 | 49.374 | 52.429 | −3.260 | 1.00 | 20.60 | A | O |
| ATOM | 2692 | CB | LYS | A | 350 | 48.757 | 54.704 | −2.079 | 1.00 | 28.78 | A | C |
| ATOM | 2693 | CG | LYS | A | 350 | 48.522 | 55.929 | −1.231 | 1.00 | 34.60 | A | C |
| ATOM | 2694 | CD | LYS | A | 350 | 47.436 | 55.639 | −0.141 | 1.00 | 37.26 | A | C |
| ATOM | 2695 | CE | LYS | A | 350 | 47.719 | 54.361 | 0.695 | 1.00 | 36.46 | A | C |
| ATOM | 2696 | NZ | LYS | A | 350 | 46.822 | 54.210 | 1.887 | 1.00 | 40.30 | A | N |
| ATOM | 2697 | N | ARG | A | 351 | 51.147 | 53.140 | −4.420 | 1.00 | 19.35 | A | N |
| ATOM | 2698 | CA | ARG | A | 351 | 51.371 | 51.855 | −5.063 | 1.00 | 17.19 | A | C |
| ATOM | 2699 | C | ARG | A | 351 | 52.842 | 51.641 | −5.383 | 1.00 | 15.48 | A | C |
| ATOM | 2700 | O | ARG | A | 351 | 53.609 | 52.576 | −5.490 | 1.00 | 17.66 | A | O |
| ATOM | 2701 | CB | ARG | A | 351 | 50.501 | 51.758 | −6.328 | 1.00 | 15.07 | A | C |
| ATOM | 2702 | CG | ARG | A | 351 | 50.851 | 52.687 | −7.388 | 1.00 | 17.01 | A | C |
| ATOM | 2703 | CD | ARG | A | 351 | 49.837 | 52.667 | −8.565 | 1.00 | 17.81 | A | C |
| ATOM | 2704 | NE | ARG | A | 351 | 50.304 | 53.485 | −9.674 | 1.00 | 17.24 | A | N |
| ATOM | 2705 | CZ | ARG | A | 351 | 49.711 | 53.543 | −10.862 | 1.00 | 23.21 | A | C |
| ATOM | 2706 | NH1 | ARG | A | 351 | 48.651 | 52.804 | −11.095 | 1.00 | 21.30 | A | N |
| ATOM | 2707 | NH2 | ARG | A | 351 | 50.213 | 54.312 | −11.831 | 1.00 | 24.26 | A | N |
| ATOM | 2708 | N | ILE | A | 352 | 53.240 | 50.376 | −5.500 | 1.00 | 17.16 | A | N |
| ATOM | 2709 | CA | ILE | A | 352 | 54.581 | 50.022 | −5.928 | 1.00 | 16.38 | A | C |
| ATOM | 2710 | C | ILE | A | 352 | 54.510 | 49.221 | −7.208 | 1.00 | 15.28 | A | C |
| ATOM | 2711 | O | ILE | A | 352 | 53.800 | 48.234 | −7.277 | 1.00 | 15.77 | A | O |
| ATOM | 2712 | CB | ILE | A | 352 | 55.303 | 49.167 | −4.857 | 1.00 | 17.10 | A | C |
| ATOM | 2713 | CG1 | ILE | A | 352 | 55.387 | 49.937 | −3.540 | 1.00 | 24.67 | A | C |
| ATOM | 2714 | CG2 | ILE | A | 352 | 56.740 | 48.790 | −5.322 | 1.00 | 17.46 | A | C |
| ATOM | 2715 | CD1 | ILE | A | 352 | 55.844 | 49.129 | −2.381 | 1.00 | 28.46 | A | C |
| ATOM | 2716 | N | GLY | A | 353 | 55.291 | 49.633 | −8.199 | 1.00 | 14.93 | A | N |
| ATOM | 2717 | CA | GLY | A | 353 | 55.345 | 48.949 | −9.481 | 1.00 | 14.98 | A | C |
| ATOM | 2718 | C | GLY | A | 353 | 56.559 | 48.090 | −9.631 | 1.00 | 15.46 | A | C |
| ATOM | 2719 | O | GLY | A | 353 | 57.649 | 48.466 | −9.185 | 1.00 | 14.60 | A | O |
| ATOM | 2720 | N | PHE | A | 354 | 56.385 | 46.936 | −10.290 | 1.00 | 15.06 | A | N |
| ATOM | 2721 | CA | PHE | A | 354 | 57.469 | 46.043 | −10.577 | 1.00 | 14.45 | A | C |
| ATOM | 2722 | C | PHE | A | 354 | 57.482 | 45.781 | −12.064 | 1.00 | 15.57 | A | C |
| ATOM | 2723 | O | PHE | A | 354 | 56.431 | 45.590 | −12.685 | 1.00 | 17.67 | A | O |
| ATOM | 2724 | CB | PHE | A | 354 | 57.285 | 44.716 | −9.860 | 1.00 | 16.68 | A | C |
| ATOM | 2725 | CG | PHE | A | 354 | 57.443 | 44.793 | −8.362 | 1.00 | 16.38 | A | C |
| ATOM | 2726 | CD1 | PHE | A | 354 | 56.371 | 45.164 | −7.563 | 1.00 | 16.54 | A | C |
| ATOM | 2727 | CD2 | PHE | A | 354 | 58.640 | 44.430 | −7.756 | 1.00 | 19.54 | A | C |
| ATOM | 2728 | CE1 | PHE | A | 354 | 56.490 | 45.231 | −6.177 | 1.00 | 20.88 | A | C |
| ATOM | 2729 | CE2 | PHE | A | 354 | 58.771 | 44.487 | −6.362 | 1.00 | 19.27 | A | C |
| ATOM | 2730 | CZ | PHE | A | 354 | 57.684 | 44.906 | −5.571 | 1.00 | 15.66 | A | C |
| ATOM | 2731 | N | ALA | A | 355 | 58.684 | 45.766 | −12.606 | 1.00 | 18.28 | A | N |
| ATOM | 2732 | CA | ALA | A | 355 | 58.922 | 45.384 | −13.999 | 1.00 | 16.49 | A | C |
| ATOM | 2733 | C | ALA | A | 355 | 60.245 | 44.644 | −14.081 | 1.00 | 19.90 | A | C |
| ATOM | 2734 | O | ALA | A | 355 | 61.106 | 44.777 | −13.211 | 1.00 | 21.15 | A | O |
| ATOM | 2735 | CB | ALA | A | 355 | 58.922 | 46.569 | −14.878 | 1.00 | 17.48 | A | C |
| ATOM | 2736 | N | VAL | A | 356 | 60.399 | 43.827 | −15.120 | 1.00 | 20.94 | A | N |
| ATOM | 2737 | CA | VAL | A | 356 | 61.650 | 43.107 | −15.305 | 1.00 | 21.72 | A | C |
| ATOM | 2738 | C | VAL | A | 356 | 62.776 | 44.111 | −15.553 | 1.00 | 19.54 | A | C |
| ATOM | 2739 | O | VAL | A | 356 | 62.672 | 44.989 | −16.402 | 1.00 | 21.35 | A | O |
| ATOM | 2740 | CB | VAL | A | 356 | 61.562 | 42.087 | −16.473 | 1.00 | 19.47 | A | C |
| ATOM | 2741 | CG1 | VAL | A | 356 | 62.936 | 41.435 | −16.724 | 1.00 | 20.92 | A | C |
| ATOM | 2742 | CG2 | VAL | A | 356 | 60.517 | 41.025 | −16.174 | 1.00 | 23.31 | A | C |
| ATOM | 2743 | N | SER | A | 357 | 63.853 | 43.982 | −14.793 | 1.00 | 24.48 | A | N |
| ATOM | 2744 | CA | SER | A | 357 | 64.963 | 44.919 | −14.883 | 1.00 | 26.30 | A | C |
| ATOM | 2745 | C | SER | A | 357 | 65.767 | 44.633 | −16.142 | 1.00 | 26.78 | A | C |
| ATOM | 2746 | O | SER | A | 357 | 66.071 | 43.481 | −16.420 | 1.00 | 30.47 | A | O |
| ATOM | 2747 | CB | SER | A | 357 | 65.896 | 44.775 | −13.676 | 1.00 | 25.63 | A | C |
| ATOM | 2748 | OG | SER | A | 357 | 67.009 | 45.645 | −13.815 | 1.00 | 30.40 | A | O |
| ATOM | 2749 | N | ALA | A | 358 | 66.128 | 45.682 | −16.867 | 1.00 | 32.11 | A | N |
| ATOM | 2750 | CA | ALA | A | 358 | 67.012 | 45.567 | −18.029 | 1.00 | 36.75 | A | C |
| ATOM | 2751 | C | ALA | A | 358 | 68.445 | 45.147 | −17.666 | 1.00 | 38.55 | A | C |
| ATOM | 2752 | O | ALA | A | 358 | 69.233 | 44.838 | −18.560 | 1.00 | 42.49 | A | O |
| ATOM | 2753 | CB | ALA | A | 358 | 67.025 | 46.881 | −18.802 | 1.00 | 37.17 | A | C |
| ATOM | 2754 | N | CYS | A | 359 | 68.782 | 45.129 | −16.374 | 1.00 | 39.61 | A | N |
| ATOM | 2755 | CA | CYS | A | 359 | 70.124 | 44.742 | −15.920 | 1.00 | 41.87 | A | C |
| ATOM | 2756 | C | CYS | A | 359 | 70.169 | 43.490 | −15.049 | 1.00 | 42.74 | A | C |
| ATOM | 2757 | O | CYS | A | 359 | 71.241 | 43.132 | −14.550 | 1.00 | 45.60 | A | O |
| ATOM | 2758 | CB | CYS | A | 359 | 70.801 | 45.913 | −15.175 | 1.00 | 41.64 | A | C |
| ATOM | 2759 | SG | CYS | A | 359 | 70.275 | 46.154 | −13.447 | 1.00 | 42.44 | A | S |
| ATOM | 2760 | N | HIS | A | 360 | 69.040 | 42.811 | −14.847 | 1.00 | 42.50 | A | N |
| ATOM | 2761 | CA | HIS | A | 360 | 69.071 | 41.569 | −14.081 | 1.00 | 43.24 | A | C |
| ATOM | 2762 | C | HIS | A | 360 | 69.903 | 40.538 | −14.848 | 1.00 | 44.08 | A | C |
| ATOM | 2763 | O | HIS | A | 360 | 69.932 | 40.545 | −16.089 | 1.00 | 37.43 | A | O |
| ATOM | 2764 | CB | HIS | A | 360 | 67.665 | 41.037 | −13.772 | 1.00 | 43.88 | A | C |
| ATOM | 2765 | CG | HIS | A | 360 | 67.018 | 40.307 | −14.909 | 1.00 | 42.46 | A | C |
| ATOM | 2766 | ND1 | HIS | A | 360 | 66.587 | 40.941 | −16.054 | 1.00 | 43.80 | A | N |
| ATOM | 2767 | CD2 | HIS | A | 360 | 66.711 | 38.997 | −15.067 | 1.00 | 43.40 | A | C |
| ATOM | 2768 | CE1 | HIS | A | 360 | 66.053 | 40.053 | −16.876 | 1.00 | 42.29 | A | C |
| ATOM | 2769 | NE2 | HIS | A | 360 | 66.107 | 38.867 | −16.295 | 1.00 | 40.67 | A | N |
| ATOM | 2770 | N | VAL | A | 361 | 70.604 | 39.688 | −14.108 | 1.00 | 46.31 | A | N |
| ATOM | 2771 | CA | VAL | A | 361 | 71.444 | 38.671 | −14.736 | 1.00 | 53.46 | A | C |
| ATOM | 2772 | C | VAL | A | 361 | 70.569 | 37.519 | −15.208 | 1.00 | 55.15 | A | C |
| ATOM | 2773 | O | VAL | A | 361 | 69.788 | 36.965 | −14.433 | 1.00 | 55.26 | A | O |
| ATOM | 2774 | CB | VAL | A | 361 | 72.584 | 38.144 | −13.812 | 1.00 | 55.33 | A | C |
| ATOM | 2775 | CG1 | VAL | A | 361 | 73.724 | 39.146 | −13.769 | 1.00 | 58.02 | A | C |
| ATOM | 2776 | CG2 | VAL | A | 361 | 72.086 | 37.824 | −12.392 | 1.00 | 57.18 | A | C |
| ATOM | 2777 | N | HIS | A | 362 | 70.687 | 37.191 | −16.491 | 1.00 | 58.23 | A | N |
| ATOM | 2778 | CA | HIS | A | 362 | 69.957 | 36.071 | −17.078 | 1.00 | 61.27 | A | C |
| ATOM | 2779 | C | HIS | A | 362 | 70.886 | 35.268 | −17.991 | 1.00 | 63.90 | A | C |
| ATOM | 2780 | O | HIS | A | 362 | 72.106 | 35.470 | −17.978 | 1.00 | 63.06 | A | O |
| ATOM | 2781 | CB | HIS | A | 362 | 68.707 | 36.570 | −17.820 | 1.00 | 61.80 | A | C |
| ATOM | 2782 | CG | HIS | A | 362 | 68.987 | 37.603 | −18.869 | 1.00 | 64.30 | A | C |
| ATOM | 2783 | ND1 | HIS | A | 362 | 69.075 | 38.949 | −18.582 | 1.00 | 65.54 | A | N |
| ATOM | 2784 | CD2 | HIS | A | 362 | 69.176 | 37.491 | −20.206 | 1.00 | 66.55 | A | C |
| ATOM | 2785 | CE1 | HIS | A | 362 | 69.318 | 39.621 | −19.694 | 1.00 | 66.26 | A | C |
| ATOM | 2786 | NE2 | HIS | A | 362 | 69.384 | 38.760 | −20.694 | 1.00 | 67.04 | A | N |
| ATOM | 2787 | N | ASP | A | 363 | 70.311 | 34.348 | −18.765 | 1.00 | 66.38 | A | N |
| ATOM | 2788 | CA | ASP | A | 363 | 71.086 | 33.477 | −19.645 | 1.00 | 68.15 | A | C |
| ATOM | 2789 | C | ASP | A | 363 | 70.180 | 32.971 | −20.779 | 1.00 | 69.51 | A | C |
| ATOM | 2790 | O | ASP | A | 363 | 69.558 | 33.787 | −21.466 | 1.00 | 68.87 | A | O |
| ATOM | 2791 | CB | ASP | A | 363 | 71.711 | 32.343 | −18.820 | 1.00 | 67.85 | A | C |
| ATOM | 2792 | CG | ASP | A | 363 | 70.722 | 31.702 | −17.869 | 1.00 | 67.03 | A | C |
| ATOM | 2793 | OD1 | ASP | A | 363 | 71.157 | 31.015 | −16.923 | 1.00 | 67.42 | A | O |
| ATOM | 2794 | OD2 | ASP | A | 363 | 69.490 | 31.839 | −17.981 | 1.00 | 67.25 | A | O |
| ATOM | 2795 | N | GLU | A | 364 | 70.111 | 31.651 | −20.981 | 1.00 | 71.44 | A | N |
| ATOM | 2796 | CA | GLU | A | 364 | 69.186 | 31.037 | −21.944 | 1.00 | 71.30 | A | C |
| ATOM | 2977 | C | GLU | A | 364 | 68.223 | 30.026 | −21.289 | 1.00 | 69.20 | A | C |
| ATOM | 2798 | O | GLU | A | 364 | 67.280 | 29.569 | −21.938 | 1.00 | 70.14 | A | O |
| ATOM | 2799 | CB | GLU | A | 364 | 69.980 | 30.351 | −23.069 | 1.00 | 73.26 | A | C |
| ATOM | 2800 | CG | GLU | A | 364 | 69.968 | 31.097 | −24.399 | 1.00 | 74.89 | A | C |
| ATOM | 2801 | CD | GLU | A | 364 | 70.651 | 32.451 | −24.320 | 1.00 | 76.70 | A | C |
| ATOM | 2802 | OE1 | GLU | A | 364 | 71.868 | 32.494 | −24.028 | 1.00 | 77.66 | A | O |
| ATOM | 2803 | OE2 | GLU | A | 364 | 69.969 | 33.476 | −24.549 | 1.00 | 79.09 | A | O |
| ATOM | 2804 | N | PHE | A | 365 | 68.455 | 29.685 | −20.017 | 1.00 | 66.67 | A | N |
| ATOM | 2805 | CA | PHE | A | 365 | 67.630 | 28.704 | −19.299 | 1.00 | 64.31 | A | C |
| ATOM | 2806 | C | PHE | A | 365 | 66.403 | 29.347 | −18.633 | 1.00 | 61.08 | A | C |
| ATOM | 2807 | O | PHE | A | 365 | 65.266 | 29.082 | −19.026 | 1.00 | 62.40 | A | O |
| ATOM | 2808 | CB | PHE | A | 365 | 68.464 | 27.959 | −18.245 | 1.00 | 65.86 | A | C |
| ATOM | 2809 | CG | PHE | A | 365 | 69.365 | 26.886 | −18.819 | 1.00 | 67.96 | A | C |
| ATOM | 2810 | CD1 | PHE | A | 365 | 70.557 | 27.227 | −19.461 | 1.00 | 68.71 | A | C |
| ATOM | 2811 | CD2 | PHE | A | 365 | 69.029 | 25.538 | −18.705 | 1.00 | 68.02 | A | C |
| ATOM | 2812 | CE1 | PHE | A | 365 | 71.395 | 26.241 | −19.989 | 1.00 | 68.45 | A | C |
| ATOM | 2813 | CE2 | PHE | A | 365 | 69.860 | 24.545 | −19.232 | 1.00 | 68.28 | A | C |
| ATOM | 2814 | CZ | PHE | A | 365 | 71.045 | 24.899 | −19.874 | 1.00 | 68.33 | A | C |
| ATOM | 2815 | N | ARG | A | 366 | 66.636 | 30.183 | −17.624 | 1.00 | 54.06 | A | N |
| ATOM | 2816 | CA | ARG | A | 366 | 65.544 | 30.804 | −16.874 | 1.00 | 49.84 | A | C |
| ATOM | 2817 | C | ARG | A | 366 | 65.747 | 32.305 | −16.729 | 1.00 | 48.18 | A | C |
| ATOM | 2818 | O | ARG | A | 366 | 66.867 | 32.816 | −16.857 | 1.00 | 47.96 | A | O |
| ATOM | 2819 | CB | ARG | A | 366 | 65.424 | 30.159 | −15.490 | 1.00 | 47.56 | A | C |
| ATOM | 2820 | CG | ARG | A | 366 | 65.240 | 28.655 | −15.525 | 1.00 | 43.64 | A | C |
| ATOM | 2821 | CD | ARG | A | 366 | 64.974 | 28.012 | −14.174 | 1.00 | 38.98 | A | C |
| ATOM | 2822 | NE | ARG | A | 366 | 66.159 | 27.987 | −13.327 | 1.00 | 41.26 | A | N |
| ATOM | 2823 | CZ | ARG | A | 366 | 66.242 | 27.371 | −12.147 | 1.00 | 36.86 | A | C |
| ATOM | 2824 | NH1 | ARG | A | 366 | 65.203 | 26.718 | −11.644 | 1.00 | 42.11 | A | N |
| ATOM | 2825 | NH2 | ARG | A | 366 | 67.375 | 27.413 | −11.471 | 1.00 | 36.33 | A | N |
| ATOM | 2826 | N | THR | A | 367 | 64.654 | 33.009 | −16.446 | 1.00 | 46.71 | A | N |
| ATOM | 2827 | CA | THR | A | 367 | 64.675 | 34.468 | −16.397 | 1.00 | 46.92 | A | C |
| ATOM | 2828 | C | THR | A | 367 | 63.746 | 35.043 | −15.331 | 1.00 | 44.72 | A | C |
| ATOM | 2829 | O | THR | A | 367 | 62.744 | 34.435 | −14.973 | 1.00 | 42.54 | A | O |
| ATOM | 2830 | CB | THR | A | 367 | 64.306 | 35.019 | −17.783 | 1.00 | 46.57 | A | C |
| ATOM | 2831 | OG1 | THR | A | 367 | 65.143 | 34.406 | −18.774 | 1.00 | 49.10 | A | O |
| ATOM | 2832 | CG2 | THR | A | 367 | 64.628 | 36.484 | −17.902 | 1.00 | 48.33 | A | C |
| ATOM | 2833 | N | ALA | A | 368 | 64.105 | 36.219 | −14.819 | 1.00 | 43.12 | A | N |
| ATOM | 2834 | CA | ALA | A | 368 | 63.206 | 36.976 | −13.962 | 1.00 | 37.88 | A | C |
| ATOM | 2835 | C | ALA | A | 368 | 61.990 | 37.417 | −14.772 | 1.00 | 31.67 | A | C |
| ATOM | 2836 | O | ALA | A | 368 | 62.091 | 37.780 | −15.946 | 1.00 | 33.05 | A | O |
| ATOM | 2837 | CB | ALA | A | 368 | 63.920 | 38.185 | −13.359 | 1.00 | 37.36 | A | C |
| ATOM | 2838 | N | ALA | A | 369 | 60.827 | 37.407 | −14.133 | 1.00 | 30.27 | A | N |
| ATOM | 2839 | CA | ALA | A | 369 | 59.608 | 37.776 | −14.828 | 1.00 | 25.65 | A | C |
| ATOM | 2840 | C | ALA | A | 369 | 58.590 | 38.454 | −13.917 | 1.00 | 18.85 | A | C |
| ATOM | 2841 | O | ALA | A | 369 | 58.574 | 38.264 | −12.707 | 1.00 | 26.84 | A | O |
| ATOM | 2842 | CB | ALA | A | 369 | 58.988 | 36.559 | −15.484 | 1.00 | 26.18 | A | C |
| ATOM | 2843 | N | VAL | A | 370 | 57.772 | 39.267 | −14.543 | 1.00 | 21.79 | A | N |
| ATOM | 2844 | CA | VAL | A | 370 | 56.623 | 39.891 | −13.921 | 1.00 | 22.63 | A | C |
| ATOM | 2845 | C | VAL | A | 370 | 55.460 | 39.580 | −14.864 | 1.00 | 23.34 | A | C |
| ATOM | 2846 | O | VAL | A | 370 | 55.491 | 39.970 | −16.007 | 1.00 | 23.37 | A | O |
| ATOM | 2847 | CB | VAL | A | 370 | 56.806 | 41.403 | −13.783 | 1.00 | 24.21 | A | C |
| ATOM | 2848 | CG1 | VAL | A | 370 | 55.606 | 42.016 | −13.069 | 1.00 | 20.64 | A | C |
| ATOM | 2849 | CG2 | VAL | A | 370 | 58.091 | 41.738 | −13.021 | 1.00 | 24.95 | A | C |
| ATOM | 2850 | N | GLU | A | 371 | 54.435 | 38.890 | −14.367 | 1.00 | 23.45 | A | N |
| ATOM | 2851 | CA | GLU | A | 371 | 53.364 | 38.364 | −15.208 | 1.00 | 26.07 | A | C |
| ATOM | 2852 | C | GLU | A | 371 | 52.005 | 38.601 | −14.556 | 1.00 | 21.52 | A | C |
| ATOM | 2853 | O | GLU | A | 371 | 51.886 | 38.584 | −13.346 | 1.00 | 22.32 | A | O |
| ATOM | 2854 | CB | GLU | A | 371 | 53.593 | 36.859 | −15.452 | 1.00 | 29.04 | A | C |
| ATOM | 2855 | CG | GLU | A | 371 | 54.667 | 36.583 | −16.508 | 1.00 | 37.79 | A | C |
| ATOM | 2856 | CD | GLU | A | 371 | 55.383 | 35.235 | −16.373 | 1.00 | 42.98 | A | C |
| ATOM | 2857 | OE1 | GLU | A | 371 | 55.957 | 34.768 | −17.389 | 1.00 | 47.96 | A | O |
| ATOM | 2858 | OE2 | GLU | A | 371 | 55.428 | 34.655 | −15.271 | 1.00 | 46.63 | A | O |
| ATOM | 2859 | N | GLY | A | 372 | 50.997 | 38.849 | −15.375 | 1.00 | 23.24 | A | N |
| ATOM | 2860 | CA | GLY | A | 372 | 49.629 | 38.995 | −14.902 | 1.00 | 20.28 | A | C |
| ATOM | 2861 | C | GLY | A | 372 | 48.652 | 39.097 | −16.060 | 1.00 | 21.84 | A | C |
| ATOM | 2862 | O | GLY | A | 372 | 49.087 | 39.076 | −17.231 | 1.00 | 23.46 | A | O |
| ATOM | 2863 | N | PRO | A | 373 | 47.355 | 39.219 | −15.790 | 1.00 | 20.69 | A | N |
| ATOM | 2864 | CA | PRO | A | 373 | 46.758 | 39.117 | −14.455 | 1.00 | 18.48 | A | C |
| ATOM | 2865 | C | PRO | A | 373 | 46.427 | 37.686 | −14.050 | 1.00 | 19.08 | A | C |
| ATOM | 2866 | O | PRO | A | 373 | 46.197 | 36.821 | −14.906 | 1.00 | 22.98 | A | O |
| ATOM | 2867 | CB | PRO | A | 373 | 45.466 | 39.923 | −14.607 | 1.00 | 20.66 | A | C |
| ATOM | 2868 | CG | PRO | A | 373 | 45.050 | 39.692 | −16.023 | 1.00 | 22.14 | A | C |
| ATOM | 2869 | CD | PRO | A | 373 | 46.328 | 39.516 | −16.811 | 1.00 | 21.78 | A | C |
| ATOM | 2870 | N | PHE | A | 374 | 46.385 | 37.445 | −12.742 | 1.00 | 15.75 | A | N |
| ATOM | 2871 | CA | PHE | A | 374 | 45.882 | 36.206 | −12.195 | 1.00 | 16.86 | A | C |
| ATOM | 2872 | C | PHE | A | 374 | 44.622 | 36.442 | −11.376 | 1.00 | 21.99 | A | C |
| ATOM | 2873 | O | PHE | A | 374 | 44.370 | 37.554 | −10.943 | 1.00 | 19.16 | A | O |
| ATOM | 2874 | CB | PHE | A | 374 | 46.973 | 35.539 | −11.360 | 1.00 | 16.65 | A | C |
| ATOM | 2875 | CG | PHE | A | 374 | 48.148 | 35.118 | −12.170 | 1.00 | 19.99 | A | C |
| ATOM | 2876 | CD1 | PHE | A | 374 | 48.097 | 33.957 | −12.936 | 1.00 | 23.38 | A | C |
| ATOM | 2877 | CD2 | PHE | A | 374 | 49.294 | 35.894 | −12.221 | 1.00 | 22.80 | A | C |
| ATOM | 2878 | CE1 | PHE | A | 374 | 49.189 | 33.565 | −13.711 | 1.00 | 27.21 | A | C |
| ATOM | 2879 | CE2 | PHE | A | 374 | 50.385 | 35.503 | −12.980 | 1.00 | 22.14 | A | C |
| ATOM | 2880 | CZ | PHE | A | 374 | 50.341 | 34.340 | −13.722 | 1.00 | 25.11 | A | C |
| ATOM | 2881 | N | VAL | A | 375 | 43.822 | 35.392 | −11.207 | 1.00 | 21.84 | A | N |
| ATOM | 2882 | CA | VAL | A | 375 | 42.614 | 35.458 | −10.407 | 1.00 | 22.25 | A | C |
| ATOM | 2883 | C | VAL | A | 375 | 42.948 | 34.876 | −9.049 | 1.00 | 20.48 | A | C |
| ATOM | 2884 | O | VAL | A | 375 | 43.281 | 33.695 | −8.943 | 1.00 | 24.24 | A | O |
| ATOM | 2885 | CB | VAL | A | 375 | 41.439 | 34.656 | −11.016 | 1.00 | 24.98 | A | C |
| ATOM | 2886 | CG1 | VAL | A | 375 | 40.206 | 34.758 | −10.119 | 1.00 | 22.92 | A | C |
| ATOM | 2887 | CG2 | VAL | A | 375 | 41.117 | 35.152 | −12.404 | 1.00 | 23.43 | A | C |
| ATOM | 2888 | N | THR | A | 376 | 42.881 | 35.710 | −8.023 | 1.00 | 22.65 | A | N |
| ATOM | 2889 | CA | THR | A | 376 | 43.104 | 35.291 | −6.637 | 1.00 | 22.10 | A | C |
| ATOM | 2890 | C | THR | A | 376 | 42.027 | 35.873 | −5.737 | 1.00 | 17.23 | A | C |
| ATOM | 2891 | O | THR | A | 376 | 41.856 | 37.081 | −5.647 | 1.00 | 20.58 | A | O |
| ATOM | 2892 | CB | THR | A | 376 | 44.490 | 35.777 | −6.137 | 1.00 | 23.66 | A | C |
| ATOM | 2893 | OG1 | THR | A | 376 | 45.515 | 35.417 | −7.080 | 1.00 | 25.31 | A | O |
| ATOM | 2894 | CG2 | THR | A | 376 | 44.873 | 35.046 | −4.844 | 1.00 | 26.58 | A | C |
| ATOM | 2895 | N | LEU | A | 377 | 41.265 | 35.010 | −5.080 | 1.00 | 22.23 | A | N |
| ATOM | 2896 | CA | LEU | A | 377 | 40.199 | 35.472 | −4.205 | 1.00 | 23.50 | A | C |
| ATOM | 2897 | C | LEU | A | 377 | 40.708 | 35.632 | −2.776 | 1.00 | 27.50 | A | C |
| ATOM | 2898 | O | LEU | A | 377 | 41.710 | 35.019 | −2.401 | 1.00 | 28.12 | A | O |
| ATOM | 2899 | CB | LEU | A | 377 | 39.046 | 34.481 | −4.217 | 1.00 | 26.48 | A | C |
| ATOM | 2900 | CG | LEU | A | 377 | 38.541 | 34.109 | −5.622 | 1.00 | 29.26 | A | C |
| ATOM | 2901 | CD1 | LEU | A | 377 | 37.314 | 33.287 | −5.496 | 1.00 | 30.60 | A | C |
| ATOM | 2902 | CD2 | LEU | A | 377 | 38.247 | 35.344 | −6.466 | 1.00 | 29.52 | A | C |
| ATOM | 2903 | N | ASP | A | 378 | 39.981 | 36.441 | −2.014 | 1.00 | 29.13 | A | N |
| ATOM | 2904 | CA | ASP | A | 378 | 40.177 | 36.631 | −0.574 | 1.00 | 35.27 | A | C |
| ATOM | 2905 | C | ASP | A | 378 | 41.540 | 37.210 | −0.251 | 1.00 | 33.60 | A | C |
| ATOM | 2906 | O | ASP | A | 378 | 42.134 | 36.858 | 0.760 | 1.00 | 36.99 | A | O |
| ATOM | 2907 | CB | ASP | A | 378 | 40.002 | 35.325 | 0.196 | 1.00 | 33.08 | A | C |
| ATOM | 2908 | CG | ASP | A | 378 | 38.627 | 34.762 | 0.070 | 1.00 | 36.04 | A | C |
| ATOM | 2909 | OD1 | ASP | A | 378 | 37.654 | 35.549 | −0.049 | 1.00 | 31.19 | A | O |
| ATOM | 2910 | OD2 | ASP | A | 378 | 38.441 | 33.532 | 0.097 | 1.00 | 41.39 | A | O |
| ATOM | 2911 | N | MET | A | 379 | 42.026 | 38.096 | −1.110 | 1.00 | 34.47 | A | N |
| ATOM | 2912 | CA | MET | A | 379 | 43.349 | 38.682 | −0.933 | 1.00 | 33.96 | A | C |
| ATOM | 2913 | C | MET | A | 379 | 43.396 | 39.613 | 0.270 | 1.00 | 34.59 | A | C |
| ATOM | 2914 | O | MET | A | 379 | 44.449 | 39.755 | 0.871 | 1.00 | 37.56 | A | O |
| ATOM | 2915 | CB | MET | A | 379 | 43.782 | 39.449 | −2.186 | 1.00 | 30.92 | A | C |
| ATOM | 2916 | CG | MET | A | 379 | 44.041 | 38.562 | −3.375 | 1.00 | 28.94 | A | C |
| ATOM | 2917 | SD | MET | A | 379 | 44.749 | 39.489 | −4.761 | 1.00 | 26.10 | A | S |
| ATOM | 2918 | CE | MET | A | 379 | 43.486 | 40.462 | −5.207 | 1.00 | 25.48 | A | C |
| ATOM | 2919 | N | GLU | A | 380 | 42.268 | 40.233 | 0.615 | 1.00 | 41.66 | A | N |
| ATOM | 2920 | CA | GLU | A | 380 | 42.182 | 41.091 | 1.805 | 1.00 | 46.13 | A | C |
| ATOM | 2921 | C | GLU | A | 380 | 42.498 | 40.308 | 3.080 | 1.00 | 47.68 | A | C |
| ATOM | 2922 | O | GLU | A | 380 | 43.208 | 40.810 | 3.957 | 1.00 | 50.01 | A | O |
| ATOM | 2923 | CB | GLU | A | 380 | 40.803 | 41.741 | 1.927 | 1.00 | 48.86 | A | C |
| ATOM | 2924 | CG | GLU | A | 380 | 40.743 | 43.189 | 1.446 | 1.00 | 51.99 | A | C |
| ATOM | 2925 | CD | GLU | A | 380 | 40.851 | 43.338 | −0.066 | 1.00 | 55.58 | A | C |
| ATOM | 2926 | OE1 | GLU | A | 380 | 40.498 | 42.385 | −0.799 | 1.00 | 56.65 | A | O |
| ATOM | 2927 | OE2 | GLU | A | 380 | 41.282 | 44.426 | −0.524 | 1.00 | 57.63 | A | O |
| ATOM | 2928 | N | ASP | A | 381 | 41.997 | 39.075 | 3.169 | 1.00 | 46.10 | A | N |
| ATOM | 2929 | CA | ASP | A | 381 | 42.296 | 38.192 | 4.309 | 1.00 | 46.90 | A | C |
| ATOM | 2930 | C | ASP | A | 381 | 43.774 | 37.804 | 4.428 | 1.00 | 44.71 | A | C |
| ATOM | 2931 | O | ASP | A | 381 | 44.167 | 37.175 | 5.402 | 1.00 | 45.68 | A | O |
| ATOM | 2932 | CB | ASP | A | 381 | 41.448 | 36.911 | 4.254 | 1.00 | 45.21 | A | C |
| ATOM | 2933 | CG | ASP | A | 381 | 40.052 | 37.059 | 3.994 | 0.00 | 50.29 | A | C |
| ATOM | 2934 | OD1 | ASP | A | 381 | 39.485 | 37.731 | 4.881 | 0.00 | 50.63 | A | O |
| ATOM | 2935 | OD2 | ASP | A | 381 | 39.440 | 36.553 | 3.030 | 0.00 | 50.70 | A | O |
| ATOM | 2936 | N | CYS | A | 382 | 44.587 | 38.137 | 3.429 | 1.00 | 43.03 | A | N |
| ATOM | 2937 | CA | CYS | A | 382 | 46.026 | 37.939 | 3.526 | 1.00 | 42.15 | A | C |
| ATOM | 2938 | C | CYS | A | 382 | 46.693 | 39.021 | 4.400 | 1.00 | 41.50 | A | C |
| ATOM | 2939 | O | CYS | A | 382 | 47.808 | 38.827 | 4.855 | 1.00 | 43.47 | A | O |
| ATOM | 2940 | CB | CYS | A | 382 | 46.669 | 37.897 | 2.137 | 1.00 | 41.83 | A | C |
| ATOM | 2941 | SG | CYS | A | 382 | 45.985 | 36.643 | 1.026 | 1.00 | 38.22 | A | S |
| ATOM | 2942 | N | GLY | A | 383 | 45.999 | 40.133 | 4.645 | 1.00 | 43.76 | A | N |
| ATOM | 2943 | CA | GLY | A | 383 | 46.521 | 41.233 | 5.447 | 1.00 | 47.67 | A | C |
| ATOM | 2944 | C | GLY | A | 383 | 46.200 | 41.165 | 6.939 | 1.00 | 51.51 | A | C |
| ATOM | 2945 | O | GLY | A | 383 | 45.034 | 41.086 | 7.329 | 1.00 | 52.22 | A | O |
| ATOM | 2946 | N | TYR | A | 384 | 47.239 | 41.220 | 7.772 | 1.00 | 55.14 | A | N |
| ATOM | 2947 | CA | TYR | A | 384 | 47.092 | 41.146 | 9.227 | 1.00 | 57.40 | A | C |
| ATOM | 2948 | C | TYR | A | 384 | 46.613 | 42.453 | 9.878 | 1.00 | 58.55 | A | C |
| ATOM | 2949 | O | TYR | A | 384 | 47.216 | 43.508 | 9.687 | 1.00 | 56.79 | A | O |
| ATOM | 2950 | CB | TYR | A | 384 | 48.414 | 40.719 | 9.865 | 1.00 | 57.72 | A | C |
| ATOM | 2951 | CG | TYR | A | 384 | 48.357 | 40.643 | 11.375 | 1.00 | 60.49 | A | C |
| ATOM | 2952 | CD1 | TYR | A | 384 | 47.657 | 39.623 | 12.015 | 1.00 | 61.73 | A | C |
| ATOM | 2953 | CD2 | TYR | A | 384 | 48.994 | 41.598 | 12.167 | 1.00 | 62.68 | A | C |
| ATOM | 2954 | CE1 | TYR | A | 384 | 47.597 | 39.551 | 13.408 | 1.00 | 61.50 | A | C |
| ATOM | 2955 | CE2 | TYR | A | 384 | 48.941 | 41.536 | 13.561 | 1.00 | 62.87 | A | C |
| ATOM | 2956 | CZ | TYR | A | 384 | 48.242 | 40.510 | 14.173 | 1.00 | 62.60 | A | C |
| ATOM | 2957 | OH | TYR | A | 384 | 48.188 | 40.443 | 15.548 | 1.00 | 62.41 | A | O |
| ATOM | 2958 | N | ASN | A | 385 | 45.540 | 42.359 | 10.666 | 1.00 | 60.14 | A | N |
| ATOM | 2959 | CA | ASN | A | 385 | 45.049 | 43.478 | 11.471 | 1.00 | 62.13 | A | C |
| ATOM | 2960 | C | ASN | A | 385 | 45.450 | 43.295 | 12.938 | 1.00 | 63.14 | A | C |
| ATOM | 2961 | 1OCT | ASN | A | 385 | 46.043 | 44.168 | 13.582 | 1.00 | 64.31 | A | O |
| ATOM | 2962 | CB | ASN | A | 385 | 43.524 | 43.592 | 11.362 | 1.00 | 62.36 | A | C |
| ATOM | 2963 | CG | ASN | A | 385 | 43.037 | 43.666 | 9.918 | 1.00 | 63.95 | A | C |
| ATOM | 2964 | OD1 | ASN | A | 385 | 42.654 | 42.654 | 9.326 | 1.00 | 64.71 | A | O |
| ATOM | 2965 | ND2 | ASN | A | 385 | 43.043 | 44.866 | 9.351 | 1.00 | 63.38 | A | N |
| ATOM | 2966 | 2OCT | ASN | A | 385 | 45.193 | 42.257 | 13.550 | 1.00 | 63.30 | A | O |
| ATOM | 2967 | O | HOH | W | 1 | 79.629 | 68.206 | 12.595 | 1.00 | 19.21 | W | O |
| ATOM | 2968 | O | HOH | W | 2 | 49.015 | 47.109 | −12.447 | 1.00 | 16.55 | W | O |
| ATOM | 2969 | O | HOH | W | 3 | 85.976 | 52.179 | 5.603 | 1.00 | 21.59 | W | O |
| ATOM | 2970 | O | HOH | W | 4 | 80.248 | 66.497 | 15.419 | 1.00 | 25.04 | W | O |
| ATOM | 2971 | O | HOH | W | 5 | 75.516 | 59.444 | −7.006 | 1.00 | 20.45 | W | O |
| ATOM | 2972 | O | HOH | W | 6 | 64.679 | 60.731 | 5.508 | 1.00 | 20.67 | W | O |
| ATOM | 2973 | O | HOH | W | 7 | 52.200 | 57.481 | −0.615 | 1.00 | 36.49 | W | O |
| ATOM | 2974 | O | HOH | W | 8 | 52.125 | 39.097 | −18.355 | 1.00 | 30.59 | W | O |
| ATOM | 2975 | O | HOH | W | 9 | 66.983 | 62.454 | 10.671 | 1.00 | 21.40 | W | O |
| ATOM | 2976 | O | HOH | W | 10 | 44.515 | 33.044 | −12.767 | 1.00 | 22.53 | W | O |
| ATOM | 2977 | O | HOH | W | 11 | 80.173 | 73.603 | 4.481 | 1.00 | 33.04 | W | O |
| ATOM | 2978 | O | HOH | W | 12 | 47.807 | 50.724 | −13.972 | 1.00 | 20.13 | W | O |
| ATOM | 2979 | O | HOH | W | 13 | 80.860 | 59.315 | 0.203 | 1.00 | 26.62 | W | O |
| ATOM | 2980 | O | HOH | W | 14 | 55.473 | 70.139 | −4.604 | 1.00 | 53.88 | W | O |
| ATOM | 2981 | O | HOH | W | 15 | 74.472 | 71.225 | −0.260 | 1.00 | 39.12 | W | O |
| ATOM | 2982 | O | HOH | W | 16 | 40.544 | 39.218 | −3.509 | 1.00 | 31.61 | W | O |
| ATOM | 2983 | O | HOH | W | 17 | 80.450 | 59.844 | 12.764 | 1.00 | 26.37 | W | O |
| ATOM | 2984 | O | HOH | W | 18 | 66.075 | 77.514 | 3.855 | 1.00 | 38.59 | W | O |
| ATOM | 2985 | O | HOH | W | 19 | 85.138 | 68.322 | 12.518 | 1.00 | 27.81 | W | O |
| ATOM | 2986 | O | HOH | W | 20 | 87.998 | 70.949 | 7.571 | 1.00 | 53.38 | W | O |
| ATOM | 2987 | O | HOH | W | 21 | 87.495 | 66.754 | 13.176 | 1.00 | 21.08 | W | O |
| ATOM | 2988 | O | HOH | W | 22 | 49.756 | 30.124 | −1.047 | 1.00 | 45.82 | W | O |
| ATOM | 2989 | O | HOH | W | 23 | 49.361 | 33.536 | 13.751 | 1.00 | 66.10 | W | O |
| ATOM | 2990 | O | HOH | W | 24 | 67.788 | 54.838 | 10.862 | 1.00 | 28.51 | W | O |
| ATOM | 2991 | O | HOH | W | 25 | 50.160 | 45.140 | −1.881 | 1.00 | 27.20 | W | O |
| ATOM | 2992 | O | HOH | W | 26 | 82.766 | 67.175 | 5.119 | 1.00 | 34.54 | W | O |
| ATOM | 2993 | O | HOH | W | 27 | 45.592 | 32.973 | −7.823 | 1.00 | 33.43 | W | O |
| ATOM | 2994 | O | HOH | W | 28 | 81.090 | 55.720 | 18.331 | 1.00 | 22.44 | W | O |
| ATOM | 2995 | O | HOH | W | 29 | 43.057 | 33.861 | 0.341 | 1.00 | 80.20 | W | O |
| ATOM | 2996 | O | HOH | W | 30 | 61.780 | 27.615 | 13.286 | 1.00 | 58.09 | W | O |
| ATOM | 2997 | O | HOH | W | 31 | 50.466 | 45.953 | 8.884 | 1.00 | 40.45 | W | O |
| ATOM | 2998 | O | HOH | W | 32 | 83.327 | 58.106 | 0.741 | 1.00 | 25.84 | W | O |
| ATOM | 2999 | O | HOH | W | 33 | 81.327 | 48.709 | 18.206 | 1.00 | 36.23 | W | O |
| ATOM | 3000 | O | HOH | W | 34 | 72.944 | 38.241 | 4.000 | 1.00 | 50.15 | W | O |
| ATOM | 3001 | O | HOH | W | 35 | 48.453 | 40.727 | −19.960 | 1.00 | 41.17 | W | O |
| ATOM | 3002 | O | HOH | W | 36 | 66.664 | 48.548 | 5.951 | 1.00 | 33.26 | W | O |
| ATOM | 3003 | O | HOH | W | 37 | 58.083 | 43.778 | −17.062 | 1.00 | 24.83 | W | O |
| ATOM | 3004 | O | HOH | W | 38 | 55.799 | 60.814 | 5.110 | 1.00 | 39.72 | W | O |
| ATOM | 3005 | O | HOH | W | 39 | 79.293 | 52.119 | 13.860 | 1.00 | 21.39 | W | O |
| ATOM | 3006 | O | HOH | W | 40 | 77.511 | 45.900 | 20.280 | 1.00 | 50.24 | W | O |
| ATOM | 3007 | O | HOH | W | 41 | 50.802 | 43.439 | −20.117 | 1.00 | 42.67 | W | O |
| ATOM | 3008 | O | HOH | W | 42 | 66.106 | 19.960 | −9.172 | 1.00 | 47.01 | W | O |
| ATOM | 3009 | O | HOH | W | 43 | 63.894 | 58.910 | −19.204 | 1.00 | 76.51 | W | O |
| ATOM | 3010 | O | HOH | W | 44 | 76.257 | 41.684 | 15.651 | 1.00 | 62.92 | W | O |
| ATOM | 3011 | O | HOH | W | 45 | 54.819 | 50.279 | −18.015 | 1.00 | 21.51 | W | O |
| ATOM | 3012 | O | HOH | W | 46 | 65.401 | 64.403 | 6.138 | 1.00 | 24.60 | W | O |
| ATOM | 3013 | O | HOH | W | 47 | 53.853 | 55.150 | −11.636 | 1.00 | 29.65 | W | O |
| ATOM | 3014 | O | HOH | W | 48 | 68.908 | 67.519 | −5.703 | 1.00 | 33.79 | W | O |
| ATOM | 3015 | O | HOH | W | 49 | 79.968 | 52.673 | 6.743 | 1.00 | 26.80 | W | O |
| ATOM | 3016 | O | HOH | W | 50 | 48.181 | 44.979 | −10.637 | 1.00 | 17.31 | W | O |
| ATOM | 3017 | O | HOH | W | 51 | 53.488 | 60.669 | −0.029 | 1.00 | 31.52 | W | O |
| ATOM | 3018 | O | HOH | W | 52 | 62.724 | 61.887 | 9.306 | 1.00 | 24.34 | W | O |
| ATOM | 3019 | O | HOH | W | 53 | 64.870 | 59.282 | 19.837 | 1.00 | 40.43 | W | O |
| ATOM | 3020 | O | HOH | W | 54 | 67.034 | 55.997 | 8.478 | 1.00 | 18.91 | W | O |
| ATOM | 3021 | O | HOH | W | 55 | 81.783 | 69.009 | 13.884 | 1.00 | 24.02 | W | O |
| ATOM | 3022 | O | HOH | W | 56 | 62.338 | 60.129 | 2.848 | 1.00 | 20.26 | W | O |
| ATOM | 3023 | O | HOH | W | 57 | 59.948 | 49.626 | 3.509 | 1.00 | 20.58 | W | O |
| ATOM | 3024 | O | HOH | W | 58 | 74.315 | 61.973 | −6.807 | 1.00 | 24.90 | W | O |
| ATOM | 3025 | O | HOH | W | 59 | 72.754 | 44.483 | 0.023 | 1.00 | 30.57 | W | O |
| ATOM | 3026 | O | HOH | W | 60 | 85.756 | 65.674 | 6.462 | 1.00 | 34.66 | W | O |
| ATOM | 3027 | O | HOH | W | 61 | 65.197 | 62.897 | 8.395 | 1.00 | 24.15 | W | O |
| ATOM | 3028 | O | HOH | W | 62 | 83.185 | 55.955 | 4.621 | 1.00 | 21.13 | W | O |
| ATOM | 3029 | O | HOH | W | 63 | 68.666 | 31.435 | 6.797 | 1.00 | 32.75 | W | O |
| ATOM | 3030 | O | HOH | W | 64 | 70.959 | 50.115 | −0.021 | 1.00 | 24.74 | W | O |
| ATOM | 3031 | O | HOH | W | 65 | 70.634 | 69.168 | 18.081 | 1.00 | 35.02 | W | O |
| ATOM | 3032 | O | HOH | W | 66 | 83.133 | 65.815 | 2.329 | 1.00 | 28.15 | W | O |
| ATOM | 3033 | O | HOH | W | 67 | 81.369 | 47.920 | 15.072 | 1.00 | 41.54 | W | O |
| ATOM | 3034 | O | HOH | W | 68 | 87.299 | 59.567 | 9.845 | 1.00 | 38.69 | W | O |
| ATOM | 3035 | O | HOH | W | 69 | 41.854 | 32.167 | −5.319 | 1.00 | 34.05 | W | O |
| ATOM | 3036 | O | HOH | W | 70 | 87.742 | 64.125 | 6.529 | 1.00 | 68.19 | W | O |
| ATOM | 3037 | O | HOH | W | 71 | 72.460 | 68.092 | 12.019 | 1.00 | 27.07 | W | O |
| ATOM | 3038 | O | HOH | W | 72 | 65.274 | 42.384 | −19.635 | 1.00 | 61.51 | W | O |
| ATOM | 3039 | O | HOH | W | 73 | 85.768 | 65.313 | 2.708 | 1.00 | 45.28 | W | O |
| ATOM | 3040 | O | HOH | W | 74 | 62.071 | 26.325 | −12.323 | 1.00 | 30.75 | W | O |
| ATOM | 3041 | O | HOH | W | 75 | 53.548 | 58.246 | 7.753 | 1.00 | 35.77 | W | O |
| ATOM | 3042 | O | HOH | W | 76 | 48.415 | 35.384 | −17.283 | 1.00 | 49.88 | W | O |
| ATOM | 3043 | O | HOH | W | 77 | 63.389 | 66.452 | 6.071 | 1.00 | 24.26 | W | O |
| ATOM | 3044 | O | HOH | W | 78 | 82.811 | 58.045 | −3.976 | 1.00 | 49.01 | W | O |
| ATOM | 3045 | O | HOH | W | 79 | 73.849 | 44.456 | −1.977 | 1.00 | 46.53 | W | O |
| ATOM | 3046 | O | HOH | W | 80 | 45.102 | 52.297 | −10.384 | 1.00 | 27.65 | W | O |
| ATOM | 3047 | O | HOH | W | 81 | 65.497 | 47.590 | −7.949 | 1.00 | 22.50 | W | O |
| ATOM | 3048 | O | HOH | W | 82 | 60.385 | 50.571 | −20.969 | 1.00 | 35.94 | W | O |
| ATOM | 3049 | O | HOH | W | 83 | 73.977 | 51.153 | −13.532 | 1.00 | 42.34 | W | O |
| ATOM | 3050 | O | HOH | W | 84 | 73.807 | 75.017 | −0.696 | 1.00 | 45.17 | W | O |
| ATOM | 3051 | O | HOH | W | 85 | 89.302 | 56.875 | 9.021 | 1.00 | 36.01 | W | O |
| ATOM | 3052 | O | HOH | W | 86 | 59.573 | 59.896 | 2.947 | 1.00 | 37.55 | W | O |
| ATOM | 3053 | O | HOH | W | 87 | 69.343 | 40.980 | 6.123 | 1.00 | 33.99 | W | O |
| ATOM | 3054 | O | HOH | W | 88 | 52.716 | 58.960 | −10.022 | 1.00 | 38.62 | W | O |
| ATOM | 3055 | O | HOH | W | 89 | 71.368 | 68.265 | 20.363 | 1.00 | 40.93 | W | O |
| ATOM | 3056 | O | HOH | W | 90 | 58.025 | 24.259 | 10.874 | 1.00 | 64.35 | W | O |
| ATOM | 3057 | O | HOH | W | 91 | 79.324 | 57.854 | −5.249 | 1.00 | 28.34 | W | O |
| ATOM | 3058 | O | HOH | W | 92 | 52.049 | 42.888 | 4.777 | 1.00 | 33.22 | W | O |
| ATOM | 3059 | O | HOH | W | 93 | 58.572 | 51.240 | −21.845 | 1.00 | 39.18 | W | O |
| ATOM | 3060 | O | HOH | W | 94 | 58.399 | 59.801 | −15.372 | 1.00 | 34.06 | W | O |
| ATOM | 3061 | O | HOH | W | 95 | 51.199 | 63.163 | −3.700 | 1.00 | 34.26 | W | O |
| ATOM | 3062 | O | HOH | W | 96 | 39.751 | 42.093 | 5.333 | 1.00 | 63.63 | W | O |
| ATOM | 3063 | O | HOH | W | 97 | 62.377 | 69.523 | 12.319 | 1.00 | 37.67 | W | O |
| ATOM | 3064 | O | HOH | W | 98 | 57.972 | 57.007 | 14.799 | 1.00 | 35.19 | W | O |
| ATOM | 3065 | O | HOH | W | 99 | 62.896 | 33.477 | −11.943 | 1.00 | 76.49 | W | O |
| ATOM | 3066 | O | HOH | W | 100 | 77.078 | 56.466 | −5.817 | 1.00 | 21.61 | W | O |
| ATOM | 3067 | O | HOH | W | 101 | 58.723 | 72.174 | 10.770 | 1.00 | 45.78 | W | O |
| ATOM | 3068 | O | HOH | W | 102 | 82.563 | 53.786 | 6.291 | 1.08 | 28.84 | W | O |
| ATOM | 3069 | O | HOH | W | 103 | 59.353 | 71.034 | 3.910 | 1.00 | 33.97 | W | O |
| ATOM | 3070 | O | HOH | W | 104 | 64.748 | 30.333 | −21.491 | 1.00 | 39.71 | W | O |
| ATOM | 3071 | O | HOH | W | 105 | 74.634 | 59.328 | −12.866 | 1.00 | 40.33 | W | O |
| ATOM | 3072 | O | HOH | W | 106 | 55.438 | 42.543 | −19.877 | 1.00 | 35.74 | W | O |
| ATOM | 3073 | O | HOH | W | 107 | 77.532 | 77.780 | −0.830 | 1.00 | 47.95 | W | O |
| ATOM | 3074 | O | HOH | W | 108 | 65.148 | 68.989 | −11.545 | 1.00 | 51.49 | W | O |
| ATOM | 3075 | O | HOH | W | 109 | 57.778 | 41.274 | −18.333 | 1.00 | 41.55 | W | O |
| ATOM | 3076 | O | HOH | W | 110 | 55.086 | 59.049 | 16.334 | 1.00 | 47.49 | W | O |
| ATOM | 3077 | O | HOH | W | 111 | 81.228 | 50.040 | 13.406 | 1.00 | 68.55 | W | O |
| ATOM | 3078 | O | HOH | W | 112 | 39.213 | 39.599 | −0.284 | 1.00 | 54.99 | W | O |
| ATOM | 3079 | O | HOH | W | 113 | 58.054 | 38.933 | −17.692 | 1.00 | 30.12 | W | O |
| ATOM | 3080 | O | HOH | W | 114 | 46.682 | 50.824 | −7.093 | 1.00 | 27.96 | W | O |
| ATOM | 3081 | O | HOH | W | 115 | 56.111 | 63.217 | −0.389 | 1.00 | 31.05 | W | O |
| ATOM | 3082 | O | HOH | W | 116 | 83.364 | 67.774 | 0.538 | 1.00 | 32.16 | W | O |
| ATOM | 3083 | O | HOH | W | 117 | 48.343 | 27.854 | 7.458 | 1.00 | 45.35 | W | O |
| ATOM | 3084 | O | HOH | W | 118 | 62.036 | 71.098 | 6.922 | 1.00 | 37.49 | W | O |
| ATOM | 3085 | O | HOH | W | 119 | 50.470 | 55.859 | 8.484 | 1.00 | 50.28 | W | O |
| ATOM | 3086 | O | HOH | W | 120 | 59.219 | 48.282 | −21.628 | 1.00 | 49.61 | W | O |
| ATOM | 3087 | O | HOH | W | 121 | 70.795 | 46.171 | 0.982 | 1.00 | 42.89 | W | O |
| ATOM | 3088 | O | HOH | W | 122 | 67.725 | 50.769 | 8.365 | 1.00 | 44.67 | W | O |
| ATOM | 3089 | O | HOH | W | 123 | 62.717 | 69.639 | −10.878 | 1.00 | 52.07 | W | O |
| ATOM | 3090 | O | HOH | W | 124 | 60.253 | 41.588 | −20.165 | 1.00 | 31.75 | W | O |
| ATOM | 3091 | O | HOH | W | 125 | 40.595 | 48.954 | −7.729 | 1.00 | 24.22 | W | O |
| ATOM | 3092 | O | HOH | W | 126 | 60.544 | 37.484 | −18.077 | 1.00 | 33.87 | W | O |
| ATOM | 3093 | O | HOH | W | 127 | 65.662 | 55.956 | 21.772 | 1.00 | 33.55 | W | O |
| ATOM | 3094 | O | HOH | W | 128 | 65.944 | 31.897 | −19.969 | 1.00 | 51.23 | W | O |
| ATOM | 3095 | O | HOH | W | 129 | 61.793 | 76.127 | 1.749 | 1.00 | 56.51 | W | O |
| ATOM | 3096 | O | HOH | W | 130 | 85.302 | 59.460 | 0.012 | 1.00 | 36.81 | W | O |
| ATOM | 3097 | O | HOH | W | 131 | 51.594 | 64.021 | −6.048 | 1.00 | 58.01 | W | O |
| ATOM | 3098 | O | HOH | W | 132 | 54.042 | 66.667 | −6.161 | 1.00 | 63.77 | W | O |
| ATOM | 3099 | O | HOH | W | 133 | 62.332 | 75.297 | 4.414 | 1.00 | 55.75 | W | O |
| ATOM | 3100 | O | HOH | W | 134 | 50.042 | 46.059 | −20.292 | 1.00 | 73.57 | W | O |
| ATOM | 3101 | O | HOH | W | 135 | 79.366 | 53.491 | −11.459 | 1.00 | 45.91 | W | O |
| ATOM | 3102 | O | HOH | W | 136 | 62.077 | 57.368 | −20.403 | 1.00 | 50.99 | W | O |
| ATOM | 3103 | O | HOH | W | 137 | 70.534 | 49.754 | 8.111 | 1.00 | 67.69 | W | O |
| ATOM | 3104 | O | HOH | W | 138 | 78.803 | 66.881 | 19.280 | 1.00 | 33.48 | W | O |
| ATOM | 3105 | O | HOH | W | 139 | 83.041 | 34.659 | −5.519 | 1.00 | 42.03 | W | O |
| ATOM | 3106 | O | HOH | W | 140 | 77.602 | 56.674 | −9.068 | 1.00 | 43.32 | W | O |
| ATOM | 3107 | O | HOH | W | 141 | 80.073 | 75.620 | 15.238 | 1.00 | 30.64 | W | O |
| ATOM | 3108 | O | HOH | W | 142 | 80.099 | 63.907 | −8.340 | 1.00 | 39.92 | W | O |
| ATOM | 3109 | O | HOH | W | 143 | 56.033 | 68.239 | −6.044 | 1.00 | 52.71 | W | O |
| ATOM | 3110 | O | HOH | W | 144 | 53.413 | 63.896 | −8.009 | 1.00 | 35.96 | W | O |
| ATOM | 3111 | O | HOH | W | 145 | 89.147 | 64.107 | 9.192 | 1.00 | 45.54 | W | O |
| ATOM | 3112 | O | HOH | W | 146 | 37.356 | 37.399 | −3.003 | 1.00 | 37.40 | W | O |
| ATOM | 3113 | O | HOH | W | 147 | 71.841 | 68.945 | −7.695 | 1.00 | 67.24 | W | O |
| ATOM | 3114 | O | HOH | W | 148 | 65.710 | 25.459 | 1.815 | 1.00 | 68.03 | W | O |
| ATOM | 3115 | O | HOH | W | 149 | 54.563 | 32.460 | 14.878 | 1.00 | 45.89 | W | O |
| ATOM | 3116 | O | HOH | W | 150 | 69.771 | 32.591 | −13.970 | 1.00 | 38.39 | W | O |
| ATOM | 3117 | O | HOH | W | 151 | 40.372 | 41.672 | −3.643 | 1.00 | 35.36 | W | O |
| ATOM | 3118 | O | HOH | W | 152 | 67.233 | 45.846 | −10.950 | 1.00 | 26.82 | W | O |
| ATOM | 3119 | O | HOH | W | 153 | 38.766 | 47.051 | −8.023 | 1.00 | 28.56 | W | O |
| ATOM | 3120 | O | HOH | W | 154 | 81.319 | 69.504 | −2.622 | 1.00 | 45.91 | W | O |
| ATOM | 3121 | O | HOH | W | 155 | 53.761 | 29.575 | −15.833 | 1.00 | 38.29 | W | O |
| ATOM | 3122 | O | HOH | W | 156 | 56.342 | 73.135 | −5.405 | 1.00 | 68.20 | W | O |
| ATOM | 3123 | O | HOH | W | 157 | 53.773 | 72.306 | −0.902 | 1.00 | 67.09 | W | O |
| ATOM | 3124 | O | HOH | W | 158 | 79.692 | 66.676 | −5.072 | 1.00 | 50.12 | W | O |
| ATOM | 3125 | O | HOH | W | 159 | 73.232 | 38.089 | −7.677 | 1.00 | 45.17 | W | O |
| ATOM | 3126 | O | HOH | W | 160 | 46.657 | 52.288 | −3.310 | 1.00 | 36.02 | W | O |
| ATOM | 3127 | O | HOH | W | 161 | 68.327 | 19.772 | −0.212 | 1.00 | 70.84 | W | O |
| ATOM | 3128 | O | HOH | W | 162 | 57.706 | 29.223 | −8.479 | 1.00 | 39.36 | W | O |
| ATOM | 3129 | O | HOH | W | 163 | 80.380 | 78.795 | 5.802 | 1.00 | 56.31 | W | O |
| ATOM | 3130 | O | HOH | W | 164 | 56.675 | 59.728 | −19.716 | 1.00 | 51.35 | W | O |
| ATOM | 3131 | O | HOH | W | 165 | 72.021 | 78.865 | 10.956 | 1.00 | 57.63 | W | O |
| ATOM | 3132 | O | HOH | W | 166 | 61.187 | 22.723 | 11.672 | 1.00 | 52.43 | W | O |
| ATOM | 3133 | O | HOH | W | 167 | 52.637 | 65.982 | −3.596 | 1.00 | 43.55 | W | O |
| ATOM | 3134 | O | HOH | W | 168 | 77.094 | 59.049 | −11.764 | 1.00 | 53.68 | W | O |
| ATOM | 3135 | O | HOH | W | 169 | 82.297 | 55.117 | −5.408 | 1.00 | 56.75 | W | O |
| ATOM | 3136 | O | HOH | W | 170 | 44.896 | 54.140 | −2.621 | 1.00 | 44.26 | W | O |
| ATOM | 3137 | O | HOH | W | 171 | 75.662 | 48.265 | 9.068 | 1.00 | 31.34 | W | O |
| ATOM | 3138 | O | HOH | W | 172 | 62.322 | 26.608 | −15.255 | 1.00 | 73.50 | W | O |
| ATOM | 3139 | O | HOH | W | 173 | 70.503 | 79.530 | 7.957 | 1.00 | 46.42 | W | O |
| ATOM | 3140 | O | HOH | W | 174 | 78.756 | 79.738 | 3.636 | 1.00 | 57.59 | W | O |
| ATOM | 3141 | O | HOH | W | 175 | 63.567 | 48.079 | 7.690 | 1.00 | 56.49 | W | O |
| ATOM | 3142 | O | HOH | W | 176 | 73.105 | 50.182 | 8.251 | 1.00 | 62.98 | W | O |
| ATOM | 3143 | O | HOH | W | 177 | 74.155 | 72.309 | −2.546 | 1.00 | 63.14 | W | O |
| ATOM | 3144 | O | HOH | W | 178 | 65.269 | 74.588 | 10.615 | 1.00 | 38.50 | W | O |
| ATOM | 3145 | O | HOH | W | 179 | 77.404 | 52.712 | −10.561 | 1.00 | 40.86 | W | O |
| ATOM | 3146 | O | HOH | W | 180 | 53.494 | 69.486 | −1.573 | 1.00 | 61.27 | W | O |
| ATOM | 3147 | O | HOH | W | 181 | 44.408 | 43.630 | 15.946 | 1.00 | 63.55 | W | O |
| ATOM | 3148 | O | HOH | W | 182 | 45.148 | 46.355 | 9.428 | 1.00 | 58.76 | W | O |
| ATOM | 3149 | O | HOH | W | 183 | 78.021 | 49.570 | −0.246 | 1.00 | 32.19 | W | O |
| ATOM | 3150 | O | HOH | W | 184 | 81.804 | 50.829 | −2.607 | 1.00 | 38.10 | W | O |
| ATOM | 3151 | O | HOH | W | 185 | 88.410 | 73.240 | 7.564 | 1.00 | 56.30 | W | O |
| ATOM | 3152 | O | HOH | W | 186 | 61.080 | 66.476 | 15.948 | 1.00 | 68.96 | W | O |
| ATOM | 3153 | O | HOH | W | 187 | 45.110 | 31.905 | 1.445 | 1.00 | 67.43 | W | O |
| ATOM | 3154 | O | HOH | W | 188 | 49.200 | 55.926 | 12.964 | 1.00 | 72.28 | W | O |
| ATOM | 3155 | O | HOH | W | 189 | 71.187 | 76.958 | 15.269 | 1.00 | 39.87 | W | O |
| ATOM | 3156 | O | HOH | W | 190 | 73.886 | 47.482 | 4.081 | 1.00 | 53.55 | W | O |
| ATOM | 3157 | O | HOH | W | 191 | 69.355 | 68.996 | −15.162 | 1.00 | 61.52 | W | O |
| ATOM | 3158 | O | HOH | W | 192 | 82.777 | 65.787 | −8.682 | 1.00 | 62.77 | W | O |
| ATOM | 3159 | O | HOH | W | 193 | 39.736 | 46.583 | 7.480 | 1.00 | 62.23 | W | O |
| ATOM | 3160 | O | HOH | W | 194 | 52.055 | 40.044 | −22.266 | 1.00 | 55.63 | W | O |
| ATOM | 3161 | O | HOH | W | 195 | 71.314 | 50.785 | −16.556 | 1.00 | 49.70 | W | O |
| ATOM | 3162 | O | HOH | W | 196 | 61.950 | 38.755 | −19.713 | 1.00 | 70.81 | W | O |
| ATOM | 3163 | O | HOH | W | 197 | 84.051 | 69.275 | 5.460 | 1.00 | 48.64 | W | O |
| ATOM | 3164 | O | HOH | W | 198 | 76.032 | 60.681 | 20.880 | 1.00 | 69.12 | W | O |
| ATOM | 3165 | O | HOH | W | 199 | 73.266 | 44.918 | 4.326 | 1.00 | 68.75 | W | O |
| ATOM | 3166 | O | HOH | W | 200 | 82.129 | 50.468 | −5.451 | 1.00 | 59.02 | W | O |
| ATOM | 3167 | O | HOH | W | 201 | 83.221 | 72.917 | 3.600 | 1.00 | 40.04 | W | O |
| ATOM | 3168 | O | HOH | W | 202 | 59.652 | 75.257 | 4.275 | 1.00 | 57.30 | W | O |
| ATOM | 3169 | O | HOH | W | 203 | 78.123 | 47.635 | 22.706 | 1.00 | 45.73 | W | O |
| ATOM | 3170 | O | HOH | W | 204 | 77.637 | 76.375 | 11.568 | 1.00 | 43.51 | W | O |
| ATOM | 3171 | O | HOH | W | 205 | 58.555 | 48.938 | 13.305 | 1.00 | 48.92 | W | O |
| ATOM | 3172 | O | HOH | W | 206 | 57.638 | 66.927 | 18.153 | 1.00 | 50.79 | W | O |
| ATOM | 3173 | O | HOH | W | 207 | 58.312 | 43.498 | 7.697 | 1.00 | 33.77 | W | O |
| ATOM | 3174 | O | HOH | W | 208 | 44.538 | 28.297 | 3.536 | 1.00 | 55.65 | W | O |
| ATOM | 3175 | O | HOH | W | 209 | 59.595 | 53.833 | 19.308 | 1.00 | 58.04 | W | O |
| ATOM | 3176 | O | HOH | W | 210 | 57.084 | 51.317 | 14.707 | 1.00 | 51.78 | W | O |
| ATOM | 3177 | O | HOH | W | 211 | 49.436 | 21.830 | −1.938 | 1.00 | 62.41 | W | O |
| ATOM | 3178 | O | HOH | W | 212 | 60.734 | 77.657 | 4.018 | 1.00 | 73.34 | W | O |
| ATOM | 3179 | O | HOH | W | 213 | 79.123 | 83.308 | 3.898 | 1.00 | 63.20 | W | O |
| ATOM | 3180 | O | HOH | W | 214 | 57.523 | 61.921 | −13.519 | 1.00 | 37.25 | W | O |
| ATOM | 3181 | O | HOH | W | 215 | 71.168 | 43.072 | 5.167 | 1.00 | 41.82 | W | O |
| ATOM | 3182 | O | HOH | W | 216 | 76.653 | 84.242 | 3.301 | 1.00 | 78.23 | W | O |
| ATOM | 3183 | O | HOH | W | 217 | 42.382 | 40.135 | 17.622 | 1.00 | 61.51 | W | O |
| ATOM | 3184 | O | HOH | W | 218 | 78.733 | 69.517 | −5.343 | 1.00 | 61.81 | W | O |
| ATOM | 3185 | O | HOH | W | 219 | 62.986 | 22.749 | −4.555 | 1.00 | 42.82 | W | O |
| ATOM | 3186 | O | HOH | W | 220 | 60.743 | 44.247 | 9.220 | 1.00 | 48.58 | W | O |
| ATOM | 3187 | O | HOH | W | 221 | 57.413 | 29.275 | −13.554 | 1.00 | 41.77 | W | O |
| ATOM | 3188 | O | HOH | W | 222 | 71.784 | 39.808 | −3.358 | 1.00 | 49.72 | W | O |
| ATOM | 3189 | O | HOH | W | 223 | 74.571 | 63.700 | −13.618 | 1.00 | 53.93 | W | O |
| ATOM | 3190 | O | HOH | W | 224 | 71.261 | 51.431 | −13.741 | 1.00 | 41.48 | W | O |
| ATOM | 3191 | O | HOH | W | 225 | 78.559 | 79.217 | 0.998 | 1.00 | 50.39 | W | O |
| ATOM | 3192 | O | HOH | W | 226 | 68.431 | 42.241 | 17.534 | 1.00 | 51.33 | W | O |
| ATOM | 3193 | O | HOH | W | 227 | 74.858 | 56.378 | 23.475 | 1.00 | 62.51 | W | O |
| ATOM | 3194 | O | HOH | W | 228 | 79.307 | 60.745 | 22.219 | 1.00 | 40.98 | W | O |
| ATOM | 3195 | O | HOH | W | 229 | 60.314 | 68.573 | 10.249 | 1.00 | 29.74 | W | O |
| ATOM | 3196 | O | HOH | W | 230 | 61.602 | 71.621 | −9.518 | 1.00 | 51.81 | W | O |
| ATOM | 3197 | O | HOH | W | 231 | 49.899 | 42.585 | 7.057 | 1.00 | 35.46 | W | O |
| ATOM | 3198 | O | HOH | W | 232 | 46.590 | 57.769 | 2.535 | 1.00 | 69.32 | W | O |
| ATOM | 3199 | O | HOH | W | 233 | 45.044 | 34.173 | −1.541 | 1.00 | 50.34 | W | O |
| ATOM | 3200 | O | HOH | W | 234 | 71.447 | 46.668 | −18.182 | 1.00 | 58.66 | W | O |
| ATOM | 3201 | O | HOH | W | 235 | 73.000 | 43.214 | −18.003 | 1.00 | 45.06 | W | O |
| ATOM | 3202 | O | HOH | W | 236 | 43.370 | 55.663 | −1.011 | 1.00 | 61.60 | W | O |
| ATOM | 3203 | O | HOH | W | 237 | 74.007 | 57.458 | −17.330 | 1.00 | 59.05 | W | O |
| ATOM | 3204 | O | HOH | W | 238 | 78.277 | 52.906 | −16.612 | 1.00 | 65.63 | W | O |
| ATOM | 3205 | O | HOH | W | 239 | 77.796 | 59.191 | −8.755 | 1.00 | 45.94 | W | O |
| ATOM | 3206 | O | HOH | W | 240 | 84.436 | 60.164 | −3.135 | 1.00 | 53.03 | W | O |
| ATOM | 3207 | O | HOH | W | 241 | 65.112 | 49.259 | 9.447 | 1.00 | 53.21 | W | O |
| ATOM | 3208 | O | HOH | W | 242 | 63.207 | 51.425 | 10.118 | 1.00 | 42.58 | W | O |
| ATOM | 3209 | O | HOH | W | 243 | 89.242 | 51.621 | 10.559 | 1.00 | 37.79 | W | O |
| ATOM | 3210 | O | HOH | W | 244 | 88.861 | 58.033 | −1.500 | 1.00 | 63.56 | W | O |
| ATOM | 3211 | O | HOH | W | 245 | 80.840 | 77.800 | 12.517 | 1.00 | 43.88 | W | O |
| ATOM | 3212 | O | HOH | W | 246 | 77.216 | 83.653 | 0.754 | 1.00 | 66.92 | W | O |
| ATOM | 3213 | O | HOH | W | 247 | 69.579 | 67.222 | 23.238 | 1.00 | 67.75 | W | O |
| ATOM | 3214 | O | HOH | W | 248 | 75.887 | 51.320 | 21.816 | 1.00 | 72.66 | W | O |
| ATOM | 3215 | O | HOH | W | 249 | 68.191 | 78.916 | 4.291 | 1.00 | 52.82 | W | O |
| ATOM | 3216 | O | HOH | W | 250 | 82.004 | 63.181 | 21.579 | 1.00 | 30.60 | W | O |
| ATOM | 3217 | O | HOH | W | 251 | 76.390 | 67.886 | 21.910 | 1.00 | 51.17 | W | O |
| ATOM | 3218 | O | HOH | W | 252 | 53.503 | 60.921 | 17.416 | 1.00 | 72.58 | W | O |
| ATOM | 3219 | O | HOH | W | 253 | 60.509 | 46.370 | −23.693 | 1.00 | 62.40 | W | O |
| ATOM | 3220 | O | HOH | W | 254 | 53.842 | 41.622 | −18.205 | 1.00 | 43.31 | W | O |
| ATOM | 3221 | O | HOH | W | 255 | 48.037 | 45.876 | −0.170 | 1.00 | 42.34 | W | O |
| ATOM | 3222 | O | HOH | W | 256 | 44.592 | 45.050 | 2.573 | 1.00 | 46.37 | W | O |
| ATOM | 3223 | O | HOH | W | 257 | 40.130 | 44.608 | 4.624 | 1.00 | 61.11 | W | O |
| ATOM | 3224 | O | HOH | W | 258 | 69.355 | 47.143 | 5.898 | 1.00 | 60.82 | W | O |
| ATOM | 3225 | O | HOH | W | 259 | 34.957 | 32.570 | 1.397 | 1.00 | 47.77 | W | O |
| ATOM | 3226 | O | HOH | W | 260 | 61.555 | 31.492 | −14.640 | 1.00 | 63.05 | W | O |
| ATOM | 3227 | O | HOH | W | 261 | 43.862 | 53.451 | −5.566 | 1.00 | 71.67 | W | O |
| ATOM | 3228 | O | HOH | W | 262 | 84.234 | 48.309 | 0.364 | 1.00 | 54.03 | W | O |
| ATOM | 3229 | O | HOH | W | 263 | 87.932 | 51.816 | −3.215 | 1.00 | 57.80 | W | O |
| ATOM | 3230 | O | HOH | W | 264 | 82.425 | 63.456 | −6.283 | 1.00 | 62.42 | W | O |
| ATOM | 3231 | O | HOH | W | 265 | 80.271 | 28.172 | 9.463 | 1.00 | 40.70 | W | O |
| ATOM | 3232 | O | HOH | W | 266 | 73.963 | 30.020 | 4.302 | 1.00 | 26.30 | W | O |
| ATOM | 3233 | O | HOH | W | 267 | 83.112 | 71.680 | 1.066 | 1.00 | 51.04 | W | O |
| ATOM | 3234 | O | HOH | W | 268 | 63.047 | 54.124 | 10.355 | 1.00 | 50.34 | W | O |
| ATOM | 3235 | O | HOH | W | 269 | 83.682 | 62.329 | −4.165 | 1.00 | 42.75 | W | O |
| ATOM | 3236 | O | HOH | W | 270 | 61.547 | 73.522 | −7.931 | 1.00 | 47.36 | W | O |
| ATOM | 3237 | O | HOH | W | 271 | 60.577 | 53.517 | 13.966 | 1.00 | 53.55 | W | O |
| ATOM | 3238 | O | HOH | W | 272 | 54.580 | 71.014 | −6.905 | 1.00 | 46.69 | W | O |
| ATOM | 3239 | O | HOH | W | 273 | 77.926 | 39.031 | −0.508 | 1.00 | 49.59 | W | O |
| ATOM | 3240 | O | HOH | W | 274 | 69.669 | 49.137 | −17.891 | 1.00 | 45.33 | W | O |
| ATOM | 3241 | O | HOH | W | 275 | 44.777 | 49.840 | 1.964 | 1.00 | 44.62 | W | O |
| ATOM | 3242 | O | HOH | W | 276 | 48.453 | 54.600 | 5.308 | 1.00 | 39.43 | W | O |
| ATOM | 3243 | O | HOH | W | 277 | 51.764 | 32.262 | 13.155 | 1.00 | 71.42 | W | O |
| ATOM | 3244 | O | HOH | W | 278 | 60.951 | 29.296 | 11.161 | 1.00 | 53.99 | W | O |
| ATOM | 3245 | O | HOH | W | 279 | 68.206 | 23.452 | 8.777 | 1.00 | 50.70 | W | O |
| ATOM | 3246 | O | HOH | W | 280 | 87.567 | 24.412 | −10.981 | 1.00 | 49.42 | W | O |
| ATOM | 3247 | O | HOH | W | 281 | 81.650 | 24.690 | −15.233 | 1.00 | 46.79 | W | O |
| ATOM | 3248 | O | HOH | W | 282 | 83.121 | 29.023 | −15.678 | 1.00 | 59.22 | W | O |
| ATOM | 3249 | O | HOH | W | 283 | 81.854 | 31.654 | −13.384 | 1.00 | 44.68 | W | O |
| ATOM | 3250 | O | HOH | W | 284 | 43.424 | 43.922 | −5.261 | 1.00 | 38.18 | W | O |
| ATOM | 3251 | O | HOH | W | 285 | 80.484 | 32.987 | −6.395 | 1.00 | 39.87 | W | O |
| ATOM | 3252 | I | IOD | J | 1 | 80.243 | 57.842 | 15.501 | 0.75 | 23.63 | J | I |
| ATOM | 3253 | I | IOD | J | 2 | 81.546 | 50.334 | 15.785 | 0.50 | 35.87 | J | I |
| ATOM | 3254 | I | IOD | J | 3 | 51.528 | 57.888 | −13.233 | 0.50 | 56.82 | J | I |
| END | | | | | | | | | | | | |

1. A BACE protein, which comprises the sequence set out in residues 45 to 455 of SEQ ID NO:2 (43 to 453 SwissProt P56817), or a fragment thereof comprising residues corresponding to 58 to 398 of SEQ ID NO:2, modified by the following changes:
substitution or deletion of at least one residue which is a proteolytic cleavage site, recognised by clostripain; and optionally
the replacement of from 1 to 30 other amino acids by an equivalent or fewer number of amino acids.

2. A protein according to claim 1 wherein at least one of residues 44, 47, 57, 58 and 59 of SEQ ID NO:2 are substituted.

3. A protein according to claim 1 wherein residues 58 and/or 59 are lysine.

4. A protein according to claim 1 wherein the asparagine residues at positions 155, 174, 225 and 356 (SwissProt P56817 153, 172, 223 and 354) are replaced by glutamine residues.

5. A protein according to claim 1 wherein the fragment is truncated at the C-terminus such that at least residues 449 et seq. of SEQ ID NO:2 are absent.

6. A method of making a truncated BACE protein, which method comprises proteolytically cleaving the protein of claim 1.

7. The method of claim 6 wherein said cleavage is at and includes one or more of residues 44, 47, 57, 58 and 59.

8. A BACE protein obtained or obtainable by the method of claim 7.

9. A protein according to claim 8 wherein the N-terminal is residue 45 of SEQ ID NO:2.

10. A protein according to claim 1 which is selected from: (a) SEQ ID 6; (b) SEQ ID 8; (c) SEQ ID 10; (d) SEQ ID 12; (e) SEQ ID 14; (f) SEQ ID 16; (g) SEQ ID 18; or a truncated BACE protein obtainable by a method comprising proteolytically cleaving the protein of claim 1, wherein said cleavage is at and includes one or more of residues 44, 47, 57, 58, and 59, wherein the protein is selected from (h) SEQ ID 19; (i) SEQ ID 20; (j) SEQ ID 21.

11. A nucleic acid encoding the protein of claim 1.

12. A vector comprising the nucleic acid of claim 11.

13. A host cell comprising the vector of claim 12.

14. A process for producing the protein of claim 1 comprising the steps of: (a) culturing a host cell comprising a vector comprising a nucleic acid encoding the protein of claim 1 under conditions suitable for expression of the protein; and optionally (b) isolating the expressed recombinant BACE protein.

15. A process for producing refolded recombinant BACE protein comprising the steps of: (a) solubilising the recombinant BACE; (b) diluting the solubilised BACE into an aqueous buffer containing 10 to 50 mM sulfobetaine; and (c) maintaining the diluted solution at low temperature and at high pH for at least 2 weeks.

16. A process for producing a crystal of BACE comprising the step of refolding recombinant BACE protein according to the process of claim 14.

17. A process for producing a crystal of a BACE protein comprising the step of growing the crystal by vapour diffusion using a reservoir buffer that contains 18-26% PEG 5000 MME, 180-220 mM ammonium iodide and 180-220 mM tri-sodium citrate pH 6.4-6.6, and optionally 0-5% glycerol.

18. A process according to claim 15 wherein the BACE protein is human BACE.

19. A process according to claim 17 wherein the BACE protein is human BACE.

20. A process according to claim 15 wherein the BACE protein is as defined in claim 1.

21. A process according to claim 17 wherein the BACE protein is as defined in claim 1.

22. A crystal of a BACE protein having a hexagonal space group P6₁22.

23. The crystal of claim 22 having unit cell dimensions of a=b=103.2 Å, c=169.1 Å, α=β=60°, γ=120°, and a unit cell variability of 5% in all dimensions.

24. A crystal of a BACE protein comprising a structure defined by all or a portion of the co-ordinates of Table 1±a root mean square deviation from the Cα atoms of less than 0.5 Å.

25. A crystal of the protein of claim 1.

26. The crystal of claim 22 having a resolution better than 2.5 Å.

27. The crystal of claim 24 having a resolution better than 2.5 Å.

28. The crystal of claim 22 which is soaked with one or more compound(s) to form co-complex structures.

29. The crystal of claim 24 which is soaked with one or more compound(s) to form co-complex structures.

30. The crystal of claim 22 wherein the BACE is co-crystallized with one or more compound(s) to form co-crystallized structures.

31. The crystal of claim 24 wherein the BACE is co-crystallized with one or more compound(s) to form co-crystallized structures.

32. The crystal of claim 22 which is an apo crystal.

33. The crystal of claim 24 which is an apo crystal.

34. A computer-based method for the analysis of the interaction of a molecular structure with a PACE protein, which comprises:
(a) providing a structure comprising a three-dimensional representation of PACE or of a portion of BACE, which representation comprises all or a portion of the coordinates of Table 1±a root mean square deviation from the Cα atoms of less than 0.5 Å.
(b) providing a molecular structure to be fitted to said BACE structure; and
(c) fitting the molecular structure to the PACE structure of (a).

35. The method of claim 34 wherein the molecular structure to be fitted is in the form of a model of a pharmacophore.

36. The method of claim 34 wherein the three-dimensional representation is a model constructed from all or a portion of the coordinates of Table 1±a root mean square deviation from the Cα atoms of less than 0.5 Å.

37. The method of claim 36 wherein the model is: (a) a wire-frame model; (b) a chicken-wire model; (c) a ball-and-stick model; (d) a space-filling model; (e) a stick-model; (f) a ribbon model; (g) a snake model; (h) an arrow and cylinder model; (i) an electron density map; (j) a molecular surface model.

38. A computer-based method for the analysis of molecular structures which comprises:
(a) providing the coordinates of at least two atoms of a PACE structure as defined in Table 1±a root mean square deviation from the Cα atoms of less than 0.5 Å (“selected coordinates”);
(b) providing the structure of a molecular structure to be fitted to the selected coordinates; and
(c) fitting the structure to the selected coordinates of the BACE structure.

39. The method of claim 38 wherein the selected coordinates are of at least 5, 10, 50, 100 or 500 atoms.

40. The method of claim 34 wherein the coordinates of Table 1 represent a binding pocket.

41. The method of claim 38 wherein the coordinates of Table 1 represent a binding pocket.

42. The method of claim 40 wherein the coordinates of Table 1 comprise those relating to residues SER71, GLY72, LEU91, ASP93, GLY95, SER96, VAL130, PRO131, TYR132, THR133, GLN134, ILE171, ILE179, ILE187, ALA188, ARG189, PRO190, TRP258, TYR259, ASP284, LYS285, ASP289, GLY291, THR292, THR293, ASN294, ARG296 and ARG368 (based on the numbering of SwissProt P56817).

43. The method of claim 41 wherein the coordinates of Table 1 comprise those relating to residues SER71, GLY72, LEU91, ASP93, GLY95, SER96, VAL130, PRO131, TYR132, THR133, GLN134, ILE171, ILE179, ILE187, ALA188, ARG189, PRO190, TRP258, TYR259, ASP284, LYS285, ASP289, GLY291, THR292, THR293, ASN294, ARG296 and ARG368 (based on the numbering of SwissProt P56817).

44. A computer-based method of rational drug design comprising the method of claim 28.

45. A computer-based method of rational drug design comprising the method of claim 38.

46. A computer-based method of rational drug design comprising comprising:
(a) providing the coordinates of at least two atoms of a BACE structure as defined in Table 1±a root mean square deviation from the Cα atoms of less than 0.5 Å (“selected coordinates”);
(b) providing the structures of a plurality of molecular fragments;
(c) fitting the structure of each of the molecular fragments to the selected coordinates; and
(d) assembling the molecular fragments into a single molecule to form a candidate modulator molecule.

47. A method for identifying a candidate modulator of BACE comprising the steps of:
(a) employing a three-dimensional structure of BACE, at least one sub-domain thereof, or a plurality of atoms thereof, to characterise at least one BACE binding cavity, the three-dimensional structure being defined by atomic coordinate data according to Table 1±a root mean square deviation from the Cα atoms of less than 0.5 Å; and
(b) identifying the candidate modulator by designing or selecting a compound for interaction with the binding cavity.

48. The method of claim 34 further comprising the step, of:
(a) obtaining or synthesising the molecular structure or modulator; and
(b) contacting the molecular structure or modulator with BACE to determine the ability of the molecular structure or modulator to interact with BACE.

49. The method of claim,38 further comprising the step of:
(a) obtaining or synthesising the molecular structure or modulator; and
(b) contacting the molecular structure or modulator with BACE to determine the ability of the molecular structure or modulator to interact with BACE.

50. The method of claim 46 further comprising the step of:
(a) obtaining or synthesising the molecular structure or modulator; and
(b) contacting the molecular structure or modulator with BACE to determine the ability of the molecular structure or modulator to interact with BACE.

51. The method of claim 47 further comprising the step of:.
(a) obtaining or synthesising the molecular structure or modulator; and
(b) contacting the molecular structure or modulator with BACE to determine the ability of the molecular structure or modulator to interact with BACE.

52. A method of assessing the ability of a candidate modulator to interact with BACE which comprises the steps of:
(a) obtaining or synthesising said candidate modulator;
(b) forming a crystallized complex of a BACE protein of claim 1 and said candidate modulator; and
(c) analysing said complex by X-ray crystallography or NMR spectroscopy to determine the ability of said candidate modulator to interact with BACE.

53. A method for determining the structure of a compound bound to BACE, said method comprising:
(a) mixing BACE with the compound to form a BACE-compound complex;
(b) crystallizing the BACE-compound complex; and
(c) determining the structure of said BACE-compound(s) complex by reference to the data of Table 1±a root mean square deviation from the Cα atoms of less than 0.5 Å.

54. A method for determining the structure of a compound bound to BACE, said method comprising:
(a) providing a crystal of BACE;
(b) soaking the crystal with one or more compound(s) to form a complex; and
(c) determining the structure of the complex by employing the data of Table 1±a root mean square deviation from the Cα atoms of less than 0.5 Å.

55. A method of determining the three dimensional structure of a BACE homologue or analogue of unknown structure, the method comprising the steps of:
(a) aligning a representation of an amino acid sequence of the BACE homologue or analogue with the amino acid sequence of the BACE of Table 1±a root mean square deviation from the Cα atoms of less than 0.5 Å to match homologous regions of the amino acid sequences;
(b) modelling the structure of the matched homologous regions of said target BACE of unknown structure on the corresponding regions of the BACE structure as defined by Table 1±a root mean square deviation from the Cα atoms of less than 0.5 Å; and
(c) determining a conformation for the BACE homologue or analogue which substantially preserves the structure of said matched homologous regions.

56. A method of providing data for generating structures and/or performing rational drug design for BACE, BACE homologues or analogues, complexes of BACE with a potential modulator, or complexes of BACE homologues or analogues with potential modulators, the method comprising (i) establishing communication with a remote device containing computer-readable data comprising at least one of:
(a) atomic coordinate data according to Table 1±a root mean square deviation from the Cα atoms of less than 0.5 Å, said data defining the three-dimensional structure of BACE, at least one sub-domain of the three-dimensional structure of BACE, or the coordinates of a plurality of atoms of BACE;
(b) structure factor data for BACE, said structure factor data being derivable from the atomic coordinate data of Table 1±a root mean square deviation from the Cα atoms of less than 0.5 Å.
(c) atomic coordinate data of a target BACE homologue or analogue generated by homology modelling of the target based on the data of Table 1±a root mean square deviation from the Cα atoms of less than 0.5 Å.
(d) atomic coordinate data of a protein generated by interpreting X-ray crystallographic data or NMR data by reference to the data of Table 1±a root mean square deviation from the Cα atoms of less than 0.5Å; and
(e) structure factor data derivable from the atomic coordinate data of (c) or (d); and
(ii) receiving said computer-readable data from said remote device.

57. A computer system containing one or more of: (a) atomic coordinate data according to Table 1±a root mean square deviation from the Cα atoms of less than 0.5 Å, said data defining the three-dimensional structure of BACE or at least selected coordinates thereof;
(b) structure factor data (where a structure factor comprises the amplitude and phase of the diffracted wave) for BACE, said structure factor data being derivable from the atomic coordinate data of Table 1±a root mean square deviation from the Cα atoms of less than 0.5 Å;
(c) atomic coordinate data of a target BACE protein generated by homology modelling of the target based on the data of Table 1±a root mean square deviation from the Cα atoms of less than 0.5 Å;
(d) atomic coordinate data of a target BACE protein generated by interpreting X-ray crystallographic data or NMR data by reference to the data of Table 1±a root mean square deviation from the Cα atoms of less than 0.5 Å; or
(e) structure factor data derivable from the atomic coordinate data of (c) or (d).

58. The computer system of claim 57 comprising: a computer-readable data storage medium comprising data storage material encoded with the computer-readable data;
(a) a working memory for storing instructions for processing said computer-readable data; and
(b) a central-processing unit coupled to said working memory and to said computer-readable data storage medium for processing said computer-readable data and thereby generating structures and/or performing rational drug design.

59. A method for determining the structure of a protein, which method comprises: providing the co-ordinates of Table 1±a root mean square deviation from the Cα atoms of less than 0.5 Å, and either
(a) positioning the co-ordinates in the crystal unit cell of said protein so as to provide a structure for said protein or
(b) assigning NMR spectra Peaks of said protein by manipulating the coordinates of Table 1±a root mean square deviation from the Cα atoms of less than 0.5 Å.

60. A method of preparing a composition comprising identifying a molecular structure or modulator according to the method of claim 34, and admixing the molecule with a carrier.

61. A method of preparing a composition comprising identifying a molecular structure or modulator according to the method of claim 38, and admixing the molecule with a carrier.

62. A method of preparing a composition comprising identifying a molecular structure or modulator according to the method of claim 46, and admixing the molecule with a carrier.

63. A method of preparing a composition comprising identifying a molecular structure or modulator according to the method of claim 47, and admixing the molecule with a carrier.

64. A method of preparing a composition comprising identifying a molecular structure or modulator according to the method of claim 52, and admixing the molecule with a carrier.

65. A process for producing a medicament, pharmaceutical composition or drug, the process comprising: (a) identifying a molecular structure or modulator according to the method as defined claim 34; and (b) preparing a medicament, pharmaceutical composition or drug containing the optimised modulator molecule.

66. A process for producing a medicament, pharmaceutical composition or drug, the process comprising: (a) identifying a molecular structure or modulator according to the method as defined claim 38; and (b) preparing a medicament, pharmaceutical composition or drug containing the optimised modulator molecule.

67. A process for producing a medicament, pharmaceutical composition or drug, the process comprising: (a),identifying a molecular structure or modulator according to the method as defined claim 46; and (b) preparing a medicament, pharmaceutical composition or drug containing the optimised modulator molecule.

68. A process for producing a medicament, pharmaceutical composition or drug, the process comprising: (a) identifying a molecular structure or modulator according to the method as defined claim 47; and (b) preparing a medicament, pharmaceutical composition or drug containing the optimised modulator molecule.

69. A process for producing a medicament, pharmaceutical composition or drug, the process comprising: (a) identifying a molecular structure or modulator according to the method as defined claim 52; and (b) preparing a medicament, pharmaceutical composition or drug containing the optimised modulator molecule.

70. A process according to claim 65 which further comprises optimising the structure of the modulator molecule; and preparing a medicament, pharmaceutical composition or drug containing the optimised modulator molecule.

71. A process according to claim 66 which further comprises optimising the structure of the modulator molecule; and preparing a medicament, pharmaceutical composition or drug containing the optimised modulator molecule.

72. A process according to claim 67 which further comprises optimising the structure of the modulator molecule; and preparing a medicament, pharmaceutical composition or drug containing the optimised modulator molecule.

73. A process according to claim 68 which further comprises optimising the structure of the modulator molecule; and preparing a medicament, pharmaceutical composition or drug containing the optimised modulator molecule.

74. A process according to claim 69 which further comprises optimising the structure of the modulator molecule; and preparing a medicament, pharmaceutical composition or drug containing the optimised modulator molecule.

75. A compound identified, produced or obtainable by the process or method of claim 34.

76. A compound identified, produced or obtainable by the process or method of claim 38.

77. A compound identified, produced or obtainable by the process or method of claim 46.

78. A compound identified, produced or obtainable by the process or method of claim 47.

79. A compound identified, produced or obtainable by the process or method of claim 52.

80. A compound of claim 75 or composition thereof for use in medicine.

81. A compound of claim 76 or composition thereof for use in medicine.

82. A compound of claim 77 or composition thereof for use in medicine.

83. A compound of claim 78 or composition thereof for use in medicine.

84. A compound of claim 79 or composition thereof for use in medicine.


SEQUENCE LISTING (46 sequences)

SEQ ID NO: 1 - DNA, 1368 bp, Homo sapiens
atggctagcatgactggtggacagcaaatgggtcgcggatccatggcgggagtgctgcctgcccacggcacccagcacggcatccggctgcccctgcgcagcggcctggggggcgcccccctggggctgcggctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcataa

SEQ ID NO: 2 - protein, 455 aa, Homo sapiens
MASMTGGQQMGRGSMAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDES

SEQ ID NO: 3 - DNA, 1386 bp, Homo sapiens
atggctagcatgactggtggacagcaaatgggtcgcggatccatggcgggagtgctgcctgcccacggcacccagcacggcatccggctgcccctgcgcagcggcctggggggcgcccccctggggctgcggctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggcccccaggtcactgtgcgtgccaacattgctgccatcactgaatcagacaagttcttcatccagggctccaactgggaaggcatcctggggctggcctatgctgagattgccaggcctgacgactccctggagcctttctttgactctctggtaaagcagacccacgttcccaacctcttctccctgcagctttgtggtgctggcttccccctccagcagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttacccagcagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcacatcaccatcatcaccactaa

SEQ ID NO: 4 - protein, 461 aa, Homo sapiens
MASMTGGQQMGRGSMAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPQVTVRANIAAITESDKFFIQGSNWEGILGLAYAEIARPDDSLEPFFDSLVKQTHVPNLFSLQLCGAGFPLQQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTQQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESHHHHHH

SEQ ID NO: 5 - DNA, 1368 bp, Homo sapiens
atggctagcatgactggtggacagcaaatgggtcgcggatccatggcgggagtgctgcctgcccacggcacccagcacggcatccggctgcccctgcgcagcggcctggggggcgcccccctggggctgcggctgccccgggagaccgacgaagagcccgaggagcccggcaagaagggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcataa

SEQ ID NO: 6 - protein, 455 aa, Homo sapiens
MASMTGGQQMGRGSMAGVLPAHGTQHGIRLPLRSGLGGAPLGLRLPRETDEEPEEPGKKGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDES

SEQ ID NO: 7 - DNA, 1368 bp, Homo sapiens
atggctagcatgactggtggacagcaaatgggtcgcggatccatggcgggagtgctgcctgcccacggcacccagcacggcatccggctgcccctgcgcagcggcctggggggcgcccccctggggctgcggctgccccgggagaccgacgaagagcccgaggagcccggccggaagggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcataa

SEQ ID NO: 8 - protein, 455 aa, Homo sapiens
MASMTGGQQMGRGSMAGVLPAHGTQHGIRLPLRSGLGGAPLGLRLPRETDEEPEEPGRKGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDES

SEQ ID NO: 9 - DNA, 1365 bp, Homo sapiens
atggctagcatgactggtggacagcaaatgggtcgcggatccatggcgggagtgctgcctgcccacggcacccagcacggcatccggctgcccctgcgcagcggcctggggggcgcccccctggggctgcggctgccccgggagaccgacgaagagcccgaggagcccggcaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcataa

SEQ ID NO: 10 - protein, 454 aa, Homo sapiens
MASMTGGQQMGRGSMAGVLPAHGTQHGIRLPLRSGLGGAPLGLRLPRETDEEPEEPGRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDES

SEQ ID NO: 11 - DNA, 1386 bp, Homo sapiens
atggctagcatgactggtggacagcaaatgggtcgcggatccatggcgggagtgctgcctgcccacggcacccagcacggcatccggctgcccctgcgcagcggcctggggggcgcccccctggggctgcggctgccccgggagaccgacgaagagcccgaggagcccggcaagaagggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggcccccaggtcactgtgcgtgccaacattgctgccatcactgaatcagacaagttcttcatccagggctccaactgggaaggcatcctggggctggcctatgctgagattgccaggcctgacgactccctggagcctttctttgactctctggtaaagcagacccacgttcccaacctcttctccctgcagctttgtggtgctggcttccccctccagcagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttacccagcagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcacatcaccatcatcaccactaa

SEQ ID NO: 12 - protein, 461 aa, Homo sapiens
MASMTGGQQMGRGSMAGVLPAHGTQHGIRLPLRSGLGGAPLGLRLPRETDEEPEEPGKKGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPQVTVRANIAAITESDKFFIQGSNWEGILGLAYAEIARPDDSLEPFFDSLVKQTHVPNLFSLQLCGAGFPLQQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTQQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESHHHHHH

SEQ ID NO: 13 - DNA, 1368 bp, Homo sapiens
atggctagcatgactggtggacagcaaatgggtcgcggatccatggcgggagtgctgcctgcccacggcacccagcacggcatccggctgcccctgcgcagcggcctggggggcgcccccctggggctgcggctgccccgggagaccgacgaagagcccgaggagcccggcaagaagggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggcccccaggtcactgtgcgtgccaacattgctgccatcactgaatcagacaagttcttcatccagggctccaactgggaaggcatcctggggctggcctatgctgagattgccaggcctgacgactccctggagcctttctttgactctctggtaaagcagacccacgttcccaacctcttctccctgcagctttgtggtgctggcttccccctccagcagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttacccagcagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcatag

SEQ ID NO: 14 - protein, 455 aa, Homo sapiens
MASMTGGQQMGRGSMAGVLPAHGTQHGIRLPLRSGLGGAPLGLRLPRETDEEPEEPGKKGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPQVTVRANIAAITESDKFFIQGSNWEGILGLAYAEIARPDDSLEPFFDSLVKQTHVPNLFSLQLCGAGFPLQQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTQQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDES

SEQ ID NO: 15 - DNA, 1386 bp, Homo sapiens
atggctagcatgactggtggacagcaaatgggtcgcggatccatggcgggagtgctgcctgcccacggcacccagcacggcatccggctgcccctgcgcagcggcctggggggcgcccccctggggctgcggctgccccgggagaccgacgaagagcccgaggagcccggccggaagggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggcccccaggtcactgtgcgtgccaacattgctgccatcactgaatcagacaagttcttcatccagggctccaactgggaaggcatcctggggctggcctatgctgagattgccaggcctgacgactccctggagcctttctttgactctctggtaaagcagacccacgttcccaacctcttctccctgcagctttgtggtgctggcttccccctccagcagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttacccagcagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcacatcaccatcatcaccactaa

SEQ ID NO: 16 - protein, 461 aa, Homo sapiens
MASMTGGQQMGRGSMAGVLPAHGTQHGIRLPLRSGLGGAPLGLRLPRETDEEPEEPGRKGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPQVTVRANIAAITESDKFFIQGSNWEGILGLAYAEIARPDDSLEPFFDSLVKQTHVPNLFSLQLCGAGFPLQQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTQQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESHHHHHH

SEQ ID NO: 17 - DNA, 1383 bp, Homo sapiens
atggctagcatgactggtggacagcaaatgggtcgcggatccatggcgggagtgctgcctgcccacggcacccagcacggcatccggctgcccctgcgcagcggcctggggggcgcccccctggggctgcggctgccccgggagaccgacgaagagcccgaggagcccggcaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggcccccaggtcactgtgcgtgccaacattgctgccatcactgaatcagacaagttcttcatccagggctccaactgggaaggcatcctggggctggcctatgctgagattgccaggcctgacgactccctggagcctttctttgactctctggtaaagcagacccacgttcccaacctcttctccctgcagctttgtggtgctggcttccccctccagcagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttacccagcagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcacatcaccatcatcaccactaa

SEQ ID NO: 18 - protein, 460 aa, Homo sapiens
MASMTGGQQMGRGSMAGVLPAHGTQHGIRLPLRSGLGGAPLGLRLPRETDEEPEEPGRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPQVTVRANIAAITESDKFFIQGSNWEGILGLAYAEIARPDDSLEPFFDSLVKQTHVPNLFSLQLCGAGFPLQQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTQQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESHHHHHH

SEQ ID NO: 19 - protein, 411 aa, Homo sapiens
LPRETDEEPEEPGKKGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDES

SEQ ID NO: 20 - protein, 411 aa, Homo sapiens
LPRETDEEPEEPGKKGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPQVTVRANIAAITESDKFFIQGSNWEGILGLAYAEIARPDDSLEPFFDSLVKQTHVPNLFSLQLCGAGFPLQQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTQQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDES

SEQ ID NO: 21 - protein, 417 aa, Homo sapiens
LPRETDEEPEEPGKKGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPQVTVRANIAAITESDKFFIQGSNWEGILGLAYAEIARPDDSLEPFFDSLVKQTHVPNLFSLQLCGAGFPLQQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTQQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESHHHHHH

SEQ ID NO: 22 - DNA, 24 bp, Artificial Sequence
  Description of Artificial Sequence Primer
agctccctctcctgagaagccacc

SEQ ID NO: 23 - DNA, 23 bp, Artificial Sequence
  Description of Artificial Sequence Primer
ccacaggtgccatctgtgtctcc

SEQ ID NO: 24 - DNA, 22 bp, Artificial Sequence
  Description of Artificial Sequence Primer
caccagcaccacccagacttgg

SEQ ID NO: 25 - DNA, 22 bp, Artificial Sequence
  Description of Artificial Sequence Primer
aaccacggaggtgtggtccagg

SEQ ID NO: 26 - DNA, 39 bp, Homo sapiens
cccgaggagcccggcaagaagggcagctttgtggagatg

SEQ ID NO: 27 - DNA, 39 bp, Homo sapiens
catctccacaaagctgcccttcttgccgggctcctcggg

SEQ ID NO: 28 - DNA, 40 bp, Homo sapiens
cccgaggagcccggccggaagggcagctttgtggagatgg

SEQ ID NO: 29 - DNA, 40 bp, Homo sapiens
ccatctccacaaagctgcccttccggccgggctcctcggg

SEQ ID NO: 30 - DNA, 42 bp, Homo sapiens
cccgaggagcccggcaggggcagctttgtggagatggtggac

SEQ ID NO: 31 - DNA, 42 bp, Homo sapiens
gtccaccatctccacaaagctgcccctgccgggctcctcggg

SEQ ID NO: 32 - DNA, 39 bp, Homo sapiens; identical to SEQ ID NO: 26

SEQ ID NO: 33 - DNA, 39 bp, Homo sapiens; identical to SEQ ID NO: 27

SEQ ID NO: 34 - DNA, 40 bp, Homo sapiens; identical to SEQ ID NO: 28

SEQ ID NO: 35 - DNA, 40 bp, Homo sapiens; identical to SEQ ID NO: 29

SEQ ID NO: 36 - DNA, 42 bp, Homo sapiens; identical to SEQ ID NO: 30

SEQ ID NO: 37 - DNA, 42 bp, Homo sapiens; identical to SEQ ID NO: 31

SEQ ID NO: 38 - DNA, 39 bp, Homo sapiens; identical to SEQ ID NO: 26

SEQ ID NO: 39 - DNA, 39 bp, Homo sapiens; identical to SEQ ID NO: 27

SEQ ID NO: 40 - DNA, 40 bp, Homo sapiens
ccacagacagatgagtcatgacaccatcatcaccactaag

SEQ ID NO: 41 - DNA, 40 bp, Homo sapiens
cttagtggtgatgatggtgtcatgactcatctgtctgtgg

SEQ ID NO: 42 - protein, 6 aa, Artificial Sequence
  Description of Artificial Sequence 6-His tag
HHHHHH

SEQ ID NO: 43 - DNA, 29 bp, Artificial Sequence
  Description of Artificial Sequence Primer
cgggatccatggcgggagtgctgcctgcc

SEQ ID NO: 44 - DNA, 40 bp, Artificial Sequence
  Description of Artificial Sequence Primer
cgggatccttatgactcatctgtctgtggaatgttgtagc

SEQ ID NO: 45 - DNA, 18 bp, Artificial Sequence
  Description of Artificial Sequence His tag oligonucleotide sequence
catcaccatcatcaccac

SEQ ID NO: 46 - protein, 12 aa, Artificial Sequence
  Description of Artificial Sequence Illustrative synthetic flourescent peptide
REEVNLDAEFKR